(19) United States
(12) Patent Application Publication
(10) Pub. No.: US 2022/0354963 A1
(43) Pub. Date: Nov. 10, 2022

(54) EXTRACELLULAR VESICLE LINKED TO MOLECULES AND USES THEREOF

(71) Applicant: Codiak BioSciences, Inc., Cambridge, MA (US)

(72) Inventors: Yi Zhang, Belmont, MA (US); Aaron R. Noyes, Melrose, MA (US); Adam T. Boutin, Arlington, MA (US)

(73) Assignee: Codiak BioSciences, Inc., Cambridge, MA (US)

(21) Appl. No.: 17/634,897

(22) PCT Filed: Aug. 14, 2020
(86) PCT No.: PCT/US2020/046560

§ 371 Date: Feb. 11, 2022

Related U.S. Application Data
(60) Provisional application No. 62/895,398, filed on Sep. 3, 2019.
(60) Provisional application No. 62/886,941, filed on Aug. 14, 2019.

(51) Int. Cl. A61K 47/69; A61K 47/28; A61K 47/22; A61K 47/54; A61K 47/55; A61P 35/00

(52) U.S. Cl. CPC A61K 47/6901; A61K 47/28; A61K 47/22; A61K 47/543; A61K 47/551; A61P 35/00

(57) ABSTRACT

The present disclosure relates to extracellular vesicles (e.g., exosomes) comprising a biologically active molecule covalently linked to the extracellular vesicle via an anchoring moiety, which may be useful as an agent for the prophylaxis or treatment of cancer or other diseases. Also provided herein are methods for producing the extracellular vesicles and methods for using the extracellular vesicles to treat diseases or disorders.

### CROSS REFERENCE TO RELATED APPLICATIONS

This PCT application claims the priority benefit of U.S. Provisional Application Nos. 62/886,941 filed Aug. 14, 2019; and 62/895,398 filed Sep. 3, 2019; each of which is incorporated herein by reference in its entirety.

### REFERENCE TO SEQUENCE LISTING SUBMITTED ELECTRONICALLY VIA EFS-WEB

The content of the electronically submitted sequence listing (Name: 4000.057PC02_Seglisting_ST25.txt, Size: 765,394 bytes; and Date of Creation: Aug. 14, 2020) submitted in this application is incorporated herein by reference in its entirety.

### TECHNICAL FIELD

The present disclosure provides extracellular vesicles (EVs), e.g., exosomes, comprising at least one biologically active molecule covalently linked to the extracellular vesicle, e.g., exosome, via an anchoring moiety, which can be useful as an agent for the prophylaxis or treatment of cancer and other diseases.

### BACKGROUND

Many bioactive compounds have potent biological activity that is of therapeutic interest. However, these compounds often exhibit toxicity in non-target organs. One way to limit exposure of non-target tissues is to chemically conjugate small molecules to affinity-based reagents such as antibodies, which can direct the therapeutic compound to specific cell types (Dosio, F. et al., Toxins (Basel) 3(7):848-883 (2011)), but this approach is limited by the number of molecules of the compound of interest that can be attached to an antibody (typically 2-6 molecules per antibody), and by the availability/existence of antibodies that specifically bind to targeted, relevant diseased/effector cells without binding to non-target cells. These two issues limit the use of antibody-drug conjugates (ADC) by decreasing potency and increasing systemic toxicity, respectively. Accordingly, there is a need for delivery systems with a higher payload than ADCs that can selectively target specific tissues or organs while at the same time limiting overall systemic exposure to the therapeutic compound.

EVs, e.g., exosomes, are important mediators of intercellular communication. They are also important biomarkers in the diagnosis and prognosis of many diseases, such as cancer. As drug delivery vehicles, EVs, e.g., exosomes, offer many advantages over traditional drug delivery methods (e.g., peptide immunization, DNA vaccines) as a new treatment modality in many therapeutic areas. However, despite its advantages, many EVs, e.g., exosomes, have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Accordingly, new and more effective engineered-EVs, e.g., exosomes, are necessary to better enable therapeutic use and other applications of EV-based technologies.

### BRIEF SUMMARY

The present disclosure is directed to an extracellular vesicle (EV) comprising a biologically active molecule (BAM) covalently linked to the EV via an anchoring moiety (AM), wherein the anchoring moiety comprises:

[AM]-[Linker]n-[BAM] Formula (I)

wherein n is any integer. In some aspects, n is any number between 0 and 10.

In other aspects, the anchoring moiety comprises a sterol, GM1, a lipid (e.g., a phospholipid or a fatty acid), a vitamin, a small molecule, a peptide, or a combination thereof. For example, the anchoring moiety comprises at least 6 carbon atoms, at least 7 carbon atoms, at least 8 carbon atoms, at least 9 carbon atoms, at least 10 carbon atoms, at least 11 carbon atoms, at least 12 carbon atoms, at least 13 carbon atoms, at least 14 carbon atoms, at least 15 carbon atoms, at least 16 carbon atoms, at least 17 carbon atoms, at least 18 carbon atoms, at least 19 carbon atoms, at least 20 carbon atoms, at least 25 carbon atoms, at least 30 carbon atoms, at least 35 carbon atoms, at least 40 carbon atoms, at least 45 carbon atoms, at least 50 carbon atoms, at least 55 carbon atoms, at least 60 carbon atoms, at least 65 carbon atoms, at least 70 carbon atoms, at least 75 carbon atoms, at least 80 carbon atoms, at least 85 carbon atoms, or at least 90 carbon atoms.

In other aspects, the anchoring moiety comprises a sterol, a steroid, a hopanoid, a hydroxysteroid, a secosteroid, an analog thereof, or any combination thereof. In some other aspects, the anchoring moiety comprises ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, stigmasterol, or any combination thereof.

In some aspects, the anchoring moiety is a cholesterol having the structure:

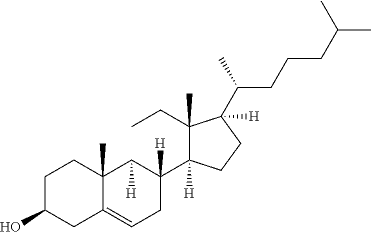

In some aspects, the anchoring moiety has the structure

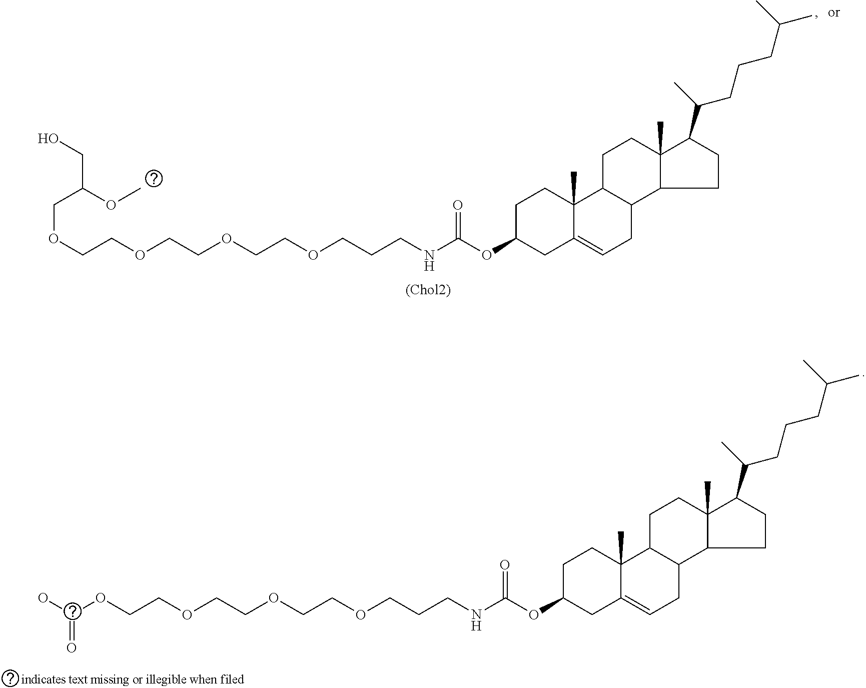 [SMILES: COC(CO)COCCOCCOCCOCCCNC(=O)O[C@H]1CC[C@@]2(C)C(=CCC3C2CC[C@@]2(C)C3CC[C@@H]2C(C)CCCC(C)C)C1 ; CC(C)CCCC(C)[C@H]1CCC2C3CC=C4C[C@@H](OC(=O)NCCCOCCOCCOCCOC(=O)O)CC[C@]4(C)C3CC[C@@]21C]

In some aspects, the anchoring moiety comprises a steroid, which is dihydrotestosterone, uvaol, hecigenin, diosgenin, progesterone, cortisol, or any combination thereof.

In some aspects, the anchoring moiety comprises a lipid. In other aspects, the anchoring moiety comprises a C₂-C₆₀ chain. In some aspects, the anchoring moiety comprises C₄-C₄₀, C₂-C₃₈, C₂-C₃₆, C₂-C₃₄, C₂-C₃₂, C₂-C₃₀, C₄-C₃₀, C₂-C₂₈, C₄-C₂₈, C₂-C₂₆, C₄-C₂₆, C₂-C₂₄, C₄-C₂₄, C₆-C₂₄, C₈-C₂₄, C₈-C₂₄, C₁₀-C₂₄, C₂-C₂₂, C₄-C₂₂, C₆-C₂₂, C₈-C₂₂, C₁₀-C₂₂, C₂-C₂₀, C₄-C₂₀, C₆-C₂₀, C₈-C₂₀, C₁₀-C₂₀, C₂-C₁₈, C₄-C₁₈, C₆-C₁₈, C₈-C₁₈, C₁₀-C₁₈, C₁₂-C₁₈, C₁₄-C₁₈, C₁₆-C₁₈, C₂- C₁₆, C₄-C₁₆, C₆-C₁₆, C₈-C₁₆, C₁₀-C₁₆, C₁₂-C₁₆, C₁₄-C₁₆, C₂-C₁₅, C₄-C₁₅, C₆-C₁₅, C₈-C₁₅, C₉-C₁₅, C₁₀-C₁₅, C₁₁-C₁₅, C₁₂-C₁₅, C₁₃-C₁₅, C₂-C₁₄, C₄-C₁₄, C₆-C₁₄, C₈-C₁₄, C₉-C₁₄, C₁₀-C₁₄, C₁₁-C₁₄, C₁₂- C₁₄, C₂-C₁₃, C₄-C₁₃, C₆-C₁₃, C₇-C₁₃, C₈-C₁₃, C₉-C₁₃, C₁₀-C₁₃, C₁₀-C₁₃, C₁₁-C₁₃, C₂-C₁₂, C₄-C₁₂, C₆-C₁₂, C₇-C₁₂, C₈-C₁₂, C₉-C₁₂, C₁₀-C₁₂, C₂-C₁₁, C₄-C₁₁, C₆-C₁₁, C₇-C₁₁, C₇-C₁₁, C₈-C₁₁, C₉-C₁₁, C₂-C₁₀, C₄-C₁₀, C₂-C₉, C₄-C₉, C₂-C₈, C₂-C₇, C₄-C₂, C₂-C₆, or C₄-C₆, chain.

In some aspects, the anchoring moiety comprises a straight chain fatty acid, a branched fatty acid, an unsaturated fatty acid, a monounsaturated fatty acid, a polyunsaturated fatty acid, a hydroxyl fatty acid, a polycarboxylic acid, or any combination thereof. In some aspects, the anchoring moiety comprises a straight chain fatty acid, which is butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid, heptacosanoic acid, or any combination thereof.

In some aspects, the anchoring moiety comprises a branched fatty acid, which is isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, α-heptylundecanoic acid, 2-decyltetradecanoic acid, 2-undecyltetradecanoic acid, 2-decylpentadecanoic acid, 2-undecylpentadecanoic acid, Fine oxocol 1800 acid (product of Nissan Chemical Industries, Ltd.), anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid, and 26-methyloctacosanoic acid, or any combination thereof.

In some aspects, the anchoring moiety comprises an unsaturated fatty acid, which is 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid, 7,10,13,16,19-docosapentaenoic acid, 4,7,10,13,16,19-docosahexaenoic acid, or any combination thereof. In some aspects, the anchoring moiety comprises a hydroxy fatty acid, which is α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid, or any combination thereof. In some aspects, the anchoring moiety comprises a polycarboxylic acid, which is oxalic acid, malonic acid, succinic acid, glutaric acid, adipic acid, pimelic acid, suberic acid, azelaic acid, sebacic acid, D,L-malic acid, or any combination thereof.

In some aspects, the anchoring moiety comprises a phospholipid. In some aspects, the phospholipid is phosphatidyl choline, phosphatidyl ethanolamine, phosphatidyl glycerol, phosphatidyl serine, phosphatidic acid, 2 lysophosphatidyl choline, sphingomyelin, or any combination thereof. In some aspects, the phospholipid is phosphatidylethanolamines, which is dilauroylphosphatidyl ethanolamine, dimyristoylphosphatidyl ethanolamine, dipalmitoylphosphatidyl ethanolamine, distearoylphosphatidyl ethanolamine, dioleoylphosphatidyl ethanolamine, 1-palmitoyl-2-oleylphosphatidyl ethanolamine, 1-oleyl-2-palmitoylphosphatidyl ethanolamine, dierucoylphosphatidyl ethanolamine, or any combination thereof. In some aspects, the phospholipid is phosphatidyl glycerol, which is dilauroylphosphatidyl glycerol, dimyristoylphosphatidyl glycerol, dipalmitoylphosphatidyl glycerol, distearoylphosphatidyl glycerol, dioleoylphosphatidyl glycerol, 1-palmitoyl-2-oleyl-phosphatidyl glycerol, 1-oleyl-2-palmitoyl-phosphatidyl glycerol, dierucoylphosphatidyl glycerol, or any combination thereof. In some aspects, the phospholipid is phosphatidyl serine, which is dilauroylphosphatidyl serine, dimyristoylphosphatidyl serine, dipalmitoylphosphatidyl serine, distearoylphosphatidyl serine, dioleoylphosphatidyl serine, 1-palmitoyl-2-oleyl-phosphatidyl serine, 1-oleyl-2-palmitoyl-phosphatidyl serine, dierucoylphosphatidyl serine, or any combination thereof. In some aspects, the phospholipid is phosphatidic acid, which is dilauroylphosphatidic acid, dimyristoylphosphatidic acid, dipalmitoylphosphatidic acid, distearoylphosphatidic acid, dioleoylphosphatidic acid, 1-palmitoyl-2-oleylphosphatidic acid, 1-oleyl-2-palmitoyl-phosphatidic acid, dierucoylphosphatidic acid, or any combination thereof. In some aspects, the phosphatidyl inositol, which is dilauroylphosphatidyl inositol, dimyristoylphosphatidyl inositol, dipalmitoylphosphatidyl inositol, distearoylphosphatidyl inositol, dioleoylphosphatidyl inositol, 1-palmitoyl-2-oleyl-phosphatidyl inositol, 1-oleyl-2-palmitoyl-phosphatidyl inositol, dierucoylphosphatidyl inositol, or any combination thereof. In some aspects, the phospholipid is a symmetric phospholipid, which is 1,2 dipropionyl sn-glycero 3 phosphocholine (03:0 PC); 1,2 dibutyryl sn glycero 3 phosphocholine (04:0 PC); 1,2 dipentanoyl sn glycero 3 phosphocholine (05:0 PC); 1,2 dihexanoyl sn glycero 3 phosphocholine (06:0 PC), 1,2 diheptanoyl sn glycero 3 phosphocholine (07:0 PC); 1,2 dioctanoyl sn glycero 3 phosphocholine (08:0 PC); 1,2 dinonanoyl sn glycero 3 phosphocholine (09:0 PC); 1,2 didecanoyl sn glycero 3 phosphocholine (10:0 PC); 1,2 diundecanoyl sn glycero 3 phosphocholine (11:0 PC, DUPC); 1,2 dilauroyl sn glycero 3 phosphocholine (12:0 PC); 1,2 ditridecanoyl sn glycero 3 phosphocholine (13:0 PC); 1,2 dimyristoyl sn glycero 3 phosphocholine (14:0 PC, DMPC); 1,2 dipentadecanoyl sn glycero 3 phosphocholine (15:0 PC); 1,2 dipalmitoyl sn glycero 3 phosphocholine (16:0 PC, DPPC); 1,2 diphytanoyl sn glycero 3 phosphocholine (4ME 16:0 PC); 1,2 diheptadecanoyl sn glycero 3 phosphocholine (17:0 PC); 1,2 distearoyl sn glycero 3 phosphocholine (18:0 PC, DSPC); 1,2 dinonadecanoyl sn glycero 3 phosphocholine (19:0 PC); 1,2 diarachidoyl sn glycero 3 phosphocholine (20:0 PC); 1,2 dihenarachidoyl sn glycero 3 phosphocholine (21:0 PC); 1,2 dibehenoyl sn glycero 3 phosphocholine (22:0 PC); 1,2 ditricosanoyl sn glycero 3 phosphocholine (23:0 PC); 1,2 dilignoceroyl sn glycero 3 phosphocholine (24:0 PC); 1,2 dimyristoleoyl sn glycero 3 phosphocholine (14:1 (Δ9-Cis) PC); 1,2 dimyristelaidoyl sn glycero 3 phosphocholine (14:1 (Δ9-Trans) PC); 1,2 dipalmitoleoyl sn glycero 3 phosphocholine (16:1 (Δ9-Cis) PC); 1,2 dipalmitelaidoyl sn glycero 3 phosphocholine (16:1 (Δ9-Trans) PC); 1,2 dipetroselenoyl sn glycero 3 phosphocholine (18:1 (Δ6-Cis) PC); 1,2 dioleoyl sn glycero 3 phosphocholine (18:1 (Δ9-Cis) PC, DOPC); 1,2 dielaidoyl sn glycero 3 phosphocholine (18:1 (Δ9-Trans) PC); 1,2 dilinoleoyl sn glycero 3 phosphocholine (18:2 (Cis) PC, DLPC); 1,2 dilinolenoyl sn glycero 3 phosphocholine (18:3 (Cis) PC, DLnPC); 1,2 dieicosenoyl sn glycero 3 phosphocholine (20:1 (Cis) PC); 1,2 diarachidonoyl sn glycero 3 phosphocholine (20:4 (Cis) PC, DAPC); 1,2 dierucoyl sn glycero 3 phosphocholine (22:1 (Cis) PC); 1,2 didocosahexaenoyl sn glycero 3 phosphocholine (22:6 (Cis) PC, DHAPC); 1,2 dinervonoyl sn glycero 3 phosphocholine (24:1 (Cis) PC); 1,2 dihexanoyl sn glycero 3 phosphoethanolamine (06:0 PE); 1,2 dioctanoyl sn glycero 3 phosphoethanolamine (08:0 PE); 1,2 didecanoyl sn glycero 3 phosphoethanolamine (10:0 PE); 1,2 dilauroyl sn glycero 3 phosphoethanolamine (12:0 PE), 1,2 dimyristoyl sn glycero 3 phosphoethanolamine (14:0 PE); 1,2 dipentadecanoyl sn glycero 3 phosphoethanolamine (15:0 PE); 1,2 dipalmitoyl sn glycero 3 phosphoethanolamine (16:0 PE); 1,2 diphytanoyl sn glycero 3 phosphoethanolamine (4ME 16:0 PE); 1,2 diheptadecanoyl sn glycero 3 phosphoethanolamine (17:0 PE); 1,2 distearoyl sn glycero 3 phosphoethanolamine (18:0 PE, DSPE); 1,2 dipalmitoleoyl sn glycero 3 phosphoethanolamine (16:1 PE); 1,2 dioleoyl sn glycero 3 phosphoethanolamine (18:1 (Δ9-Cis) PE, DOPE); 1,2 dielaidoyl sn glycero 3 phosphoethanolamine (18:1 (Δ9-Trans) PE); 1,2 dilinoleoyl sn glycero 3 phosphoethanolamine (18:2 PE, DLPE); 1,2 dilinolenoyl sn glycero 3 phosphoethanolamine (18:3 PE, DLnPE); 1,2 diarachidonoyl sn glycero 3 phosphoethanolamine (20:4 PE, DAPE); 1,2 didocosahexaenoyl sn glycero 3 phosphoethanolamine (22:6 PE, DHAPE); 1,2 di O octadecenyl sn glycero 3 phosphocholine (18:0 Diether PC); 1,2 dioleoyl sn glycero 3 phospho rac (1 glycerol) sodium salt (DOPG), or any combination thereof.

In some aspects, the phospholipid is a asymmetric phospholipid, which is 1 myristoyl 2 palmitoyl sn glycero 3 phosphocholine (14:0-16:0 PC, MPPC); 1 myristoyl 2 stearoyl sn glycero 3 phosphocholine (14:0-18:0 PC, MSPC); 1 palmitoyl 2 acetyl sn glycero 3 phosphocholine (16:0-02:0 PC); 1 palmitoyl 2 myristoyl sn glycero 3 phosphocholine (16:0-14:0 PC, PMPC); 1 palmitoyl 2 stearoyl sn glycero 3 phosphocholine (16:0-18:0 PC, PSPC); 1 palmitoyl 2 oleoyl sn glycero 3 phosphocholine (16:0-18:1 PC, POPC); 1 palmitoyl 2 linoleoyl sn glycero 3 phosphocholine (16:0-18:2 PC, PLPC); 1 palmitoyl 2 arachidonoyl sn glycero 3 phosphocholine (16:0-20:4 PC); 1 palmitoyl 2 docosahexaenoyl sn glycero 3 phosphocholine (14:0-22:6 PC); 1 stearoyl 2 myristoyl sn glycero 3 phosphocholine (18:0-14:0 PC, SMPC); 1 stearoyl 2 palmitoyl sn glycero 3 phosphocholine (18:0-16:0 PC, SPPC); 1 stearoyl 2 oleoyl sn glycero 3 phosphocholine (18:0-18:1 PC, SOPC); 1 stearoyl 2 linoleoyl sn glycero 3 phosphocholine (18:0-18:2 PC); 1 stearoyl 2 arachidonoyl sn glycero 3 phosphocholine (18:0-20:4 PC); 1 stearoyl 2 docosahexaenoyl sn glycero 3 phosphocholine (18:0-22:6 PC); 1 oleoyl 2 myristoyl sn glycero 3 phosphocholine (18:1-14:0 PC, OMPC); 1 oleoyl 2 palmitoyl sn glycero 3 phosphocholine (18:1-16:0 PC, OPPC); 1 oleoyl 2 stearoyl sn glycero 3 phosphocholine (18:1-18:0 PC, OSPC); 1 palmitoyl 2 oleoyl sn glycero 3 phosphoethanolamine (16:0-18:1 PE, POPE); 1 palmitoyl 2 linoleoyl sn glycero 3 phosphoethanolamine (16:0-18:2 PE); 1 palmitoyl 2 arachidonoyl sn glycero 3 phosphoethanolamine (16:0-20:4 PE); 1 palmitoyl 2 docosahexaenoyl sn glycero 3 phosphoethanolamine (16:0-22:6 PE); 1 stearoyl 2 oleoyl sn glycero 3 phosphoethanolamine (18:0-18:1 PE); 1 stearoyl 2 linoleoyl sn glycero 3 phosphoethanolamine (18:0-18:2 PE); 1 stearoyl 2 arachidonoyl sn glycero 3 phosphoethanolamine (18:0-20:4 PE); 1 stearoyl 2 docosahexaenoyl sn glycero 3 phosphoethanolamine (18:0-22:6 PE); 1 oleoyl 2 cholesterylhemisuccinoyl sn glycero 3 phosphocholine (OChemsPC), or any combination thereof.

In some aspects, the phospholipid is a lysolipid. In some aspects, the phospholipid is a lysoglycerophospholipid, a lysoglycosphingoliopid, a lysophosphatidylcholine, a lysophosphatidylethanolamine, a lysophosphatidylinositol, a lysophosphatidylserine, or any combination thereof. In some aspects, the phospholipid is 1 hexanoyl 2 hydroxy sn glycero 3 phosphocholine (06:0 Lyso PC); 1 heptanoyl 2 hydroxy sn glycero 3 phosphocholine (07:0 Lyso PC); 1 octanoyl 2 hydroxy sn glycero 3 phosphocholine (08:0 Lyso PC); 1 nonanoyl 2 hydroxy sn glycero 3 phosphocholine (09:0 Lyso PC); 1 decanoyl 2 hydroxy sn glycero 3 phosphocholine (10:0 Lyso PC); 1 undecanoyl 2 hydroxy sn glycero 3 phosphocholine (11:0 Lyso PC); 1 lauroyl 2 hydroxy sn glycero 3 phosphocholine (12:0 Lyso PC); 1 tridecanoyl 2 hydroxy sn glycero 3 phosphocholine (13:0 Lyso PC); 1 myristoyl 2 hydroxy sn glycero 3 phosphocholine (14:0 Lyso PC); 1 pentadecanoyl 2 hydroxy sn glycero 3 phosphocholine (15:0 Lyso PC); 1 palmitoyl 2 hydroxy sn glycero 3 phosphocholine (16:0 Lyso PC); 1 heptadecanoyl 2 hydroxy sn glycero 3 phosphocholine (17:0 Lyso PC); 1 stearoyl 2 hydroxy sn glycero 3 phosphocholine (18:0 Lyso PC); 1 oleoyl 2 hydroxy sn glycero 3 phosphocholine (18:1 Lyso PC); 1 nonadecanoyl 2 hydroxy sn glycero 3 phosphocholine (19:0 Lyso PC); 1 arachidoyl 2 hydroxy sn glycero 3 phosphocholine (20:0 Lyso PC); 1 behenoyl 2 hydroxy sn glycero 3 phosphocholine (22:0 Lyso PC); 1 lignoceroyl 2 hydroxy sn glycero 3 phosphocholine (24:0 Lyso PC); 1 hexacosanoyl 2 hydroxy sn glycero 3 phosphocholine (26:0 Lyso PC); 1 myristoyl 2 hydroxy sn glycero 3 phosphoethanolamine (14:0 Lyso PE); 1 palmitoyl 2 hydroxy sn glycero 3 phosphoethanolamine (16:0 Lyso PE); 1 stearoyl 2 hydroxy sn glycero 3 phosphoethanolamine (18:0 Lyso PE); 1 oleoyl 2 hydroxy sn glycero 3 phosphoethanolamine (18:1 Lyso PE); 1 hexadecyl sn glycero 3 phosphocholine (C16 Lyso PC); or any combination thereof.

In some aspects, the anchoring moiety comprises a vitamin. In some aspects, the anchoring moiety comprises vitamin D, vitamin K, vitamin E, or any combination thereof. In some aspects, the anchoring moiety further comprises a linker between the biologically active molecule and the anchoring moiety.

In some aspects, the linker comprises a non-cleavable linker. In some aspects, the non-cleavable linker comprises polyethylene glycol (PEG), glycerol, alkyl, succinimide, maleimide, or any combination thereof. In some aspects, the non-cleavable linker comprises polyethylene glycol (PEG) characterized by a formula R3-(O—CH₂—CH₂)ₙ— or R3-(0-CH₂—CH₂)ₙ—O—, wherein R3 being hydrogen, methyl or ethyl and n is an integer between 2 and 200. In some aspects, the non-cleavable linker comprises diethylene glycol, triethylene glycol, tetraethylene glycol (TEG), hexaethylene glycol (HEG), pentaethylene glycol, or any combination thereof.

In some aspects, the linker comprises a polyglycerol (PG) having the formula ((R3-O—(CH₂—CHOH—CH₂O)ₙ—), wherein R3 is hydrogen, methyl or ethyl, and n is an integer between 3 and 200. In some aspects, the linker comprises a diglycerol, triglycerol, tetraglycerol (TG), pentaglycerol, a hexaglycerol (HG), or any combination thereof.

In some aspects, the e linker comprises alkyl. In some aspects, the linker comprises alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, Aryl, heteroaryl, heterocyclyl, cycloalkyl, cycloalkenyl, alkylarylalkyl, alkylarylalkenyl, alkylarylalkynyl, alkenylarylalkyl, alkenyl Reyl alkenyl, alkenyl aryl alkynyl, alkynyl aryl alkyl, alkynyl aryl alkenyl, alkynyl aryl alkynyl, alkyl heteroaryl alkyl, alkyl heteroaryl alkyl, alkyl heteroaryl alkenyl, alkyl heteroaryl alkynyl, alkenyl heteroaryl alkyl, alkenyl heteroaryl alkenyl, alkenyl heteroaryl alkynyl, alkynyl Heteroarylalkyl, alkynylheteroarylalkenyl, alkynylheteroarylalkynyl, alkylheterocyclylalkyl, alkylheterocyclylalkenyl, alkylheterocyclylalkynyl, alkenylheterocyclylalkyl, alkenylheterocyclylalkenyl, alkenylheterocyclylalkynyl, or any combination thereof.

In some aspects, the linker comprises a cleavable linker. In some aspects, the cleavable linker is a redox cleavable linker, a reactive oxygen species cleavable linker, a pH dependent cleavable linker, an enzymatic cleavable linker, a protease cleavable linker, an esterase cleavable linker, a phosphatase cleavable linker, a photoactivated cleavable linker, a self-immolative linker, or any combination thereof. In some aspects, the cleavable linker is a self-immolative linker. In some aspects, the cleavable linker is a cinnamyl group, a naphthyl group, a biphenyl group, a heterocyclic ring, a homoaromatic group, coumarin, furan, thiophene, thiazole, oxazole, isoxazole, pyrrole, pyrazole, pyridine, imidazone, triazole, or any combination thereof.

In some aspects, the linker has the formula:

-Aₐ-Y_{y}-

wherein each -A- is independently an amino acid unit, a is independently an integer from 1 to 12; -Y- is a spacer unit, and y is 0, 1, or 2. In some aspects, the -Aₐ- is a dipeptide, a tripeptide, a tetrapeptide, a pentapeptide, or a hexapeptide. In some aspects, a is 2 and -Aₐ- is selected from the group consisting of valine-alanine, valine-citrulline, phenylalanine-lysine, N-methylvaline-citrulline, cyclohexylalanine-lysine, and beta-alanine-lysine. In some aspects, the -Aₐ- is valine-alanine or valine-citrulline. In some aspects, y is 1. In some aspects, -Y- is a self-immolative spacer. In some aspects, -Y_{y}- has the formula (V):

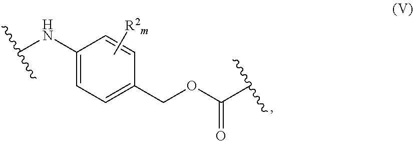

wherein each R² is independently C₁₋₈ alkyl, —O—(C₁₋₈ alkyl), halogen, nitro, or cyano; and m is an integer from 0 to 4. In some aspects, m is 0, 1, or 2. In some aspects, m is 0.

In some aspects, the cleavable linker is valine-alanine-p-aminobenzylcarbamate or valine-citrulline-p-aminobenzylcarbamate. In some aspects, -Y- is a non self-immolative spacer. In some aspects, the non self-immolative spacer is -Gly- or -Gly-Gly-.

In some aspects, the anchoring moiety comprises:

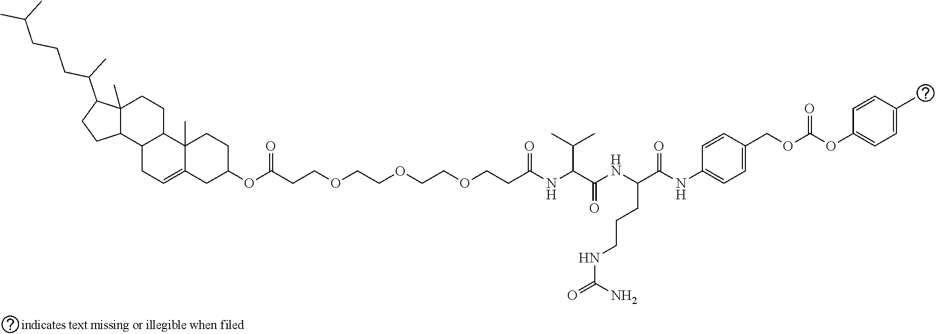 [SMILES: Cc1ccc(OC(=O)OCc2ccc(NC(=O)C(CCCNC(N)=O)NC(=O)C(NC(=O)CCOCCOCCOCCC(=O)OC3CCC4(C)C(=CCC5C4CCC4(C)C(C(C)CCCC(C)C)CCC54)C3)C(C)C)cc2)cc1]

In some aspects, the EV comprises an anchoring moiety selected from SEQ ID NOS: 301-324, 401-567, a fragment thereof, or a combination thereof, and a linker selected from the linker combinations of TABLE 1 and TABLE 2.

In some aspects, the anchoring moiety comprises a scaffold protein. In some aspects, the EV further comprises a scaffold moiety. In some aspects, the anchoring moiety and/or the scaffold moiety is scaffold X. In some aspects, the Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins); a functional fragment thereof, and any combination thereof. In some aspects, the Scaffold X is PTGFRN protein or a functional fragment thereof. In some aspects, the Scaffold X comprises an amino acid sequence as set forth in SEQ ID NOS: 301-324, fragments thereof, and combinations thereof.

In some aspects, the anchoring moiety and/or the scaffold moiety is scaffold Y. In some aspects, the Scaffold Y is a scaffold protein that is capable of anchoring the biologically active molecule on the luminal surface of the extracellular vesicle and/or on the exterior surface of the extracellular vesicle. In some aspects, the Scaffold Y is selected from the group consisting of myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a functional fragment thereof, and any combination thereof. In some aspects, the Scaffold Y is a BASP1 protein or a functional fragment thereof. In some aspects, the Scaffold X comprises an amino acid sequence as set forth in SEQ ID NOS: 401-567, fragments thereof, and combinations thereof.

In some aspects, the biologically active molecule is linked to the anchoring moiety and/or the scaffold moiety on the exterior surface of the EV. In some aspects, the biologically active molecule is linked to the anchoring moiety and/or the scaffold moiety on the luminal surface of the EV. In some aspects, the biologically active molecule is a polypeptide, a peptide, a polynucleotide (DNA and/or RNA), a chemical compound, or any combination thereof. In some aspects, the biologically active molecule is a chemical compound. In some aspects, the chemical compound is a small molecule. In some aspects, the biologically active molecule comprises an antisense oligonucleotide (ASO), a siRNA, a miRNA, a shRNA, a nucleic acid, or any combination thereof. In some aspects, the biologically active molecule comprises a peptide, a protein, an antibody or an antigen binding fragment thereof, or any combination thereof. In some aspects, the antigen binding fragment thereof comprises scFv, (scFv)2, Fab, Fab′, F(ab′)2, F(ab1)2, Fv, dAb, and Fd fragment, diabodys, antibody-related polypeptide, or any fragment thereof. In some aspects, the biologically active molecule comprises an ASO. In some aspects, the ASO targets a transcript, which is a STAT6 transcript, a CEBP/β transcript, a STAT3 transcript, a KRAS transcript, a NRAS transcript, an NLPR3 transcript, a PMP22 transcript, or any combination thereof. In some aspects, the STAT6 transcript comprises SEQ ID NO: 11 or SEQ ID NO: 13. In some aspects, the STAT6 ASO comprises a sequence selected from the group consisting of SEQ ID NO: 601 to SEQ ID NO: 703. In some aspects, the CEBP/β transcript comprises SEQ ID NO: 21 or SEQ ID NO: 23. In some aspects, the CEBP/β ASO comprises a sequence selected from the group consisting of SEQ ID NO: 704 to SEQ ID NO: 806. In some aspects, the STAT3 transcript comprises SEQ ID NO: 41 or SEQ ID NO: 43. In some aspects, the STAT3 ASO comprises a sequence selected from the group consisting of SEQ ID NO: 889 to SEQ ID NO: 988. In some aspects, the NRAS transcript comprises SEQ ID NO: 51 or SEQ ID NO: 53 In some aspects, the NRAS ASO comprises a sequence selected from the group consisting of SEQ ID NO: 989 to SEQ ID NO: 1088. In some aspects, the NLPR3 transcript comprises SEQ ID NO: 1 or SEQ ID NO: 3. In some aspects, the ASO comprises a sequence selected from the group consisting of SEQ ID NO: 101 to SEQ ID NO: 200. In some aspects, the KRAS transcript is a KRAS mutant transcript. In some aspects, the KRAS mutant is KRAS G12D. In some aspects, the KRAS transcript comprises SEQ ID NO: 31 or SEQ ID NO: 33. In some aspects, the ASO comprises a sequence selected from the group consisting of SEQ ID NO: 807 to SEQ ID NO: 888. In some aspects, the PMP22 transcript comprises SEQ ID NO: 58. In some aspects, the ASO comprises a sequence selected from the group consisting of SEQ ID NOS: 62-95 and 201-270.

In some aspects, the EV is an exosome.

In some aspects, the disclosure is directed to a pharmaceutical composition comprising the extracellular vesicle and a pharmaceutically acceptable carrier.

In some aspects, the disclosure is directed to a method of conjugating a biologically active molecule to an EV, comprising linking an anchoring moiety to the EV, a kit comprising the EV and instructions for use.

In some aspects, the disclosure is directed to a method of treating or preventing a disease or disorder in a subject in need thereof comprising administering the EV to the subject. In some aspects, the disease or disorder is a cancer, an inflammatory disorder, a neurodegenerative disorder, a central nervous diseases, or a metabolic disease. In some aspects, the EV is administered intravenously, intraperitoneally, nasally, orally, intramuscularly, subcutaneously, parenterally, or intratumorally.

### BRIEF DESCRIPTION OF THE DRAWINGS/FIGURES

FIG. 1A shows a table listing various ASO sequences that target the NLPR3 transcript. The table includes the following information (from left to right): (i) SEQ ID number designated for the ASO sequence only, (ii) the target start and end positions on the NLPR3 genomic sequence (SEQ ID NO: 1), (iii) the target start and end positions on the NLPR3 mRNA sequence (SEQ ID NO: 2), (iv) the ASO sequence without any particular design or chemical structure, and (v) ASO sequence with a chemical structure. The ASOs are from 5′ to 3′. The symbols in the chemical structures are as follows: Nb means LNA; dN means DNA; 5MdC means 5-Methyl-dC; Nm means MOE; and s means phosphorothioate.

FIG. 1B shows a table listing various STAT6 ASO sequences described herein and the location of the complimentary sequence for each in the mRNA sequence. The ASOs are from 5′ to 3′. The symbols in the chemical structures are as follows: Nb means LNA; dN means DNA; 5MdC means 5-Methyl-dC; Nm means MOE; and s means phosphorothioate.

FIG. 1C shows a table listing various CEBP/β ASO sequences described herein and the location of the complimentary sequence for each in the mRNA sequence. The ASOs are from 5′ to 3′. The symbols in the chemical structures are as follows: Nb means LNA; dN means DNA; 5MdC means 5-Methyl-dC; Nm means MOE; and s means phosphorothioate.

FIG. 1D shows a table listing various STAT3 ASO sequences described herein and the location of the complimentary sequence for each in the mRNA sequence. The ASOs are from 5′ to 3′. The symbols in the chemical structures are as follows: Nb means LNA; dN means DNA; 5MdC means 5-Methyl-dC; Nm means MOE; and s means phosphorothioate.

FIG. 1E shows a table listing various NRas ASO sequences described herein and the location of the complimentary sequence for each in the mRNA sequence. The ASOs are from 5′ to 3′. The symbols in the chemical structures are as follows: Nb means LNA; dN means DNA; 5MdC means 5-Methyl-dC; Nm means MOE; and s means phosphorothioate.

FIG. 1F shows a table listing various KRAS ASO sequences described herein and the location of the complimentary sequence for each in the pre-mRNA (SEQ ID NO: 30) or mRNA sequence (SEQ ID NO: 32). The ASOs are from 5′ to 3′. The symbols in the chemical structures are as follows: Nb means LNA; dN means DNA; 5MdC means 5-Methyl-dC; Nm means MOE; and s means phosphorothioate.

FIGS. 2A-2C are graphical representations of IL-1β production in monocytes (FIG. 2A), M0 macrophages (FIG. 2B), and mouse BMDM (FIG. 2C). The NLRP3 pathway was activated in each sample type by treatment with LPS for 3 hours and ATP for three hours. Samples were then treated with an increasing concentration of MCC950 (log μM), as indicated, and IL-1β levels were measured (pg/mL).

FIG. 3A is a timeline illustrating the dosing and sample collection schedule for intraperitoneal LPS challenge of mice. FIG. 3B is a graphical representation of serum IL-1β levels in mouse serum following administration of increasing amounts of MCC950.

FIGS. 4A-4D are graphical representations of Cy5 levels, as detected by fluorescence (MFI) and normalized to PBS controls. Cy5 is used as a marker of uptake of exosomes comprising Cy5 ASOs (“Exo ASO”; left) or free ASOs (right), as indicated, in various cell types isolated from the blood (FIG. 4A), liver (FIG. 4B), spleen (FIG. 4C), and a tumor (CT26; FIG. 4D). Horizontal lines indicate the average MFI. FIGS. 4E-4J are fluorescent images of bone marrow tissue samples taken from two donors each, showing uptake of exosomes comprising Cy5-reporter ASOs (FIGS. 4E-4F) or free ASO (FIGS. 4G-4H), as compared to PBS negative controls (FIGS. 4I-4J).

FIGS. 5A-5B are graphical representations of the normalized gene expression (%) of STAT6 (FIG. 5A) and CD163 (FIG. 5B) in polarized macrophages following treatment with STAT6 Exo ASO, STAT6 free ASO, or a scrambled Exo ASO (negative control), as indicated (FIGS. 5A-5B).

FIGS. 5C-5D are graphical representations of the normalized gene expression (%) of STAT6 (FIG. 5C) and CD163 (FIG. 5D) in polarized macrophages following treatment with STAT6 Exo ASO, STAT6 free ASO, or a scrambled Exo ASO (negative control), as indicated (FIGS. 5C-5D).

FIGS. 6A-6J are graphical representations of the expression of TGFβ1 (FIG. 6A), CD163 (FIG. 6B), STAT5b (FIG. 6C), STAT6 (FIG. 6D), CEBP/β (FIG. 6E), IL12β (FIG. 6F), AIF1 (FIG. 6G), MYC (FIG. 6H), HLA DQA (FIG. 6I), and CD74 (MIF) (FIG. 6A) in primary human macrophages untreated or treated with scramble Exo ASO, STAT6-Exo-ASO, STAT-6 free ASO, CEBP/β-Exo-ASO, or CEBP/β free ASO, as indicated.

FIGS. 7A-7F are graphical representations of the results of flow cytometry to isolate CD11b⁺ cells. FIGS. 7A-7C show CD45 expression pre-treatment (FIG. 7A), following treatment with a negative control (scramble Exo ASO; FIG. 7B), or post-treatment with an Exo-ASO (FIG. 7C). FIGS. 7D-7F show CD11b expression pre-treatment (FIG. 7D), following treatment with a negative control (scramble Exo ASO; FIG. 7E), or post-treatment with an Exo-ASO (FIG. 7F).

FIGS. 8A-8C are graphical representations of the expression of STAT6 (FIG. 8A), CEBP/β (FIG. 8B) and ARG1 (FIG. 8C) in CD11b-enriched cells as compared to non-enriched cells following exposure to scramble Exo-ASO (FIGS. 8A-8C), STAT6 free ASO (FIGS. 8A and 8C), CEBP/β free ASO (FIG. 8B), STAT6-Exo-ASO (FIGS. 8A and 8C), or CEBP/β-Exo-ASO (FIGS. 8B-8C).

FIGS. 9A-9L are graphical representations of the expression of STAT6 (FIG. 9A), CEBP/β (FIG. 9B), TGFβ1 (FIG. 9C), STAT3 (FIG. 9D), SIRP-α (FIG. 9E), CD47 (FIG. 9F), NOS2 (FIG. 9G), ARG1 (FIG. 9H), CD206 (FIG. 9I), CD274 (FIG. 9J), NLRP3 (FIG. 9K), CSF1R (FIG. 9L), CD36 (FIG. 9M), STAB1 (FIG. 9N), IL13 (FIG. 9O), PI3KG (FIG. 9P), LY6C (FIG. 9Q), LY6G (FIG. 9R), IFNβ1 (FIG. 9S), IFNγ (FIG. 9T), IFNα1 (FIG. 9U), and IL6Rα (FIG. 9V) in CD11b-enriched cells treated with scramble Exo ASO, STAT6-Exo-ASO, STAT-6 free ASO, or CEBP/β-Exo-ASO, as indicated.

FIGS. 10A and 10C are graphical representations of the normalized gene expression (%) of STAT6 (FIG. 10A) and TGFβ1 (FIG. 10C) in primary human M2 macrophages were polarized with IL-13/TGFβ treatment subsequently treated with STAT6 Exo ASO, STAT6 free ASO, or a scrambled Exo ASO (negative control), as indicated.

FIGS. 10B and 10D are graphical representations of the normalized gene expression (%) of CEBP/β (FIG. 10B) and TGFβ1 (FIG. 10D) in primary human M2 macrophages were polarized with IL-13/TGFβ treatment subsequently treated with CEBP/β Exo ASO, CEBP/β free ASO, or a scrambled Exo ASO (negative control), as indicated.

FIG. 11 is a graphical representation of exosome uptake, as evidenced by Cy5 levels, in Lung TD2 following nasal administration of a negative control (−C) or Exo-ASO-Cy5 (“IN”) to naïve mice or mice were treated with bleomycin to induce pulmonary fibrosis (“bleo”).

FIGS. 12A-12H are images of fluorescent in situ hybridization to detect exosome uptake by normal and induced fibrotic lung tissue.

FIGS. 13A-13H are images of in situ hybridization to detect exosome uptake by normal and induced fibrotic lung tissue.

FIG. 13I is a graphical representation showing the level of saturation in the in situ hybridization images, indicating the level of exosome uptake in normal and fibrotic tissue.

FIGS. 14A-14F are images of fluorescent in situ hybridization to detect exosome uptake by lung tissue in Hepa1-6 mice.

FIGS. 15A-15F are images of in situ hybridization to detect exosome uptake by lung tissue in Hepa1-6 mice.

FIG. 16A-16J shows the results of various IC50 experiments using NRas ASOs.

FIG. 17A-17N shows the results of various IC50 experiments using STAT3 ASOs.

FIG. 18 shows a graph depicting the results of STAT3 ASO experiments showing overall mRNA depletion.

FIG. 19 shows a graph depicting the results of NRas ASO experiments showing overall mRNA depletion.

FIG. 20 shows a tumor volume response curve post-inoculation with STAT3 Exo-ASO and STAT3 Free ASO.

FIG. 21 shows a STAT3 gene expression profile response using STAT3, Exo-ASO, STAT3 Free ASO, and STAT3 Free ASO 2X.

FIG. 22 shows percentages of infiltrating MDSCs/CD45s (CD11bHigh F40/80High/CD45) after exposure to PBS, Scramble Exo-ASO, STAT3 Exo-ASO MOE, and STAT3 Free ASO MOE.

FIG. 23 shows percentages of infiltrating MDSCs/Total MDSCs (Ly6GHigh CD11bHigh/IA/IELow) after exposure to PBS, Scramble Exo-ASO, STAT3 Exo-ASO MOE, and STAT3 Free ASO MOE.

FIG. 24 shows a normalized mRNA count after treatment with PBS, Scramble Exo-ASO, STAT3 Exo-ASO, and STAT3 Free ASO.

FIG. 25 presents a table showing that the amount of ASO molecules loaded per engineered exosome is affected by linker structure. Also shown are the structures of the constructs used.

FIG. 26 presents a table showing that the amount of ASO molecules loaded per native exosome is affected by linker structure. Also shown are the structures of the constructs used.

FIGS. 27A-27C show that the potency of Exo-ASO is affected by the ASO linker structure. The structures of the constructs C1-C9, T1-T9 and L1-L3 are those described in FIG. 25. FIG. 27A shows the potency of constructs with a cholesterol-C6 lipid anchor. FIG. 27B shows the potency of constructs with a tocopherol-C8 (L1) or tocopherol palmitate-C6 (L2 and L3) lipid anchor. FIG. 27C shows the potenty of constructs with a cholesterol-TEG lipid anchor.

FIG. 28 is a table showing the sequence of ASO molecules targeting Pmp22.

FIG. 29 is a table showing additional sequences of ASO molecules targeting Pmp22.

### DETAILED DESCRIPTION

The present disclosure is directed to extracellular vesicles (EVs), e.g., exosomes, comprising at least one biologically active molecule covalently linked to the EV, e.g., exosome, via an anchoring moiety and uses thereof. Non-limiting examples of the various aspects are shown in the present disclosure.

Before the present disclosure is described in greater detail, it is to be understood that this invention is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

Accordingly, the terms defined immediately below are more fully defined by reference to the specification in its entirety.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “aryl” refers to a carbocyclic aromatic group. Examples of aryl groups include, but are not limited to, phenyl, naphthyl and anthracenyl. A carbocyclic aromatic group can be unsubstituted or substituted with one or more groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂—, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

The term “arylene” refers to an aryl group which has two covalent bonds and can be in the ortho, meta, or para configurations as shown in the following structures:

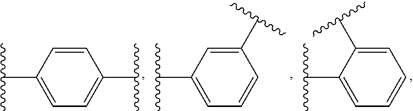

in which the phenyl group can be unsubstituted or substituted with up to four groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂—, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

The term “biologically active molecule” as use herein refers to any molecule that can be attached to an EV, e.g., exosome, via an anchoring moiety, wherein the molecule can have a therapeutic or prophylactic effect in a subject in need thereof, or be used for diagnostic purposes. Accordingly, by way of example, the term biologically active molecule include proteins (e.g., antibodies, proteins, polypeptides, and derivatives, fragments, and variants thereof), lipids and derivatives thereof, carbohydrates (e.g., glycan portions in glycoproteins), or small molecules. In some aspects, the biologically active molecule is a radioisotope. In some aspects, the biologically active molecule is a detectable moiety, e.g., a radionuclide, a fluorescent molecule, or a contrast agent.

The term “C₁₋₈ alkyl” as used herein refers to a straight chain or branched, saturated hydrocarbon having from 1 to 8 carbon atoms. Representative “C₁₋₈ alkyl” groups include, but are not limited to, methyl, ethyl, n-propyl, n-butyl, n-pentyl, n-hexyl, n-heptyl, n-octyl, isopropyl, sec-butyl, isobutyl, tert-butyl, isopentyl, and 2-methylbutyl.

The term “C₁₋₁₀ alkylene” refers to a saturated, straight chain hydrocarbon group of the formula —(CH₂)₁₋₁₀—. Examples of C₁₋₁₀ alkylene include methylene, ethylene, propylene, butylene, pentylene, hexylene, heptylene, octylene, nonylene, and decalene.

The term “C₃₋₈ carbocycle” refers to a 3-, 4-, 5-, 6-, 7- or 8-membered saturated or unsaturated non-aromatic carbocyclic ring. Representative C₃₋₈ carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and -cyclooctadienyl. A C₃₋₈ carbocycle group can be unsubstituted or substituted with one or more groups including, but not limited to, —C₁₋₈ alkyl, —O—(C₁₋₈ alkyl), aryl, —C(O)R′, —OC(O)R′, —C(O)OR′, —C(O)NH₂, —C(O)NHR′, —C(O)N(R′)₂—, NHC(O)R′, —S(O)₂R′, —S(O)R′, —OH, -halogen, —N₃, —NH₂, —NH(R′), —N(R′)₂ and —CN, where each R′ is independently H, —C₁₋₈ alkyl, or aryl.

The term “C₃₋₈ carbocyclo” refers to a C₃₋₈ carbocycle group defined above wherein one or more of the carbocycle's hydrogen atoms is replaced with a bond.

The term “C₃₋₈ heterocycle” refers to an aromatic or non-aromatic C₃₋₈ carbocycle in which one to four of the ring carbon atoms are independently replaced with a heteroatom selected from the group consisting of O, S and N. Representative examples of a C₃₋₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃₋₈ heterocycle can be unsubstituted or substituted with up to seven groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂, —NHC(O)R′, —S(O)₂R′, —S(O)R′, —OH, -halogen, —N₃, —NH₂, —NH(R′), —N(R′)₂, and —CN, wherein each R′ is independently H, —C₁₋₈ alkyl, or aryl.

The term “C₃₋₈ heterocyclo” refers to a C₃₋₈ heterocycle group defined above wherein one of the heterocycle group's hydrogen atoms is replaced with a bond. A C₃₋₈ heterocyclo can be unsubstituted or substituted with up to six groups including, but not limited to, —C₁₋₈ alkyl, —O—(C₁₋₈ alkyl), -aryl, —C(O)R′, —OC(O)R′, —C(O)OR′, —C(O)NH₂, —C(O)NHR′, —C(O)N(R′)₂, —NHC(O)R′, —S(O)₂R′, —S(O)R′, —OH, -halogen, —N₃, —NH₂, —NH(R′), —N(R′)₂ and —CN, wherein each R′ is independently H, —C₁₋₈ alkyl, or aryl.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

As used herein, the term “conventional EV protein” means a protein previously known to be enriched in EVs.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

The term “derivative” as used herein refers to an EV, e.g., exosome, component (e.g., a protein, such as Scaffold X and/or Scaffold Y, a lipid, or a carbohydrate) or to a biologically active molecule (e.g., a polypeptide, polynucleotide, lipid, carbohydrate, antibody or fragment thereof, PROTAC, etc.) that has been chemically modified to either introduce a reactive maleimide group or a thiol group susceptible of reaction with a maleimide group. For example, an antibody modified with a bifunctional reagent comprising (i) a group reacting, e.g., with free amino groups, and (ii) a maleimide group, could result in antibody derivative comprising a reactive maleimide group that may react with free thiol groups in a Scaffold X protein on the EV, e.g., exosome. Conversely, an Scaffold X on the EV, e.g., exosome, could be modified with a bifunctional reagent comprising (i) a group reacting, e.g., with free amino groups, and (ii) a maleimide group, resulting in a Scaffold X derivative comprising a reactive maleimide group that may react with free thiol groups in a biologically active molecule, e.g., an antibody.

The terms “excipient” and “carrier” are used interchangeably and refer to an inert substance added to a pharmaceutical composition to further facilitate administration of a compound.

As used herein, the terms “extracellular vesicle,” “EV,” and grammatical variants thereof, are used interchangeably and refer to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the exosomes of the present disclosure are produced by cells that express one or more transgene products.

In some aspects, EVs, e.g., exosomes, e.g., nanovesicles, of the present disclosure are engineered by covalently linking at least one biologically active molecule (e.g., a protein such as an antibody or ADC, a RNA or DNA such as an antisense oligonucleotide, a small molecule drug, a toxin) to the EV, e.g., exosome, e.g., nanovesicle, via an anchoring moiety.

In some aspects, the EVs, e.g., exosomes or nanovesicles, of the present disclosure can comprise various macromolecular payloads either within the internal space (i.e., lumen), displayed on the external (exterior) surface or internal (luminal) surface of the EV, and/or spanning the membrane. In some aspects, the payload can comprise, e.g., nucleic acids, proteins, carbohydrates, lipids, small molecules, and/or combinations thereof. In certain aspects, an EV, e.g, an exosome, comprises a scaffold moiety (e.g., Scaffold X). EVs, e.g., exosomes, can be derived from a living or dead organism, explanted tissues or organs, prokaryotic or eukaryotic cells, and/or cultured cells. In some aspects, the EVs, e.g., exosomes, are produced by cells that express one or more transgene products. In other aspects, the EVs of the present disclosure are without limitation nanovesicles, microsomes, microvesicles, extracellular bodies, or apoptotic bodies.

As used herein, the term “fragment” of a protein (e.g., a biologically active molecule such as a therapeutic protein, or an scaffold protein such as Scaffold X or Scaffold Y) refers to an amino acid sequence of a protein that is shorter than the naturally-occurring sequence, N- and/or C-terminally deleted or any part of the protein deleted in comparison to the naturally occurring protein.

As used herein, the term “functional fragment” refers to a protein fragment that retains protein function. Accordingly, in some aspects, a functional fragment of a Scaffold protein, e.g., Scaffold X protein, retains the ability to anchor a biologically active molecule on the luminal surface or on the external surface of the EV, e.g., exosome, via a maleimide moiety. Similarly, in certain aspects, a functional fragment of a Scaffold Y protein retains the ability to anchor a moiety on the luminal surface of the EV, e.g., exosome.

Whether a fragment is a functional fragment can be assessed by any art known methods to determine the protein content of EVs, e.g., exosomes, including Western Blots, FACS analysis and fusions of the fragments with autofluorescent proteins like, e.g., GFP. In certain aspects, a functional fragment of a Scaffold X protein retains, e.g., at least about 50%, at least about 60%, at least about 70%, at least about 80%, at least about 90% or at least about 100% of the ability of the naturally occurring Scaffold X protein to anchor a biologically active molecule on the luminal or on the external surface of the EV, e.g., exosome, via a maleimide moiety.

As used herein “anchoring” a biologically active molecule on the luminal or external surface of an EV (e.g., exosome) of the present disclosure via a scaffold protein refers to attaching covalently the biologically active molecule to the portion of the scaffold molecule located on the luminal or external surface of the EV (e.g., exosome), respectively.

In certain aspects, a functional fragment of a Scaffold Y protein retains, e.g., at least about 50%, at least about 60%, at least about 70%, at least about 80%, at least about 90% or at least about 100% of the ability of the naturally occurring Scaffold Y protein to anchor a moiety on the luminal surface of the EV, e.g., exosome.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least 25%, 30%, 35%, 40%, 45%, 50%, 55%, 60%, 65%, 70%, 75%, 80%, 85%, 90%, 95%, or 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

As used herein, the term “identity” refers to the overall monomer conservation between polymeric molecules, e.g., between polypeptide molecules or polynucleotide molecules (e.g. DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions are then compared.

When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the term “immune modulator” refers to an agent that acts on a target (e.g., a target cell) that is contacted with the EV (e.g., exosome), and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA or DNA, or a small molecule. In some aspects of the present disclosure, the biologically active molecule is an immune modulator.

An “immune response”, as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects of the present disclosure, the biologically active molecule is a molecule capable of eliciting an immune response.

In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

The term “immunoconjugate” as used herein refers to a compound comprising a binding molecule (e.g., an antibody) and one or more moieties, e.g., therapeutic or diagnostic moieties, chemically conjugated to the binding molecule. In general an immunoconjugate is defined by a generic formula: A-(L-M)n wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In other aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate.

As used herein, the terms “isolated,” “purified,” “extracted,” and grammatical variants thereof are used interchangeably and refer to the state of a preparation of desired EVs (e.g., a plurality of EVs of known or unknown amount and/or concentration), that has undergone one or more processes of purification, e.g., a selection or an enrichment of the desired EV, e.g., exosome, preparation. In some aspects, isolating or purifying as used herein is the process of removing, partially removing (e.g., a fraction) of the EVs, e.g., exosomes, from a sample containing producer cells. In some aspects, an isolated EV, e.g., exosome, composition has no detectable undesired activity or, alternatively, the level or amount of the undesired activity is at or below an acceptable level or amount. In other aspects, an isolated EV, e.g., exosome, composition has an amount and/or concentration of desired EVs, e.g., exosomes, at or above an acceptable amount and/or concentration. In other aspects, the isolated EVs, e.g., exosome, composition is enriched as compared to the starting material (e.g., producer cell preparations) from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material. In some aspects, isolated EV, e.g. exosome, preparations are substantially free of residual biological products. In some aspects, the isolated EV, e.g., exosome, preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites. Substantially free of residual biological products can also mean that the EV, e.g., exosome, composition contains no detectable producer cells and that only EVs, e.g., exosomes, are detectable.

The terms “linked,” “fused,” and grammatical variants thereof are used interchangeably and refer to a first moiety, e.g., a first amino acid sequence or nucleotide sequence, covalently or non-covalently joined to a second moiety, e.g., a second amino acid sequence or nucleotide sequence, respectively. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In one aspect, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules). The term “linked” is also indicated by a hyphen (-). In some aspects, a Scaffold X protein on an EV, e.g., exosome, can be linked or fused to a biologically active molecule via a maleimide moiety.

As used herein the term “lumen-engineered EV” refers to an EV, e.g., exosome with the luminal surface of the membrane or the lumen of the EV, e.g., exosome, modified in its composition so that the luminal surface or the lumen of the engineered EV, e.g., exosome, is different from that of the EV, e.g., exosome, prior to the modification or of the naturally occurring EV, e.g., exosome.

The engineering can be directly in the lumen (i.e., the void within the EV) or in the membrane of the EV (e.g., exosome), in particular the luminal surface of the EV, so that the lumen and/or the luminal surface of the EV, e.g., exosome is changed. For example, the membrane is modified in its composition of a protein, a lipid, a small molecule, a carbohydrate, etc. so that the luminal surface of the EV, e.g., exosome is modified. Similarly, the contents in the lumen can be modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered EV, e.g., lumen-engineered exosome, comprises an exogenous protein (i.e., a protein that the EV, e.g., exosome, does not naturally express) or a fragment or variant thereof that can be exposed on the luminal surface or lumen of the EV, e.g., exosome, or can be an anchoring point (attachment) for a moiety exposed on the inner layer of the EV, e.g., exosome. In other aspects, a lumen-engineered EV, e.g., a lumen-engineered exosome, comprises a higher expression of a natural EV, e.g., exosome, protein (e.g., Scaffold X or Scaffold Y) or a fragment or variant thereof that can be exposed to the lumen of the EV, e.g., exosome, or can be an anchoring point (attachment) for a moiety exposed on the luminal surface of the EV, e.g., exosome.

As used herein, the term “macromolecule” refers to nucleic acids, proteins, lipids, carbohydrates, metabolites, or combinations thereof.

As used herein, the term “macromolecule” refers to nucleic acids, proteins, lipids, carbohydrates, metabolites, or combinations thereof.

The term “modified,” when used in the context of EVs, e.g., exosomes, described herein, refers to an alteration or engineering of an EV, e.g., exosome and/or its producer cell, such that the modified EV, e.g., exosome, is different from a naturally-occurring EV, e.g., exosome. In some aspects, a modified EV, e.g., exosome, described herein comprises a membrane that differs in composition of a protein, a lipid, a small molecular, a carbohydrate, etc. compared to the membrane of a naturally-occurring EV, e.g., exosome. E.g., the membrane comprises higher density or number of natural EV, e.g., exosome, proteins and/or membrane comprises proteins that are not naturally found in EV, e.g., exosomes. In certain aspects, such modifications to the membrane change the exterior surface of the EV, e.g., exosome (e.g., surface-engineered EVs and exosomes described herein). In certain aspects, such modifications to the membrane change the luminal surface of the EV, e.g., exosome (e.g., lumen-engineered EV and exosomes described herein).

As used herein the terms “modified protein” or “protein modification” refers to a protein having at least 15% identity to the non-mutant amino acid sequence of the protein. A modification of a protein includes a fragment or a variant of the protein. A modification of a protein can further include chemical, or physical modification to a fragment or a variant of the protein.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety, e.g., Scaffold X and/or Scaffold Y. Nanovesicles, once derived from a producer cell, can be isolated from the producer cell based on its size, density, biochemical parameters, or a combination thereof.

As used herein, the term “payload” refers to a biologically active molecule (e.g., a therapeutic agent) that acts on a target (e.g., a target cell) that is contacted with the EV, e.g., exosome, of the present disclosure. Non-limiting examples of payloads that can be introduced into an EV, e.g., exosome, include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an antigen. As used herein, the term “antigen” refers to any agent that when introduced into a subject elicits an immune response (cellular or humoral) to itself. In some aspects, the payload molecules are covalently linked to the EV, e.g., exosome, via a maleimide moiety. In other aspects, a payload comprises an adjuvant.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as exosome of the present disclosure, mixed or intermingled with, or suspended in one or more other chemical components, such as pharmaceutically-acceptable carriers and excipients. One purpose of a pharmaceutical composition is to facilitate administration of preparations of EVs, e.g., exosomes, to a subject.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV, e.g., exosome, via a maleimide moiety is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV, e.g., exosome, via a maleimide moiety is a polypeptide, e.g., an antibody or a derivative thereof such as an ADC, a PROTAC, a toxin, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, the Scaffold X and/or Scaffold Y proteins present in EVs, e.g., exosomes, are recombinantly produced by overexpressing the scaffold proteins in the producer cells, so that levels of scaffold proteins in the resulting EVs, e.g., exosomes are significantly increased with respect to the levels of scaffold proteins present in EVs, e.g., exosomes, of producer cells not overexpressing such scaffold proteins.

As used herein, the term “scaffold moiety” refers to a molecule, e.g., a protein such as Scaffold X or Scaffold Y, that can be used to anchor a payload, e.g., a biologically active molecule, to the EV, e.g., exosome, either on the luminal surface or on the external surface of the EV, e.g., exosome. In certain aspects, a scaffold moiety comprises a synthetic molecule. In some aspects, a scaffold moiety comprises a non-polypeptide moiety. In other aspects, a scaffold moiety comprises, e.g., a lipid, carbohydrate, protein, or combination thereof (e.g., a glycoprotein or a proteolipid) that naturally exists in the EV, e.g., exosome. In some aspects, a scaffold moiety comprises a lipid, carbohydrate, or protein that does not naturally exist in the EV, e.g., exosome. In some aspects, a scaffold moiety comprises a lipid or carbohydrate which naturally exists in the EV, e.g., exosome, but has been enriched in the EV, e.g., exosome with respect to basal/native/wild type levels. In some aspects, a scaffold moiety comprises a protein which naturally exists in the EV, e.g., exosome but has been enriched in the EV, e.g., exosome, for example, by recombinant overexpression in the producer cell, with respect to basal/native/wild type levels. In certain aspects, a scaffold moiety is Scaffold X. In some aspects, a scaffold moiety is Scaffold Y. In further aspects, a scaffold moiety comprises both Scaffold X and Scaffold Y.

As used herein, the term “Scaffold X” refers to EV, e.g., exosome, proteins that have been identified on the surface of EVs, e.g., exosomes. See, e.g., U.S. Pat. No. 10,195,290, which is incorporated herein by reference in its entirety. Non-limiting examples of Scaffold X proteins include: prostaglandin F2 receptor negative regulator (“PTGFRN”); basigin (“BSG”); immunoglobulin superfamily member 2 (“IGSF2”); immunoglobulin superfamily member 3 (“IGSF3”); immunoglobulin superfamily member 8 (“IGSF8”); integrin beta-1 (“ITGB1”); integrin alpha-4 (“ITGA4”); 4F2 cell-surface antigen heavy chain (“SLC3A2”); and a class of ATP transporter proteins (“ATP1A1,” “ATP1A2,” “ATP1A3,” “ATP1A4,” “ATP1B3,” “ATP2B1,” “ATP2B2,” “ATP2B3,” “ATP2B”). In some aspects, a Scaffold X protein can be a whole protein or a fragment thereof (e.g., functional fragment, e.g., the smallest fragment that is capable of anchoring another moiety on the external surface or on the luminal surface of the EV, e.g., exosome). In some aspects, a Scaffold X can anchor a biologically active molecule to the external surface or the lumen of the EV, e.g. an exosome. In some aspects of the present disclosure, a biologically active molecule can be covalently attached to a Scaffold X via a maleimide moiety. In some aspects, the biologically active molecule can be attached to Scaffold X via a maleimide moiety on the luminal surface of the EV, e.g., exosome. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B.

As used herein, the term “Scaffold Y” refers to EV, e.g., exosome, proteins that have been identified within the lumen of EV, e.g., exosomes. See, e.g., International Appl. No. PCT/US2018/061679, which is incorporated herein by reference in its entirety. Non-limiting examples of Scaffold Y proteins include: myristoylated alanine rich Protein Kinase C substrate (“MARCKS”); myristoylated alanine rich Protein Kinase C substrate like 1 (“MARCKSL1”); and brain acid soluble protein 1 (“BASP1”). In some aspects, a Scaffold Y protein can be a whole protein or a fragment thereof (e.g., functional fragment, e.g., the smallest fragment that is capable of anchoring a moiety on the luminal surface of the EV, e.g., exosome). In some aspects, a Scaffold Y can anchor a moiety to the luminal surface of the EV, e.g., exosome. In some aspects of the present disclosure, a moiety can be covalently attached to a Scaffold Y. In some aspects, the moiety can be attached to Scaffold Y on the luminal surface of the EV, e.g., exosome.

The term “self-immolative spacer” as used herein refers to a spacer as defined below that will spontaneously separate from the second moiety (e.g., a biologically active molecule) if its bond to the first moiety (e.g., a cleavable linker) is cleaved.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

The term “spacer” as used herein refers to a bifunctional chemical moiety which is capable of covalently linking together two spaced moieties (e.g., a cleavable linker and a biologically active molecule) into a normally stable dipartate molecule.

Unless otherwise indicated, reference to a compound that has one or more stereocenters intends each stereoisomer, and all combinations of stereoisomers, thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the term “substantially free” means that the sample comprising EVs, e.g., exosomes, comprises less than 10% of macromolecules, e.g., contaminants, by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein the term “surface-engineered EV” (e.g., Scaffold X-engineered exosome) refers to an EV with the membrane or the surface of the EV modified in its composition so that the surface of the engineered EV is different from that of the EV prior to the modification or of the naturally occurring EV.

As used herein the term “surface-engineered exosome” (e.g., Scaffold X-engineered exosome) refers to an exosome with the membrane or the surface of the exosome (external surface or luminal surface) modified in its composition so that the surface of the engineered exosome is different from that of the exosome prior to the modification or of the naturally occurring exosome.

The engineering can be on the surface of the EV, e.g., exosome or in the membrane of the EV, e.g., exosome, so that the surface of the EV, e.g., exosome is changed. For example, the membrane can be modified in its composition of, e.g., a protein, a lipid, a small molecule, a carbohydrate, or a combination thereof. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome or can be an anchoring point (attachment) for a moiety exposed on the surface of the EV, e.g., exosome. In other aspects, a surface-engineered EV, e.g., exosome comprises a higher expression (e.g., higher number) of a natural EV, e.g., exosome protein (e.g., Scaffold X) or a fragment or variant thereof that can be exposed to the surface of the EV, e.g., exosome or can be an anchoring point (attachment) for a moiety exposed on the surface of the EV, e.g., exosome. In a specific aspect, a surface-engineered EV, e.g., exosome, comprises the modification of one or more membrane components, e.g., a protein such as Scaffold X, a lipid, a small molecule, a carbohydrate, or a combination thereof, wherein at least one of the components is covalently attached to a biologically active molecule via a maleimide moiety.

As used herein the term “therapeutically effective amount” is the amount of reagent or pharmaceutical compound comprising an EV or exosome of the present disclosure that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

As used herein, the term “variant” of a molecule (e.g., functional molecule, antigen, or Scaffold X and/or Scaffold Y) refers to a molecule that shares certain structural and functional identities with another molecule upon comparison by a method known in the art. For example, a variant of a protein can include a substitution, insertion, deletion, frame shift or rearrangement in another protein.

In some aspects, a variant of a Scaffold X or derivative comprises a Scaffold X variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins.

In some aspects, the variant or variant of a fragment of Scaffold X protein disclosed herein, or derivatives thereof, retains the ability to be specifically targeted to EVs, e.g., exosomes. In some aspects, the Scaffold X or Scaffold X derivative includes one or more mutations, for example, conservative amino acid substitutions.

In some aspects, a variant of a Scaffold Y or derivative thereof comprises a variant having at least 70% identity to MARCKS, MARCKSL1, BASP1 or a fragment of MARCKS, MARCKSL1, or BASP1.

In some aspects, the variant or variant of a fragment of Scaffold Y protein, or derivatives thereof, retains the ability to be specifically targeted to the luminal surface of EVs, e.g., exosomes. In some aspects, the Scaffold Y includes one or more mutations, e.g., conservative amino acid substitutions.

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, variants or derivatives include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells.

Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination.

In some aspects, Scaffold X and/or Scaffold Y can modified at any convenient location. In some aspects, a biologically active molecule can be modified at any convenient location. In particular aspects of the present disclosure, an EV, e.g., exosome, component (e.g., a protein such as Scaffold X and/or Scaffold Y, a lipid, or a glycan) and/or a biologically active molecule (e.g., an antibody or ADC, a PROTAC, a small molecule such as a cyclic dinucleotide, a toxin such as MMAE, a STING agonist, a tolerizing agent, or an antisense oligonucleotide) can be modified to yield a derivative comprising at least one maleimide moiety.

### II. Conjugated EVs (e.g., Exosomes) of the Disclosure

Extracellular vesicles (EVs) typically have 20 nm to 1000 nm in diameter; e.g., exosomes, which are small extracellular vesicles, have typically 100-200 nm in diameter. EVs, e.g., exosomes, are composed of a limiting lipid bilayer and a diverse set of proteins and nucleic acids (Maas, S. L. N., et al., Trends. Cell Biol. 27(3):172-188 (2017)). EVs, e.g., exosomes, exhibit preferential uptake in discrete cell types and tissues, and their tropism can be directed by adding proteins to their surface that interact with receptors on the surface of target cells (Alvarez-Erviti, L., et al., Nat. Biotechnol. 29(4):341-345 (2011)).

Unlike antibodies, EVs (e.g., exosomes) can accommodate large numbers of molecules attached to their surface, on the order of thousands to tens of thousands of molecules per EV (e.g., exosome). EV (e.g., exosome)-drug conjugates thus represent a platform to deliver a high concentration of therapeutic compound to discrete cell types, while at the same time limiting overall systemic exposure to the compound, which in turn reduces off-target toxicity.

In some aspects, the present disclosure provides a “modified biologically active molecules” (MBAM), e.g., an ASO, comprising a “biologically active molecule” (BAM), e.g., an ASO, modified by binding, e.g., covalently, one or more anchoring moieties to the BAM, e.g., an ASO, either directly or indirectly, e.g., via one or more linker combinations. A modified BAM disclosed here can comprise a “anchoring moiety” (AM) and optionally one or more linkers (“linker combination”) which connect the AM to the BAM as schematically represented below

[AM]-[Linker]n-[BAM]

wherein n is an integer between 0 and 10.

The BAM can be attached to an anchoring moiety or linker combination via reaction between a “reactive group” (RG; e.g., amine, thiol, hydroxy, carboxylic acid, or azide) with a “reactive moiety” (RM; e.g., maleimide, succinate, NHS). Several potential synthetic routes are envisioned, for example.

[AM]-/Reactive moiety/+/Reactive group/-[BAM]

[AM]-[Linker]n-/Reactive moiety/+/Reactive group/-[BAM]

[AM]-/Reactive moiety/+/Reactive group/-[Linker]n-[BAM]

[AM]-[Linker]n-/Reactive moiety/+/Reactive group/-[Linker]n-[BAM]

The anchoring moiety can insert into the lipid bilayer of an EV, e.g., an exosome, allowing the loading of the exosome with a BAM, e.g., an ASO. Currently, a predominant obstacle to the commercialization of exosomes as a delivery vehicle for polar BAMs, e.g., ASOs, is highly inefficient loading. This obstacle can be overcome by modifying BAMs, e.g., ASOs, prior to loading them into exosomes. Thus, as described herein, modification of BAMs, e.g., ASOs, facilitates their loading into exosomes.

The methods of loading exosomes with modified BAMs, e.g., ASOs, set forth herein significantly improve loading efficiency as compared to the loading efficiency previously reported for introducing unmodified BAMs into exosomes by, for example, electroporation or cationic lipid transfection.

In some aspects, the modifications increase the hydrophobicity of the BAM, e.g., an ASO, by at least about 1, at least about 2, at least about 3, at least about 4, at least about 5, at least about 6, at least about 7, at least about 8, at least about 9, or at least about 10 fold relative to native (non-modified) BAM, e.g., the corresponding unmodified ASO. In some aspects, the modifications increase the hydrophobicity of the BAM, e.g., an ASO, by at least about 1, at least about 2, at least about 3, at least about 4, at least about 5, at least about 6, at least about 7, at least about 8, at least about 9, or at least about 10 orders of magnitude relative to native (non-modified) BAM, e.g., the corresponding unmodified ASO.

In some aspects, the modifications increase the hydrophobicity of the BAM, e.g., an ASO, by at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, at least about 450%, at least about 500%, at least about 600%, at least about 700%, at least about 800%, at least about 900%, or at least about 1000% relative to native (non-modified) BAM, e.g., the corresponding unmodified ASO. Increases in hydrophobicity can be assessed using any suitable method. For example, hydrophobicity can be determined by measuring the percentage solubility in an organic solvent, such as octanol, as compared to solubility in an aqueous solvent, such as water.

In some aspect, an anchoring moiety can be chemically conjugated to a BAM, e.g., an ASO, to enhance its hydrophobic character. In exemplary aspects, the anchoring moiety is a sterol (e.g., cholesterol), GM1, a lipid, a vitamin, a small molecule, a peptide, or a combination thereof. In some aspects, the moiety is a lipid. In some aspects, the anchoring moiety is a sterol, e.g., cholesterol. Additional moieties include, for example, phospholipids, lysophospholipids, fatty acids, or vitamins (e.g., vitamin D or vitamin E).

In some aspects, the anchoring moiety is conjugated at the termini of the BAM, e.g., an ASO, either directly or via one or more linkers (i.e., “terminal modification”). In other aspects, the anchoring moiety is conjugated to other portions of BM, e.g., an ASO.

In some aspects, the ASO comprises a contiguous nucleotide sequence of from about 10 to about 30, such as 10-20, 14-20, 16-20, or 15-25, nucleotides in length. In certain aspects, the ASO is 20 nucleotides in length. In certain aspects, the ASO is 18 nucleotides in length. In certain aspects, the ASO is 19 nucleotides in length. In certain aspects, the ASO is 17 nucleotides in length. In certain aspects, the ASO is 16 nucleotides in length. In certain aspects, the ASO is 15 nucleotides in length. In certain aspects, the ASO is 14 nucleotides in length. In certain aspects, the ASO is 13 nucleotides in length. In certain aspects, the ASO is 12 nucleotides in length. In certain aspects, the ASO is 11 nucleotides in length. In certain aspects, the ASO is 10 nucleotides in length.

In some aspects, the ASO comprises a contiguous nucleotide sequence of from about 10 to about 50 nucleotides in length, e.g., about 10 to about 45, about 10 to about 40, about 10 or about 35, or about 10 to about 30. In certain aspects, the ASO is 21 nucleotides in length. In certain aspects, the ASO is 22 nucleotides in length. In certain aspects, the ASO is 23 nucleotides in length. In certain aspects, the ASO is 24 nucleotides in length. In certain aspects, the ASO is 25 nucleotides in length. In certain aspects, the ASO is 26 nucleotides in length. In certain aspects, the ASO is 27 nucleotides in length. In certain aspects, the ASO is 28 nucleotides in length. In certain aspects, the ASO is 29 nucleotides in length. In certain aspects, the ASO is 30 nucleotides in length. In certain aspects, the ASO is 31 nucleotides in length. In certain aspects, the ASO is 32 nucleotides in length. In certain aspects, the ASO is 33 nucleotides in length. In certain aspects, the ASO is 34 nucleotides in length. In certain aspects, the ASO is 35 nucleotides in length. In certain aspects, the ASO is 36 nucleotides in length. In certain aspects, the ASO is 37 nucleotides in length. In certain aspects, the ASO is 38 nucleotides in length. In certain aspects, the ASO is 39 nucleotides in length. In certain aspects, the ASO is 40 nucleotides in length. In certain aspects, the ASO is 41 nucleotides in length. In certain aspects, the ASO is 42 nucleotides in length. In certain aspects, the ASO is 43 nucleotides in length. In certain aspects, the ASO is 44 nucleotides in length. In certain aspects, the ASO is 45 nucleotides in length. In certain aspects, the ASO is 46 nucleotides in length. In certain aspects, the ASO is 47 nucleotides in length. In certain aspects, the ASO is 48 nucleotides in length. In certain aspects, the ASO is 49 nucleotides in length. In certain aspects, the ASO is 50 nucleotides in length.

In some aspects, the modified BAM, e.g., an ASO, can include a detectable label. Exemplary labels include fluorescent labels and/or radioactive labels. In some aspects, where modified BAMs, e.g., ASOs, are fluorescently labeled, the detectable label can be, for example, Cy3. Adding a detectable label to modified BAMs, e.g., ASOs, can be used as a way of labeling exosomes, and following their biodistribution. In other aspects, a detectable label can be attached to exosomes directly, for example, by way of labeling an exosomal lipid and/or an exosomal peptide.

The different components of a modified BAM (i.e., anchoring moieties, linkers and linker combinations, and BAMs such as ASOs) can be linked by amide, ester, ether, thioether, disulfide, phosphoramidate, phosphotriester, phosphorodithioate, methyl phosphonate, phosphodiester, or phosphorothioate linkages or, alternatively any or other linkage.

In some aspects, the different components of a modified BAM, can be linker using bifunctional linkers (i.e., linkers containing two functional groups), such as N-succinimidyl-3-(2-pyridyldithio)propionate, N-4-maleimiide butyric acid, S-(2-pyridyldithio)cysteamine, iodoacetoxysuccinimide, N-(4-maleimidebutyloxy) succinimide, N-[5-(3-maleimide propylamide)-1-carboxypentyl]iminodiacetic acid, N-(5-aminopentyl)-iminodiacetic acid, and the like.

II.A. Anchoring Moieties

Suitable anchoring moieties capable of anchoring a BAM to the surface of an EV, e.g., an exosome, comprise for example sterols (e.g., cholesterol), lipids, lysophospholipids, fatty acids, or fat-soluble vitamins, as described in detail below.

In some aspects, the anchoring moiety can be a lipid. A lipid anchoring moiety can be any lipid known in the art, e.g., palmitic acid or glycosylphosphatidylinositols. In some aspects, the lipid, is a fatty acid, phosphatide, phospholipid (e.g., phosphatidyl choline, phosphatidyl serine, or phosphatidyl ethanolamine), or analogue thereof (e.g. phophatidylcholine, lecithin, phosphatidylethanolamine, cephalin, or phosphatidylserine or analogue or portion thereof, such as a partially hydrolyzed portion thereof).

Generally, anchoring moieties are chemically attached. However, an anchoring moiety can be attached to a BAM enzymatically. In some aspects, in the possible to attach an anchoring moiety to a BAM via modification of cell culture conditions. For example, by using a culture medium where myristic acid is limiting, some other fatty acids including shorter-chain and unsaturated, can be attached to an N-terminal glycine. For example, in BK channels, myristate has been reported to be attached posttranslationally to internal serine/threonine or tyrosine residues via a hydroxyester linkage.

The anchoring moiety can be conjugated to a BAM directly or indirectly via a linker combination, at any chemically feasible location, e.g., at the 5′ and/or 3′ end of a nucleotide sequence, e.g., an ASO. In one aspect, the anchoring moiety is conjugated only to the 3′ end of the BAM. In one aspect, the anchoring moiety is conjugated only to the 5′ end of a nucleotide sequence, e.g., an ASO. In one aspect, the anchoring moiety is conjugated at a location which is not the 3′ end or 5′ end of a nucleotide sequence, e.g., an ASO.

Some types of membrane anchors that can be used to practice the methods of the present disclosure presented in the following table:

| Modification | Modifying Group |
|---|---|
| S-Palmitoylation | 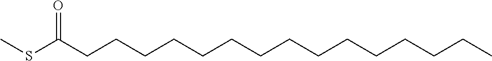 [SMILES: CCCCCCCCCCCCCCCC(=O)SC] |
| N-Palmitoylation | 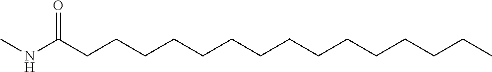 [SMILES: CCCCCCCCCCCCCCCC(=O)NC] |
| N-Myristoylation | 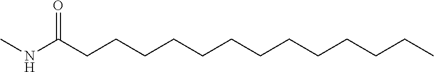 [SMILES: CCCCCCCCCCCCCC(=O)NC] |
| O-Acylation | 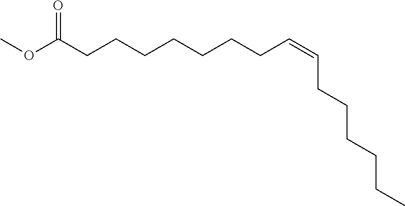 [SMILES: CCCCCC/C=C\CCCCCCCC(=O)OC] |
| Farnesylation | 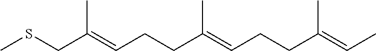 [SMILES: C/C=C(\C)CC/C=C(\C)CC/C=C(\C)CSC] |
| Geranylgeranylation | 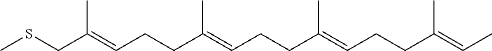 [SMILES: C/C=C(\C)CC/C=C(\C)CC/C=C(\C)CC/C=C(\C)CSC] |
| Cholesterol | 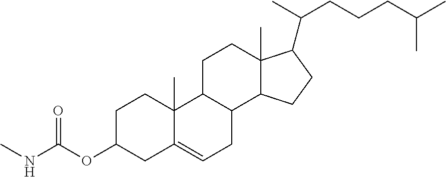 [SMILES: CNC(=O)OC1CCC2(C)C(=CCC3C2CCC2(C)C(C(C)CCCC(C)C)CCC32)C1] |

In some aspects, an anchoring moiety of the present disclosure can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin, etc. which taken together have 6-80 carbon atoms (i.e., an equivalent carbon number (ECN) of 6-80).

In some aspects, the combination of anchoring moieties, e.g., a combination of the lipids (e.g., fatty acids) has an ECN of 6-80, 8-80, 10-80, 12-80, 14-80, 16-80, 18-80, 20-80, 22-80, 24-80, 26-80, 28-80, 30-80, 4-76, 6-76, 8-76, 10-76, 12-76, 14-76, 16-76, 18-76, 20-76, 22-76, 24-76, 26-76, 28-76, 30-76, 6-72, 8-72, 10-72, 12-72, 14-72, 16-72, 18-72, 20-72, 22-72, 24-72, 26-72, 28-72, 30-72, 6-68, 8-68, 10-68, 12-68, 14-68, 16-68, 18-68, 20-68, 22-68, 24-68, 26-68, 28-68, 30-68, 6-64, 8-64, 10-64, 12-64, 14-64, 16-64, 18-64, 20-64, 22-64, 24-64, 26-64, 28-64, 30-64, 6-60, 8-60, 10-60, 12-56, 14-56, 16-56, 18-56, 20-56, 22-56, 24-56, 26-56, 28-56, 30-56, 6-52, 8-52, 10-52, 12-52, 14-52, 16-52, 18-52, 20-52, 22-52, 24-52, 26-52, 28-52, 30-52, 6-48, 8-48, 10-48, 12-48, 14-48, 16-48, 18-48, 20-48, 22-48, 24-48, 26-48, 28-48, 30-48, 6-44, 8-44, 10-44, 12-44, 14-44, 16-44, 18-44, 20-44, 22-44, 24-44, 26-44, 28-44, 30-44, 6-40, 8-40, 10-40, 12-40, 14-40, 16-40, 18-40, 20-40, 22-40, 24-40, 26-40, 28-40, 30-40, 6-36, 8-36, 10-36, 12-36, 14-36, 16-36, 18-36, 20-36, 22-36, 24-36, 26-36, 28-36, 30-36, 6-32, 8-32, 10-32, 12-32, 14-32, 16-32, 18-32, 20-32, 22-32, 24-32, 26-32, 28-32, or 30-32.

II.A.1 Cholesterol and Other Sterols

In some aspects, the anchoring moiety comprises a sterol, steroid, hopanoid, hydroxysteroid, secosteroid, or analog thereof with lipophilic properties. In some aspects, the anchoring moiety comprises a sterol, such as a phytosterol, mycosterol, or zoosterol. Exemplary zoosterols include cholesterol and 24S-hydroxycholesterol; exemplary phytosterols include ergosterol (mycosterol), campesterol, sitosterol, and stigmasterol. In some aspects, the sterol is selected from ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, or stigmasterol. Sterols may be found either as free sterols, acylated (sterol esters), alkylated (steryl alkyl ethers), sulfated (sterol sulfate), or linked to a glycoside moiety (steryl glycosides), which can be itself acylated (acylated sterol glycosides).

In some aspects, the anchoring moiety comprises a steroid. In some aspects, the steroid is selected from dihydrotestosterone, uvaol, hecigenin, diosgenin, progesterone, or cortisol.

For example, sterols may be conjugated to the BAM directly or via a linker combination at the available OH group of the sterol. Exemplary sterols have the general skeleton shown below:

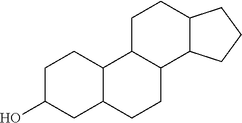 [SMILES: OC1CCC2C(CCC3C4CCCC4CCC23)C1]

As a further example, ergosterol as t e structure below:

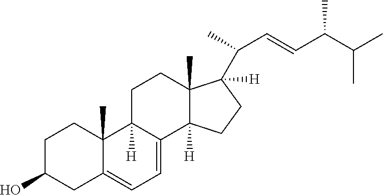

Cholesterol has the structure below:

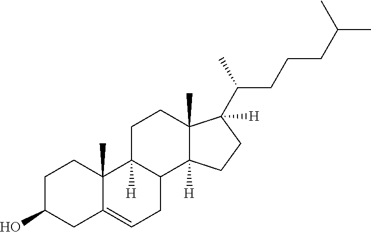

Accordingly, in some aspects, the free OH group of a sterol or steroid is used to conjugate the ASO directly or via a linker combination, to the sterol (e.g., cholesterol) or steroid.

II.A.2. Fatty Acids

In some aspects, the anchoring moiety is a fatty acid. In some aspects, the fatty acid is a shortchain, medium-chain, or long-chain fatty acid. In some aspects, the fatty acid is a saturated fatty acid. In some aspects, the fatty acid is an unsaturated fatty acid. In some aspects, the fatty acid is a monounsaturated fatty acid. In some aspects, the fatty acid is a polyunsaturated fatty acid, such as an ω-3 (omega-3) or ω-6 (omega-6) fatty acid.

In some aspects, the lipid, e.g., fatty acid, has a C₂-C₆₀ chain. In some aspects, the lipid, e.g., fatty acid, has a C₂-C₂₈ chain. In some aspects, the fatty acid, has a C₂-C₄₀ chain. In some aspects, the fatty acid, has a C₂-C₁₂ or C₄-C₁₂ chain. In some aspects, the fatty acid, has a C₄-C₄₀ chain. In some aspects, the fatty acid, has a C₄-C₄₀, C₂-C₃₈, C₂-C₃₆, C₂-C₃₄, C₂-C₃₂, C₂-C₃₀, C₄-C₃₀, C₂-C₂₈, C₄-C₂₈, C₂-C₂₆, C₄-C₂₆, C₂-C₂₄, C₄-C₂₄, C₆-C₂₄, C₈-C₂₄, C₁₀-C₂₄, C₂-C₂₂, C₄-C₂₂, C₆-C₂₂, C₈-C₂₂, C₁₀-C₂₂, C₂-C₂₀, C₄-C₂₀, C₆-C₂₀, C₈-C₂₀, C₁₀-C₂₀, C₂-C₁₈, C₄-C₁₈, C₆-C₁₈, C₈-C₁₈, C₁₀-C₁₈, C₁₂-C₁₈, C₁₄-C₁₈, C₁₆-C₁₈, C₂-C₁₆, C₄-C₁₆, C₆-C₁₆, C₈-C₁₆, C₁₀-C₁₆, C₁₂-C₁₆, C₁₄-C₁₆, C₂-C₁₅, C₄-C₁₅, C₆-C₁₅, C₈-C₁₅, C₉-C₁₅, C₁₀-C₁₅, C₁₁-C₁₅, C₁₂-C₁₅, C₁₃-C₁₅, C₂-C₁₄, C₄-C₁₄, C₆-C₁₄, C₈-C₁₄, C₉-C₁₄, C₁₀-C₁₄, C₁₁-C₁₄, C₁₂-C₁₄, C₂-C₁₃, C₄-C₁₃, C₆-C₁₃, C₇-C₁₃, C₈-C₁₃, C₉-C₁₃, C₁₀-C₁₃, C₁₀-C₁₃, C₁₁-C₁₃, C₂-C₁₃, C₄-C₁₂, C₆-C₁₂, C₇-C₁₂, C₈-C₁₂, C₉-C₁₂, C₁₀-C₁₂, C₂-C₁₁, C₄-C₁₁, C₆-C₁₁, C₇-C₁₁, C₈-C₁₁, C₉-C₁₁, C₂-C₁₀, C₄-C₁₀, C₂-C₉, C₄-C₉, C₂-C₈, C₂-C₇, C₄-C₇, C₂-C₆, or C₄-C₆, chain. In some aspects, the fatty acid, has a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, C₅₀, C₅₁, C₅₂, C₅₃, C₅₄, C₅₅, C₅₆, C₅₇, C₅₈, C₅₉, or C₆₀ chain.

In some aspects, the anchoring moiety comprises two fatty acids, each of which is independently selected from a fatty acid having a chain with any one of the foregoing ranges or numbers of carbon atoms. In some aspects, one of the fatty acids is independently a fatty acid with a C6-C21 chain and one is independently a fatty acid with a C12-C36 chain. In some aspects, each fatty acid independently has a chain of 11, 12, 13, 14, 15, 16, or 17 carbon atoms.

Suitable fatty acids include saturated straight-chain fatty acids, saturated branched fatty acids, unsaturated fatty acids, hydroxy fatty acids, and polycarboxylic acids. In some aspects, such fatty acids have up to 32 carbon atoms.

Examples of useful saturated straight-chain fatty acids include those having an even number of carbon atoms, such as butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid, and heptacosanoic acid.

Examples of suitable saturated branched fatty acids include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, α-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.). Suitable saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid, and 26-methyloctacosanoic acid.

Examples of suitable unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid, 7,10,13,16,19-docosapentaenoic acid, 4,7,10,13,16,19-docosahexaenoic acid, and the like.

Examples of suitable hydroxy fatty acids include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like.

Examples of suitable polycarboxylic acids include oxalic acid, malonic acid, succinic acid, glutaric acid, adipic acid, pimelic acid, suberic acid, azelaic acid, sebacic acid, D,L-malic acid, and the like.

In some aspects, each fatty acid is independently selected from propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, or octatriacontanoic acid.

In some aspects, each fatty acid is independently selected from r-linolenic acid, stearidonic acid, eicosapentaenoic acid, docosahexaenoic acid, linoleic acid, gamma-linoleic acid, dihomno-gamma-linoleic acid, arachidonic acid, docosatetraenoic acid, palmitoleic acid, vaccenic acid, paullinic acid, oleic acid, elaidic acid, gondoic acid, eurcic acid, nervonic acid, mead acid, adrenic acid, bosseopentaenoic acid, ozubondo acid, sardine acid, herring acid, docosahexaenoic acid, or tetracosanolpentaenoic acid, or another monounsaturated or polyunsaturated fatty acid.

In some aspects, one or both of the fatty acids is an essential fatty acid. In view of the beneficial health effects of certain essential fatty acids, the therapeutic benefits of disclosed therapeutic-loaded exosomes may be increased by including such fatty acids in the therapeutic agent. In some aspects, the essential fatty acid is an n-6 or n-3 essential fatty acid selected from the group consisting of linolenic acid, gamma-linolenic acid, dihomo-gamma-linolenic acid, arachidonic acid, adrenic acid, docosapentaenoic n-6 acid, alpha-linolenic acid, stearidonic acid, the 20:4n-3 acid, eicosapentaenoic acid, docosapentaenoic n-3 acid, or docosahexaenoic acid.

In some aspects, each fatty acid is independently selected from all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, or lipoic acid. In other aspects, the fatty acid is selected from eicosapentaenoic acid, docosahexaenoic acid, or lipoic acid. Other examples of fatty acids include all-cis-7,10,13-hexadecatrienoic acid, fc-linolenic acid (ALA or all-cis-9,12,15-octadecatrienoic acid), stearidonic acid (SID or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA), docosapentaenoic acid (DPA, clupadonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In some aspects, the fatty acid is a medium-chain fatty acid such as lipoic acid.

Fatty acid chains differ greatly in the length of their chains and may be categorized according to chain length, e.g as short to very long Short-chain fatty acids (SCFA) are fatty acids with chains of about five or less carbons (e.g. butyric acid). In some aspects, the fatty acid is a SCFA Medium-chain fatty acids (MCFA) include fatty acids with chains of about 6-12 carbons, which can form medium-chain triglycerides. In some aspects, the fatty acid is a MCFA. Long-chain fatty acids (LCFA) include fatty acids with chains of 13-21 carbons. In some aspects, the fatty acid is a LCFA. In some aspects, the fatty acid is a LCFA. Very long chain fatty acids (VLCFA) include fatty acids with chains of 22 or more carbons, such as 22-60, 22-50, or 22-40 carbons. In some aspects, the fatty acid is a VLCFA.

II.A.3. Phospholipids

In some aspects, the anchoring moiety comprises a phospholipid. Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. For example, a phospholipid can be a lipid according to the following formula:

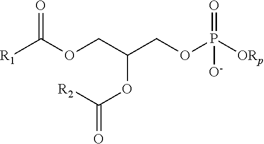

in which Rₚ represents a phospholipid moiety and R₁ and R₂ represent fatty acid moieties with or without unsaturation that may be the same or different.

A phospholipid moiety may be selected, for example, from the non-limiting group consisting of phosphatidyl choline, phosphatidyl ethanolamine, phosphatidyl glycerol, phosphatidyl serine, phosphatidic acid, 2 lysophosphatidyl choline, and a sphingomyelin.

Particular phospholipids may facilitate fusion to a lipid bilayer, e.g., the lipid bilayer of an exosomal membrane. For example, a cationic phospholipid may interact with one or more negatively charged phospholipids of a membrane. Fusion of a phospholipid to a membrane may allow one or more elements of a lipid-containing composition to bind to the membrane or to pass through the membrane.

A fatty acid moiety may be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

The phospholipids using as anchoring moieties in the present disclosure can be natural or non-natural phospholipids. Non-natural phospholipid species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide.

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Examples of phospholipids that can be used in the anchoring moieties disclosed herein include phosphatidylethanolamines (e.g., dilauroylphosphatidyl ethanolamine, dimyristoylphosphatidyl ethanolamine, dipalmitoylphosphatidyl ethanolamine, distearoylphosphatidyl ethanolamine, dioleoylphosphatidyl ethanolamine, 1-palmitoyl-2-oleylphosphatidyl ethanolamine, 1-oleyl-2-palmitoylphosphatidyl ethanolamine, and dierucoylphosphatidyl ethanolamine), phosphatidyl glycerols (e.g., dilauroylphosphatidyl glycerol, dimyristoylphosphatidyl glycerol, dipalmitoylphosphatidyl glycerol, distearoylphosphatidyl glycerol, dioleoylphosphatidyl glycerol, 1-palmitoyl-2-oleyl-phosphatidyl glycerol, 1-oleyl-2-palmitoyl-phosphatidyl glycerol, and dierucoylphosphatidyl glycerol); phosphatidyl serines (e.g., such as dilauroylphosphatidyl serine, dimyristoylphosphatidyl serine, dipalmitoylphosphatidyl serine, distearoylphosphatidyl serine, dioleoylphosphatidyl serine, 1-palmitoyl-2-oleyl-phosphatidyl serine, 1-oleyl-2-palmitoyl-phosphatidyl serine, and dierucoylphosphatidyl serine); phosphatidic acids (e.g., dilauroylphosphatidic acid, dimyristoylphosphatidic acid, dipalmitoylphosphatidic acid, distearoylphosphatidic acid, dioleoylphosphatidic acid, 1-palmitoyl-2-oleylphosphatidic acid, 1-oleyl-2-palmitoyl-phosphatidic acid, and dierucoylphosphatidic acid); and phosphatidyl inositols (e.g., dilauroylphosphatidyl inositol, dimyristoylphosphatidyl inositol, dipalmitoylphosphatidyl inositol, distearoylphosphatidyl inositol, dioleoylphosphatidyl inositol, 1-palmitoyl-2-oleyl-phosphatidyl inositol, 1-oleyl-2-palmitoyl-phosphatidyl inositol, and dierucoylphosphatidyl inositol.

Phospholipids may be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some aspects, the anchoring moiety comprises at least one symmetric phospholipid. Symmetric phospholipids may be selected from the non-limiting group consisting of 1,2 dipropionyl sn-glycero 3 phosphocholine (03:0 PC), 1,2 dibutyryl sn glycero 3 phosphocholine (04:0 PC), 1,2 dipentanoyl sn glycero 3 phosphocholine (05:0 PC), 1,2 dihexanoyl sn glycero 3 phosphocholine (06:0 PC), 1,2 diheptanoyl sn glycero 3 phosphocholine (07:0 PC), 1,2 dioctanoyl sn glycero 3 phosphocholine (08:0 PC), 1,2 dinonanoyl sn glycero 3 phosphocholine (09:0 PC), 1,2 didecanoyl sn glycero 3 phosphocholine (10:0 PC), 1,2 diundecanoyl sn glycero 3 phosphocholine (11:0 PC, DUPC), 1,2 dilauroyl sn glycero 3 phosphocholine (12:0 PC), 1,2 ditridecanoyl sn glycero 3 phosphocholine (13:0 PC), 1,2 dimyristoyl sn glycero 3 phosphocholine (14:0 PC, DMPC), 1,2 dipentadecanoyl sn glycero 3 phosphocholine (15:0 PC), 1,2 dipalmitoyl sn glycero 3 phosphocholine (16:0 PC, DPPC), 1,2 diphytanoyl sn glycero 3 phosphocholine (4ME 16:0 PC), 1,2 diheptadecanoyl sn glycero 3 phosphocholine (17:0 PC), 1,2 distearoyl sn glycero 3 phosphocholine (18:0 PC, DSPC), 1,2 dinonadecanoyl sn glycero 3 phosphocholine (19:0 PC), 1,2 diarachidoyl sn glycero 3 phosphocholine (20:0 PC), 1,2 dihenarachidoyl sn glycero 3 phosphocholine (21:0 PC), 1,2 dibehenoyl sn glycero 3 phosphocholine (22:0 PC), 1,2 ditricosanoyl sn glycero 3 phosphocholine (23:0 PC), 1,2 dilignoceroyl sn glycero 3 phosphocholine (24:0 PC), 1,2 dimyristoleoyl sn glycero 3 phosphocholine (14:1 (Δ9-Cis) PC), 1,2 dimyristelaidoyl sn glycero 3 phosphocholine (14:1 (Δ9-Trans) PC), 1,2 dipalmitoleoyl sn glycero 3 phosphocholine (16:1 (Δ9-Cis) PC), 1,2 dipalmitelaidoyl sn glycero 3 phosphocholine (16:1 (Δ9-Trans) PC), 1,2 dipetroselenoyl sn glycero 3 phosphocholine (18:1 (Δ6-Cis) PC), 1,2 dioleoyl sn glycero 3 phosphocholine (18:1 (Δ9-Cis) PC, DOPC), 1,2 dielaidoyl sn glycero 3 phosphocholine (18:1 (Δ9-Trans) PC), 1,2 dilinoleoyl sn glycero 3 phosphocholine (18:2 (Cis) PC, DLPC), 1,2 dilinolenoyl sn glycero 3 phosphocholine (18:3 (Cis) PC, DLnPC), 1,2 dieicosenoyl sn glycero 3 phosphocholine (20:1 (Cis) PC), 1,2 diarachidonoyl sn glycero 3 phosphocholine (20:4 (Cis) PC, DAPC), 1,2 dierucoyl sn glycero 3 phosphocholine (22:1 (Cis) PC), 1,2 didocosahexaenoyl sn glycero 3 phosphocholine (22:6 (Cis) PC, DHAPC), 1,2 dinervonoyl sn glycero 3 phosphocholine (24:1 (Cis) PC), 1,2 dihexanoyl sn glycero 3 phosphoethanolamine (06:0 PE), 1,2 dioctanoyl sn glycero 3 phosphoethanolamine (08:0 PE), 1,2 didecanoyl sn glycero 3 phosphoethanolamine (10:0 PE), 1,2 dilauroyl sn glycero 3 phosphoethanolamine (12:0 PE), 1,2 dimyristoyl sn glycero 3 phosphoethanolamine (14:0 PE), 1,2 dipentadecanoyl sn glycero 3 phosphoethanolamine (15:0 PE), 1,2 dipalmitoyl sn glycero 3 phosphoethanolamine (16:0 PE), 1,2 diphytanoyl sn glycero 3 phosphoethanolamine (4ME 16:0 PE), 1,2 diheptadecanoyl sn glycero 3 phosphoethanolamine (17:0 PE), 1,2 distearoyl sn glycero 3 phosphoethanolamine (18:0 PE, DSPE), 1,2 dipalmitoleoyl sn glycero 3 phosphoethanolamine (16:1 PE), 1,2 dioleoyl sn glycero 3 phosphoethanolamine (18:1 (Δ9-Cis) PE, DOPE), 1,2 dielaidoyl sn glycero 3 phosphoethanolamine (18:1 (Δ9-Trans) PE), 1,2 dilinoleoyl sn glycero 3 phosphoethanolamine (18:2 PE, DLPE), 1,2 dilinolenoyl sn glycero 3 phosphoethanolamine (18:3 PE, DLnPE), 1,2 diarachidonoyl sn glycero 3 phosphoethanolamine (20:4 PE, DAPE), 1,2 didocosahexaenoyl sn glycero 3 phosphoethanolamine (22:6 PE, DHAPE), 1,2 di O octadecenyl sn glycero 3 phosphocholine (18:0 Diether PC), 1,2 dioleoyl sn glycero 3 phospho rac (1 glycerol) sodium salt (DOPG), and any combination thereof.

In some aspects, the anchoring moiety comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof.

In some aspects, the anchoring moiety comprises at least one asymmetric phospholipid. Asymmetric phospholipids may be selected from the non-limiting group consisting of 1 myristoyl 2 palmitoyl sn glycero 3 phosphocholine (14:0-16:0 PC, MPPC), 1 myristoyl 2 stearoyl sn glycero 3 phosphocholine (14:0-18:0 PC, MSPC), 1 palmitoyl 2 acetyl sn glycero 3 phosphocholine (16:0-02:0 PC), 1 palmitoyl 2 myristoyl sn glycero 3 phosphocholine (16:0-14:0 PC, PMPC), 1 palmitoyl 2 stearoyl sn glycero 3 phosphocholine (16:0-18:0 PC, PSPC), 1 palmitoyl 2 oleoyl sn glycero 3 phosphocholine (16:0-18:1 PC, POPC), 1 palmitoyl 2 linoleoyl sn glycero 3 phosphocholine (16:0-18:2 PC, PLPC), 1 palmitoyl 2 arachidonoyl sn glycero 3 phosphocholine (16:0-20:4 PC), 1 palmitoyl 2 docosahexaenoyl sn glycero 3 phosphocholine (14:0-22:6 PC), 1 stearoyl 2 myristoyl sn glycero 3 phosphocholine (18:0-14:0 PC, SMPC), 1 stearoyl 2 palmitoyl sn glycero 3 phosphocholine (18:0-16:0 PC, SPPC), 1 stearoyl 2 oleoyl sn glycero 3 phosphocholine (18:0-18:1 PC, SOPC), 1 stearoyl 2 linoleoyl sn glycero 3 phosphocholine (18:0-18:2 PC), 1 stearoyl 2 arachidonoyl sn glycero 3 phosphocholine (18:0-20:4 PC), 1 stearoyl 2 docosahexaenoyl sn glycero 3 phosphocholine (18:0-22:6 PC), 1 oleoyl 2 myristoyl sn glycero 3 phosphocholine (18:1-14:0 PC, OMPC), 1 oleoyl 2 palmitoyl sn glycero 3 phosphocholine (18:1-16:0 PC, OPPC), 1 oleoyl 2 stearoyl sn glycero 3 phosphocholine (18:1-18:0 PC, OSPC), 1 palmitoyl 2 oleoyl sn glycero 3 phosphoethanolamine (16:0-18:1 PE, POPE), 1 palmitoyl 2 linoleoyl sn glycero 3 phosphoethanolamine (16:0-18:2 PE), 1 palmitoyl 2 arachidonoyl sn glycero 3 phosphoethanolamine (16:0-20:4 PE), 1 palmitoyl 2 docosahexaenoyl sn glycero 3 phosphoethanolamine (16:0-22:6 PE), 1 stearoyl 2 oleoyl sn glycero 3 phosphoethanolamine (18:0-18:1 PE), 1 stearoyl 2 linoleoyl sn glycero 3 phosphoethanolamine (18:0-18:2 PE), 1 stearoyl 2 arachidonoyl sn glycero 3 phosphoethanolamine (18:0-20:4 PE), 1 stearoyl 2 docosahexaenoyl sn glycero 3 phosphoethanolamine (18:0-22:6 PE), 1 oleoyl 2 cholesterylhemisuccinoyl sn glycero 3 phosphocholine (OChemsPC), and any combination thereof.

To provide more remarkable nuclease resistance, cellular uptake efficiency, and a more remarkable RNA interference effect, phosphatidylethanolamines may be used as anchoring moieties, for example, dimyristoylphosphatidyl ethanolamine, dipalmitoylphosphatidyl ethanolamine, 1-palmitoyl-2-oleyl-phosphatidyl ethanolamine, and dioleoylphosphatidyl ethanolamine.

The binding site of lipid (e.g., a phospholipid) and a linker combination or BAM, e.g., an ASO, may be suitably selected according to the types of lipid and linker or BAM. Any position other than hydrophobic groups of the lipid may be linked to the linker or BAM by a chemical bond. For example, when using a phosphatidylethanolamine, the linkage may be made by forming an amide bond, etc. between the amino group of phosphatidylethanolamine and the linker or BAM. When using a phosphatidylglycerol, the linkage may be made by forming an ester bond, an ether bond, etc. between the hydroxyl group of the glycerol residue and the linker or BAM. When using a phosphatidylserine, the linkage may be made by forming an amide bond or an ester bond, etc. between the amino group or carboxyl group of the serine residue and the linker or BAM. When using a phosphatidic acid, the linkage may be made by forming a phosphoester bond, etc. between the phosphate residue and the linker or BAM. When using a phosphatidylinositol, the linkage may be made by forming an ester bond, an ether bond, etc. between the hydroxyl group of the inositol residue and the linker or BAM.

II.A.4. Lysolipids (e.g., Lysophospholipids)

In some aspects, the anchoring moiety comprises a lysolipid, e.g., a lysophospholipid. Lysolipids are derivatives of a lipid in which one or both fatty acyl chains have been removed, generally by hydrolysis. Lysophospholipids are derivatives of a phospholipid in which one or both fatty acyl chains have been removed by hydrolysis.

In some aspects, the anchoring moiety comprises any of the phospholipids disclosed above, in which one or both acyl chains have been removed via hydrolysis, and therefore the resulting lysophospholipid comprises one or no fatty acid acyl chain.

In some aspects, the anchoring moiety comprises a lysoglycerophospholipid, a lysoglycosphingoliopid, a lysophosphatidylcholine, a lysophosphatidylethanolamine, a lysophosphatidylinositol, or a lysophosphatidylserine.

In some aspect, the anchoring moiety comprises a lysolipid selected from the non-limiting group consisting of 1 hexanoyl 2 hydroxy sn glycero 3 phosphocholine (06:0 Lyso PC), 1 heptanoyl 2 hydroxy sn glycero 3 phosphocholine (07:0 Lyso PC), 1 octanoyl 2 hydroxy sn glycero 3 phosphocholine (08:0 Lyso PC), 1 nonanoyl 2 hydroxy sn glycero 3 phosphocholine (09:0 Lyso PC), 1 decanoyl 2 hydroxy sn glycero 3 phosphocholine (10:0 Lyso PC), 1 undecanoyl 2 hydroxy sn glycero 3 phosphocholine (11:0 Lyso PC), 1 lauroyl 2 hydroxy sn glycero 3 phosphocholine (12:0 Lyso PC), 1 tridecanoyl 2 hydroxy sn glycero 3 phosphocholine (13:0 Lyso PC), 1 myristoyl 2 hydroxy sn glycero 3 phosphocholine (14:0 Lyso PC), 1 pentadecanoyl 2 hydroxy sn glycero 3 phosphocholine (15:0 Lyso PC), 1 palmitoyl 2 hydroxy sn glycero 3 phosphocholine (16:0 Lyso PC), 1 heptadecanoyl 2 hydroxy sn glycero 3 phosphocholine (17:0 Lyso PC), 1 stearoyl 2 hydroxy sn glycero 3 phosphocholine (18:0 Lyso PC), 1 oleoyl 2 hydroxy sn glycero 3 phosphocholine (18:1 Lyso PC), 1 nonadecanoyl 2 hydroxy sn glycero 3 phosphocholine (19:0 Lyso PC), 1 arachidoyl 2 hydroxy sn glycero 3 phosphocholine (20:0 Lyso PC), 1 behenoyl 2 hydroxy sn glycero 3 phosphocholine (22:0 Lyso PC), 1 lignoceroyl 2 hydroxy sn glycero 3 phosphocholine (24:0 Lyso PC), 1 hexacosanoyl 2 hydroxy sn glycero 3 phosphocholine (26:0 Lyso PC), 1 myristoyl 2 hydroxy sn glycero 3 phosphoethanolamine (14:0 Lyso PE), 1 palmitoyl 2 hydroxy sn glycero 3 phosphoethanolamine (16:0 Lyso PE), 1 stearoyl 2 hydroxy sn glycero 3 phosphoethanolamine (18:0 Lyso PE), 1 oleoyl 2 hydroxy sn glycero 3 phosphoethanolamine (18:1 Lyso PE), 1 hexadecyl sn glycero 3 phosphocholine (C16 Lyso PC), and any combination thereof.

II.A.5 Vitamins

In some aspects, the anchoring moiety comprises a lipophilic vitamin, e.g., folic acid, vitamin A, vitamin E, or vitamin K In some aspects, the anchoring moiety comprises vitamin A. Vitamin A is a group of unsaturated nutritional organic compounds that includes retinol, retinal, retinoic acid, and several provitamin A carotenoids (most notably beta-carotene). In some aspects, the anchoring moiety comprises retinol. In some aspects, the anchoring moiety comprises a retinoid. Retinoids are a class of chemical compounds that are vitamers of vitamin A or are chemically related to it. In some aspects, the anchoring moiety comprises a first generation retinoid (e.g., retinol, tretinoin, isotreatinoin, or alitretinoin), a second-generation retinoid (e.g., etretinate or acitretin), a third-generation retinoid (e.g., adapalene, bexarotene, or tazarotene), or any combination thereof

| | First-generation retinoids |
|---|---|
| retinol | 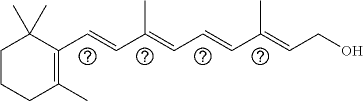 [SMILES: CC1=C(/C=C/C(C)=C/C=C/C(C)=C/CO)C(C)(C)CCC1] |
| tretinoin (alt-trans-retinoic acid) | 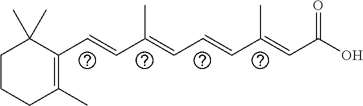 [SMILES: CC1=C(/C=C/C(C)=C/C=C/C(C)=C/C(=O)O)C(C)(C)CCC1] |
| isotretinoin (13-cis-retinoic acid) | 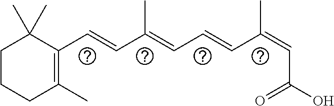 [SMILES: CC1=C(/C=C/C(C)=C/C=C/C(C)=C\C(=O)O)C(C)(C)CCC1] |
| alitretinoin (9-cis-retinoic acid) | 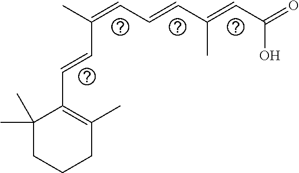 [SMILES: CC1=C(/C=C/C(C)=C\C=C\C(C)=C\C(=O)O)C(C)(C)CCC1] |
| | Second-generation retinoids |
| etretinate | 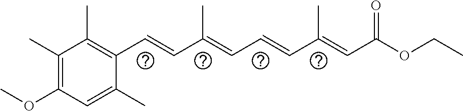 [SMILES: CCOC(=O)/C=C(C)/C=C/C=C(C)/C=C/c1c(C)cc(OC)c(C)c1C] |
| acitretin | 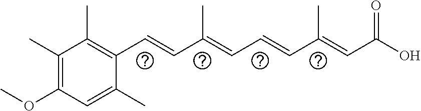 [SMILES: COc1cc(C)c(/C=C/C(C)=C/C=C/C(C)=C/C(=O)O)c(C)c1C] |
| | Third-generation retinoids |
| adapalene | 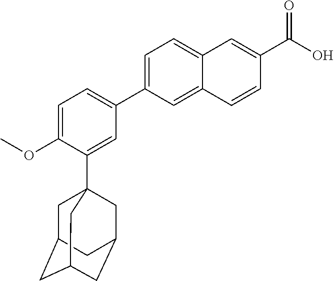 [SMILES: COc1ccc(-c2ccc3cc(C(=O)O)ccc3c2)cc1C12CC3CC(CC(C3)C1)C2] |
| bexarotene | 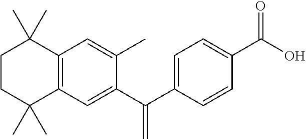 [SMILES: C=C(c1ccc(C(=O)O)cc1)c1cc2c(cc1C)C(C)(C)CCC2(C)C] |
| tazarotene | 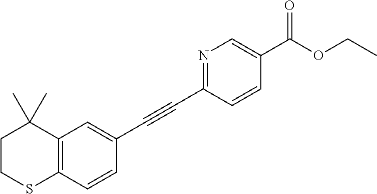 [SMILES: CCOC(=O)c1ccc(C#Cc2ccc3c(c2)C(C)(C)CCS3)nc1] |

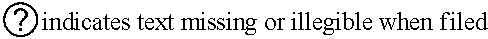

In some aspects, the anchoring moiety comprises vitamin E. Tocopherols are a class of methylated phenols many of which have vitamin E activity. Thus, in some aspects, the anchoring moiety comprises alpha-tocopherol, beta-tocopherol, gamma-tocopherol, delta-tocopherol, or a combination thereof.

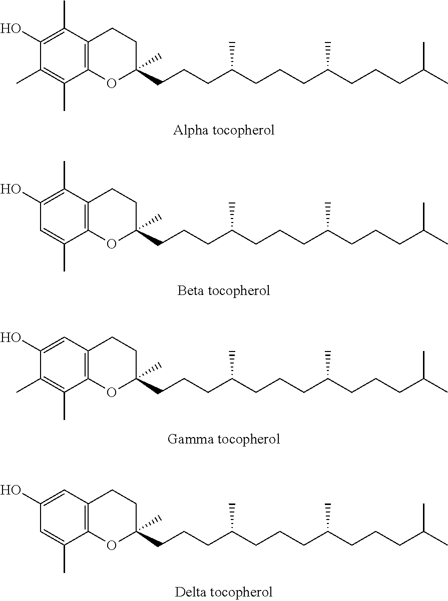 [SMILES: Cc1c(C)c2c(c(C)c1O)CC[C@@](C)(CCC[C@H](C)CCC[C@H](C)CCCC(C)C)O2 ; Cc1cc(O)c(C)c2c1O[C@](C)(CCC[C@H](C)CCC[C@H](C)CCCC(C)C)CC2 ; Cc1c(O)cc2c(c1C)O[C@](C)(CCC[C@H](C)CCC[C@H](C)CCCC(C)C)CC2 ; Cc1cc(O)cc2c1O[C@](C)(CCC[C@H](C)CCC[C@H](C)CCCC(C)C)CC2]

Tocotrienols also have vitamin E activity. The critical chemical structural difference between tocotrienols and tocoherols is that tocotrienols have unsaturated isorenoid side chain with three carbon-carbon double bonds versus saturated side chains for tocopherols. In some aspects, the anchoring moiety comprises alpha-tocotrienol, beta-tocotrienol, gamma-tocotrienol, delta-tocotrienol, or a combination thereof. Tocotrienols can be represented by the formula below

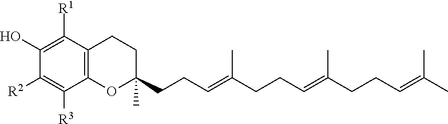

alpha(α)-Tocotrienol: R1=Me, R2=Me, R3=Me;
beta(β)-Tocotrienol: R1=Me, R2=H, R3=Me;
gamma(γ)-Tocotrienol: R1=H, R2=Me, R3=Me;
delta(δ)-Tocotrienol: R1=H, R2=H, R3=Me.

In some aspects, the anchoring moiety comprises vitamin K. Chemically, the vitamin K family comprises 2-methyl-1.4-naphthoquinone (3-) derivatives. Vitamin K includes two natural vitamers: vitamin K₁ and vitamin K₂. The structure of vitamin K₁ (also known as phytonadione, phylloquinone, or (E)-phytonadione) is marked by the presence of a phytyl group. The structures of vitamin K₂ (menaquinones) are marked by the polyisoprenyl side chain present in the molecule that can contain six to 13 isoprenyl units. Thus, vitamin K₂ consists of a number of related chemical subtypes, with differing lengths of carbon side chains made of isoprenoid groups of atoms. MK-4 is the most common form of vitamin K₂. Long chain forms, such as MK-7, MK-8 and MK-9 are predominant in fermented foods. Longer chain forms of vitamin K₂ such as MK-10 to MK-13 are synthesized by bacteria, but they are not well absorbed and have little biological function. In addition to the natural forms of vitamin K, there is a number of synthetic forms of vitamin K such as vitamin K₃ (menadione; 2-methylnaphthalene-1,4-dione), vitamin K₄, and vitamin K₅.

Accordingly, in some aspects, the anchoring moiety comprises vitamin K₁, K2 (e.g., MK-4, MK-5, MK-6, MK-7, MK-8, MK-9, MK-10, MK-11, MK-12, or MK-13), K3, K4, K5, or any combination thereof.

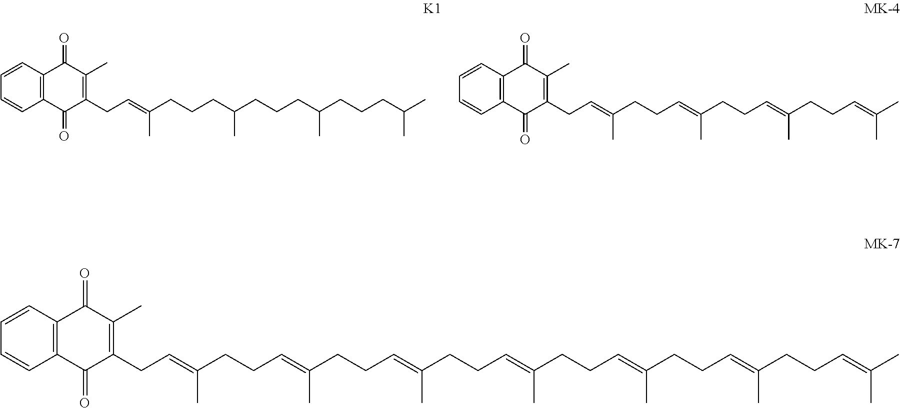

II.B. Linker Combinations

In some aspects, a BAM is linked to a hydrophobic membrane anchoring moiety disclosed herein via a linker combination, which can comprise any combination of cleavable and/or non-cleavable linkers. The main function of a linker combination is to provide the optimal spacing between the anchoring moiety or moieties and the BAM target. For example, in the case of an ASO, the linker combination should reduce steric hindrances and position the ASO so it can interact with a target nucleic acid, e.g., a mRNA or a miRNA.

Linkers may be susceptible to cleavage (“cleavable linker”) thereby facilitating release of the biologically active molecule. Thus, in some aspects, a linker combination disclosed herein can comprise a cleavable linker. Such cleavable linkers may be susceptible, for example, to acid-induced cleavage, photo-induced cleavage, peptidase-induced cleavage, esterase-induced cleavage, and disulfide bond cleavage, at conditions under which the biologically active molecule remains active. Alternatively, linkers may be substantially resistant to cleavage (“non-cleavable linker”). In some aspects, the cleavable linker comprises a spacer. In some aspects the spacer is PEG.

In some aspects, a linker combination comprises at least 2, at least 3, at least 4, at least 5, or at least 6 or more different linkers disclosed herein. In some aspects, linkers in a linker combination can be linked by an ester linkage (e.g., phosphodiester or phosphorothioate ester).

In some aspects, the linker is direct bond between an anchoring moiety and a BAM, e.g., an ASO.

II.B.1 Non-Cleavable Linkers

In some aspects, the linker combination comprises a “non-cleavable liker.” Non-cleavable linkers are any chemical moiety capable of linking two or more components of a modified biologically active molecule of the present disclosure (e.g., a biologically active molecule and an anchoring moiety; a biologically active molecule and a cleavable linker; an anchoring moiety and a cleavable linker) in a stable, covalent manner and does not fall off under the categories listed above for cleavable linkers. Thus, non-cleavable linkers are substantially resistant to acid-induced cleavage, photo-induced cleavage, peptidase-induced cleavage, esterase-induced cleavage and disulfide bond cleavage.

Furthermore, non-cleavable refers to the ability of the chemical bond in the linker or adjoining to the linker to withstand cleavage induced by an acid, photolabile-cleaving agent, a peptidase, an esterase, or a chemical or physiological compound that cleaves a disulfide bond, at conditions under which a cyclic dinucleotide and/or the antibody does not lose its activity. In some aspects, the biologically active molecule is attached to the linker via another linker, e.g., a self-immolative linker.

In some aspects, the linker combination comprises a non-cleavable linker comprising, e.g., tetraethylene glycol (TEG), hexaethylene glycol (HEG), polyethylene glycol (PEG), succinimide, or any combination thereof. In some aspects, the non-cleavable linker comprises a spacer unit to link the biologically active molecule to the non-cleavable linker.

In some aspects, one or more non-cleavable linkers comprise smaller units (e.g., HEG, TEG, glycerol, C2 to C12 alkyl, and the like) linked together. In one aspect, the linkage is an ester linkage (e.g., phosphodiester or phosphorothioate ester) or other linkage.

II.B.1.a. Ethylene Glycols (HEG, TEG, PEG)

In some aspects, the linker combination comprises a non-cleavable linker, wherein the non-cleavable linker comprises a polyethylene glycol (PEG) characterized by a formula R³—(O—CH₂—CH₂)ₙ— or R³—(O—CH₂—CH₂)ₙ—O— with R3 being hydrogen, methyl or ethyl and n having a value from 2 to 200. In some aspects, the linker comprises a spacer, wherein the spacer is PEG.

In some aspects, the PEG linker is an oligo-ethylene glycol, e.g., diethylene glycol, triethylene glycol, tetra ethylene glycol (TEG), pentaethylene glycol, or a hexaethylene glycol (HEG) linker.

In some aspects, n has a value of 2, 3, 4, 5, 6, 7, 8, 9, 10, 11, 12, 13, 14, 15, 16, 17,18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200. In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200. In some specific aspects, n has a value from 3 to 200, from 3 to 20, from 10 to 30, or from 9 to 45. In some aspects, the PEG is a branched PEG. Branched PEGs have three to ten PEG chains emanating from a central core group.

In certain aspects, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group. In some aspects, the PEG is a Comb PEGs. Comb PEGs have multiple PEG chains normally grafted onto a polymer backbone.

In certain aspects, the PEG has a molar mass between 100 g/mol and 3000 g/mol, particularly between 100 g/mol and 2500 g/mol, more particularly of approx. 100 g/mol to 2000 g/mol. In certain aspects, the PEG has a molar mass between 200 g/mol and 3000 g/mol, particularly between 300 g/mol and 2500 g/mol, more particularly of approx. 400 g/mol to 2000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₈₀₀, PEG₂₉₀₀, or PEG₃₀₀₀. In one particular aspect, the PEG is PEG₄₀₀. In another particular aspect, the PEG is PEG₂₀₀₀.

In some aspects, a linker combination of the present disclosure can comprise several PEG linkers, e.g., a cleavable linker flanked by PEG, HEG, or TEG linkers. In some aspects, the linker combination comprises (HEG)n and/or (TEG)n, wherein n is an integer between 1 and 50, and each unit is connected, e.g., via a phosphate ester linker, a phosphorothioate ester linkage, or a combination thereof.

II.B.1.b. Glycerol and Polyglycerols (PG)

In some aspects, the linker combination comprises a non-cleavable linker comprising a glycerol unit or a polyglycerol (PG) described by the formula ((R3-O—(CH₂—CHOH—CH₂O)ₙ—) with R3 being hydrogen, methyl or ethyl, and n having a value from 3 to 200. In some aspects, n has a value from 3 to 20. In some aspects, n has a value from 10 to 30.

In some aspects, the PG linker is a diglycerol, triglycerol, tetraglycerol (TG), pentaglycerol, or a hexaglycerol (HG) linker. In some aspects, n has a value of 2, 3, 4, 5, 6, 7, 8, 9, 10, 11, 12, 13, 14, 15, 16, 17,18,19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200. In some aspects, n is between 2 and 10, between 10 and 20, between 20 and 30, between 30 and 40, between 40 and 50, between 50 and 60, between 60 and 70, between 70 and 80, between 80 and 90, between 90 and 100, between 100 and 110, between 110 and 120, between 120 and 130, between 130 and 140, between 140 and 150, between 150 and 160, between 160 and 170, between 170 and 180, between 180 and 190, or between 190 and 200.

In some alternatives of these aspects, n has a value from 9 to 45. In some aspects, the heterologous moiety is a branched polyglycerol described by the formula (R³—O—(CH₂—CHOR⁵—CH₂—O)ₙ—) with R⁵ being hydrogen or a linear glycerol chain described by the formula (R³—O—(CH₂—CHOH—CH₂—O)ₙ—) and R3 being hydrogen, methyl or ethyl. In some aspects, the heterologous moiety is a hyperbranched polyglycerol described by the formula (R³—O—(CH₂—CHOR⁵—CH₂—O)ₙ—) with R⁵ being hydrogen or a glycerol chain described by the formula (R³—O—(CH₂—CHOR⁶—CH₂—O)ₙ—), with R⁶ being hydrogen or a glycerol chain described by the formula (R³—O—(CH₂—CHOR⁷—CH₂—O)ₙ—), with R⁷ being hydrogen or a linear glycerol chain described by the formula (R³—O—(CH₂—CHOH—CH₂—O)ₙ—) and R3 being hydrogen, methyl or ethyl. Hyperbranched glycerol and methods for its synthesis are described in Oudshorn et al. (2006) Biomaterials 27:5471-5479; Wilms et al. (20100 Acc. Chem. Res. 43, 129-41, and references cited therein.

In certain aspects, the PG has a molar mass between 100 g/mol and 3000 g/mol, particularly between 100 g/mol and 2500 g/mol, more particularly of approx. 100 g/mol to 2000 g/mol. In certain aspects, the PG has a molar mass between 200 g/mol and 3000 g/mol, particularly between 300 g/mol and 2500 g/mol, more particularly of approx. 400 g/mol to 2000 g/mol.

In some aspects, the PG is PG₁₀₀, PG₂₀₀, PG₃₀₀, PG₄₀₀, PG₅₀₀, PG₆₀₀, PG₇₀₀, PG₈₀₀, PG₉₀₀, PG₁₀₀₀, PG₁₁₀₀, PG₁₂₀₀, PG₁₃₀₀, PG₁₄₀₀, PG₁₅₀₀, PG₁₆₀₀, PG₁₇₀₀, PG₁₈₀₀, PG₁₀₀₀, PG₂₀₀₀, PG₂₁₀₀, PG₂₂₀₀, PG₂₃₀₀, PG₂₄₀₀, PG₂₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₂₇₀₀, PG₂₈₀₀, PG₂₉₀₀, or PG₃₀₀₀. In one particular aspect, the PG is PG₄₀₀. In another particular aspect, the PG is PG₂₀₀₀.

In some aspects, the linker combination comprises (glycerol)n, and/or (HG)n and/or (TG)n, wherein n is an integer between 1 and 50, and each unit is connected, e.g., via a phosphate ester linker, a phosphorothioate ester linkage, or a combination thereof.

II.B.1.c. Aliphatic (Alkyl) Linkers

In some aspects, the linker combination comprises at least one aliphatic (alkyl) linker, e.g., propyl, butyl, hexyl, or C2-C12 alkyl, such as C2-C10 alkyl or C2-C6 alkyl.

In some aspects, the linker combination comprises an alkyl chain, e.g., an unsubstituted alkyl. In some aspects, the linker combination comprises an substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, Aryl, heteroaryl, heterocyclyl, cycloalkyl, cycloalkenyl, alkylarylalkyl, alkylarylalkenyl, alkylarylalkynyl, alkenylarylalkyl, alkenyl Reyl alkenyl, alkenyl aryl alkynyl, alkynyl aryl alkyl, alkynyl aryl alkenyl, alkynyl aryl alkynyl, alkyl heteroaryl alkyl, alkyl heteroaryl alkyl, alkyl heteroaryl alkenyl, alkyl heteroaryl alkynyl, alkenyl heteroaryl alkyl, alkenyl heteroaryl alkenyl, alkenyl heteroaryl alkynyl, alkynyl Heteroarylalkyl, alkynylheteroarylalkenyl, alkynylheteroarylalkynyl, alkylheterocyclylalkyl, alkylheterocyclylalkenyl, alkylheterocyclylalkynyl, alkenylheterocyclylalkyl, alkenylheterocyclylalkenyl, or alkenylheterocyclylalkynyl.

Optionally these components are substituted. Substituents include alcohol, alkoxy (such as methoxy, ethoxy, and propoxy), straight or branched chain alkyl (such as C1-C12 alkyl), amine, aminoalkyl (such as amino C1-C12 alkyl), phosphoramidite, phosphate, phosphoramidate, phosphorodithioate, thiophosphate, hydrazide, hydrazine, halogen, (such as F, Cl, Br, or I), amide, alkylamide (such as amide C1-C12 alkyl), carboxylic acid, carboxylic ester, carboxylic anhydride, carboxylic acid halide, ether, sulfonyl halide, imidate ester, isocyanate, isothiocyanate, haloformate, carboduimide adduct, aldehydes, ketone, sulfhydryl, haloacetyl, alkyl halide, alkyl sulfonate, C(═O)CH═CHC(═O) (maleimide), thioether, cyano, sugar (such as mannose, galactose, and glucose), α,β-unsaturated carbonyl, alkyl mercurial, or α,β-unsaturated sulfone.

The term “alkyl,” by itself or as part of another substituent, means, unless otherwise stated, a straight or branched chain hydrocarbon radical having the number of carbon atoms designated (e.g., C₁-C₁₀ means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one aspect, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁₀, or C₂-C₈) and at least one heteroatom chosen, e.g., from N, O, S, Si, B and P (in one aspect, N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —CH₂—Si(CH₃)₃, —CH₂—CH═N—OCH₃, and —CH═CH—N(CH₃)—CH₃. Up to two heteroatoms can be consecutive, such as, for example, —CH₂—NH—OCH₃ and —CH₂—O—Si(CH₃)₃.

Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring selected from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl,” “heterocyclic,” “heterocycle,” or “heterocyclyl,” by itself or in combination with other terms, represents a carbocyclic, non-aromatic ring (e.g., 3- to 8-membered ring and for example, 4-, 5-, 6- or 7-membered ring) containing at least one and up to 5 heteroatoms selected from, e.g., N, O, S, Si, B and P (for example, N, O and S), wherein the nitrogen, sulfur and phosphorus atoms are optionally oxidized, and the nitrogen atom(s) are optionally quaternized (e.g., from 1 to 4 heteroatoms selected from nitrogen, oxygen and sulfur), or a fused ring system of 4- to 8-membered rings, containing at least one and up to 10 heteroatoms (e.g., from 1 to 5 heteroatoms selected from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule.

Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is bonded to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. In one aspects, the aryl group is selected from phenyl, benzo[d][1,3]dioxolyl and naphthyl. The aryl group, in yet another aspect, is phenyl.

The term “arylalkyl” or “aralkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” or “aralkyl” groups include benzyl, phenethyl, pyridylmethyl and the like.

By “aryloxy” is meant the group —O-aryl, where aryl is as defined herein. In one example, the aryl portion of the aryloxy group is phenyl or naphthyl. The aryl portion of the aryloxy group, in one aspect, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) selected from N, O, S, Si and B (for example, N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom.

In one example, the heteroaryl group has from 4 to 10 carbon atoms and from 1 to 5 heteroatoms selected from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable aryl group substituents described below.

Examples of aliphatic linkers include the following structures: —O—CO—O—; —NH—CO—O—; —NH—CONH—; —NH—(CH₂)ₙ₁—; —S(CH₂)ₙ₁—; —CO—(CH₂)ₙ₁—CO—; CO—(CH₂)ₙ₁—NH—; —NH—(CH₂)ₙ₁—NH—; —CO—NH—(CH₂)ₙ₁—NH—CO—; —C(═S)—NH—(CH₂)ₙ₁—NH—CO—; —C(═S)—NH—(CH₂)ₙ₁—NH—C-(═S); —CO—O—(CH₂)ₙ₁—O—CO; —C(═S)—O—(CH₂)ₙ₁—O—CO—; —C(═S)—O—(CH₂)ₙ₁—O—C(═S)—; —CO—NH—(CH₂)ₙ₁—O—CO—; —C(═S)—NH—(CH₂)ₙ₁—O—CO—; —C(═S)—NH—(CH₂)ₙ₁—O—C(═S)—; —CO—NH—(CH₂)ₙ₁—O—CO—; C(═S)—NH—(CH₂)ₙ₁—CO—; —C(═S)—O—(CH₂)ₙ₁−NH—CO—; —C(═S)—NH—(CH₂)ₙ₁—O—C(═S)—; —NH—(CH₂CH₂O)ₙ₂—CH(CH₂OH)—; —NH—(CH₂CH₂O)ₙ₂—CH₂—; —NH—(CH₂CH₂O)ₙ₂—CH₂—CO—; —O—(CH₂)ₙ₃—S—S—(CH₂)ₙ₄—O—P(═O)₂—; —CO—(CH₂)ₙ₃—O—CO—NH—(CH₂)ₙ₄—; —CO—(CH₂)ₙ₃—CO—NH—(CH₂)ₙ₄—; —(CH₂)ₙ₁NH—; —C(O)(CH2)ₙ₁NH—; —C(O)—(CH2)ₙ₁—C(O)—; —C(O)—(CH2)ₙ₁—C(O)O—; —C(O)—O—; —C(O)—(CH2)ₙ₁—NH—C(O)—; —C(O)—(CH₂)ₙ₁—; —C(O)—NH—; —C(O)—; —(CH2)ₙ₁—C(O)—; —(CH2)ₙ₁—C(O)O—; —(CH2)ₙ₁—; —(CH2)ₙ₁—NH—C(O)—; wherein n1 is an integer between 1 and 40 (e.g., 2 to 20, or 2 to 12); n2 is an integer between 1 and 20 (e.g., 1 to 10, or 1 to 6); n3 and n4 may be the same or different, and are an integer between 1 and 20 (e.g., 1 to 10, or 1 to 6).

In some aspects, the linker combination comprises (C3)n, (C4)n, (C5)n, (C6)n, (C7)n, or (C8)n, or a combination thereof, wherein n is an integer between 1 and 50, and each unit is connected, e.g., via a phosphate ester linker, a phosphorothioate ester linkage, or a combination thereof.

II.B.2. Cleavable Linkers

In some aspects, different components of an ASO disclosed herein can be linker by a cleavable linker. The term cleavable linker refers to a linker comprising at least one linkage or chemical bond that can be broken or cleaved. As used herein, the term cleave refers to the breaking of one or more chemical bonds in a relatively large molecule in a manner that produces two or more relatively smaller molecules. Cleavage may be mediated, e.g., by a nuclease, peptidase, protease, phosphatase, oxidase, or reductase, for example, or by specific physicochemical conditions, e.g., redox environment, pH, presence of reactive oxygen species, or specific wavelengths of light.

In some aspects, the term “cleavable,” as used herein, refers, e.g., to rapidly degradable linkers, such as, e.g., phosphodiester and disulfides, while the term “non-cleavable” refers, e.g., to more stable linkages, such as, e.g., nuclease-resistant phosphorothioates.

In some aspects, the cleavable linker is a dinucleotide or trinucleotide linker, a disulfide, an imine, a thioketal, a val-cit dipeptide, or any combination thereof.

In some aspects, the cleavable linker comprises valine-alanine-p-aminobenzylcarbamate or valine-citrulline-p-aminobenzylcarbamate.

II.B.2.a. Redox Cleavable Linkers

In some aspects, the linker combination comprises a redox cleavable linker. As a non-limiting example, one type of cleavable linker is a redox cleavable linking group that is cleaved upon reduction or upon oxidation. In some aspects, the redox cleavable linker contains a disulfide bond, i.e., it is a disulfide cleavable linker. Redox cleavable linkers can be reduced, e.g., by intracellular mercaptans, oxidases, or reductases.

II.B.2.b. Reactive Oxygen Species (ROS) Cleavable Linkers

In some aspects, the linker combination can comprise a cleavable linker which may be cleaved by a reactive oxygen species (ROS), such as superoxide (Of) or hydrogen peroxide (H2O2), generated, e.g., by inflammation processes such as activated neutrophils. In some aspects, the ROS cleavable linker is a thioketal cleavable linker. See, e.g., U.S. Pat. No. 8,354,455B2, which is herein incorporated by reference in its entirety.

II.B.2.c. pH Dependent Cleavable Linkers

In some aspects, the linker is an “acid labile linker” comprising an acid cleavable linking group, which is a linking group that is selectively cleaved under acidic conditions (pH<7).

As a non-limiting example, the acid cleavable linking group is cleaved in an acidic environment, e.g., about 6.0, 5.5, 5.0 or less. In some aspects, the pH is about 6.5 or less. In some aspects, the linker is cleaved by an agent such as an enzyme that can act as a general acid, e.g., a peptidase (which may be substrate specific) or a phosphatase. Within cells, certain low pH organelles, such as endosomes and lysosomes, can provide a cleaving environment to the acid cleavable linking group. Although the pH of human serum is 7.4, the average pH in cells is slightly lower, ranging from about 7.1 to 7.3. Endosomes also have an acidic pH, ranging from 5.5 to 6.0, and lysosomes are about 5.0 at an even more acidic pH. Accordingly, pH dependent cleavable linkers are sometimes called endosomically labile linkers in the art.

The acid cleavable group may have the general formula —C═NN—, C(O)O, or —OC(O). In another non-limiting example, when the carbon attached to the ester oxygen (alkoxy group) is attached to an aryl group, a substituted alkyl group, or a tertiary alkyl group such as dimethyl pentyl or t-butyl, for example. Examples of acid cleavable linking groups include, but are not limited to amine, imine, amino ester, benzoic imine, diortho ester, polyphosphoester, polyphosphazene, acetal, vinyl ether, hydrazone, cis-aconitate, hydrazide, thiocarbamoyl, imizine, azidomethyl-methylmaleic anhydride, thiopropionate, a masked endosomolytic agent, a citraconyl group, or any combination thereof. Disulfide linkages are also susceptible to pH.

In some aspects, the linker comprises a low pH-labile hydrazone bond. Such acid-labile bonds have been extensively used in the field of conjugates, e.g., antibody-drug conjugates. See, for example, Zhou et al (2011) Biomacromolecules 12:1460-7; Yuan et al (2008) Acta Biomater. 4:1024-37; Zhang et al (2007) Acta Biomater. 6:838-50; Yang et al (2007) J. Pharmacol. Exp. Ther. 321:462-8; Reddy et al (2006) Cancer Chemother. Pharmacol. 58:229-36; Doronina et al (2003) Nature Biotechnol. 21:778-84.

In certain aspects, the linker comprises a low pH-labile bond selected from the following: ketals that are labile in acidic environments (e.g., pH less than 7, greater than about 4) to form a diol and a ketone; acetals that are labile in acidic environments (e.g., pH less than 7, greater than about 4) to form a diol and an aldehyde; imines or iminiums that are labile in acidic environments (e.g., pH less than 7, greater than about 4) to form an amine and an aldehyde or a ketone; silicon-oxygen-carbon linkages that are labile under acidic condition; silicon-nitrogen (silazane) linkages; silicon-carbon linkages (e.g., arylsilanes, vinylsilanes, and allylsilanes); maleamates (amide bonds synthesized from maleic anhydride derivatives and amines); ortho esters; hydrazones; activated carboxylic acid derivatives (e.g., esters, amides) designed to undergo acid catalyzed hydrolysis); or vinyl ethers.

Further examples may be found in U.S. Pat. Nos. 9,790,494B2 and 8,137,695B2, the contents of which are incorporated herein by reference in their entireties.

II.B.2.d. Enzymatic Cleavable Linkers

In some aspects, the linker combination can comprise a linker cleavable by intracellular or extracellular enzymes, e.g., proteases, esterases, nucleases, amidades. The range of enzymes that can cleave a specific linker in a linker combination depends on the specific bonds and chemical structure of the linker. Accordingly, peptidic linkers can be cleaved, e.g., by peptidades, linkers containing ester linkages can be cleaved, e.g., by esterases; linkers containing amide linkages can be cleaved, e.g., by amidades; etc.

II.B.2.e. Protease Cleavable Linkers

In some aspects, the linker combination comprises a protease cleavable linker, i.e., a linker that can be cleaved by an endogenous protease. Only certain peptides are readily cleaved inside or outside cells. See, e.g., Trout et al., 79 Proc. Natl. Acad. Sci. USA, 626-629 (1982) and Umemoto et al. 43 Int. J. Cancer, 677-684 (1989). Cleavable linkers can contain cleavable sites composed of α-amino acid units and peptidic bonds, which chemically are amide bonds between the carboxylate of one amino acid and the amino group of a second amino acid. Other amide bonds, such as the bond between a carboxylate and the α-amino acid group of lysine, are understood not to be peptidic bonds and are considered non-cleavable.

In some aspects, the protease-cleavable linker comprises a cleavage site for a protease, e.g., neprilysin (CALLA or CDlO), thimet oligopeptidase (TOP), leukotriene A4 hydrolase, endothelin converting enzymes, ste24 protease, neurolysin, mitochondrial intermediate peptidase, interstitial collagenases, collagenases, stromelysins, macrophage elastase, matrilysin, gelatinases, meprins, procollagen C-endopeptidases, procollagen N-endopeptidases, ADAMs and ADAMTs metalloproteinases, myelin associated metalloproteinases, enamelysin, tumor necrosis factor α-converting enzyme, insulysin, nardilysin, mitochondrial processing peptidase, magnolysin, dactylysin-like metalloproteases, neutrophil collagenase, matrix metallopeptidases, membrane-type matrix metalloproteinases, SP2 endopeptidase, prostate specific antigen (PSA), plasmin, urokinase, human fibroblast activation protein (FAPα), trypsin, chymotrypsins, caldecrin, pancreatic elastases, pancreatic endopeptidase, enteropeptidase, leukocyte elastase, myeloblasts, chymases, tryptase, granzyme, stratum corneum chymotryptic enzyme, acrosin, kallikreins, complement components and factors, alternative-complement pathway c3/c5 convertase, mannose-binding protein-associated serine protease, coagulation factors, thrombin, protein c, u and t-type plasminogen activator, cathepsin G, hepsin, prostasin, hepatocyte growth factor-activating endopeptidase, subtilisin/kexin type proprotein convertases, furin, proprotein convertases, prolyl peptidases, acylaminoacyl peptidase, peptidyl-glycaminase, signal peptidase, n-terminal nucleophile aminohydrolases, 20s proteasome, γ-glutamyl transpeptidase, mitochondrial endopeptidase, mitochondrial endopeptidase Ia, htra2 peptidase, matriptase, site 1 protease, legumain, cathepsins, cysteine cathepsins, calpains, ubiquitin isopeptidase T, caspases, glycosylphosphatidylinositoliprotein transamidase, cancer procoagulant, prohormone thiol protease, γ-Glutamyl hydrolase, bleomycin hydrolase, seprase, cathepsin B, cathepsin D, cathepsin L, cathepsin M, proteinase K, pepsins, chymosyn, gastricsin, renin, yapsin and/or mapsins, Prostate-Specific antigen (PSA), or any Asp-N, Glu-C, Lys-C or Arg-C proteases in general. See, e.g., Cancer Res. 77(24):7027-7037 (2017), which is herein incorporated by reference in its entirety.

In some aspects, the cleavable linker component comprises a peptide comprising one to ten amino acid residues. In these aspects, the peptide allows for cleavage of the linker by a protease, thereby facilitating release of the biologically active molecule upon exposure to intracellular proteases, such as lysosomal enzymes (Doronina et al. (2003) Nat. Biotechnol. 21:778-784). Exemplary peptides include, but are not limited to, dipeptides, tripeptides, tetrapeptides, pentapeptides, and hexapeptides.

A peptide may comprise naturally-occurring and/or non-natural amino acid residues. The term “naturally-occurring amino acid” refer to Ala, Asp, Cys, Glu, Phe, Gly, His, He, Lys, Leu, Met, Asn, Pro, Gin, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like. Peptides can be designed and optimized for enzymatic cleavage by a particular enzyme, for example, a tumor-associated protease, cathepsin B, C and D, or a plasmin protease.

Amino acids also include the D-forms of natural and non-natural amino acids. “D-” designates an amino acid having the “D” (dextrorotary) configuration, as opposed to the configuration in the naturally occurring (“L-”) amino acids. Natural and non-natural amino acids can be purchased commercially (Sigma Chemical Co., Advanced Chemtech) or synthesized using methods known in the art. Exemplary dipeptides include, but are not limited to, valine-alanine, valine-citrulline, phenylalanine-lysine, N-methyl-valine-citrulline, cyclohexylalanine-lysine, and beta-alanine-lysine. Exemplary tripeptides include, but are not limited to, glycine-valine-citrulline (gly-val-cit) and glycine-glycine-glycine (gly-gly-gly).

II.B.2.f. Esterase Cleavable Linkers

Some linkers are cleaved by esterases (“esterase cleavable linkers”). Only certain esters can be cleaved by esterases and amidases present inside or outside of cells. Esters are formed by the condensation of a carboxylic acid and an alcohol. Simple esters are esters produced with simple alcohols, such as aliphatic alcohols, and small cyclic and small aromatic alcohols. Examples of ester-based cleavable linking groups include, but are not limited to, esters of alkylene, alkenylene and alkynylene groups. The ester cleavable linking group has the general formula —C(O)O— or —OC (O)—.

II.B.2.g. Phosphatase Cleavable Linkers

In some aspects, a linker combination can includes a phosphate-based cleavable linking group is cleaved by an agent that degrades or hydrolyzes phosphate groups. An example of an agent that cleaves intracellular phosphate groups is an enzyme such as intracellular phosphatase. Examples of phosphate-based linking groups are —O—P(O)(ORₖ)—O—, —O—P(S)(ORₖ)—O—, —O—P(S)(SRₖ)—O—, —S—P(O)(ORₖ)—O—, —O—P(O)(ORₖ)—S—, —S—P(O)(ORₖ)—S—, —O—P(S)(ORₖ)—S—, —SP (S)(ORₖ)—O—, —OP(O)(Rₖ)—O—, —OP(S)(Rₖ)—O—, —SP(O)(Rₖ)—O—, —SP(S)(Rₖ)—O—, —SP(O)(Rₖ)—S—, or —OP(S)(Rₖ)—S—. In various aspects, Rₖ is any of the following: NH₂, BH₃, CH₃, C₁₋₆ alkyl, C₆₋₁₀ aryl, C₁₋₆ alkoxy and C₆₋₁₀ aryl-oxy. In some aspects, C₁₋₆ alkyl and C₆₋₁₀ aryl are unsubstituted. Further non-limiting examples are —O—P(O)(OH)—O—, —O—P(S)(OH)—O—, —O—P(S)(SH)—O—, —S—P(O)(OH)—O—, O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —SP(S)(H)—O—, —SP(O)(H)—S—, —OP(S)(H)—S—, or —O—P(O)(OH)—O—.

II.B.2.h. Photoactivated Cleavable Linkers

In some aspects, the combination linker comprises a photoactivated cleavable linker, e.g., a nitrobenzyl linker or a linker comprising a nitrobenzyl reactive group.

II.B.2.i. Self-Immolative Linker

In some aspects, the linker combination comprises a self-immolative linker In some aspects, the self-immolative linker in the EV (e.g., exosome) of the present disclosure undergoes 1,4 elimination after the enzymatic cleavage of the protease-cleavable linker. In some aspects, the self-immolative linker in the EV (e.g., exosome) of the present disclosure undergoes 1,6 elimination after the enzymatic cleavage of the protease-cleavable linker. In some aspects, the self-immolative linker is, e.g., a p-aminobenzyl (pAB) derivative, such as a p-aminobenzyl carbamate (pABC), a p-amino benzyl ether (PABE), a p-amino benzyl carbonate, or a combination thereof. In certain aspects, the self-immolative linker comprises an aromatic group. In some aspects, the aromatic group is selected from the group consisting of benzyl, cinnamyl, naphthyl, and biphenyl. In some aspects, the aromatic group is heterocyclic. In other aspects, the aromatic group comprises at least one substituent. In some aspects, the at least one substituent is selected from the group consisting of F, Cl, I, Br, OH, methyl, methoxy, NO₂, NH₂, NO³⁺, NHCOCH₃, N(CH₃)₂, NHCOCF₃, alkyl, haloalkyl, C₁-C₈ alkylhalide, carboxylate, sulfate, sulfamate, and sulfonate. In other aspects, at least one C in the aromatic group is substituted with N, O, or C—R*, wherein R* is independently selected from H, F, Cl, I, Br, OH, methyl, methoxy, NO₂, NH₂, NO³⁺, NHCOCH₃, N(CH₃)₂, NHCOCF₃, alkyl, haloalkyl, C₁-C₈ alkylhalide, carboxylate, sulfate, sulfamate, and sulfonate.

In some aspects, the self-immolative linker comprises an aminobenzyl carbamate group (e.g., para-aminobenzyl carbamate), an aminobenzyl ether group, or an aminobenzyl carbonate group. In one aspect, the self-immolative linker is p-amino benzyl carbamate (pABC). pABC is the most efficient and most widespread connector linkage for self-immolative site-specific prodrug activation (see, e.g., Carl et al. J. Med. Chem. 24:479-480 (1981); WO 1981/001145; Rautio et al, Nature Rev. Drug Disc. 7:255-270 (2008); Simplicio et al., Molecules 13:519-547 (2008)).

In some aspects, the self-immolative linker connects a biologically active molecule (e.g., an ASO) to a protease-cleavable substrate (e.g, Val-Cit). In specific aspects, the carbamate group of a pABC self-immolative linker is connected to an amino group of a biologically active molecule (e.g., ASO), and the amino group of the pABC self-immolative linker is connected to a protease-cleavable substrate.

The aromatic ring of the aminobenzyl group can optionally be substituted with one or more (e.g., R₁ and/or R₂) substituents on the aromatic ring, which replace a hydrogen that is otherwise attached to one of the four non-substituted carbons that form the ring. As used herein, the symbol “Rₓ” (e.g., R₁, R₂, R₃, R₄) is a general abbreviation that represents a substituent group as described herein. Substituent groups can improve the self-immolative ability of the p-aminobenzyl group (Hay et al., J. Chem Soc., Perkin Trans. 1:2759-2770 (1999); see also, Sykes et al. J. Chem. Soc., Perkin Trans. 1:1601-1608 (2000)).

Self-immolative elimination can take place, e.g., via 1,4 elimination, 1,6 elimination (e.g., pABC), 1,8 elimination (e.g., p-amino-cinnamyl alcohol), β-elimination, cyclisation-elimination (e.g., 4-aminobutanol ester and ethylenediamines), cyclization/lactonization, cyclization/lactolization, etc. See, e.g., Singh et al. Curr. Med. Chem. 15:1802-1826 (2008); Greenwald et al. J. Med. Chem. 43:475-487 (2000).

In some aspects, the self-immolative linker can comprise, e.g., cinnamyl, naphthyl, or biphenyl groups (see, e.g., Blencowe et al. Polym. Chem. 2:773-790 (2011)). In some aspects, the self-immolative linker comprises a heterocyclic ring (see, e.g., U.S. Pat. Nos. 7,375,078; 7,754,681). Numerous homoaromatic (see, e.g., Carl et al. J. Med. Chem. 24:479 (1981); Senter et al. J. Org. Chem. 55:2975 (1990); Taylor et al. J. Org. Chem. 43:1197 (1978); Andrianomenjanahary et al. Bioorg. Med. Chem. Lett. 2:1903 (1992)), and coumarin (see, e.g., Weinstein et al. Chem. Commun. 46:553 (2010)), furan, thiophene, thiazole, oxazole, isoxazole, pyrrole, pyrazole (see, e.g., Hay et al. J. Med. Chem. 46:5533 (2003)), pyridine (see, e.g., Perry-Feigenbaum et al. Org. Biomol. Chem. 7:4825 (2009)), imidazone (see, e.g., Nailor et al. Bioorg. Med. Chem. Lett. Z: 1267 (1999); Hay and Denny, Tetrahedron Lett. 38:8425 (1997)), and triazole (see, e.g., Bertrand and Gesson, J. Org. Chem. 72:3596 (2007)) based heteroaromatic groups that are self-immolative under both aqueous and physiological conditions are known in the art. See also, U.S. Pat. Nos. 7,691,962; 7,091,186; U.S. Pat. Publ. Nos. US2006/0269480; US2010/0092496; US2010/0145036; US2003/0130189; US2005/0256030)

In some aspects, a linker combination disclosed herein comprises more than one self-immolative linker in tandem, e.g., two or more pABC units. See, e.g., de Groot et al. J. Org. Chem. 66:8815-8830 (2001). In some aspects, a linker combination disclosed herein can comprise a self-immolative linker (e.g., a p-aminobenzylalcohol or a hemithioaminal derivative of p-carboxybenzaldehyde or glyoxilic acid) linked to a fluorigenic probe (see, e.g., Meyer et al. Org. Biomol. Chem. 8:1777-1780 (2010)).

Where substituent groups in the self-immolative linker s are specified by their conventional chemical formulae, written from left to right, they equally encompass the chemically identical substituents, which would result from writing the structure from right to left. For example, “—CH₂O—” is intended to also recite “—OCH₂—”.

Substituent groups in self-immolative, for example, R₁ and/or R₂ substituents in a p-aminobenzyl self-immolative linker as discuss above can include, e.g., alkyl, alkylene, alkenyl, alkynyl, alkoxy, alkylamino, alkylthio, heteroalkyl, cycloalkyl, heterocycloalkyl, aryl, arylalkyl, aryloxy, heteroaryl, etc. When a compound of the present disclosure includes more than one substituent, then each of the substituents is independently chosen.

In some specific aspects, the self-immolative linker is attached to cleavable peptide linker has the following formula, the combination having the following formula:

-Aₐ-Y_{y}-

wherein each -A- is independently an amino acid unit, a is independently an integer from 1 to 12; and -Y- is a self-immolative spacer, and y is 1, or 2. In some aspects, -Aₐ- is a dipeptide, a tripeptide, a tetrapeptide, a pentapeptide, or a hexapeptide. In some aspects, -Aₐ- is selected from the group consisting of valine-alanine, valine-citrulline, phenylalanine-lysine, N-methylvaline-citrulline, cyclohexylalanine-lysine, and beta-alanine-lysine. In some aspects, -Aₐ- is valine-alanine or valine-citrulline.

In some aspects, the self-immolative linker -Y_{y}- has the following formula:

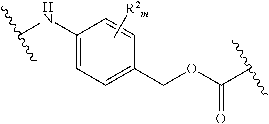

wherein each R² is independently C₁₋₈ alkyl, —O—(C₁₋₈ alkyl), halogen, nitro, or cyano; and m is an integer from 0 to 4. In some aspects, m is 0, 1, or 2. In some aspects, m is 0.

In some aspects, the cleavable linker is valine-alanine-p-aminobenzylcarbamate or valine-citrulline-p-aminobenzylcarbamate.

I.B.3. Reactive Moieties (RM)

The ASOs of the present disclosure are generated either via chemical synthesis or via chemical reaction between their components. For example, in some aspects, an anchoring moiety comprising a reactive group (e.g., maleimide) can react with a BAM comprising a maleimide-reacting group, to yield a modified BAM of the present disclosure, where the anchoring moiety may insert into the lipid bilayer of the membrane of an exosome, thereby attaching the BAM to the surface of the exosome.

Any component or group of components of a modified BAM of the present disclosure can comprise at least a RG and/or an RM, which would allow the attachment of the components through one reaction or series of reactions, to yield a modified BAM of the present disclosure. Exemplary synthesis schemas for the production of modified BAMs include:

[AM]-/RG/+/RM/-[BAM]→[AM]-[BAM]

[AM]-/RM/+/RG/-[BAM]→[AM]-[BAM]

[AM]-[L]-/RM/+/RG/-[BAM]→[AM]-[L]-[BAM]

[AM]-[L]-/RG/+/RM/-[BAM]→[AM]-[L]-[BAM]

[AM]-/RM/+/RG/-[L]-[BAM]→[AM]-[L]-[BAM]

[AM]-/RG/+/RM/-[L]-[BAM]→[AM]-[L]-[BAM]

[AM]-[L]-/RM/+/RG/-[L]-[BAM]→[AM]-[L]-[L]-[BAM]

[AM]-[L]-/RG/+/RM/-[L]-[BAM]→[AM]-[L]-[L]-[BAM]

wherein [AM] is an anchoring moiety, [BAM] is a biologically active molecule, [L] is a linker or linker combination, /RM/ is a reactive moiety, and /RG/ is a reactive group. In any of the schematic representations provided, the BAM can be attached, e.g., via its 5′ end or 3′ end.

Exemplary synthesis schemas for the production of intermediates in the synthesis of BAMs include:

[AM]-/RM/+/RG/-[L]→[AM]-[L]

[AM]-/RG/+/RM/-[L]→[AM]-[L]

[L]-/RM/+/RG/-[L]→[L]-[L]

[L]-/RG/+/RM/-[L]→[L]-[L]

[L]-/RM/+/RG/-[BAM]→[L]-[BAM]

[L]-/RG/+/RM/-[BAM]→[L]-[BAM]

wherein [AM] is an anchoring moiety, [BAM] is a biologically active molecule, [L] is a linker or linker combination, /RM/ is a reactive moiety, and /RG/ is a reactive group. In any of the schematic representations provided, the BAM can be attached, e.g., via its 5′ end or 3′ end.

In some aspects, the reactive group “/RG/” can be, e.g., an amino group, a thiol group, a hydroxyl group, a carboxylic acid group, or an azide group. Specific reactive moieties “/RM/” that can react with these reactive groups are described in more detail below.

[AM]-(/RM/)n+(/RG/-[L]-[BAM])n→[AM]-[L]-[BAM]

Any of the anchoring moieties, linker or linker combinations, or BAM disclosed herein can be conjugated to a reactive moiety, e.g., an amino reactive moiety (e.g., NHS-ester, p-nitrophenol, isothiocyanate, isocyanate, or aldehyde), a thiol reactive moiety (e.g., acrylate, maleimide, or pyridyl disulfide), a hydroxy reactive moiety (e.g., isothiocyanate or isocyanate), a carboxylic acid reactive moiety (e.g., epoxide), or an azide reactive moiety (e.g., alkyne).

Exemplary reactive moieties that can be used to covalent bind two components disclosed herein (e.g., an anchoring moiety and a BAM, or an anchoring moiety and a linker, or an anchoring moiety and a linker, or two linkers, or a linker and a BAM, or a two anchoring moieties) include, e.g., N-succinimidyl-3-(2-pyridyldithio)propionate, N-4-maleimide butyric acid, S-(2-pyridyldithio)cysteamine, iodoacetoxysuccinimide, N-(4-maleimidebutyryl oxy)succinimide, N-[5-(3′-maleimide propylamide)-1-carboxypentyl]iminodiacetic acid, N-(5-aminopentyl)iminodiacetic acid, and 1′-[(2-cyanoethyl)-(N,N-diisopropyl)]-phosphoramidite). Bifunctional linkers (linkers containing two functional groups) are also usable.

In some aspects, an anchoring moiety, linker, or BAM can comprise a terminal oxyamino group, e.g., ONH₂, an hydrazino group, NHNH₂, a mercapto group (i.e., SH or thiol), or an olefin (e g. CH═CH₂). In some aspects, an anchoring moiety, linker, or BAM can comprise an electrophilic moiety, e.g., at a terminal position, e.g., an aldehyde, alkyl halide, mesylate, tosylate, nosylate, or brosylate, or an activated carboxylic acid ester, e.g. an NHS ester, a phosphoramidite, or a pentafluorophenyl ester. In some aspects, a covalent bond can be formed by coupling a nucleophilic group of a ligand, e.g., a hydroxyl, a thiol or amino group, with an electrophilic group The present invention is amenable to all manner of reactive groups and reactive moieties including but not limited to those known in the art.

The term “protecting group,” as used herein, refers to a labile chemical moiety which is known in the art to protect reactive groups including without limitation, hydroxyl amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene and Wits, Protective Groups in Organic Synthesis, 3rd edition, John Wiley & Sons, New York (1999).

Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989), T. W. Greene and P. G. M. Wuts, Protective Groups in Organic Synthesis, 2d Ed, John Wiley and Sons (1991); L. Fieser and M. Fieser, Fieser and Fieser's Reagents for Organic Synthesis, John Wiley and Sons (1994); and L Paquette, ed., Encyclopedia of Reagents for Organic Synthesis, John Wiley and Sons (1995), and subsequent editions thereof.

Solid phase synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991) and Toy, P. H.; Lam, Y (ed.), Solid-Phase Organic synthesis, concepts, Strategies, and Applications, John Wiley & Sons, Inc. New Jersey (2012).

In some aspects, the reactive group can alternatively react with more than one of the reactive moieties described below.

II.B.3.a. Amine Reactive Moieties

In some aspects, the reactive moiety is an amine reactive moiety. As used herein the term “amine reactive moiety” refers to a chemical groups which can react with a reactive group having an amino moiety, e.g., primary amines. Exemplary amine reactive moieties are N-hydroxysuccinimide esters (NHS-ester), p-nitrophenol, isothiocyanate, isocyanate, and aldehyde. Alternative reactive moieties that react with primary amines are also well known in the art. In some aspects, an amine reactive moiety can be attached to a terminal position of an anchoring moiety, linker combination, or BAM of the present disclosure.

In some aspects, the amine reactive moiety is a NHS-ester. Typically, a NHS-ester reactive moiety reacts with a primary amine of a reactive group to yield a stable amide bond and N-hydroxysuccinimide (NHS).

In some aspects, the amine reactive moiety is a p-nitrophenol group. Typically, a p-nitrophenol reactive moiety is an activated carbamate that reacts with a primary amine of a reactive group to yield a stable carbamate moiety and p-nitrophenol.

In some aspects, the amine reactive moiety is an isothiocyanate. Typically, a isothiocyanate reacts with a primary amine of a reactive group to yield a stable thiourea moiety.

In some aspects, the amine reactive moiety is an isocyanate. Typically, a isocyanate reacts with a primary amine of a reactive group to yield a stable urea moiety.

In some aspects, amine the reactive moiety is an aldehyde. Typically, aldehydes react with primary amines to form Schiff bases which can be further reduced to form a covalent bond through reductive amination.

II.B.3.b. Thiol Reactive Moieties

In some aspects, the reactive moiety is a thiol reactive moiety. As used herein the term “thiol reactive moiety” refers to a chemical groups which can react with a reactive group having a thiol moiety (or mercapto group). Exemplary thiol reactive moieties are acrylates, maleimides, and pyridyl disulfides. Alternative reactive moieties that react with thiols are also well known in the art. In some aspects, a thiol reactive moiety can be attached to a terminal position of an anchoring moiety, linker combination, or BAM of the present disclosure.

In some aspects, the thiol reactive moiety is an acrylate. Typically, acrylates react with thiols at the carbon p to the carbonyl of the acrylate to form a stable sulfide bond. In some aspects, the thiol reactive moiety is a maleimide. Typically, maleimides react with thiols at either of at the carbon p the to the carbonyls to form a stable sulfide bond. In some aspects, the thiol reactive moiety is a pyridyl disulfide. Typically, pyridyl disulfides react with thiols at the sulfur atom p to the pyridyl to form a stable disulfide bond and pyridine-2-thione.

II.B.3.c. Hydroxy Reactive Moieties

In some aspects, the reactive moiety is a hydroxyl reactive moiety. As used herein the term “hydroxyl reactive moiety” refers to a chemical group which can react with a reactive group having an hydroxyl moiety. Exemplary hydroxyl reactive moieties are isothiocyanates and isocyanates. Alternative reactive moieties that react with hydroxyl moieties are also well known in the art. In some aspects, a hydroxyl reactive moiety can be attached to a terminal position of an anchoring moiety, linker combination, or BAM of the present disclosure.

In some aspects, the hydroxyl reactive moiety is an isothiocyanate. Typically, an isothiocyanate reacts with a hydroxyl of a reactive group to yield a stable carbamothioate moiety. In some aspects, amine the reactive moiety is a isocyanate. Typically, an isocyante reacts with a hydroxyl of a reactive group to yield a stable carbamate moiety.

II.B.3.d. Carboxylic Acid Reactive Moieties

In some aspects, the reactive moiety is a carboxylic acid reactive moiety. As used herein the term “carboxylic acid reactive moiety” refers to a chemical groups which can react with a reactive group having an carboxylic acid moiety. An exemplary carboxylic acid reactive moieties is an epoxide. Alternative reactive moieties that react with carboxylic acid moieties are also well known in the art. In some aspects, an carboxylic acid reactive moiety can be attached to a terminal position of an anchoring moiety, linker combination, or BAM of the present disclosure.

In some aspects, the carboxylic acid reactive moiety is an epoxide. Typically, an epoxide reacts with the carboxylic acid of a reactive group at either of the carbon atoms of the epoxide to form a 2-hydroxyethyl acetate moiety.

II.B.3.e. Azide Reactive Moieties

In some aspects, the reactive moiety is an azide reactive moiety. As used herein the term “azide reactive moiety” refers to a chemical groups which can react with a reactive group having an azide moiety. An exemplary azide reactive moieties is an alkyne. Alternative reactive moieties that react with azide moieties are also well known in the art. In some aspects, a carboxylic acid reactive moiety can be attached to a terminal position of an anchoring moiety, linker combination, or BAM of the present disclosure.

In some aspects, the azide reactive moiety is an alkyne. Typically, an alkyne reacts with the azide of a reactive group through a 1,3-dipolar cycloaddition reaction, also referred to “click chemistry,” to form a 1,2,3-triazole moiety.

II.B.4. Specific Examples and Topologies

In specific aspects of the present disclosure, the linker combination consists of a linker of formula

[Alkyl linker]m-[PEG1]n-[PEG2]o

wherein m, n, and o are 0 or 1, and at least one of m, n, or o is not zero. Exemplary linker combinations according to such formula are C6-TEG-HEG, C6-HEG, C6-TEG, C6, TEG-HEG, TEG, C8-TEG-HEG, C8-HEG, C8-TEG, and C8.

In some aspects, the linker combination comprises a non-cleavable linker (e.g., TEG or HEG) in combination with one or more cleavable linkers, e.g., an enzymatic cleavable linker and a self immolative linker.

In a specific aspect, the linker combination comprises the linker combination TEG (non-cleavable linker)-Val-Cit(cleavable linker)-pAB(self-immolative linker), as shown below

### [Cholesterol]-[TEG]-[Val-Cit]-[pAB]

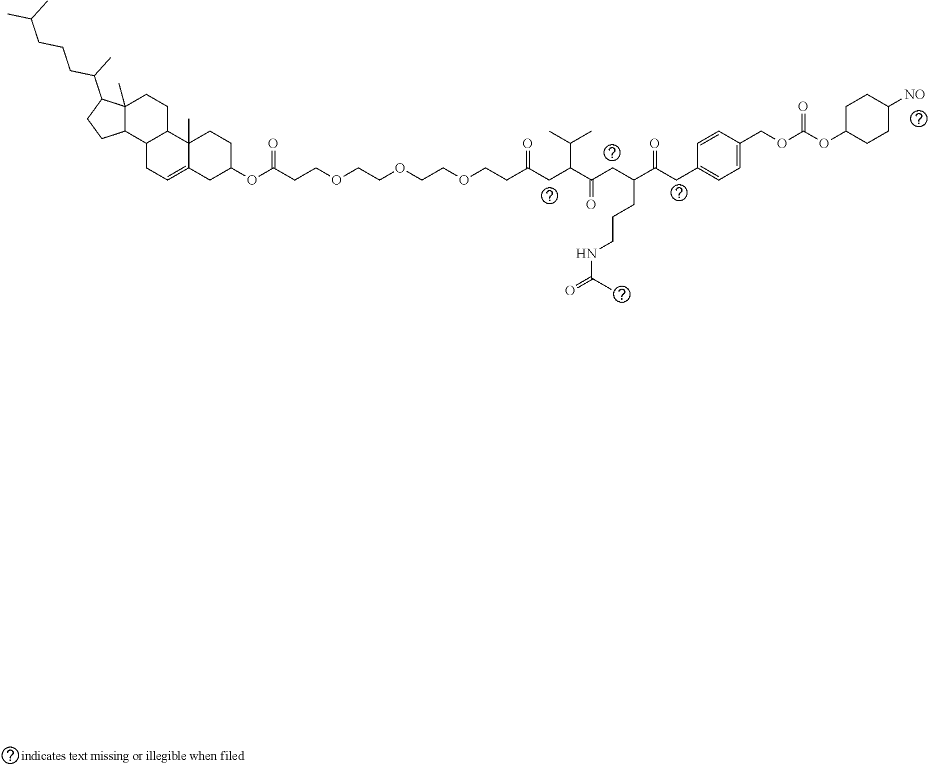

Specific combinations of anchoring moieties and linker combinations are illustrated in the tables below.

TABLE 1

| | Linker combination | | |
|---|---|---|---|
| Anchoring moiety | 1^{st} Linker | 2^{nd} Linker | 3^{rd} Linker |
| Cholesterol | C6 | TEG | HEG |
| Cholesterol | C6 | HEG | No |
| Cholesterol | C6 | TEG | No |
| Cholesterol | C6 | No | No |
| Cholesterol | TEG | HEG | No |
| Cholesterol | TEG | No | No |
| Tocopherol | C8 | TEG | HEG |
| Tocopherol | C8 | HEG | No |
| Tocopherol | C8 | TEG | No |
| Tocopherol | C8 | No | No |
| Tocopherol | TEG | HEG | No |
| Tocopherol | HEG | No | No |
| Tocopherol | TEG | No | No |
| Tocopherol | No | No | No |
| Palmitate | C6 | TEG | HEG |
| Palmitate | C6 | HEG | No |
| Palmitate | C6 | TEG | No |
| Palmitate | C6 | No | No |
| Cholesterol | TEG | Glycerol | HEG |

TABLE 2
Linker Combination

| Linker 1 | Cleavable Linker 2 | Linker 3 |
|---|---|---|
| C6 | Disulfide | C6 |
| None | Imine | None |
| TEG | Thioketal | TEG |
| HEG | Tri/Dinucleotide | HEG |
| TEG-HEG | Val-Cit | TEG-HEG |

In some aspects, the linker combination has the general structure [AM]-[Linker1]-[Linker2]-[BAM], wherein the anchoring moiety [AM] is selected from cholesterol, palmitate and tocopherol, the first linker [Linker1] is a hydrophobic linker, and the second linker [Linker2] is a hydrophilic linker. In other aspects, the linker combination has the general structure [AM]-[Linker1]-[Linker2]-[BAM], wherein the anchoring moiety [AM] is selected from cholesterol, palmitate and tocopherol, the first linker [Linker1] is a hydrophilic linker, and the second linker [Linker2] is a hydrophilic linker. In other aspects, In some aspects, the linker combination has the general structure [AM]-[Linker1]-[Linker2]-[ASO], wherein the anchoring moiety [AM] is selected from cholesterol, palmitate and tocopherol, the first linker [Linker1] is selected from a C6 linker, a C8 linker, a TEG linker, and a HEG linker, and the second linker [Linker2] is a hydrophilic linker selected from TEG and HEG.

In some aspects, the linker combination has the general structure [AM]-[Linker1]-[Linker2]-[Linker3]-[ASO], where in the anchoring moiety [AM] is a lipid (e.g., a phospholipid), the first linker [Linker1] is selected from the group consisting of HEG, TEG, TEG-HEG, and C6, or it is absent, the second linker [Linker2] is selected from the group consisting of disulfide, imine, thioketal, tri/dinucleotide, and Val-Cit, and the third linker [Linker3] is selected from the group consisting of HEG, TEG, TEG-HEG, and C6, or it is absent.

Specific linker combinations of the present disclosure are exemplified below

### [Cholesterol]-[Teg]-[Heg]-[Bam]

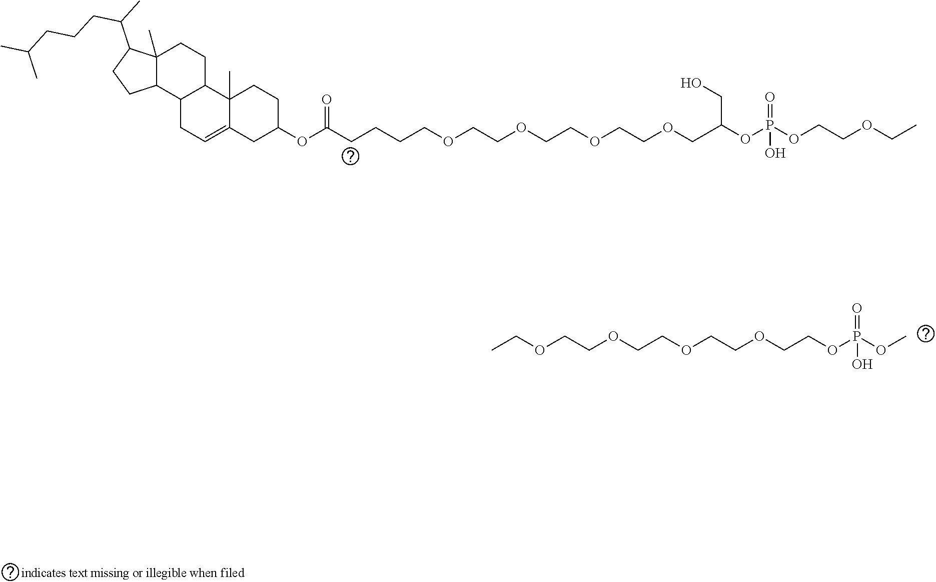

### [Cholesterol]-[SMal]-[Val-Cit]-[pAB]-[BAM]

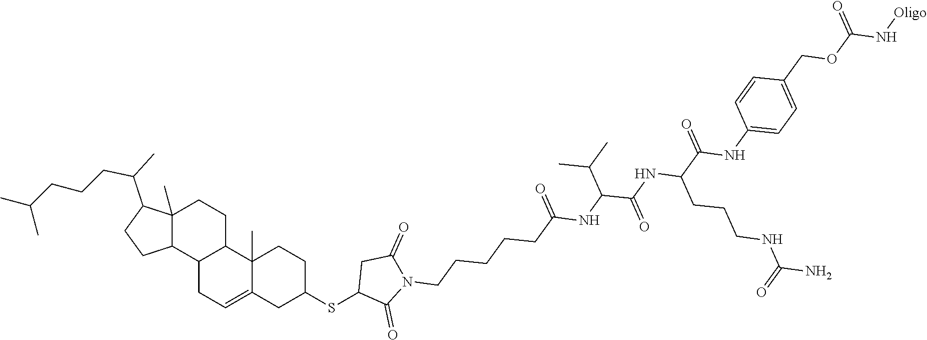

### [Cholesterol]-[TEG]-[Val-Cit]-[C6]-[BAM]

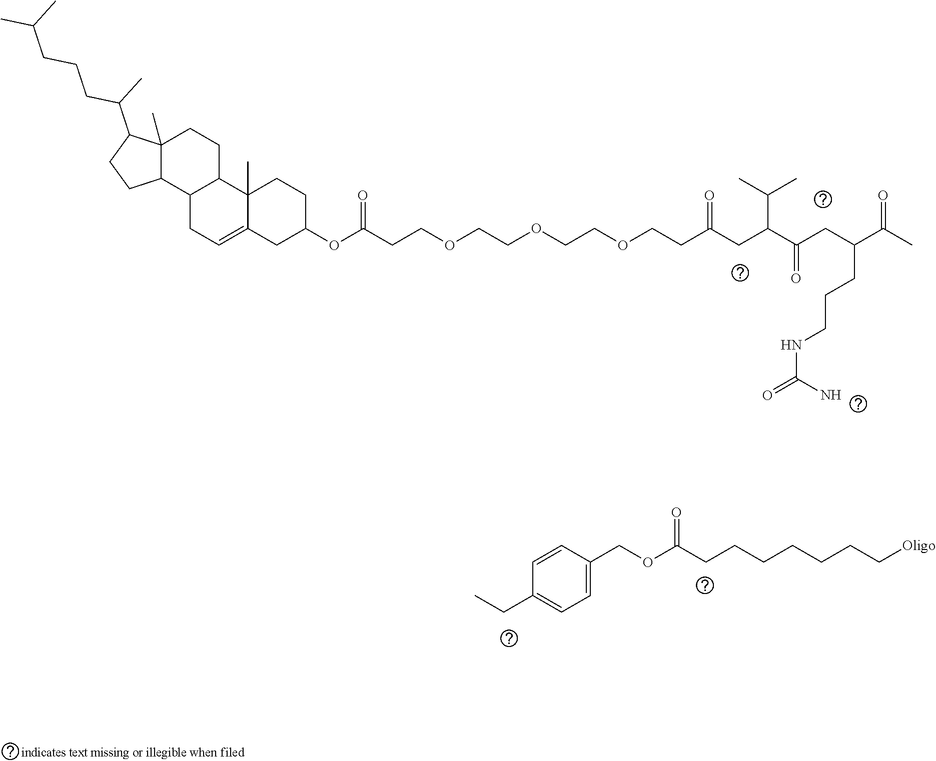

### [Cholesterol]-[Teg]-[Ss]-[C6]-[Bam]

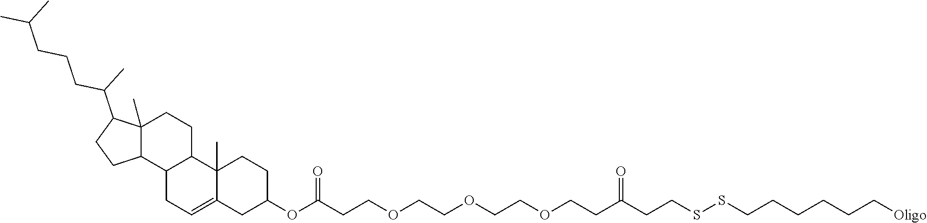

wherein [Cholesterol] is a cholesterol anchoring moiety, [TEG] is a TEG non-cleavable linker, [HEG] is a HEG non-cleavable linker, [SS] is a disulfide redox cleavable linker, [C6] is an alkyl non-cleavable linker, [SMal] is S-maleimide, [Val-Cit] is a valine-citrulline cleavable linker, [pAB] is a pAB self-immolative linker. In some aspects, an ASO of the present disclosure has a structure according to the exemplary structures provided above, in which one or more components has been replaced by a component in the same class as those depicted in the example. For example, the [cholesterol] anchoring moiety can be substituted by another anchoring moiety disclosed herein, a [TEG] can be substituted by another polymeric non-cleavable linker disclosed herein (e.g., HEG, PEG, PG), [Val-Cit] can be replaced by another peptidase cleavable linker, or [pAB] can be substituted by another self-immolative linker.

Additional specific linker combinations of the present disclosure are exemplified below:

### [Saturated Phospholipid; Ethanolamine; DLPE]-[TEG]-[BAM]

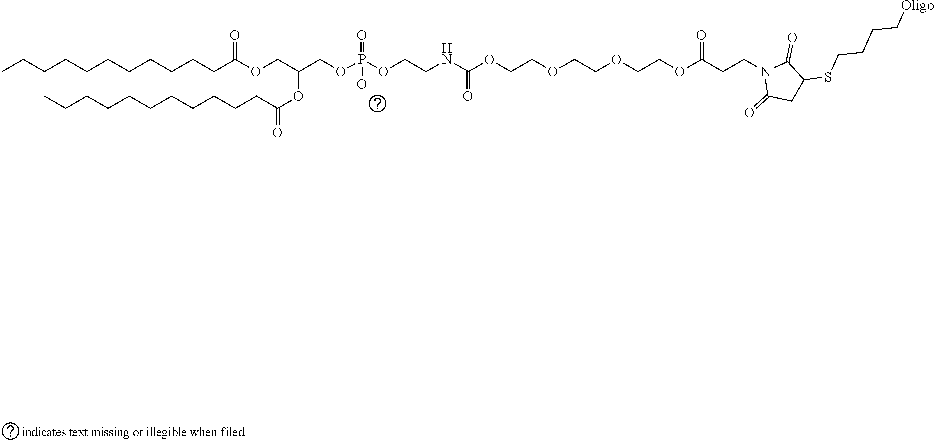

### [Unsaturated Phospholipid; Ethanolamine; DOPE]-[BAM]

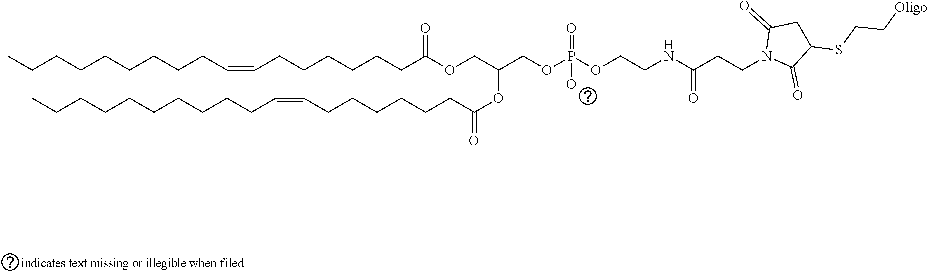

### [Saturated Fatty Acid; Laurate]-[TEG]-[C6]-[BAM]

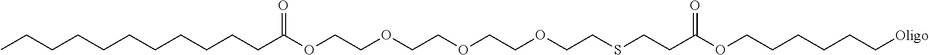

### [Unsaturated Fatty Acid; Linoleate]-[TEG]-[C6]-[BAM]

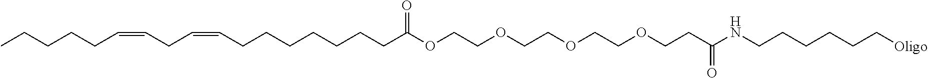

### [Stearate]-[TEG]-[HEG]-[BAM]

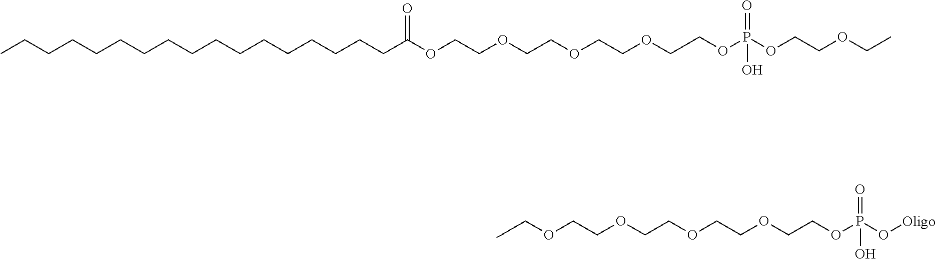

II.C Biologically Active Molecule

In some aspects, an EV (e.g., exosome) disclosed herein is capable of delivering a payload (a biologically active molecule attached to the EV, e.g., exosome, via an anchoring moiety) to a target. The payload is an agent that acts on a target (e.g., a target cell) that is contacted with the EV (e.g., exosome). Contacting can occur in vitro or in a subject. Non-limiting examples of payloads that can attached to an EV (e.g., exosome) via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), amino acids (e.g., amino acids comprising a detectable moiety or a toxin that disrupt translation), polypeptides (e.g., enzymes), lipids, carbohydrates, and small molecules (e.g., small molecule drugs and toxins). In some aspects, a payload is in the lumen of the EV (e.g., exosome). In some aspects, an EV (e.g., exosome) can comprise more than one payload, e.g., a first payload in solution the lumen of EV (e.g., exosome), and a second payload attached, e.g., to the external surface of the EV (e.g., exosome) via an anchoring moiety.

In some aspects, the biologically active molecule (payload, BAM) is not naturally occurring with the EV, e.g., exosome. In some aspects, the payload (BAM) is non naturally occurring. In some aspects, the EVs comprising a BAM is non-naturally occurring.

In some aspects, the payload targets a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, or combinations thereof.

In some aspects, the payload is a small molecule. In some aspects, the small molecule is a proteolysis-targeting chimera (PROTAC).

In some aspects, the payload comprises a nucleotide, wherein the nucleotide is a stimulator of interferon genes protein (STING) agonist. STING is a cytosolic sensor of cyclic dinucleotides that is typically produced by bacteria. Upon activation, it leads to the production of type I interferons and initiates an immune response

In some aspects, the EV (e.g., exosome) of the present disclosure comprises one or more STING agonists covalently linked to the EV (e.g., exosome) via an anchoring moiety. In some aspects, the STING agonist comprises a cyclic nucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs may have 2′2′, 2′3′, 2′5′, 3′3′, or 3′5′ bonds linking the cyclic dinucleotides, or any combination thereof.

Cyclic purine dinucleotides may be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides may be modified analogues. Any suitable modification known in the art may be used, including, but not limited to, phosphorothioate, biphosphorothioate, fluorinate, and difluorinate modifications.

Non cyclic dinucleotide agonists may also be used, such as 5,6-Dimethylxanthenone-4-acetic acid (DMXAA), or any other non-cyclic dinucleotide agonist known in the art.

It is contemplated that any STING agonist may be used. Among the STING agonists are DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, c-di-AMP, 2′3′-c-di-AMP, 2′3′-c-di-AM(PS)2, c-di-GMP, 2′3′-c-di-GMP, c-di-IMP, c-di-UMP or any combination thereof. In a specific aspect, the STING agonist is 3′3′-cAIMPdFSH, alternatively named 3-3 cAIMPdFSH. Additional STING agonists known in the art may also be used.

In some aspects, the biologically active molecule is an antibody or antigen binding fragment thereof. In some aspects, the biologically active molecule is an ADC. In some aspects, the biologically active molecule is a small molecule comprising a synthetic antineoplastic agent (e.g., monomethyl auristatin E (MMAE) (vedotin)), a cytokine release inhibitor (e.g., MCC950), an mTOR inhibitor (e.g., Rapamycin and its analogs (Rapalogs)), an autotaxin inhibitor (e.g., PAT409 or PAT505), a lysophosphatidic acid receptor antagonist (e.g., BMS-986020), a STING antagonist (e.g., CL656), or any combination thereof.). In some aspects, the biologically active molecule is a fusogenic peptide.

In some aspects, the biologically active molecule comprises an antisense oligonucleotide (ASO). In some aspects, the ASO targets various genes (transcripts) expressed in vivo. In some aspects, a biologically active molecule of the present disclosure comprises morpholino backbone structures disclosed in U.S. Pat. No. 5,034,506, which is herein incorporated by reference in its entirety. In some aspects, a biologically active molecule of the present disclosure includes phosphorodiamidate morpholino oligomers (PMO), in which the deoxyribose moiety is replaced by a morpholine ring, and the charged phosphodiester inter-subunit linkage is replaced by an uncharged phosphorodiamidate linkage, as described in Summerton, et al., Antisense Nucleic Acid Drug Dev. 1997, 7:63-70. Thus, in some aspects, the biologically active molecule is an antisense oligonucleotide, a phosphorodiamidate morpholino oligomer (PMO), or a peptide-conjugated phosphorodiamidate morpholino oligomer (PPMO).

In some aspects, the biologically active molecule targets macrophages. In other aspects, the biologically active molecule induces macrophage polarization. Macrophage polarization is a process by which macrophages adopt different functional programs in response to the signals from their microenvironment. This ability is connected to their multiple roles in the organism: they are powerful effector cells of innate immune system, but also important in removal of cellular debris, embryonic development and tissue repair.

By simplified classification, macrophage phenotype has been divided into 2 groups: M1 (classically activated macrophages) and M2 (alternatively activated macrophages). This broad classification was based on in vitro studies, in which cultured macrophages were treated with molecules that stimulated their phenotype switching to particular state. In addition to chemical stimulation, it has been shown that the stiffness of the underlying substrate a macrophage is grown on can direct polarization state, functional roles and migration mode. M1 macrophages were described as the pro-inflammatory type, important in direct host-defense against pathogens, such as phagocytosis and secretion of pro-inflammatory cytokines and microbicidal molecules. M2 macrophages were described to have quite the opposite function: regulation of the resolution phase of inflammation and the repair of damaged tissues. Later, more extensive in vitro and ex vivo studies have shown that macrophage phenotypes are much more diverse, overlapping with each other in terms of gene expression and function, revealing that these many hybrid states form a continuum of activation states which depend on the microenvironment. Moreover, in vivo, there is a high diversity in gene expression profile between different populations of tissue macrophages. Macrophage activation spectrum is thus considered to be wider, involving complex regulatory pathway to response to plethora of different signals from the environment. The diversity of macrophage phenotypes still remain to be fully characterized in vivo.

The imbalance of the macrophage types is related to a number of immunity-related diseases. For example, increased M1/M2 ratio may correlate with development of inflammatory bowel disease, as well as obesity in mice. On the other side, in vitro experiments implicated M2 macrophages as the primary mediators of tissue fibrosis. Several studies have associated the fibrotic profile of M2 macrophages with the pathogenesis of systemic sclerosis. Non-limiting examples of the macrophage targeting biologically active molecules are: PI3Kγ (phosphatidylinositol-4,5-bisphosphate 3-kinase catalytic subunit gamma), RIP1 (Receptor Interacting Protein (RIP) kinase 1, RIPK1), HIF-1α (Hypoxia-inducible factor 1-alpha), AHR1 (Adhesion and hyphal regulator 1), miR146a, miR155, IRF4 (Interferon regulatory factor 4), PPAR7 (Peroxisome proliferator-activated receptor gamma), IL-4RA (Interleukin-4 receptor subunit alpha), TLR8 (Toll-like receptor 8), and TGF-β1 (Transforming growth factor beta-1 proprotein)

In some aspects, the biologically active molecule targets PI3Kγ protein or transcript (PI3Kγ antagonist). In some aspects, the PI3Kγ antagonist is an antisense oligonucleotide. In other aspects, the PI3Kγ antagonist is a small molecule. In some aspects, the ASO targets a transcript, e.g., mRNA, encoding PI3Kγ. The sequence for the PI3Kγ gene can be found at chromosomal location 7q22.3 and under publicly available GenBank Accession Number NC_000007.14 (106865282 . . . 106908980), which is incorporated by reference in its entirety. The sequence for human PI3Kγ protein can be found under publicly available UniProt Accession Number P48736, which is incorporated by reference herein in its entirety.

In some aspects, the biologically active molecule targets RIP1 protein or transcript (RIP1 antagonist). In some aspects, the RIP1 antagonist is an antisense oligonucleotide. In other aspects, the RIP1 antagonist is a small molecule. In some aspects, the ASO targets a transcript, e.g., mRNA, encoding RIP1. The sequence for the RIP1 gene can be found at chromosomal location 6p25.2 and under publicly available GenBank Accession Number NC_000006.12 (3063967 . . . 3115187), which is incorporated by reference in its entirety. The sequence for human RIP1 protein can be found under publicly available UniProt Accession Number Q13546, which is incorporated by reference herein in its entirety.

In some aspects, the biologically active molecule targets HIF-1α protein or transcript (HIF-1α antagonist). In some aspects, the HIF-1α antagonist is an antisense oligonucleotide. In other aspects, the HIF-1α antagonist is a small molecule. In some aspects, the ASO targets a transcript, e.g., mRNA, encoding HIF-1α. The sequence for the HIF-1α gene can be found at chromosomal location 14q23.2 and under publicly available GenBank Accession Number NC_000014.9 (61695513 . . . 61748259), which is incorporated by reference in its entirety. The sequence for human HIF-1α protein can be found under publicly available UniProt Accession Number Q16665, which is incorporated by reference herein in its entirety. In some aspects, the ASO targets a mRNA encoding HIF-2α. The sequence for the HIF-2α gene can be found at chromosomal location 2p21 and under publicly available GenBank Accession Number NC_000002.12 (46297407 . . . 46386697), which is incorporated by reference in its entirety. The sequence for human HIF-2α protein can be found under publicly available UniProt Accession Number Q99814, which is incorporated by reference herein in its entirety

In some aspects, the biologically active molecule targets AHR1 protein or transcript (AHR1 antagonist). In other aspects, the AHR1 antagonist is a small molecule.

In some aspects, the biologically active molecule targets miR146a (miR146a antagomir). In some aspects, the miR146a antagomir is an antisense oligonucleotide. In some aspects, the ASO binds to miR146a-5p (ugagaacugaauuccauggguu) (SEQ ID NO:54). In some aspects, the ASO binds to miR146a-3p (ccucugaaauucaguucuucag) (SEQ ID NO:55).

In some aspects, the biologically active molecule mimics miR155 (miR155 mimic). In some aspects, the miR155 mimic is an RNA or DNA. In some aspects, the miR155 mimic comprises the nucleotide sequence of miR155-5p (uuaaugcuaaucgugauaggggu) (SEQ ID NO:56). In some aspects, the miR155 mimic comprises the nucleotide sequence of miR155-3p (cuccuacauauuagcauuaaca) (SEQ ID NO:57).

In some aspects, the biologically active molecule targets IRF-4 protein or transcript (IRF4 antagonist). In some aspects, the IRF4 antagonist is an antisense oligonucleotide. In some aspects, the ASO targets a transcript, e.g., mRNA, encoding IRF-4. The sequence for the IRF-4 gene can be found at chromosomal location 6p25.3 and under publicly available GenBank Accession Number NC_000006.12 (391739 . . . 411443), which is incorporated by reference in its entirety. The sequence for human IRF-4 protein can be found under publicly available UniProt Accession Number Q15306, which is incorporated by reference herein in its entirety.

In some aspects, the biologically active molecule targets PPARγ protein or transcript (PPARγ antagonist). In some aspects, the PPARγ antagonist is an antisense oligonucleotide. In other aspects, the PPARγ antagonist is a small molecule. In some aspects, the ASO targets a transcript, e.g., mRNA, encoding PPARγ. The sequence for the PPARγ gene can be found at chromosomal location 3p25.2 and under publicly available GenBank Accession Number NC_000003.12 (12287485 . . . 12434356), which is incorporated by reference in its entirety. The sequence for human PPARγ protein can be found under publicly available UniProt Accession Number P37231, which is incorporated by reference herein in its entirety.

In some aspects, the biologically active molecule targets IL-4RA protein or transcript (IL-4RA antagonist). In some aspects, the IL-4RA antagonist is an antisense oligonucleotide. In some aspects, the ASO targets a transcript, e.g., mRNA, encoding IL-4RA. The sequence for the IL-4RA gene can be found at chromosomal location 16p12.1 and under publicly available GenBank Accession Number NC_000016.10 (27313668 . . . 27364778), which is incorporated by reference in its entirety. The sequence for human IL-4RA protein can be found under publicly available UniProt Accession Number P24394, which is incorporated by reference herein in its entirety.

In some aspects, the biologically active molecule is an agonist of Toll-like receptor 8 (TLR8). TLR8 is also referred to as CD288. TLR8 is a key component of innate and adaptive immunity. TLRs (Toll-like receptors) control host immune response against pathogens through recognition of molecular patterns specific to microorganisms. It acts via MYD88 and TRAF6, leading to NF-kappa-B activation, cytokine secretion and the inflammatory response. The sequence for human TLR8 protein can be found under publicly available UniProt Accession Number Q9NR97, which is incorporated by reference herein in its entirety.

In some aspects, the biologically active molecule targets TGF-β1 protein or transcript (TGF-β1 antagonist). In some aspects, the TGF-β1 antagonist is an antisense oligonucleotide. In some aspects, the ASO targets a transcript, e.g., mRNA, encoding TGF-β1. The sequence for the TGF-β31 gene can be found at chromosomal location 19q13.2 and under publicly available GenBank Accession Number NC_000019.10 (41330323 . . . 41353922, complement), which is incorporated by reference in its entirety. The sequence for human TGF-β1 protein can be found under publicly available UniProt Accession Number P01137, which is incorporated by reference herein in its entirety.

In some aspects, the ASO is a gapmer, a mixmer, or a totalmer. The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

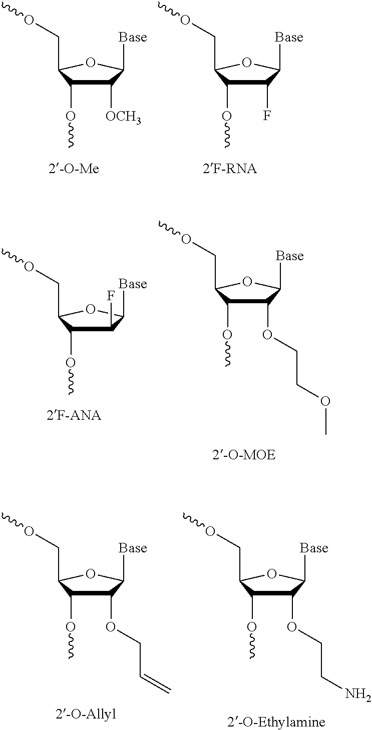

LNA nucleosides are modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., J. Org. Chem. 2010, Vol 75(5) pp. 1569-81, and Mitsuoka et al., Nucleic Acids Research 2009, 37(4), 1225-1238. In some aspects, the modified nucleoside or the LNA nucleosides of the ASO of the disclosure has a general structure of the formula I or II:

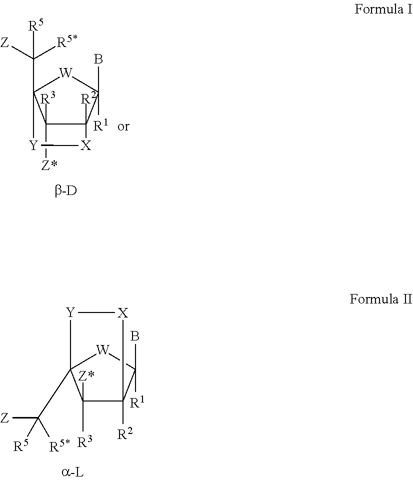

wherein

W is selected from —O—, —S—, —N(R^{a})—, —C(R^{a}R^{b})—, in particular —O—;

B is a nucleobase or a modified nucleobase moiety;

Z is an internucleoside linkage to an adjacent nucleoside or a 5′-terminal group;

Z* is an internucleoside linkage to an adjacent nucleoside or a 3′-terminal group;

R¹, R², R³, R⁵ and R^{5*} are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocycle and aryl; and

X, Y, R^{a} and R^{b} are as defined herein.

II.C.1. ASO Targeting NLRP3

NLRP3 (NLRP3) is also known as NLR family pyrin domain containing 3. Unless indicated otherwise, the term “NLRP3,” as used herein, can refer to NLRP3 from one or more species (e.g., humans, non-human primates, dogs, cats, guinea pigs, rabbits, rats, mice, horses, cattle, and bears). Synonyms of NLRP3/NLRP3 are known and include NLRP3; C1orf7; CIAS1; NALP3; PYPAF1; nucleotide-binding oligomerization domain, leucine rich repeat and pyrin domain containing 3; cold-induced autoinflammatory syndrome 1 protein; cryopyin; NACHT, LRR and PYD domains-containing protein 3; angiotensin/vasopressin receptor AII/AVP-like; caterpiller protein 1.1; CLR1.1; cold-induced autoinflammatory syndrome 1 protein; and PYRIN-containing APAF1-like protein 1. The sequence for the human NLRP3 gene can be found under publicly available GenBank Accession Number NC_000001.11:247416156-247449108. The human NLRP3 gene is found at chromosome location 1q44 at 247,416,156-247,449,108.

The sequence for the human NLRP3 pre-mRNA transcript (SEQ ID NO: 1) corresponds to the reverse complement of residues 247,416,156-247,449,108 of chromosome 1q44. The NLRP3 mRNA sequence (GenBank Accession No. NM_001079821.2) is provided in SEQ ID NO: 3, except that the nucleotide “t” in SEQ ID NO: 3 is shown as “u” in the mRNA. The sequence for human NLRP3 protein can be found under publicly available Accession Numbers: Q96P20, (canonical sequence, SEQ ID NO: 2), Q96P20-2 (SEQ ID NO: 4), Q96P20-3 (SEQ ID NO: 5), Q96P20-4 (SEQ ID NO: 6), Q96P20-5 (SEQ ID NO: 7), and Q96P20-6 (SEQ ID NO: 8), each of which is incorporated by reference herein in its entirety.

Natural variants of the human NLRP3 gene product are known. For example, natural variants of human NLRP3 protein can contain one or more amino acid substitutions selected from: D21H, I174T, V200M, R262L, 4262P, R262W, L266H, D305G, D305N, L307P, Q308K, F311S, T350M, A354V, L355P, E356D, H360R, T407P, T4381, T438N, A441T, A441V, R490K, F525C, F525L, G571R, Y572C, F575S, E629G, L634F, M664T, Q705K, Y861C, and R920Q, and any combinations thereof. Additional variants of human NLRP3 protein resulting from alternative splicing are also known in the art. NLRP3 Isoform 1 (identifier: Q96P20-2 at UniProt) differs from the canonical sequence (SEQ ID NO: 3) as follows: deletion of residues 721-777 and 836-892 relative to SEQ ID NO: 3. The sequence of NLRP3 Isoform 3 (identifier: Q96P20-3) differs from the canonical sequence (SEQ ID NO: 3) as follows: deletion of residues 720-1036 relative to SEQ ID NO: 3. The sequence of NLRP3 Isoform 4 (identifier: Q96P20-4) differs from the canonical sequence (SEQ ID NO: 3) as follows: deletion of residues 721-777 relative to SEQ ID NO: 3. The sequence of NLRP3 Isoform 5 (identifier: Q96P20-5) differs from the canonical sequence (SEQ ID NO: 3) as follows: deletion of residues 836-892 relative to SEQ ID NO: 3. The sequence of NLRP3 Isoform 6 (identifier: Q96P20-6) differs from the canonical sequence (SEQ ID NO: 3) as follows: deletion of residues 776-796 relative to SEQ ID NO: 3. Therefore, the ASOs of the present disclosure can be designed to reduce or inhibit expression of the natural variants of the NLRP3 protein.

An example of a target nucleic acid sequence of the ASOs is NLRP3 pre-mRNA. SEQ ID NO: 1 represents a human NLRP3 genomic sequence (i.e., reverse complement of nucleotides 247,416,156-247,449,108 of chromosome 1q44). SEQ ID NO: 1 is identical to a NLRP3 pre-mRNA sequence except that nucleotide “t” in SEQ ID NO: 1 is shown as “u” in pre-mRNA. In certain aspects, the “target nucleic acid” comprises an intron of a NLRP3 protein-encoding nucleic acids or naturally occurring variants thereof, and RNA nucleic acids derived therefrom, e.g., pre-mRNA. In other aspects, the target nucleic acid comprises an exon region of a NLRP3 protein-encoding nucleic acids or naturally occurring variants thereof, and RNA nucleic acids derived therefrom, e.g., pre-mRNA. In yet other aspects, the target nucleic acid comprises an exon-intron junction of a NLRP3 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human NLRP3 protein sequence encoded by the NLRP3 pre-mRNA is shown as SEQ ID NO: 3. In other aspects, the target nucleic acid comprises an untranslated region of a NLRP3 protein-encoding nucleic acids or naturally occurring variants thereof, e.g., 5′ UTR, 3′ UTR, or both.

In some aspects, an ASO of the disclosure hybridizes to a region within the introns of a NLRP3 transcript, e.g., SEQ ID NO: 1. In certain aspects, an ASO of the disclosure hybridizes to a region within the exons of a NLRP3 transcript, e.g., SEQ ID NO: 1. In other aspects, an ASO of the disclosure hybridizes to a region within the exon-intron junction of a NLRP3 transcript, e.g., SEQ ID NO: 1. In some aspects, an ASO of the disclosure hybridizes to a region within a NLRP3 transcript (e.g., an intron, exon, or exon-intron junction), e.g., SEQ ID NO: 1, wherein the ASO has a design according to formula: 5′ A-B-C 3′ as described elsewhere herein.

In some aspects, the ASO targets a mRNA encoding a particular isoform of NLRP3 protein (e.g., Isoform 1). In some aspects, the ASO targets all isoforms of NLRP3 protein. In other aspects, the ASO targets two isoforms (e.g., Isoform 1 and Isoform 2, Isoform 3 and Isoform 4, and Isoform 5 and Isoform 6) of NLRP3 protein.

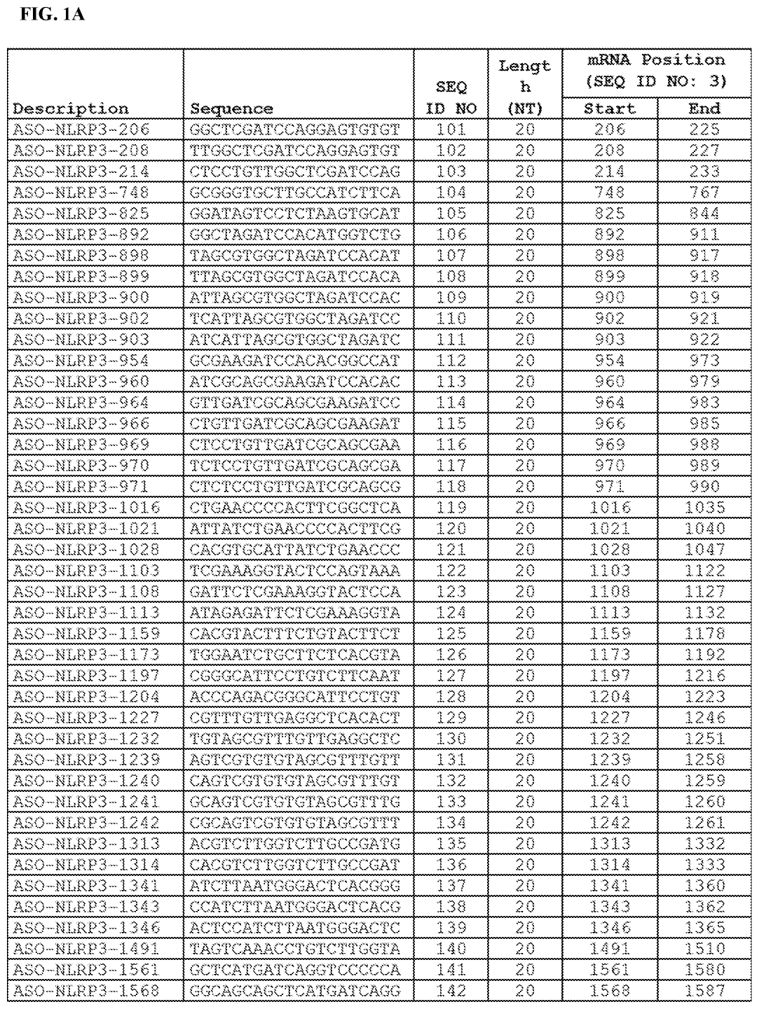

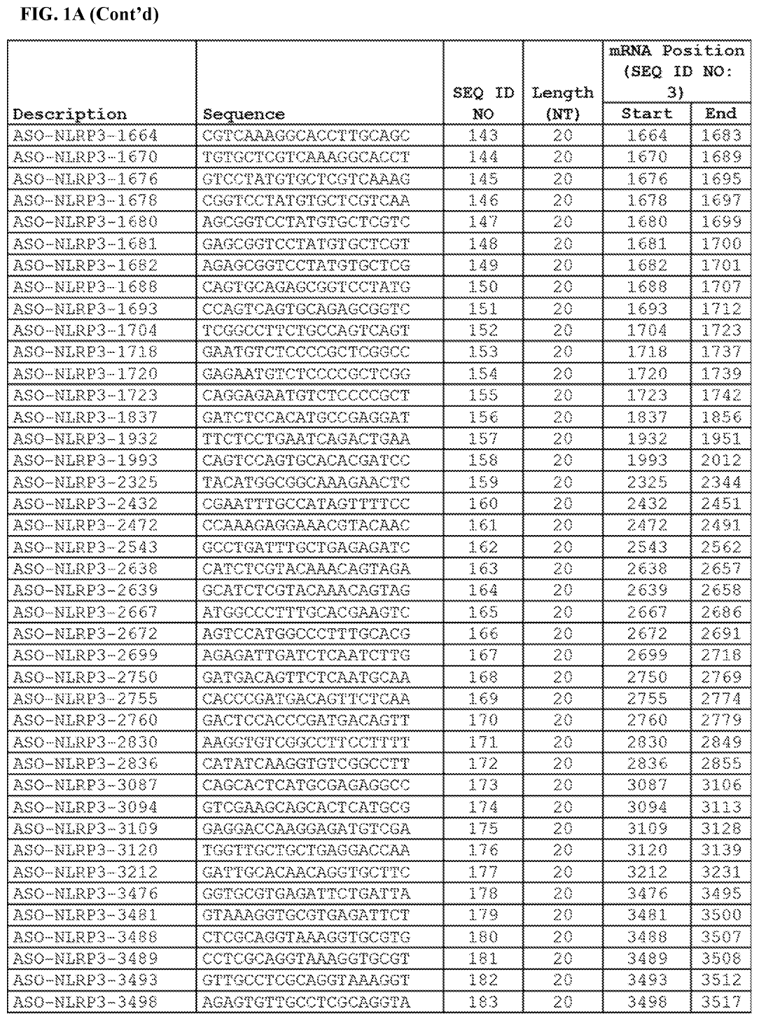

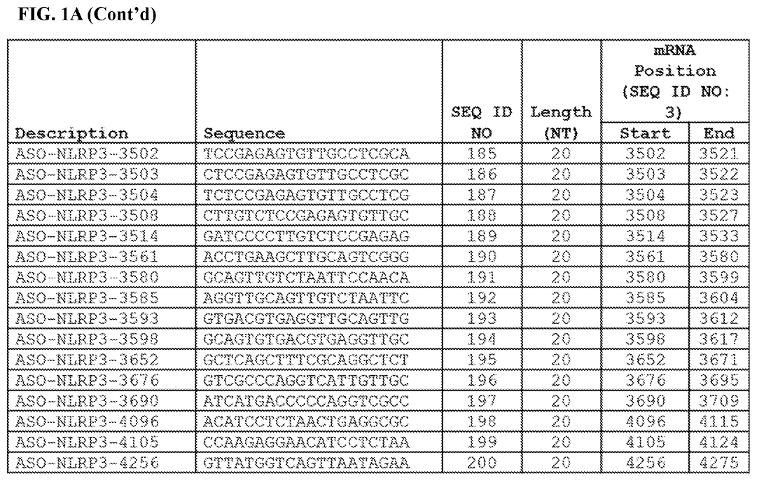

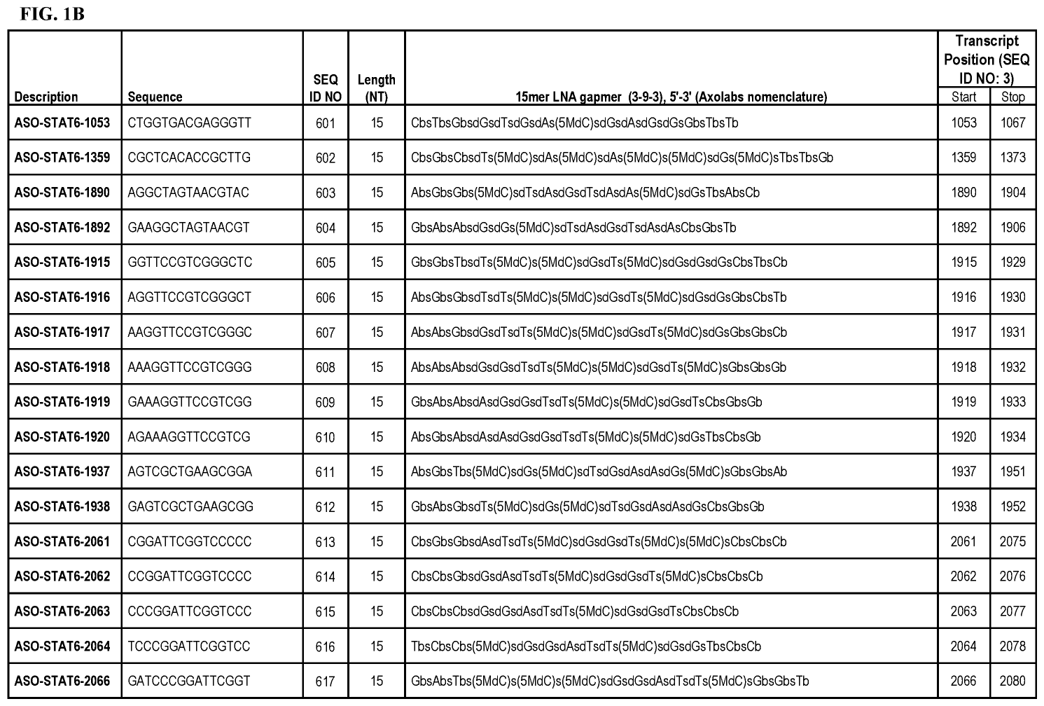

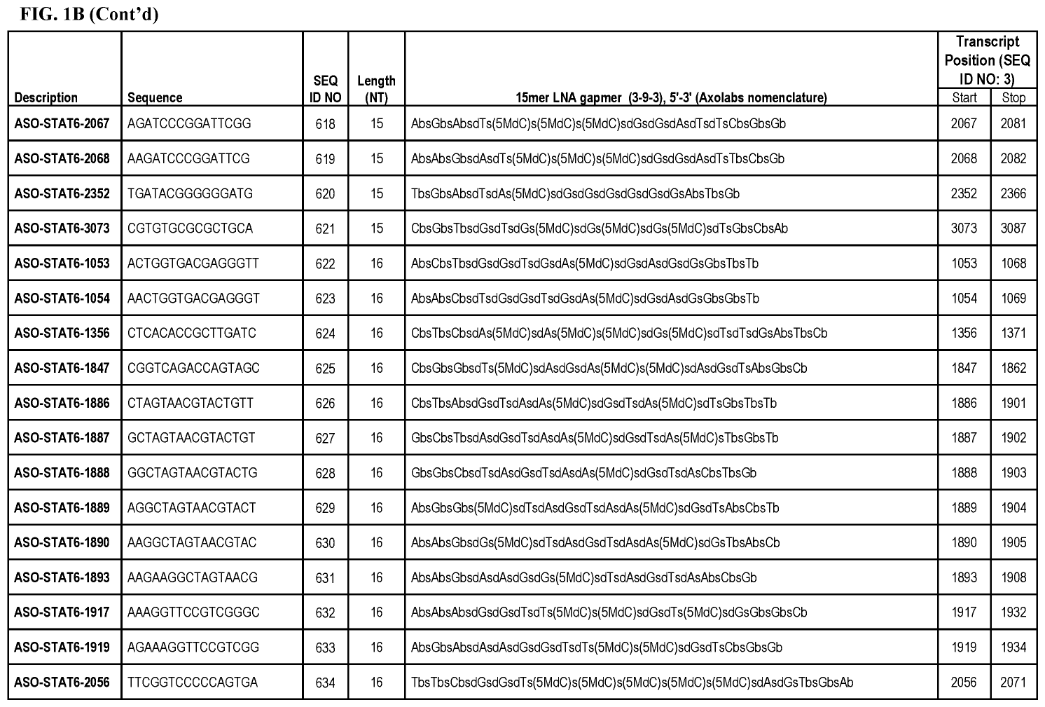

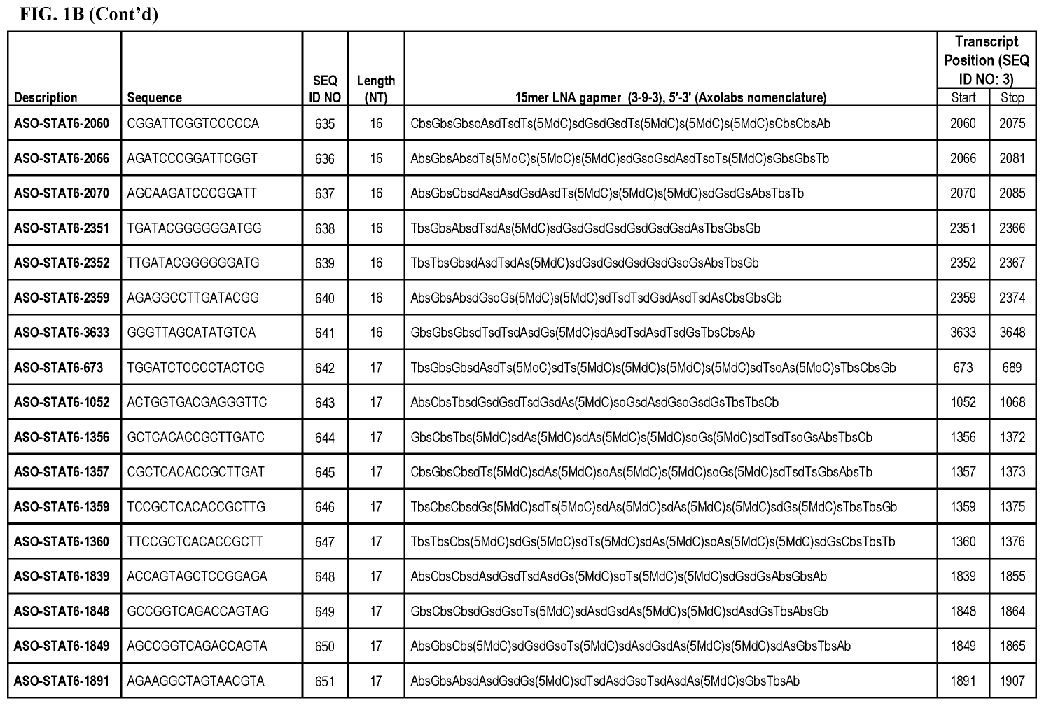

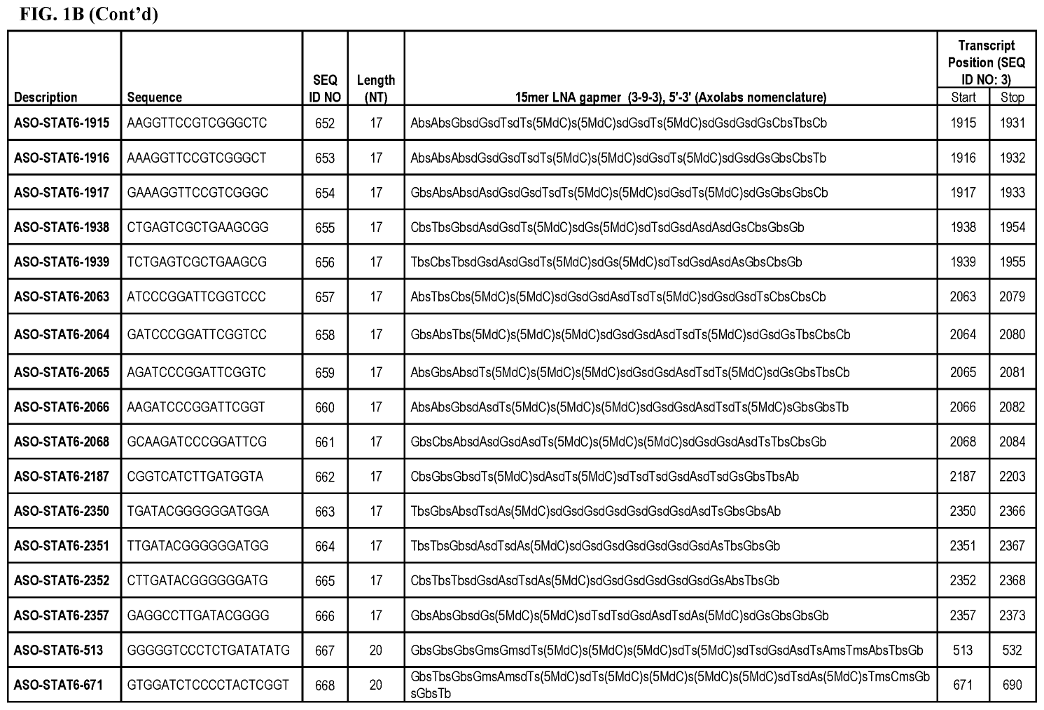

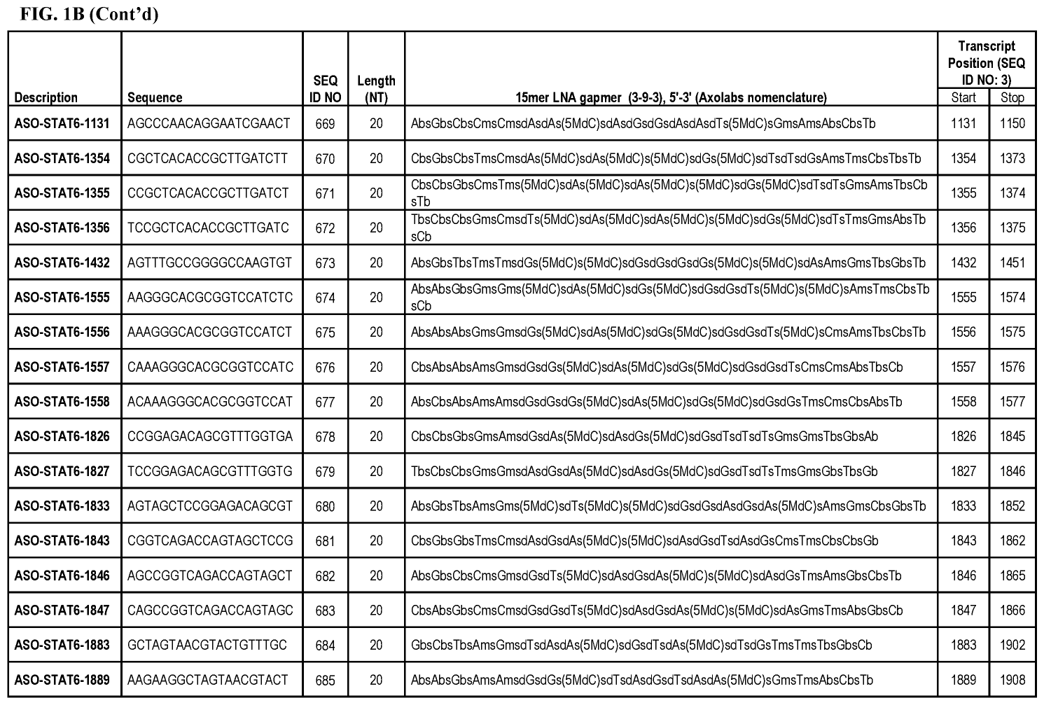

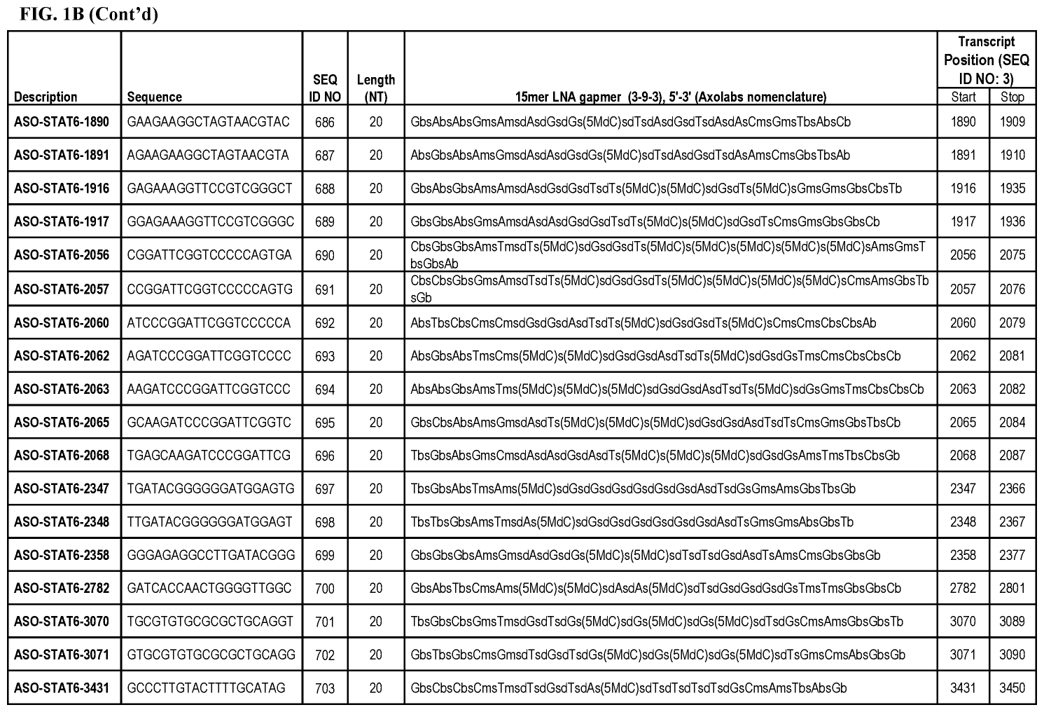

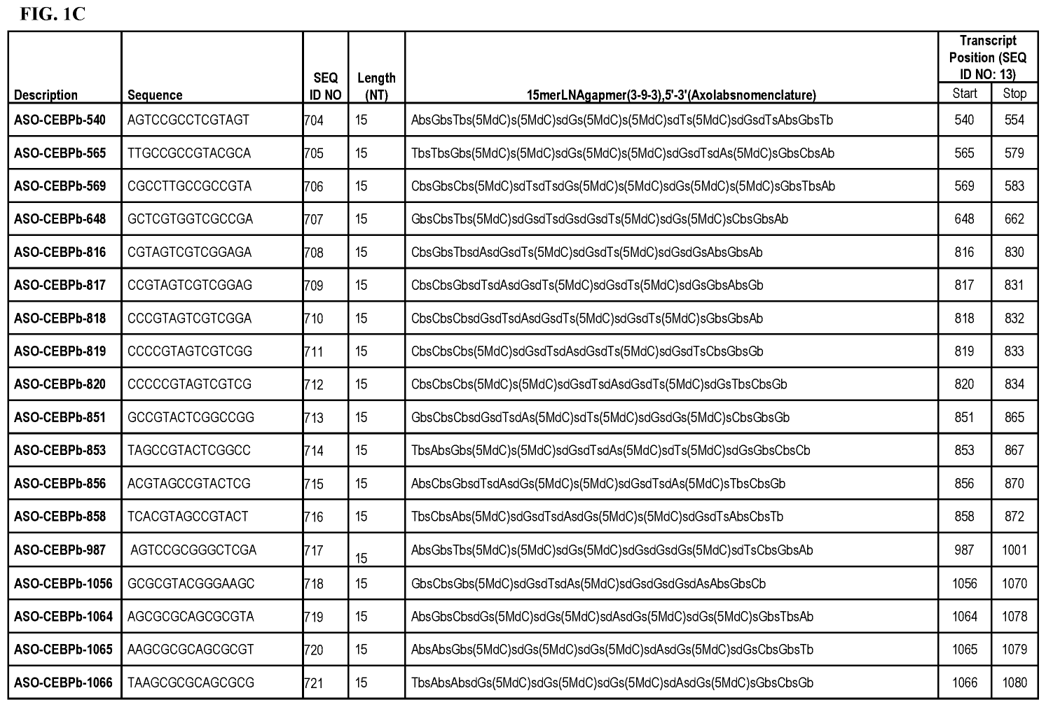

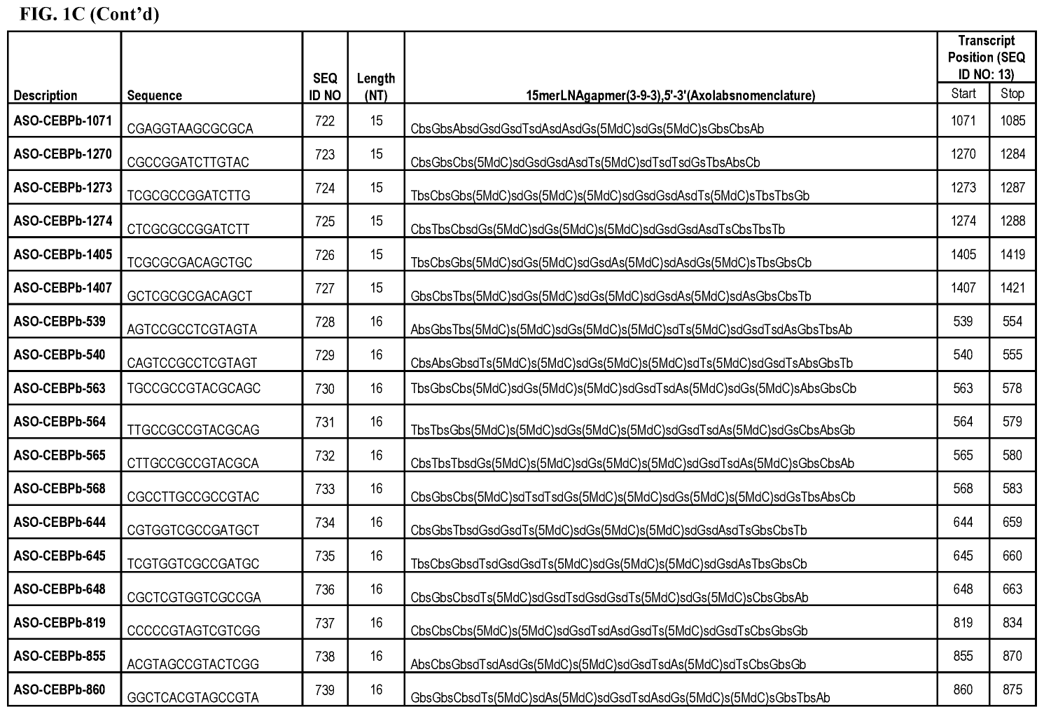

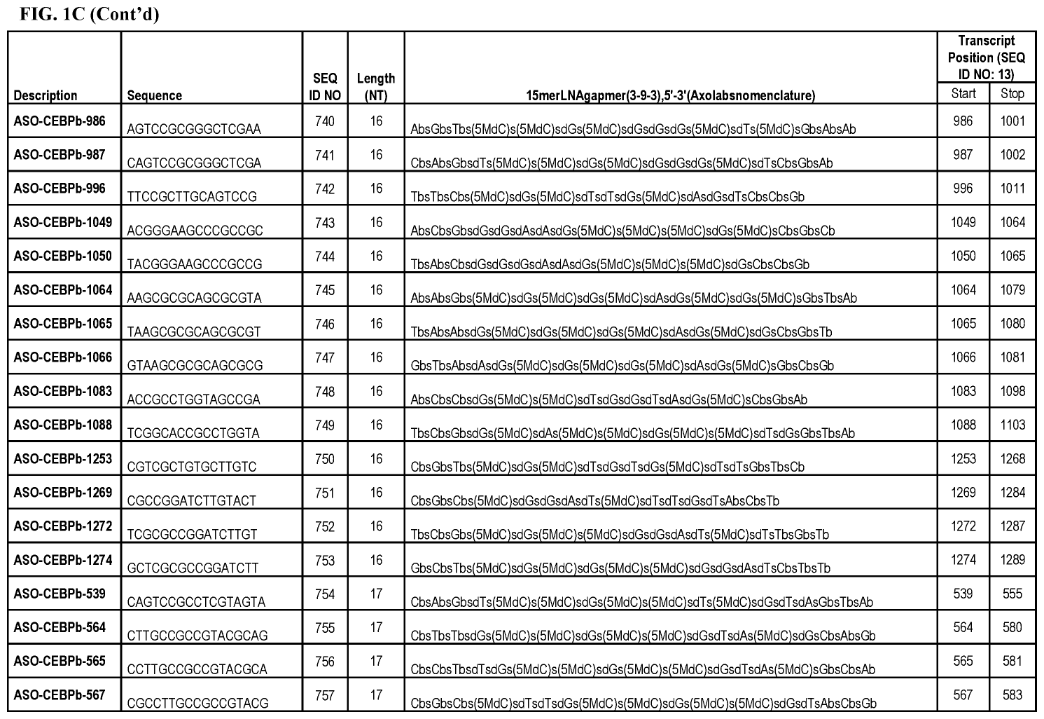

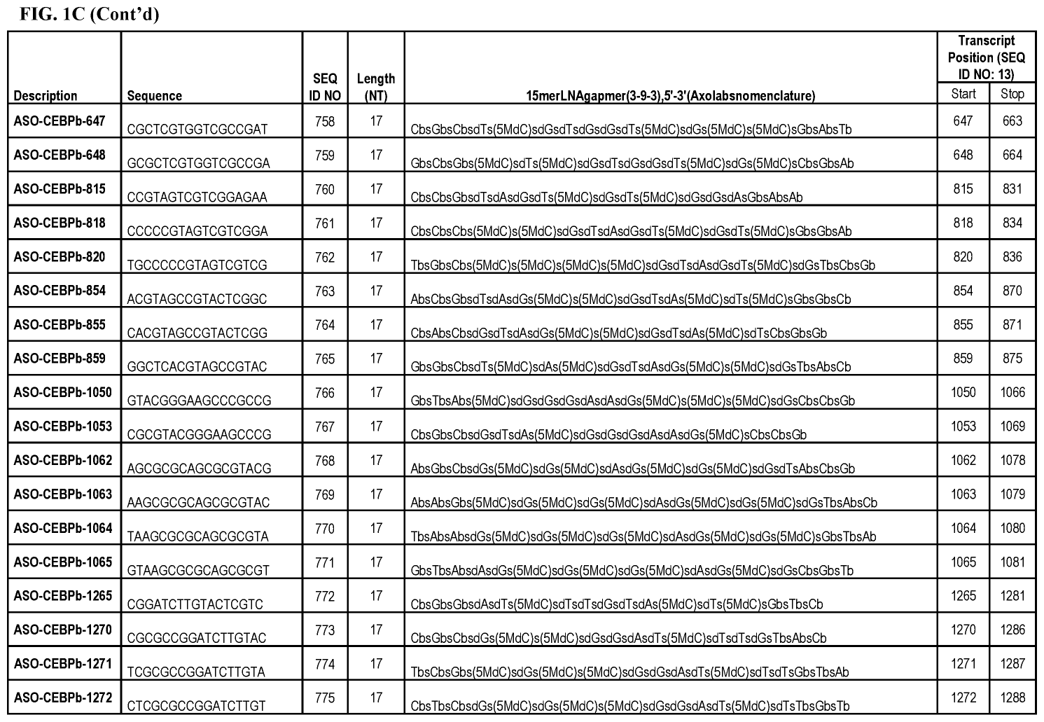

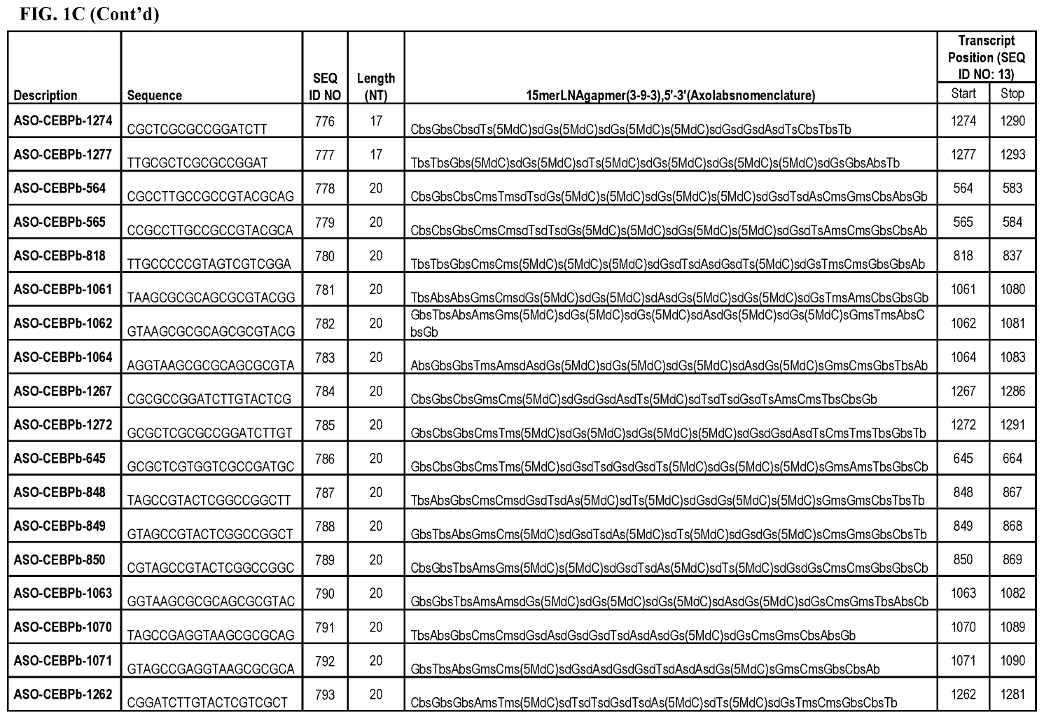

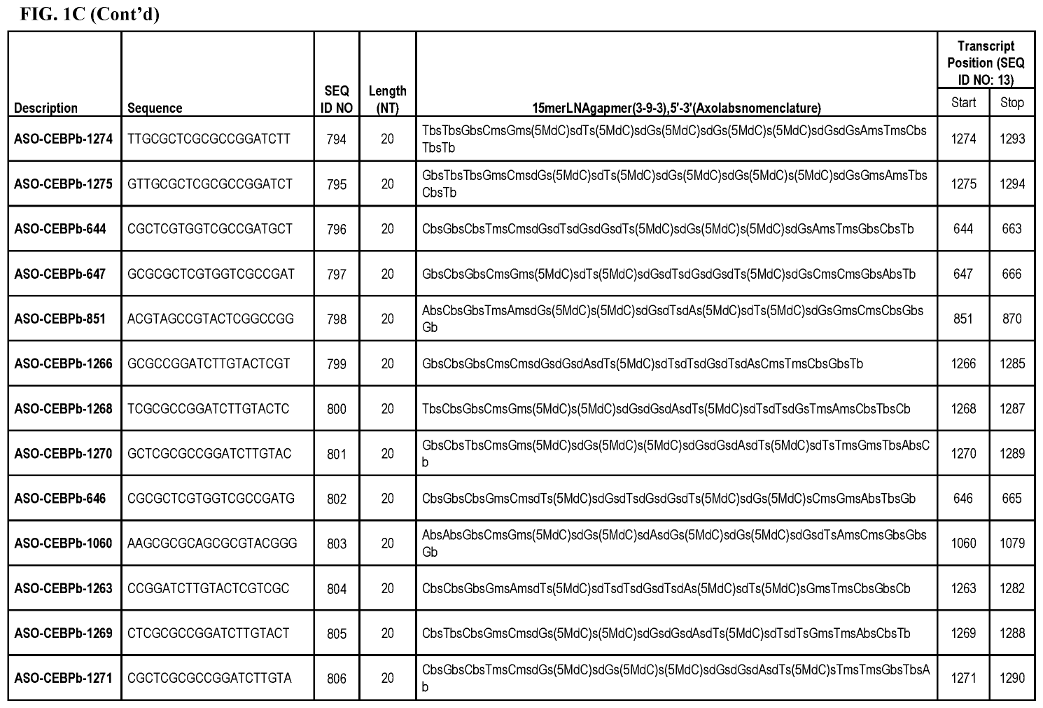

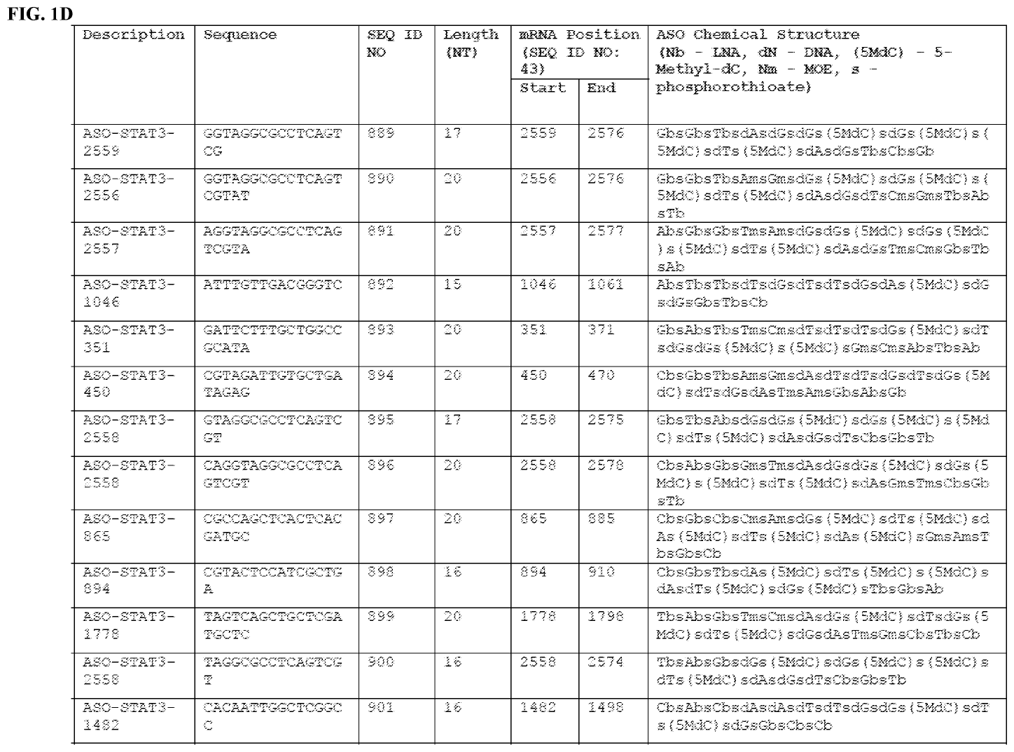

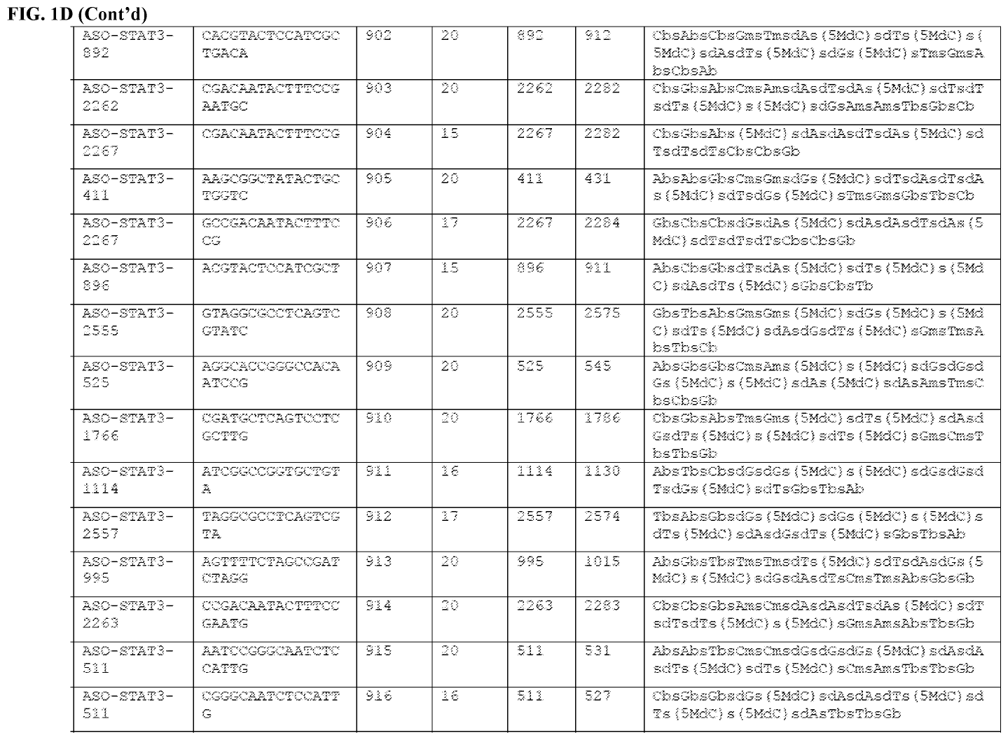

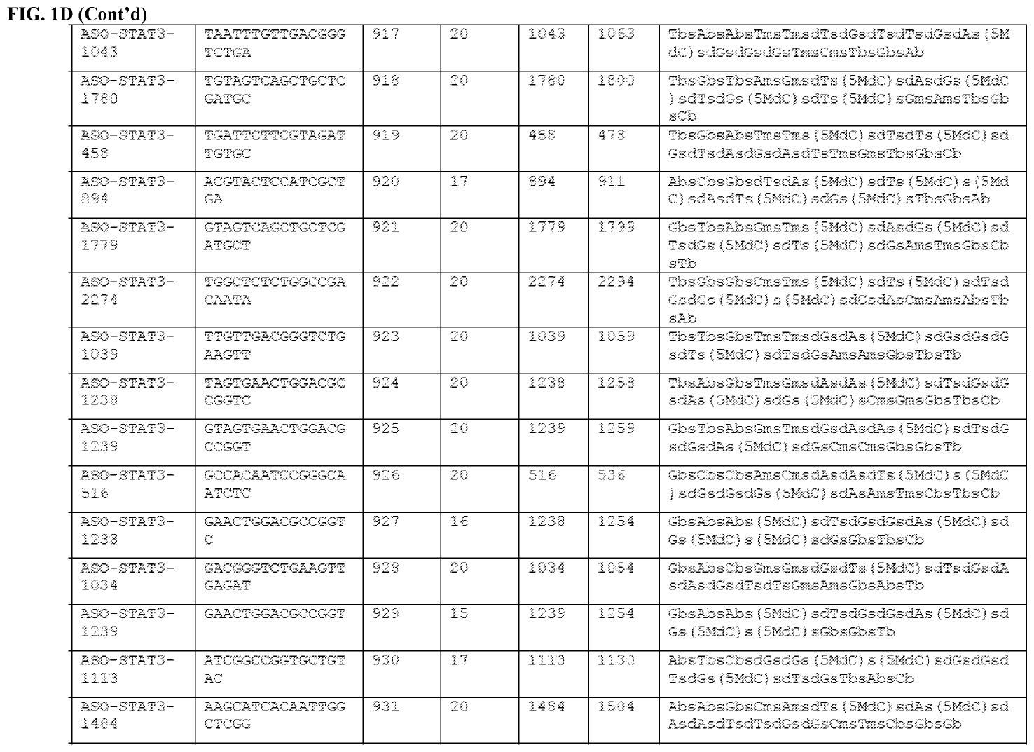

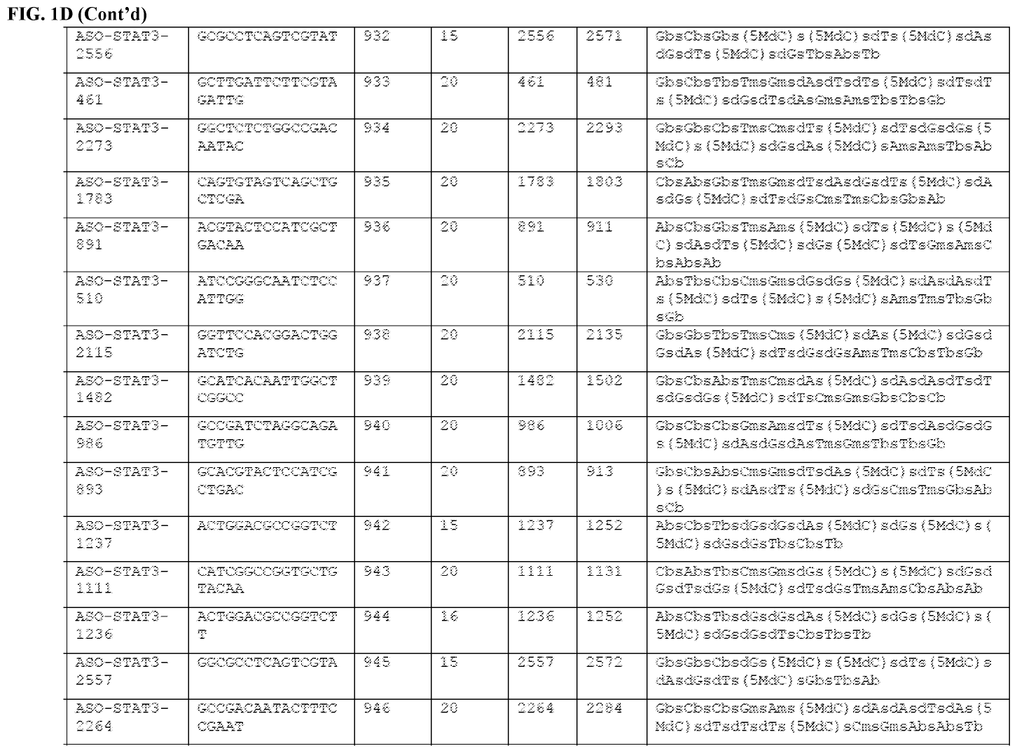

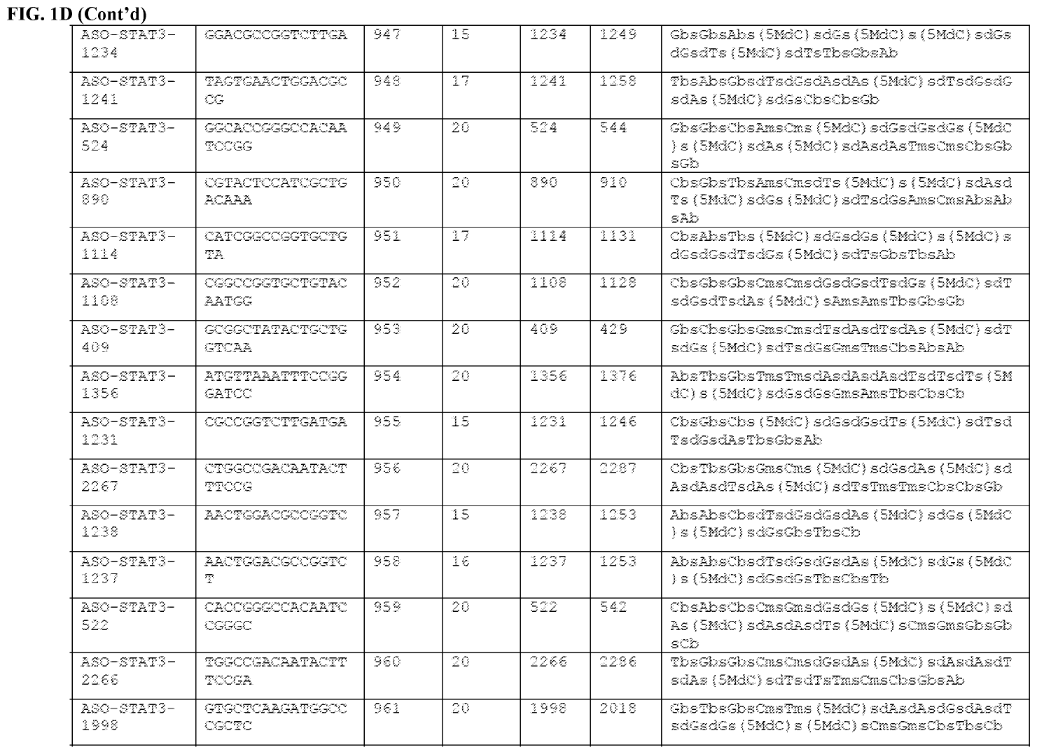

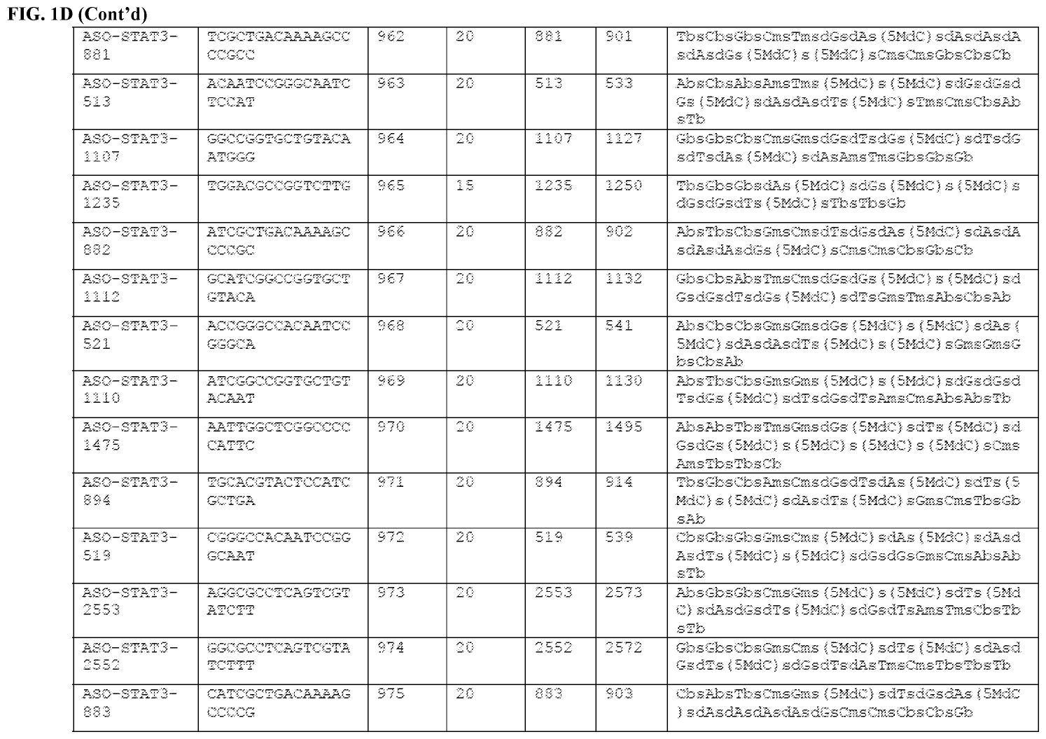

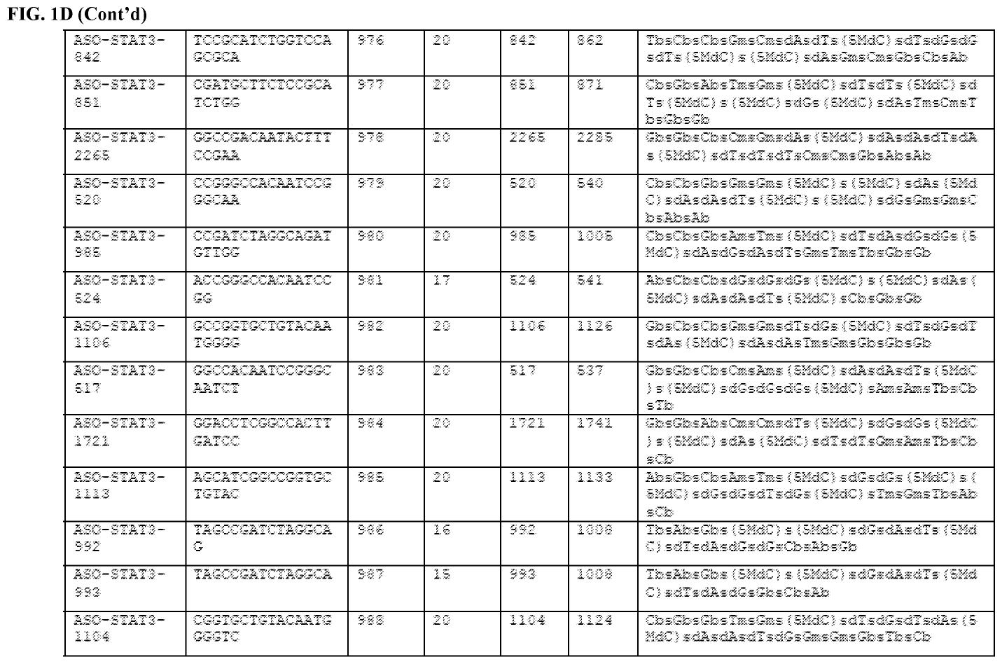

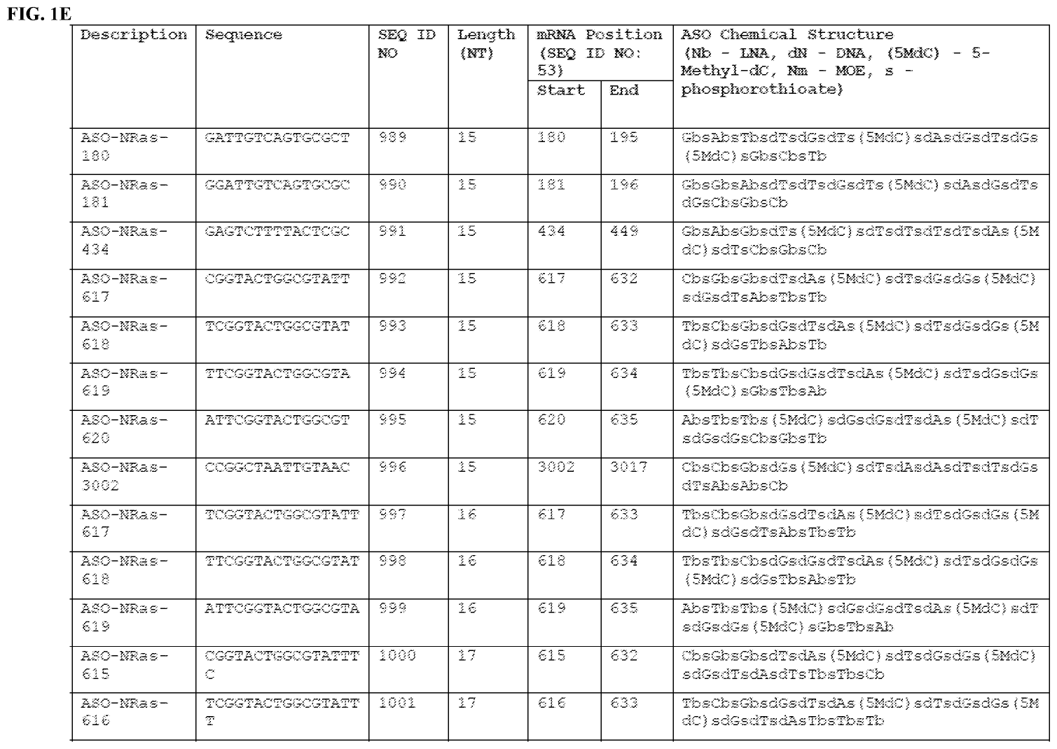

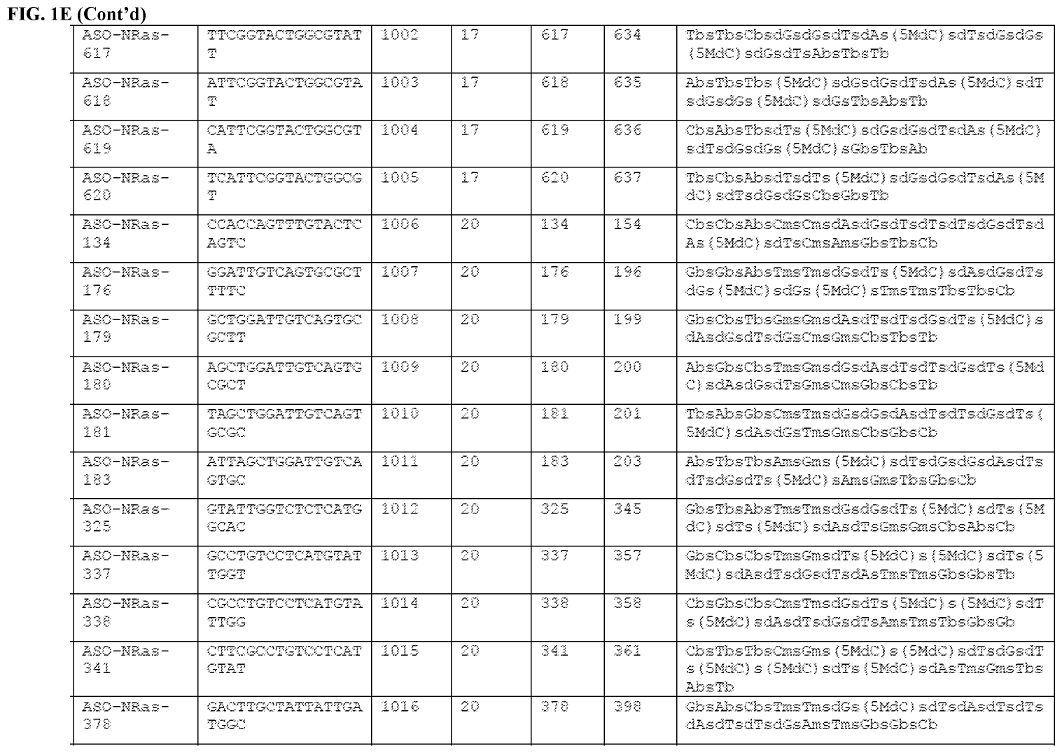

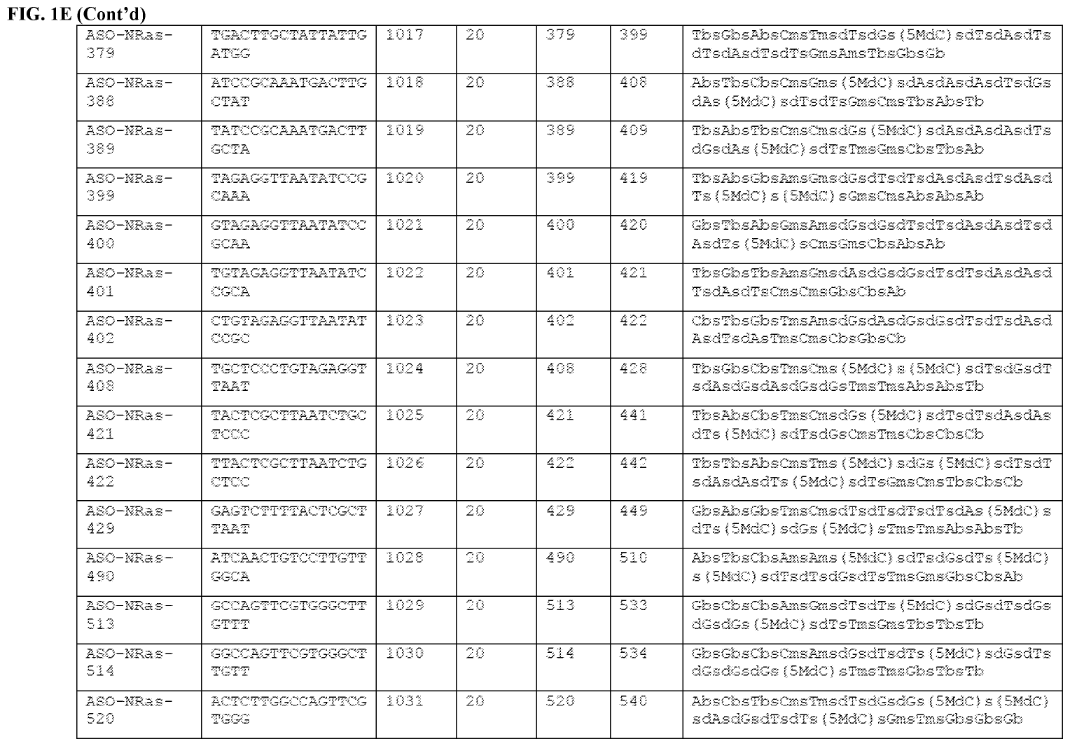

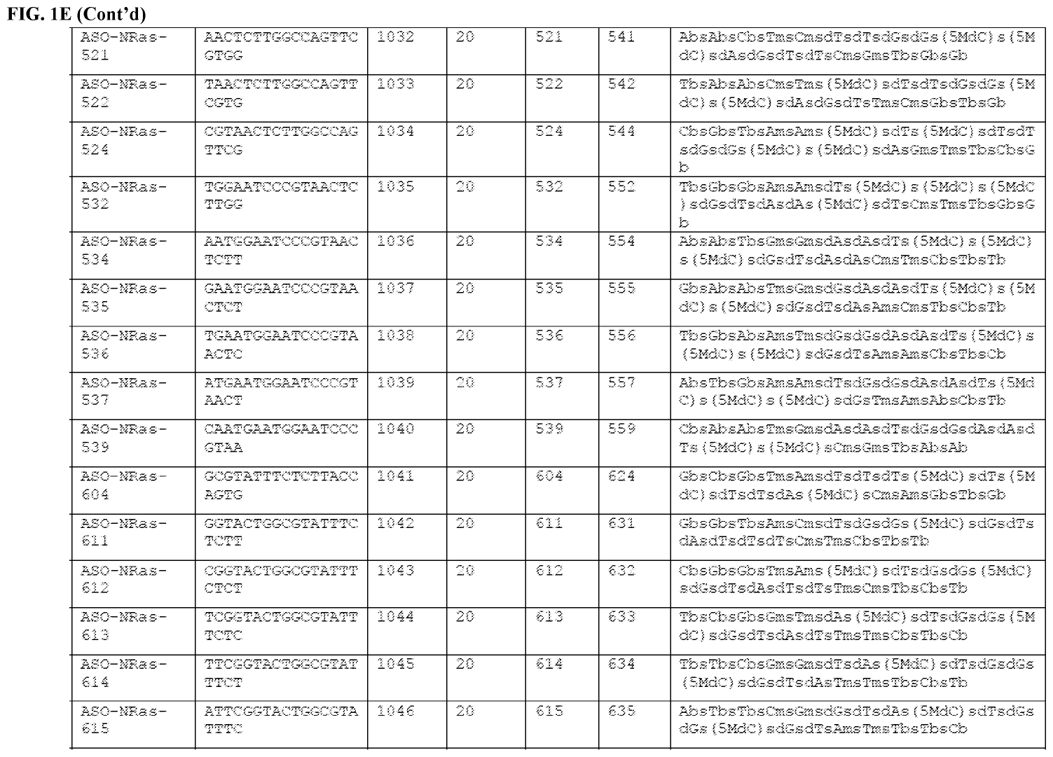

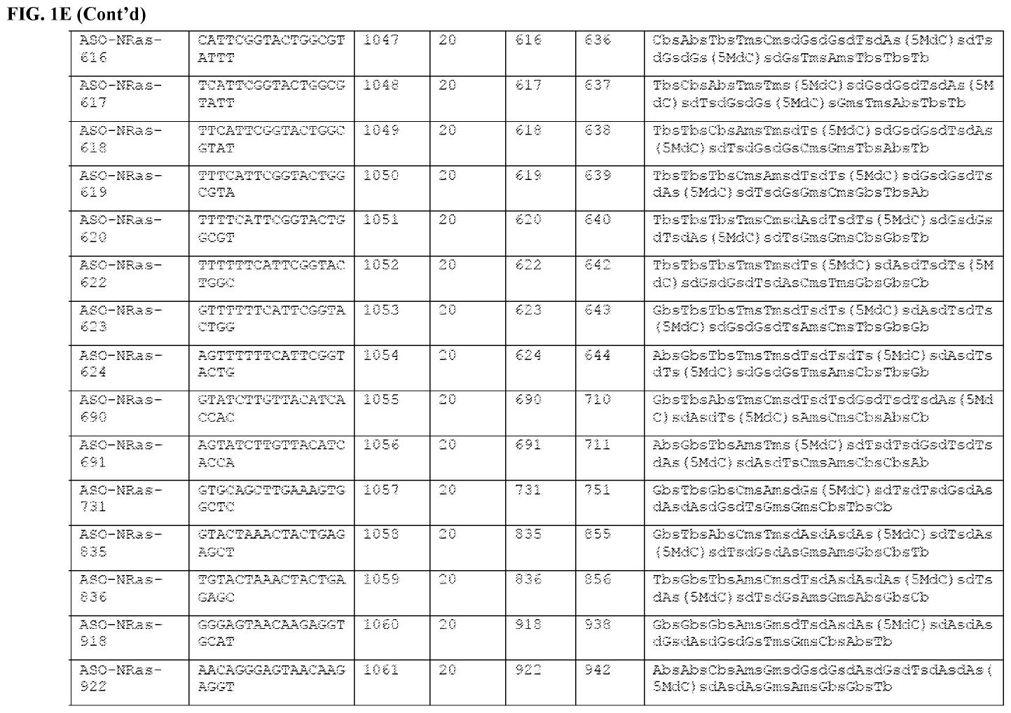

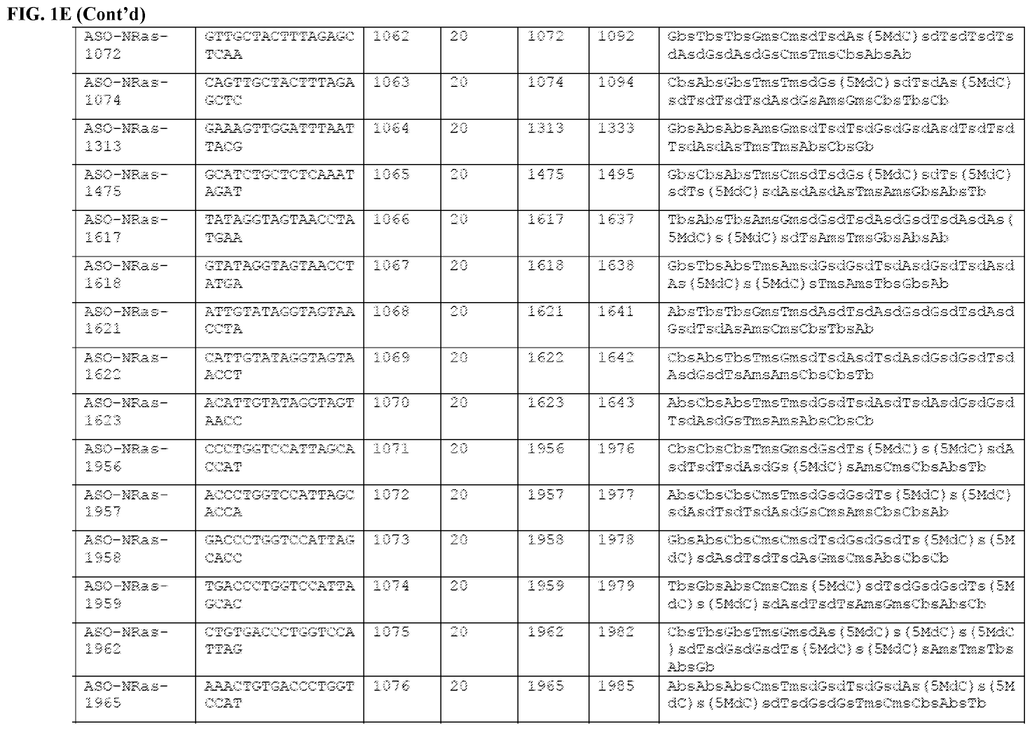

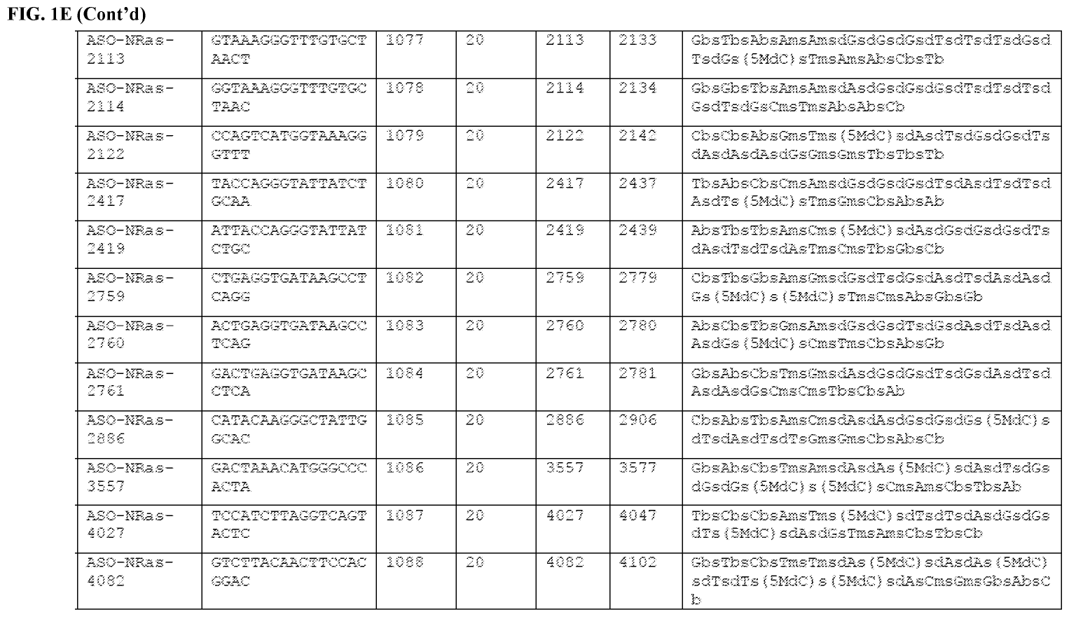

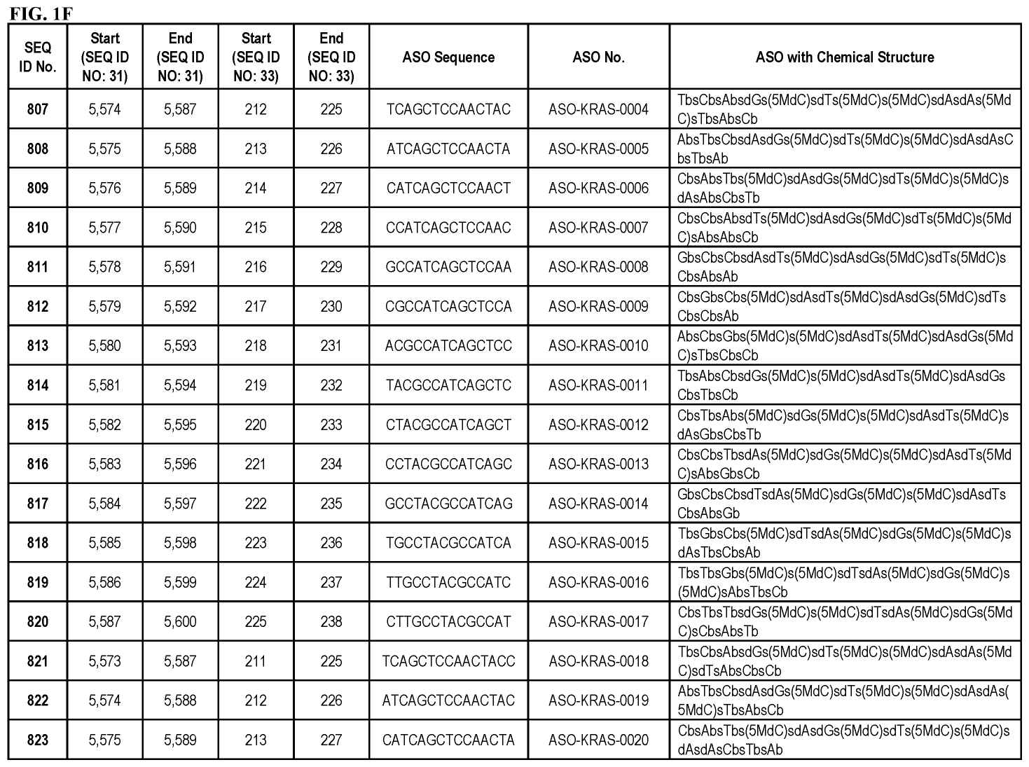

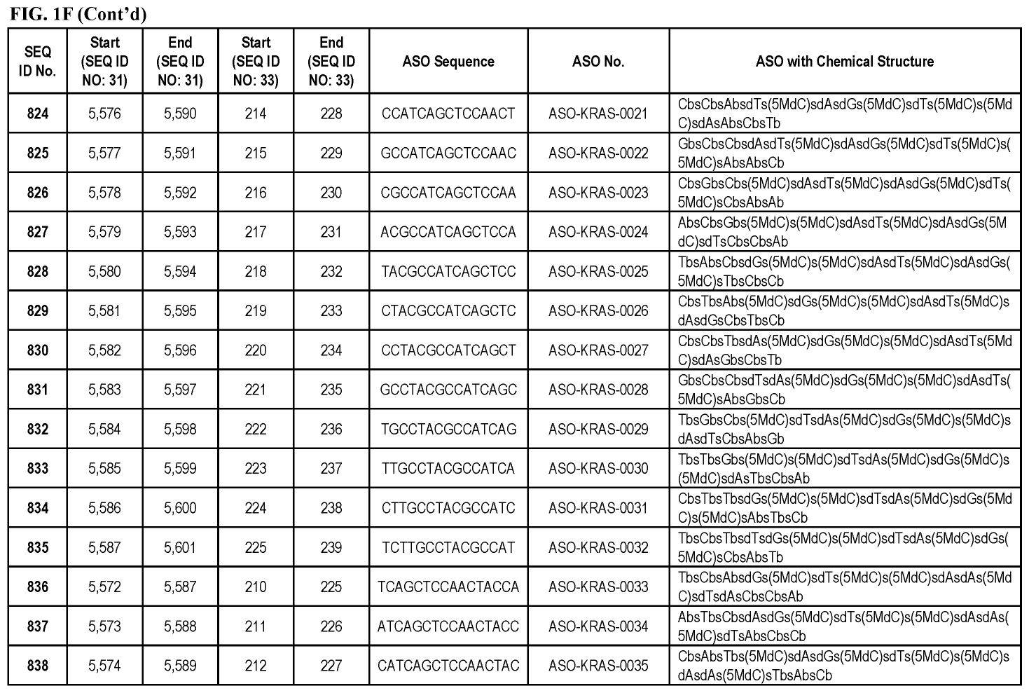

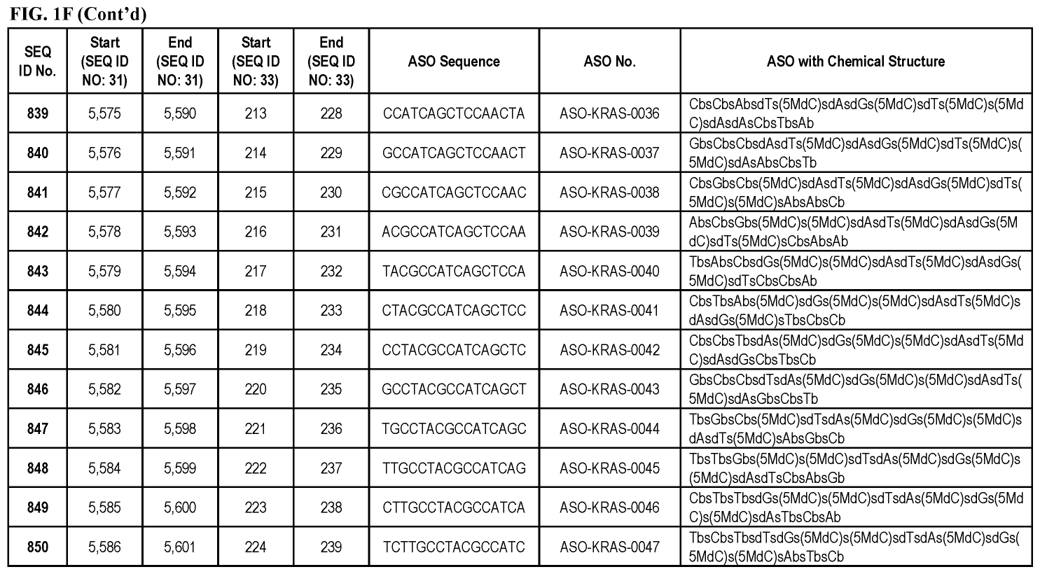

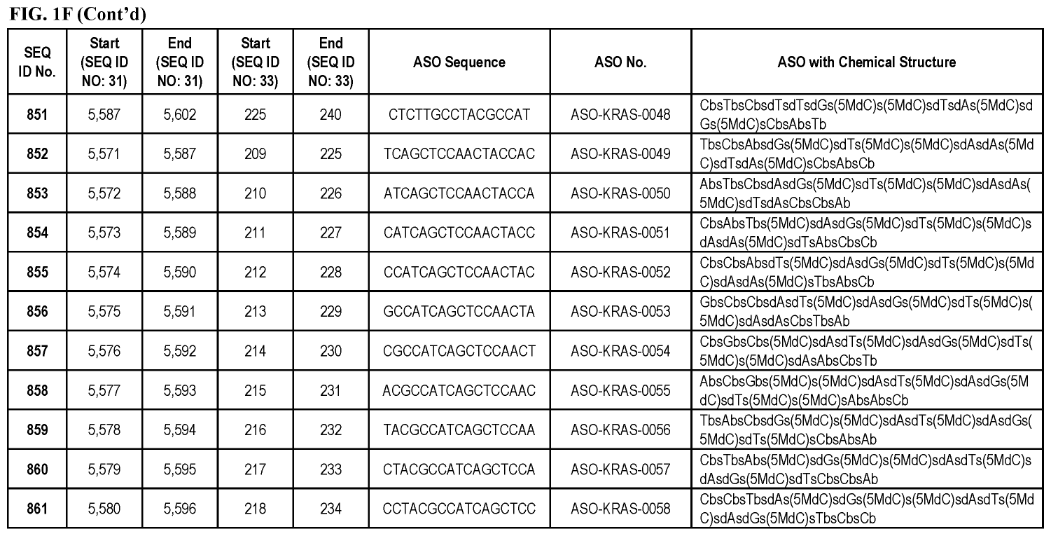

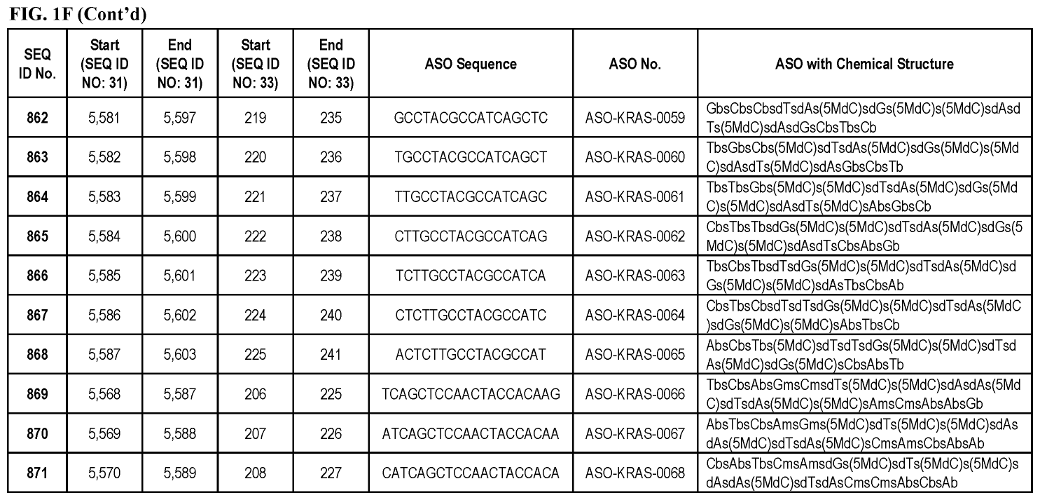

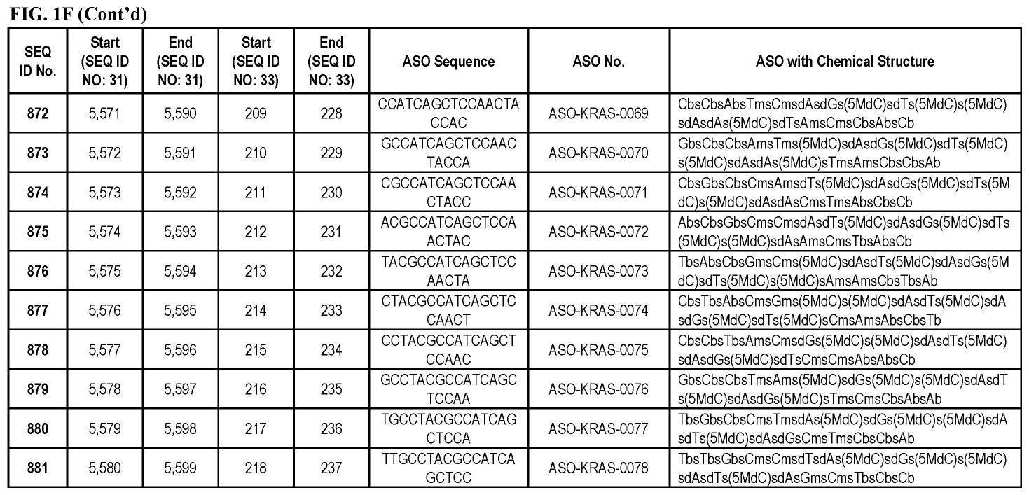

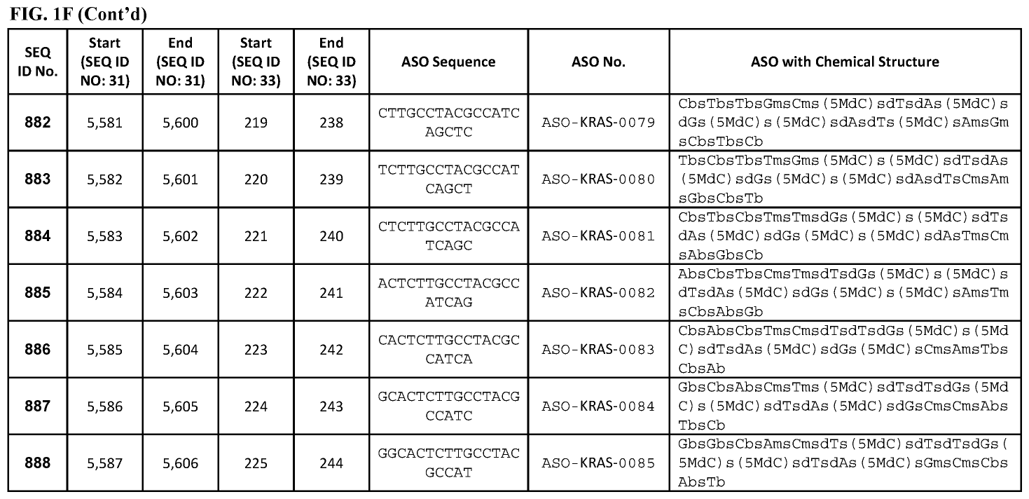

In some aspects, the nucleotide sequence of the ASOs of the disclosure or the contiguous nucleotide sequence has at least about 80% sequence identity to a sequence selected from SEQ ID NOs: 101 to 200 (i.e., the sequences in FIG. 1A), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1A).

In some aspects the ASO (or contiguous nucleotide portion thereof) is selected from, or comprises, one of the sequences selected from the group consisting of SEQ ID NOs: 101 to 200 or a region of at least 10 contiguous nucleotides thereof, wherein the ASO (or contiguous nucleotide portion thereof) can optionally comprise one, two, three, or four mismatches when compared to the corresponding NLRP3 transcript.

In some aspects, the ASO comprises a sequence selected from the group consisting of SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, SEQ ID NO: 199, or SEQ ID NO: 200.

In some aspects, the ASO comprises the sequence as set forth in SEQ ID NO: 101. In some aspects, the ASO comprises the sequence as set forth in SEQ ID NO: 102. In some aspects, the ASO comprises the sequence as set forth in SEQ ID NO: 103. In some aspects, the ASO comprises the sequence as set forth in SEQ ID NO: 104. In some aspects, the ASO comprises the sequence as set forth in SEQ ID NO: 105. In some aspects, the ASO comprises the sequence as set forth in SEQ ID NO: 106. In some aspects, the ASO comprises the sequence as set forth in SEQ ID NO: 107. In some aspects, the ASO comprises the sequence as set forth in SEQ ID NO: 108. In some aspects, the ASO comprises the sequence as set forth in SEQ ID NO: 109. In some aspects, the ASO comprises the sequence as set forth in SEQ ID NO: 110. In some aspects, the ASO comprises the sequence as set forth in SEQ ID NO: 111. In some aspects, the ASO comprises the sequence as set forth in SEQ ID NO: 112. In some aspects, the ASO comprises the sequence as set forth in SEQ ID NO: 113. In some aspects, the ASO comprises the sequence as set forth in SEQ ID NO: 114. In some aspects, the ASO comprises the sequence as set forth in SEQ ID NO: 115. In some aspects, the ASO comprises the sequence as set forth in SEQ ID NO: 116. In some aspects, the ASO comprises the sequence as set forth in SEQ ID NO: 117. In some aspects, the ASO comprises the sequence as set forth in SEQ ID NO: 118. In some aspects, the ASO comprises the sequence as set forth in SEQ ID NO: 119. In some aspects, the ASO comprises the sequence as set forth in SEQ ID NO: 120. In some aspects, the ASO comprises the sequence as set forth in SEQ ID NO: 121. In some aspects, the ASO comprises the sequence as set forth in SEQ ID NO: 122. In some aspects, the ASO comprises the sequence as set forth in SEQ ID NO: 123. In some aspects, the ASO comprises the sequence as set forth in SEQ ID NO: 124. In some aspects, the ASO comprises the sequence as set forth in SEQ ID NO: 125. In some aspects, the ASO comprises the sequence as set forth in SEQ ID NO: 126. In some aspects, the ASO comprises the sequence as set forth in SEQ ID NO: 127. In some aspects, the ASO comprises the sequence as set forth in SEQ ID NO: 128. In some aspects, the ASO comprises the sequence as set forth in SEQ ID NO: 129. In some aspects, the ASO comprises the sequence as set forth in SEQ ID NO: 130. In some aspects, the ASO comprises the sequence as set forth in SEQ ID NO: 131. In some aspects, the ASO comprises the sequence as set forth in SEQ ID NO: 132. In some aspects, the ASO comprises the sequence as set forth in SEQ ID NO: 133. In some aspects, the ASO comprises the sequence as set forth in SEQ ID NO: 134. In some aspects, the ASO comprises the sequence as set forth in SEQ ID NO: 135. In some aspects, the ASO comprises the sequence as set forth in SEQ ID NO: 136. In some aspects, the ASO comprises the sequence as set forth in SEQ ID NO: 137. In some aspects, the ASO comprises the sequence as set forth in SEQ ID NO: 138. In some aspects, the ASO comprises the sequence as set forth in SEQ ID NO: 139. In some aspects, the ASO comprises the sequence as set forth in SEQ ID NO: 140. In some aspects, the ASO comprises the sequence as set forth in SEQ ID NO: 141. In some aspects, the ASO comprises the sequence as set forth in SEQ ID NO: 142. In some aspects, the ASO comprises the sequence as set forth in SEQ ID NO: 143. In some aspects, the ASO comprises the sequence as set forth in SEQ ID NO: 144. In some aspects, the ASO comprises the sequence as set forth in SEQ ID NO: 145. In some aspects, the ASO comprises the sequence as set forth in SEQ ID NO: 146. In some aspects, the ASO comprises the sequence as set forth in SEQ ID NO: 147. In some aspects, the ASO comprises the sequence as set forth in SEQ ID NO: 148. In some aspects, the ASO comprises the sequence as set forth in SEQ ID NO: 149. In some aspects, the ASO comprises the sequence as set forth in SEQ ID NO: 150. In some aspects, the ASO comprises the sequence as set forth in SEQ ID NO: 151. In some aspects, the ASO comprises the sequence as set forth in SEQ ID NO: 152. In some aspects, the ASO comprises the sequence as set forth in SEQ ID NO: 153. In some aspects, the ASO comprises the sequence as set forth in SEQ ID NO: 154. In some aspects, the ASO comprises the sequence as set forth in SEQ ID NO: 155. In some aspects, the ASO comprises the sequence as set forth in SEQ ID NO: 156. In some aspects, the ASO comprises the sequence as set forth in SEQ ID NO: 157. In some aspects, the ASO comprises the sequence as set forth in SEQ ID NO: 158. In some aspects, the ASO comprises the sequence as set forth in SEQ ID NO: 159. In some aspects, the ASO comprises the sequence as set forth in SEQ ID NO: 160. In some aspects, the ASO comprises the sequence as set forth in SEQ ID NO: 161. In some aspects, the ASO comprises the sequence as set forth in SEQ ID NO: 162. In some aspects, the ASO comprises the sequence as set forth in SEQ ID NO: 163. In some aspects, the ASO comprises the sequence as set forth in SEQ ID NO: 164. In some aspects, the ASO comprises the sequence as set forth in SEQ ID NO: 165. In some aspects, the ASO comprises the sequence as set forth in SEQ ID NO: 166. In some aspects, the ASO comprises the sequence as set forth in SEQ ID NO: 167. In some aspects, the ASO comprises the sequence as set forth in SEQ ID NO: 168. In some aspects, the ASO comprises the sequence as set forth in SEQ ID NO: 169. In some aspects, the ASO comprises the sequence as set forth in SEQ ID NO: 170. In some aspects, the ASO comprises the sequence as set forth in SEQ ID NO: 171. In some aspects, the ASO comprises the sequence as set forth in SEQ ID NO: 172. In some aspects, the ASO comprises the sequence as set forth in SEQ ID NO: 173. In some aspects, the ASO comprises the sequence as set forth in SEQ ID NO: 174. In some aspects, the ASO comprises the sequence as set forth in SEQ ID NO: 175. In some aspects, the ASO comprises the sequence as set forth in SEQ ID NO: 176. In some aspects, the ASO comprises the sequence as set forth in SEQ ID NO: 177. In some aspects, the ASO comprises the sequence as set forth in SEQ ID NO: 178. In some aspects, the ASO comprises the sequence as set forth in SEQ ID NO: 179. In some aspects, the ASO comprises the sequence as set forth in SEQ ID NO: 180. In some aspects, the ASO comprises the sequence as set forth in SEQ ID NO: 181. In some aspects, the ASO comprises the sequence as set forth in SEQ ID NO: 182. In some aspects, the ASO comprises the sequence as set forth in SEQ ID NO: 183. In some aspects, the ASO comprises the sequence as set forth in SEQ ID NO: 184. In some aspects, the ASO comprises the sequence as set forth in SEQ ID NO: 185. In some aspects, the ASO comprises the sequence as set forth in SEQ ID NO: 186. In some aspects, the ASO comprises the sequence as set forth in SEQ ID NO: 187. In some aspects, the ASO comprises the sequence as set forth in SEQ ID NO: 188. In some aspects, the ASO comprises the sequence as set forth in SEQ ID NO: 189. In some aspects, the ASO comprises the sequence as set forth in SEQ ID NO: 190. In some aspects, the ASO comprises the sequence as set forth in SEQ ID NO: 191. In some aspects, the ASO comprises the sequence as set forth in SEQ ID NO: 192. In some aspects, the ASO comprises the sequence as set forth in SEQ ID NO: 193. In some aspects, the ASO comprises the sequence as set forth in SEQ ID NO: 194. In some aspects, the ASO comprises the sequence as set forth in SEQ ID NO: 195. In some aspects, the ASO comprises the sequence as set forth in SEQ ID NO: 196. In some aspects, the ASO comprises the sequence as set forth in SEQ ID NO: 197. In some aspects, the ASO comprises the sequence as set forth in SEQ ID NO: 198. In some aspects, the ASO comprises the sequence as set forth in SEQ ID NO: 199. In some aspects, the ASO comprises the sequence as set forth in SEQ ID NO: 200.

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 101 to 200. In some aspects the ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding NLRP3 transcript. In some aspects the ASO (or contiguous nucleotide portion thereof) is selected from, or comprises, one of the sequences selected from the group consisting of SEQ ID NOs: 101 to 200 except for 1, 2, 3, 4, 5, 6, 7, 8, 9 or 10 substitutions, wherein the substituted ASO can bind to the NLRP3 transcript. In some aspects the ASO (or contiguous nucleotide portion thereof) is selected from, or comprises, one of the sequences selected from the group consisting of SEQ ID NOs: 101 to 200 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding NLRP3 transcript.

In some aspects, binding of an ASO targeting a NLRP3 transcript disclosed herein to a mRNA transcript encoding NLRP3 can reduce expression levels and/or activity levels of NLRP3.

II.C2. ASO Targeting STAT6

STAT6 (STAT6) is also known as signal transducer and activator of transcription 6. Synonyms of STAT6/STAT6 are known and include IL-4 STAT; STAT, Interleukin4-Induced; Transcription Factor IL-4 STAT; STAT6B; STAT6C; and D12S1644. The sequence for the human STAT6 gene can be found under publicly available GenBank Accession Number NC_000012.12:c57111413-57095404. The human STAT6 gene is found at chromosome location 12q13.3 at 57111413-57095404, complement. Unless indicated otherwise, the term “STAT6,” as used herein, can refer to STAT6 from one or more species (e.g., humans, non-human primates, dogs, cats, guinea pigs, rabbits, rats, mice, horses, cattle, and bears).

The sequence for the human STAT6 pre-mRNA transcript (SEQ ID NO: 11) corresponds to the reverse complement of residues 57111413-57095404, complement, of chromosome 12q13.3. The STAT6 mRNA sequence (GenBank Accession No. NM_001178078.1) is provided in SEQ ID NO: 13, except that the nucleotide “t” in SEQ ID NO: 13 is shown as “u” in the mRNA. The sequence for human STAT6 protein can be found under publicly available Accession Numbers: P42226-1, (canonical sequence, SEQ ID NO: 12), P42226-2 (SEQ ID NO: 14), and P42226-3 (SEQ ID NO: 15), each of which is incorporated by reference herein in its entirety.

Natural variants of the human STAT6 gene product are known. For example, natural variants of human STAT6 protein can contain one or more amino acid substitutions selected from: M118R, D419N, and any combination thereof. Additional variants of human STAT6 protein resulting from alternative splicing are also known in the art. STAT6 Isoform 2 (identifier: P42226-2 at UniProt) differs from the canonical sequence (SEQ ID NO: 13) as follows: deletion of residues 1-174 and substitution of ₁₇₅PSE₁₇₇ with ₁₇₅MEQ₁₇₇ relative to SEQ ID NO: 13. The sequence of STAT6 Isoform 3 (identifier: P42226-3) differs from the canonical sequence (SEQ ID NO: 13) as follows: deletion of residues 1-110 relative to SEQ ID NO: 13. Therefore, the ASOs of the present disclosure can be designed to reduce or inhibit expression of the natural variants of the STAT6 protein.

An example of a target nucleic acid sequence of the ASOs is STAT6 pre-mRNA. SEQ ID NO: 11 represents a human STAT6 genomic sequence (i.e., reverse complement of nucleotides 57111413-57095404, complement, of chromosome 12q13.3). SEQ ID NO: 11 is identical to a STAT6 pre-mRNA sequence except that nucleotide “t” in SEQ ID NO: 11 is shown as “u” in pre-mRNA. In certain aspects, the “target nucleic acid” comprises an intron of a STAT6 protein-encoding nucleic acids or naturally occurring variants thereof, and RNA nucleic acids derived therefrom, e.g., pre-mRNA. In other aspects, the target nucleic acid comprises an exon region of a STAT6 protein-encoding nucleic acids or naturally occurring variants thereof, and RNA nucleic acids derived therefrom, e.g., pre-mRNA. In yet other aspects, the target nucleic acid comprises an exon-intron junction of a STAT6 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human STAT6 protein sequence encoded by the STAT6 pre-mRNA is shown as SEQ ID NO: 13. In other aspects, the target nucleic acid comprises an untranslated region of a STAT6 protein-encoding nucleic acids or naturally occurring variants thereof, e.g., 5′ UTR, 3′ UTR, or both.

In some aspects, an ASO of the disclosure hybridizes to a region within the introns of a STAT6 transcript, e.g., SEQ ID NO: 11. In certain aspects, an ASO of the disclosure hybridizes to a region within the exons of a STAT6 transcript, e.g., SEQ ID NO: 11. In other aspects, an ASO of the disclosure hybridizes to a region within the exon-intron junction of a STAT6 transcript, e.g., SEQ ID NO: 11. In some aspects, an ASO of the disclosure hybridizes to a region within a STAT6 transcript (e.g., an intron, exon, or exon-intron junction), e.g., SEQ ID NO: 11, wherein the ASO has a design according to formula: 5′ A-B-C 3′ as described elsewhere herein.

In some aspects, the ASO targets a mRNA encoding a particular isoform of STAT6 protein (e.g., Isoform 1). In some aspects, the ASO targets all isoforms of STAT6 protein. In other aspects, the ASO targets two isoforms (e.g., Isoform 1 and Isoform 2, Isoform 1 and Isoform 3, or Isoform 2 and Isoform 3) of STAT6 protein.

In some aspects, the nucleotide sequence of the ASOs of the disclosure or the contiguous nucleotide sequence has at least about 80% sequence identity to a sequence selected from SEQ ID NOs: 601 to 703 (i.e., the sequences in FIG. 1B), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1B).

In some aspects the ASO (or contiguous nucleotide portion thereof) is selected from, or comprises, one of the sequences selected from the group consisting of SEQ ID NOs: 601 to 703 or a region of at least 10 contiguous nucleotides thereof, wherein the ASO (or contiguous nucleotide portion thereof) can optionally comprise one, two, three, or four mismatches when compared to the corresponding STAT6 transcript.

In some aspects, the ASO comprises the sequence as set forth in SEQ ID NO: 601 (e.g., ASO-STAT6-1053). In some aspects, the ASO comprises the sequence as set forth in SEQ ID NO: 602 (e.g., ASO-STAT6-1359). In some aspects, the ASO comprises the sequence as set forth in SEQ ID NO: 603 (e.g., ASO-STAT6-1890). In some aspects, the ASO comprises the sequence as set forth in SEQ ID NO: 604 (e.g., ASO-STAT6-1892). In some aspects, the ASO comprises the sequence as set forth in SEQ ID NO: 605 (e.g., ASO-STAT6-1915). In some aspects, the ASO comprises the sequence as set forth in SEQ ID NO: 606 (e.g., ASO-STAT6-1916). In some aspects, the ASO comprises the sequence as set forth in SEQ ID NO: 607 (e.g., ASO-STAT6-1917). In some aspects, the ASO comprises the sequence as set forth in SEQ ID NO: 608 (e.g., ASO-STAT6-1918). In some aspects, the ASO comprises the sequence as set forth in SEQ ID NO: 609 (e.g., ASO-STAT6-1919). In some aspects, the ASO comprises the sequence as set forth in SEQ ID NO: 610 (e.g., ASO-STAT6-1920). In some aspects, the ASO comprises the sequence as set forth in SEQ ID NO: 611 (e.g., ASO-STAT6-1937). In some aspects, the ASO comprises the sequence as set forth in SEQ ID NO: 612 (e.g., ASO-STAT6-1938). In some aspects, the ASO comprises the sequence as set forth in SEQ ID NO: 613 (e.g., ASO-STAT6-2061). In some aspects, the ASO comprises the sequence as set forth in SEQ ID NO: 614 (e.g., ASO-STAT6-2062). In some aspects, the ASO comprises the sequence as set forth in SEQ ID NO: 615 (e.g., ASO-STAT6-2063). In some aspects, the ASO comprises the sequence as set forth in SEQ ID NO: 616 (e.g., ASO-STAT6-2064). In some aspects, the ASO comprises the sequence as set forth in SEQ ID NO: 617 (e.g., ASO-STAT6-2066). In some aspects, the ASO comprises the sequence as set forth in SEQ ID NO: 618 (e.g., ASO-STAT6-2067). In some aspects, the ASO comprises the sequence as set forth in SEQ ID NO: 619 (e.g., ASO-STAT6-2068). In some aspects, the ASO comprises the sequence as set forth in SEQ ID NO: 620 (e.g., ASO-STAT6-2352). In some aspects, the ASO comprises the sequence as set forth in SEQ ID NO: 621 (e.g., ASO-STAT6-3073). In some aspects, the ASO comprises the sequence as set forth in SEQ ID NO: 622 (e.g., ASO-STAT6-1053). In some aspects, the ASO comprises the sequence as set forth in SEQ ID NO: 623 (e.g., ASO-STAT6-1054). In some aspects, the ASO comprises the sequence as set forth in SEQ ID NO: 624 (e.g., ASO-STAT6-1356). In some aspects, the ASO comprises the sequence as set forth in SEQ ID NO: 625 (e.g., ASO-STAT6-1847). In some aspects, the ASO comprises the sequence as set forth in SEQ ID NO: 626 (e.g., ASO-STAT6-1886). In some aspects, the ASO comprises the sequence as set forth in SEQ ID NO: 627 (e.g., ASO-STAT6-1887). In some aspects, the ASO comprises the sequence as set forth in SEQ ID NO: 628 (e.g., ASO-STAT6-1888). In some aspects, the ASO comprises the sequence as set forth in SEQ ID NO: 629 (e.g., ASO-STAT6-1889). In some aspects, the ASO comprises the sequence as set forth in SEQ ID NO: 630 (e.g., ASO-STAT6-1890). In some aspects, the ASO comprises the sequence as set forth in SEQ ID NO: 631 (e.g., ASO-STAT6-1893). In some aspects, the ASO comprises the sequence as set forth in SEQ ID NO: 632 (e.g., ASO-STAT6-1917). In some aspects, the ASO comprises the sequence as set forth in SEQ ID NO: 633 (e.g., ASO-STAT6-1919). In some aspects, the ASO comprises the sequence as set forth in SEQ ID NO: 634 (e.g., ASO-STAT6-2056). In some aspects, the ASO comprises the sequence as set forth in SEQ ID NO: 635 (e.g., ASO-STAT6-2060). In some aspects, the ASO comprises the sequence as set forth in SEQ ID NO: 636 (e.g., ASO-STAT6-2066). In some aspects, the ASO comprises the sequence as set forth in SEQ ID NO: 637 (e.g., ASO-STAT6-2070). In some aspects, the ASO comprises the sequence as set forth in SEQ ID NO: 638 (e.g., ASO-STAT6-2351). In some aspects, the ASO comprises the sequence as set forth in SEQ ID NO: 639 (e.g., ASO-STAT6-2352). In some aspects, the ASO comprises the sequence as set forth in SEQ ID NO: 640 (e.g., ASO-STAT6-2359). In some aspects, the ASO comprises the sequence as set forth in SEQ ID NO: 641 (e.g., ASO-STAT6-3633). In some aspects, the ASO comprises the sequence as set forth in SEQ ID NO: 642 (e.g., ASO-STAT6-673). In some aspects, the ASO comprises the sequence as set forth in SEQ ID NO: 643 (e.g., ASO-STAT6-1052). In some aspects, the ASO comprises the sequence as set forth in SEQ ID NO: 644 (e.g., ASO-STAT6-1356). In some aspects, the ASO comprises the sequence as set forth in SEQ ID NO: 645 (e.g., ASO-STAT6-1357). In some aspects, the ASO comprises the sequence as set forth in SEQ ID NO: 646 (e.g., ASO-STAT6-1359). In some aspects, the ASO comprises the sequence as set forth in SEQ ID NO: 647 (e.g., ASO-STAT6-1360). In some aspects, the ASO comprises the sequence as set forth in SEQ ID NO: 648 (e.g., ASO-STAT6-1839). In some aspects, the ASO comprises the sequence as set forth in SEQ ID NO: 649 (e.g., ASO-STAT6-1848). In some aspects, the ASO comprises the sequence as set forth in SEQ ID NO: 650 (e.g., ASO-STAT6-1849). In some aspects, the ASO comprises the sequence as set forth in SEQ ID NO: 651 (e.g., ASO-STAT6-1891). In some aspects, the ASO comprises the sequence as set forth in SEQ ID NO: 652 (e.g., ASO-STAT6-1915). In some aspects, the ASO comprises the sequence as set forth in SEQ ID NO: 653 (e.g., ASO-STAT6-1916). In some aspects, the ASO comprises the sequence as set forth in SEQ ID NO: 654 (e.g., ASO-STAT6-1917). In some aspects, the ASO comprises the sequence as set forth in SEQ ID NO: 655 (e.g., ASO-STAT6-1938). In some aspects, the ASO comprises the sequence as set forth in SEQ ID NO: 656 (e.g., ASO-STAT6-1939). In some aspects, the ASO comprises the sequence as set forth in SEQ ID NO: 657 (e.g., ASO-STAT6-2063). In some aspects, the ASO comprises the sequence as set forth in SEQ ID NO: 658 (e.g., ASO-STAT6-2064). In some aspects, the ASO comprises the sequence as set forth in SEQ ID NO: 659 (e.g., ASO-STAT6-2065). In some aspects, the ASO comprises the sequence as set forth in SEQ ID NO: 660 (e.g., ASO-STAT6-2066). In some aspects, the ASO comprises the sequence as set forth in SEQ ID NO: 661 (e.g., ASO-STAT6-2068). In some aspects, the ASO comprises the sequence as set forth in SEQ ID NO: 662 (e.g., ASO-STAT6-2187). In some aspects, the ASO comprises the sequence as set forth in SEQ ID NO: 663 (e.g., ASO-STAT6-2350). In some aspects, the ASO comprises the sequence as set forth in SEQ ID NO: 664 (e.g., ASO-STAT6-2351). In some aspects, the ASO comprises the sequence as set forth in SEQ ID NO: 665 (e.g., ASO-STAT6-2352). In some aspects, the ASO comprises the sequence as set forth in SEQ ID NO: 666 (e.g., ASO-STAT6-2357). In some aspects, the ASO comprises the sequence as set forth in SEQ ID NO: 667 (e.g., ASO-STAT6-513). In some aspects, the ASO comprises the sequence as set forth in SEQ ID NO: 668 (e.g., ASO-STAT6-671). In some aspects, the ASO comprises the sequence as set forth in SEQ ID NO: 669 (e.g., ASO-STAT6-1131). In some aspects, the ASO comprises the sequence as set forth in SEQ ID NO: 670 (e.g., ASO-STAT6-1354). In some aspects, the ASO comprises the sequence as set forth in SEQ ID NO: 671 (e.g., ASO-STAT6-1355). In some aspects, the ASO comprises the sequence as set forth in SEQ ID NO: 672 (e.g., ASO-STAT6-1356). In some aspects, the ASO comprises the sequence as set forth in SEQ ID NO: 673 (e.g., ASO-STAT6-1432). In some aspects, the ASO comprises the sequence as set forth in SEQ ID NO: 674 (e.g., ASO-STAT6-1555). In some aspects, the ASO comprises the sequence as set forth in SEQ ID NO: 675 (e.g., ASO-STAT6-1556). In some aspects, the ASO comprises the sequence as set forth in SEQ ID NO: 676 (e.g., ASO-STAT6-1557). In some aspects, the ASO comprises the sequence as set forth in SEQ ID NO: 677 (e.g., ASO-STAT6-1558). In some aspects, the ASO comprises the sequence as set forth in SEQ ID NO: 678 (e.g., ASO-STAT6-1826). In some aspects, the ASO comprises the sequence as set forth in SEQ ID NO: 679 (e.g., ASO-STAT6-1827). In some aspects, the ASO comprises the sequence as set forth in SEQ ID NO: 680 (e.g., ASO-STAT6-1833). In some aspects, the ASO comprises the sequence as set forth in SEQ ID NO: 681 (e.g., ASO-STAT6-1843). In some aspects, the ASO comprises the sequence as set forth in SEQ ID NO: 682 (e.g., ASO-STAT6-1846). In some aspects, the ASO comprises the sequence as set forth in SEQ ID NO: 683 (e.g., ASO-STAT6-1847). In some aspects, the ASO comprises the sequence as set forth in SEQ ID NO: 684 (e.g., ASO-STAT6-1883). In some aspects, the ASO comprises the sequence as set forth in SEQ ID NO: 685 (e.g., ASO-STAT6-1889). In some aspects, the ASO comprises the sequence as set forth in SEQ ID NO: 686 (e.g., ASO-STAT6-1890). In some aspects, the ASO comprises the sequence as set forth in SEQ ID NO: 687 (e.g., ASO-STAT6-1891). In some aspects, the ASO comprises the sequence as set forth in SEQ ID NO: 688 (e.g., ASO-STAT6-1916). In some aspects, the ASO comprises the sequence as set forth in SEQ ID NO: 689 (e.g., ASO-STAT6-1917). In some aspects, the ASO comprises the sequence as set forth in SEQ ID NO: 690 (e.g., ASO-STAT6-2056). In some aspects, the ASO comprises the sequence as set forth in SEQ ID NO: 691 (e.g., ASO-STAT6-2057). In some aspects, the ASO comprises the sequence as set forth in SEQ ID NO: 692 (e.g., ASO-STAT6-2060). In some aspects, the ASO comprises the sequence as set forth in SEQ ID NO: 693 (e.g., ASO-STAT6-2062). In some aspects, the ASO comprises the sequence as set forth in SEQ ID NO: 694 (e.g., ASO-STAT6-2063). In some aspects, the ASO comprises the sequence as set forth in SEQ ID NO: 695 (e.g., ASO-STAT6-2065). In some aspects, the ASO comprises the sequence as set forth in SEQ ID NO: 696 (e.g., ASO-STAT6-2068). In some aspects, the ASO comprises the sequence as set forth in SEQ ID NO: 697 (e.g., ASO-STAT6-2347). In some aspects, the ASO comprises the sequence as set forth in SEQ ID NO: 698 (e.g., ASO-STAT6-2348). In some aspects, the ASO comprises the sequence as set forth in SEQ ID NO: 699 (e.g., ASO-STAT6-2358). In some aspects, the ASO comprises the sequence as set forth in SEQ ID NO: 700 (e.g., ASO-STAT6-2782). In some aspects, the ASO comprises the sequence as set forth in SEQ ID NO: 701 (e.g., ASO-STAT6-3070). In some aspects, the ASO comprises the sequence as set forth in SEQ ID NO: 702 (e.g., ASO-STAT6-3071). In some aspects, the ASO comprises the sequence as set forth in SEQ ID NO: 703 (e.g., ASO-STAT6-3431).

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 601 to 703. In some aspects the ASO (or contiguous nucleotide portion thereof) is selected from, or comprises, one of the sequences selected from the group consisting of SEQ ID NOs: 601 to 703 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 601 to 703 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding STAT6 transcript. In some aspects the ASO (or contiguous nucleotide portion thereof) is selected from, or comprises, one of the sequences selected from the group consisting of SEQ ID NOs: 601 to 703 except for 1, 2, 3, 4, 5, 6, 7, 8, 9 or 10 substitutions, wherein the substituted ASO can bind to the STAT6 transcript. In some aspects the ASO (or contiguous nucleotide portion thereof) is selected from, or comprises, one of the sequences selected from the group consisting of SEQ ID NOs: 601 to 703 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding STAT6 transcript.

In some aspects, binding of an ASO targeting a STAT6 transcript disclosed herein to a mRNA transcript encoding STAT6 can reduce expression levels and/or activity levels of STAT6.

II.C.3. ASO Targeting CEBP/β

Unless indicated otherwise, the term “CEBP/β,” as used herein, can refer to CEBP/β from one or more species (e.g., humans, non-human primates, dogs, cats, guinea pigs, rabbits, rats, mice, horses, cattle, and bears).

CEBP/β (CEBP/β) is also known as CCAAT/enhancer-binding protein beta. Synonyms of CEBP/β/CEBP/β are known and include C/EBP beta; Liver activator protein; LAP; Liver-enriched inhibitory protein; LIP; Nuclear factor NF-IL6; transcription factor 5; TCF-5; CEBPB; CEBPb; CEBPβ; CEBP/B; and TCF5. The sequence for the human CEBP/β gene can be found under publicly available GenBank Accession Number NC_000020.11 (50190583 . . . 50192690). The human CEBP/β gene is found at chromosome location 20q13.13 at 50190583-50192690.

The sequence for the human CEBP/β pre-mRNA transcript (SEQ ID NO: 21) corresponds to the reverse complement of residues 50190583-50192690 of chromosome 20q13.13. The CEBP/β mRNA sequence (GenBank Accession No. NM_001285878.1) is provided in SEQ ID NO: 23, except that the nucleotide “t” in SEQ ID NO: 23 is shown as “u” in the mRNA. The sequence for human CEBP/β protein can be found under publicly available Accession Numbers: P17676, (canonical sequence, SEQ ID NO: 22), P17676-2 (SEQ ID NO: 24), and P17676-3 (SEQ ID NO: 25), each of which is incorporated by reference herein in its entirety.

Natural variants of the human CEBP/β gene product are known. For example, natural variants of human CEBP/β protein can contain one or more amino acid substitutions selected from: A241P, A253G, G195S, and any combination thereof. Additional variants of human CEBP/β protein resulting from alternative splicing are also known in the art. CEBP/β Isoform 2 (identifier: P17676-2 at UniProt) differs from the canonical sequence (SEQ ID NO: 23) as follows: deletion of residues 1-23 relative to SEQ ID NO: 23. The sequence of CEBP/β Isoform 3 (identifier: P17676-3) differs from the canonical sequence (SEQ ID NO: 23) as follows: deletion of residues 1-198 relative to SEQ ID NO: 23. Therefore, the ASOs of the present disclosure can be designed to reduce or inhibit expression of the natural variants of the protein.

An example of a target nucleic acid sequence of the ASOs is CEBP/β pre-mRNA. SEQ ID NO: 21 represents a human CEBP/β genomic sequence (i.e., reverse complement of nucleotides 50190583-50192690 of chromosome 20q13.13). SEQ ID NO: 21 is identical to a CEBP/β pre-mRNA sequence except that nucleotide “t” in SEQ ID NO: 21 is shown as “u” in pre-mRNA. In certain aspects, the “target nucleic acid” comprises an intron of a CEBP/β protein-encoding nucleic acids or naturally occurring variants thereof, and RNA nucleic acids derived therefrom, e.g., pre-mRNA. In other aspects, the target nucleic acid comprises an exon region of a CEBP/β protein-encoding nucleic acids or naturally occurring variants thereof, and RNA nucleic acids derived therefrom, e.g., pre-mRNA. In yet other aspects, the target nucleic acid comprises an exon-intron junction of a CEBP/β protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human CEBP/β protein sequence encoded by the CEBP/β pre-mRNA is shown as SEQ ID NO: 23. In other aspects, the target nucleic acid comprises an untranslated region of a CEBP/β protein-encoding nucleic acids or naturally occurring variants thereof, e.g., 5′ UTR, 3′ UTR, or both.

In some aspects, an ASO of the disclosure hybridizes to a region within the introns of a CEBP/β transcript, e.g., SEQ ID NO: 21. In certain aspects, an ASO of the disclosure hybridizes to a region within the exons of a CEBP/β transcript, e.g., SEQ ID NO: 21. In other aspects, an ASO of the disclosure hybridizes to a region within the exon-intron junction of a CEBP/β transcript, e.g., SEQ ID NO: 21. In some aspects, an ASO of the disclosure hybridizes to a region within a CEBP/β transcript (e.g., an intron, exon, or exon-intron junction), e.g., SEQ ID NO: 21, wherein the ASO has a design according to formula: 5′ A-B-C 3′ as described elsewhere herein.

In some aspects, the ASO targets a mRNA encoding a particular isoform of CEBP/β protein (e.g., Isoform 1). In some aspects, the ASO targets all isoforms of CEBP/β protein. In other aspects, the ASO targets two isoforms (e.g., Isoform 1 and Isoform 2, Isoform 1 and Isoform 3, or Isoform 2 and Isoform 3) of CEBP/β protein.

In some aspects, the nucleotide sequence of the ASOs of the disclosure or the contiguous nucleotide sequence has at least about 80% sequence identity to a sequence selected from SEQ ID NOs: 704-806 (i.e., the sequences in FIG. 1C), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1C).

In some aspects the ASO (or contiguous nucleotide portion thereof) is selected from, or comprises, one of the sequences selected from the group consisting of SEQ ID NOs: 704-806 or a region of at least 10 contiguous nucleotides thereof, wherein the ASO (or contiguous nucleotide portion thereof) can optionally comprise one, two, three, or four mismatches when compared to the corresponding CEBP/B transcript.

In some aspects, the ASO comprises the sequence as set forth in SEQ ID NO: 704 (e.g., ASO-CEBPb-540). In some aspects, the ASO comprises the sequence as set forth in SEQ ID NO: 705 (e.g., ASO-CEBPb-565). In some aspects, the ASO comprises the sequence as set forth in SEQ ID NO: 706 (e.g., ASO-CEBPb-569). In some aspects, the ASO comprises the sequence as set forth in SEQ ID NO: 707 (e.g., ASO-CEBPb-648). In some aspects, the ASO comprises the sequence as set forth in SEQ ID NO: 708 (e.g., ASO-CEBPb-816). In some aspects, the ASO comprises the sequence as set forth in SEQ ID NO: 709 (e.g., ASO-CEBPb-817). In some aspects, the ASO comprises the sequence as set forth in SEQ ID NO: 710 (e.g., ASO-CEBPb-818). In some aspects, the ASO comprises the sequence as set forth in SEQ ID NO: 711 (e.g., ASO-CEBPb-819). In some aspects, the ASO comprises the sequence as set forth in SEQ ID NO: 712 (e.g., ASO-CEBPb-820). In some aspects, the ASO comprises the sequence as set forth in SEQ ID NO: 713 (e.g., ASO-CEBPb-851). In some aspects, the ASO comprises the sequence as set forth in SEQ ID NO: 714 (e.g., ASO-CEBPb-853). In some aspects, the ASO comprises the sequence as set forth in SEQ ID NO: 715 (e.g., ASO-CEBPb-856). In some aspects, the ASO comprises the sequence as set forth in SEQ ID NO: 716 (e.g., ASO-CEBPb-858). In some aspects, the ASO comprises the sequence as set forth in SEQ ID NO: 717 (e.g., ASO-CEBPb-987). In some aspects, the ASO comprises the sequence as set forth in SEQ ID NO: 718 (e.g., ASO-CEBPb-1056). In some aspects, the ASO comprises the sequence as set forth in SEQ ID NO: 719 (e.g., ASO-CEBPb-1064). In some aspects, the ASO comprises the sequence as set forth in SEQ ID NO: 720 (e.g., ASO-CEBPb-1065). In some aspects, the ASO comprises the sequence as set forth in SEQ ID NO: 721 (e.g., ASO-CEBPb-1066). In some aspects, the ASO comprises the sequence as set forth in SEQ ID NO: 722 (e.g., ASO-CEBPb-1071). In some aspects, the ASO comprises the sequence as set forth in SEQ ID NO: 723 (e.g., ASO-CEBPb-1270). In some aspects, the ASO comprises the sequence as set forth in SEQ ID NO: 724 (e.g., ASO-CEBPb-1273). In some aspects, the ASO comprises the sequence as set forth in SEQ ID NO: 725 (e.g., ASO-CEBPb-1274). In some aspects, the ASO comprises the sequence as set forth in SEQ ID NO: 726 (e.g., ASO-CEBPb-1405). In some aspects, the ASO comprises the sequence as set forth in SEQ ID NO: 727 (e.g., ASO-CEBPb-1407). In some aspects, the ASO comprises the sequence as set forth in SEQ ID NO: 728 (e.g., ASO-CEBPb-539). In some aspects, the ASO comprises the sequence as set forth in SEQ ID NO: 729 (e.g., ASO-CEBPb-540). In some aspects, the ASO comprises the sequence as set forth in SEQ ID NO: 730 (e.g., ASO-CEBPb-563). In some aspects, the ASO comprises the sequence as set forth in SEQ ID NO: 731 (e.g., ASO-CEBPb-564). In some aspects, the ASO comprises the sequence as set forth in SEQ ID NO: 732 (e.g., ASO-CEBPb-565). In some aspects, the ASO comprises the sequence as set forth in SEQ ID NO: 733 (e.g., ASO-CEBPb-568). In some aspects, the ASO comprises the sequence as set forth in SEQ ID NO: 734 (e.g., ASO-CEBPb-644). In some aspects, the ASO comprises the sequence as set forth in SEQ ID NO: 735 (e.g., ASO-CEBPb-645). In some aspects, the ASO comprises the sequence as set forth in SEQ ID NO: 736 (e.g., ASO-CEBPb-648). In some aspects, the ASO comprises the sequence as set forth in SEQ ID NO: 737 (e.g., ASO-CEBPb-819). In some aspects, the ASO comprises the sequence as set forth in SEQ ID NO: 738 (e.g., ASO-CEBPb-855). In some aspects, the ASO comprises the sequence as set forth in SEQ ID NO: 739 (e.g., ASO-CEBPb-860). In some aspects, the ASO comprises the sequence as set forth in SEQ ID NO: 740 (e.g., ASO-CEBPb-986). In some aspects, the ASO comprises the sequence as set forth in SEQ ID NO: 741 (e.g., ASO-CEBPb-987). In some aspects, the ASO comprises the sequence as set forth in SEQ ID NO: 742 (e.g., ASO-CEBPb-996). In some aspects, the ASO comprises the sequence as set forth in SEQ ID NO: 743 (e.g., ASO-CEBPb-1049). In some aspects, the ASO comprises the sequence as set forth in SEQ ID NO: 744 (e.g., ASO-CEBPb-1050). In some aspects, the ASO comprises the sequence as set forth in SEQ ID NO: 745 (e.g., ASO-CEBPb-1064). In some aspects, the ASO comprises the sequence as set forth in SEQ ID NO: 746 (e.g., ASO-CEBPb-1065). In some aspects, the ASO comprises the sequence as set forth in SEQ ID NO: 747 (e.g., ASO-CEBPb-1066). In some aspects, the ASO comprises the sequence as set forth in SEQ ID NO: 748 (e.g., ASO-CEBPb-1083). In some aspects, the ASO comprises the sequence as set forth in SEQ ID NO: 749 (e.g., ASO-CEBPb-1088). In some aspects, the ASO comprises the sequence as set forth in SEQ ID NO: 750 (e.g., ASO-CEBPb-1253). In some aspects, the ASO comprises the sequence as set forth in SEQ ID NO: 751 (e.g., ASO-CEBPb-1269). In some aspects, the ASO comprises the sequence as set forth in SEQ ID NO: 752 (e.g., ASO-CEBPb-1272). In some aspects, the ASO comprises the sequence as set forth in SEQ ID NO: 753 (e.g., ASO-CEBPb-1274). In some aspects, the ASO comprises the sequence as set forth in SEQ ID NO: 754 (e.g., ASO-CEBPb-539). In some aspects, the ASO comprises the sequence as set forth in SEQ ID NO: 755 (e.g., ASO-CEBPb-564). In some aspects, the ASO comprises the sequence as set forth in SEQ ID NO: 756 (e.g., ASO-CEBPb-565). In some aspects, the ASO comprises the sequence as set forth in SEQ ID NO: 757 (e.g., ASO-CEBPb-567). In some aspects, the ASO comprises the sequence as set forth in SEQ ID NO: 758 (e.g., ASO-CEBPb-647). In some aspects, the ASO comprises the sequence as set forth in SEQ ID NO: 759 (e.g., ASO-CEBPb-648). In some aspects, the ASO comprises the sequence as set forth in SEQ ID NO: 760 (e.g., ASO-CEBPb-815). In some aspects, the ASO comprises the sequence as set forth in SEQ ID NO: 761 (e.g., ASO-CEBPb-818). In some aspects, the ASO comprises the sequence as set forth in SEQ ID NO: 762 (e.g., ASO-CEBPb-820). In some aspects, the ASO comprises the sequence as set forth in SEQ ID NO: 763 (e.g., ASO-CEBPb-854). In some aspects, the ASO comprises the sequence as set forth in SEQ ID NO: 764 (e.g., ASO-CEBPb-855). In some aspects, the ASO comprises the sequence as set forth in SEQ ID NO: 765 (e.g., ASO-CEBPb-859). In some aspects, the ASO comprises the sequence as set forth in SEQ ID NO: 766 (e.g., ASO-CEBPb-1050). In some aspects, the ASO comprises the sequence as set forth in SEQ ID NO: 767 (e.g., ASO-CEBPb-1053). In some aspects, the ASO comprises the sequence as set forth in SEQ ID NO: 768 (e.g., ASO-CEBPb-1062). In some aspects, the ASO comprises the sequence as set forth in SEQ ID NO: 769 (e.g., ASO-CEBPb-1063). In some aspects, the ASO comprises the sequence as set forth in SEQ ID NO: 770 (e.g., ASO-CEBPb-1064). In some aspects, the ASO comprises the sequence as set forth in SEQ ID NO: 771 (e.g., ASO-CEBPb-1065). In some aspects, the ASO comprises the sequence as set forth in SEQ ID NO: 772 (e.g., ASO-CEBPb-1265). In some aspects, the ASO comprises the sequence as set forth in SEQ ID NO: 773 (e.g., ASO-CEBPb-1270). In some aspects, the ASO comprises the sequence as set forth in SEQ ID NO: 774 (e.g., ASO-CEBPb-1271). In some aspects, the ASO comprises the sequence as set forth in SEQ ID NO: 775 (e.g., ASO-CEBPb-1272). In some aspects, the ASO comprises the sequence as set forth in SEQ ID NO: 776 (e.g., ASO-CEBPb-1274). In some aspects, the ASO comprises the sequence as set forth in SEQ ID NO: 777 (e.g., ASO-CEBPb-1277). In some aspects, the ASO comprises the sequence as set forth in SEQ ID NO: 778 (e.g., ASO-CEBPb-564). In some aspects, the ASO comprises the sequence as set forth in SEQ ID NO: 779 (e.g., ASO-CEBPb-565). In some aspects, the ASO comprises the sequence as set forth in SEQ ID NO: 780 (e.g., ASO-CEBPb-818). In some aspects, the ASO comprises the sequence as set forth in SEQ ID NO: 781 (e.g., ASO-CEBPb-1061). In some aspects, the ASO comprises the sequence as set forth in SEQ ID NO: 782 (e.g., ASO-CEBPb-1062). In some aspects, the ASO comprises the sequence as set forth in SEQ ID NO: 783 (e.g., ASO-CEBPb-1064). In some aspects, the ASO comprises the sequence as set forth in SEQ ID NO: 784 (e.g., ASO-CEBPb-1267). In some aspects, the ASO comprises the sequence as set forth in SEQ ID NO: 785 (e.g., ASO-CEBPb-1272). In some aspects, the ASO comprises the sequence as set forth in SEQ ID NO: 786 (e.g., ASO-CEBPb-645). In some aspects, the ASO comprises the sequence as set forth in SEQ ID NO: 787 (e.g., ASO-CEBPb-848). In some aspects, the ASO comprises the sequence as set forth in SEQ ID NO: 788 (e.g., ASO-CEBPb-849). In some aspects, the ASO comprises the sequence as set forth in SEQ ID NO: 789 (e.g., ASO-CEBPb-850). In some aspects, the ASO comprises the sequence as set forth in SEQ ID NO: 790 (e.g., ASO-CEBPb-1063). In some aspects, the ASO comprises the sequence as set forth in SEQ ID NO: 791 (e.g., ASO-CEBPb-1070). In some aspects, the ASO comprises the sequence as set forth in SEQ ID NO: 792 (e.g., ASO-CEBPb-1071). In some aspects, the ASO comprises the sequence as set forth in SEQ ID NO: 793 (e.g., ASO-CEBPb-1262). In some aspects, the ASO comprises the sequence as set forth in SEQ ID NO: 794 (e.g., ASO-CEBPb-1274). In some aspects, the ASO comprises the sequence as set forth in SEQ ID NO: 795 (e.g., ASO-CEBPb-1275). In some aspects, the ASO comprises the sequence as set forth in SEQ ID NO: 796 (e.g., ASO-CEBPb-644). In some aspects, the ASO comprises the sequence as set forth in SEQ ID NO: 797 (e.g., ASO-CEBPb-647). In some aspects, the ASO comprises the sequence as set forth in SEQ ID NO: 798 (e.g., ASO-CEBPb-851). In some aspects, the ASO comprises the sequence as set forth in SEQ ID NO: 799 (e.g., ASO-CEBPb-1266). In some aspects, the ASO comprises the sequence as set forth in SEQ ID NO: 800 (e.g., ASO-CEBPb-1268). In some aspects, the ASO comprises the sequence as set forth in SEQ ID NO: 801 (e.g., ASO-CEBPb-1270). In some aspects, the ASO comprises the sequence as set forth in SEQ ID NO: 802 (e.g., ASO-CEBPb-646). In some aspects, the ASO comprises the sequence as set forth in SEQ ID NO: 803 (e.g., ASO-CEBPb-1060). In some aspects, the ASO comprises the sequence as set forth in SEQ ID NO: 804 (e.g., ASO-CEBPb-1263). In some aspects, the ASO comprises the sequence as set forth in SEQ ID NO: 805 (e.g., ASO-CEBPb-1269). In some aspects, the ASO comprises the sequence as set forth in SEQ ID NO: 806 (e.g., ASO-CEBPb-1271).

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 704 to 806. In some aspects the ASO (or contiguous nucleotide portion thereof) is selected from, or comprises, one of the sequences selected from the group consisting of SEQ ID NOs: 704 to 806 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 704 to 806 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding CEBPb transcript. In some aspects the ASO (or contiguous nucleotide portion thereof) is selected from, or comprises, one of the sequences selected from the group consisting of SEQ ID NOs: 704 to 806 except for 1, 2, 3, 4, 5, 6, 7, 8, 9 or 10 substitutions, wherein the substituted ASO can bind to the CEBPb transcript. In some aspects the ASO (or contiguous nucleotide portion thereof) is selected from, or comprises, one of the sequences selected from the group consisting of SEQ ID NOs: 704 to 806 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding CEBPb transcript.

In some aspects, binding of an ASO targeting a CEBPb transcript disclosed herein to a mRNA transcript encoding CEBPb can reduce expression levels and/or activity levels of CEBPb.

II.C4. ASO Targeting STAT3

Signal Transducer and Activator of Transcription 3 (STAT3) is a signal transducer and activator of transcription that transmits signals from cell surface receptors to the nucleus. STAT3 is frequently hyperactivated in many human cancers. STAT3-encoding genomic DNA can be found at Chromosomal position 17q21.2 (i.e., nucleotides 5,001 to 80,171 of GenBank Accession No. NG_007370.1) High levels of activated STAT3 are often found to correlate with poor prognosis in human breast cancer patients in terms of metastatic progression (Ranger et al. 2009). Therefore, STAT3 represents a promising target for the prevention and treatment of both ER-positive and ER-negative breast cancer and also other cancers such as pancreatic, head/neck, prostate and lung cancers. However, current strategies of inhibiting STAT3 activity by means of blocking peptides, blockade of translocation, disrupting dimerization, or modulating phosphatase activity have not sufficiently inhibited STAT3 activity in cancer cells. Under normal conditions, STAT3 activation is transient and tightly regulated. Upon cellular stimulation by ligands such as growth factors or cytokines, STAT3 becomes phosphorylated on a critical tyrosine residue (Tyr705) and consequently induces STAT3 dimerization through two reciprocal phosphotyrosine (pTyr)-Src-homology 2 (SH2) interactions. The STAT3 dimers then translocate to the nucleus and bind to specific DNA-response elements in the promoters of target genes thereby activating transcription. The association of aberrant STAT3 activation with many types of human malignancies and solid tumors has made STAT3 an attractive molecular target for the development of novel cancer therapeutics.

STAT3 is often found to be constitutively activated in tumor cells and contribute to tumor progression through the modulation of target genes, such as antiapoptotic genes Bcl-xL, Bcl-2, Mcl-1 and survivin along with genes driving cell cycle progression, c-Myc and cyclin-D1. Aberrant activation of STAT3 is frequent in almost all blood malignancies and solid tumors, including lymphoma and leukemia, breast, prostate, lung head and neck, brain and colon cancer, which have made STAT3 an attractive target for the development of anticancer agents. The specificity of STAT activation is due to specific cytokines, i.e. each STAT is responsive to a small number of specific cytokines. Other non-cytokine signaling molecules, such as growth factors, have also been found to activate STATs. Binding of these factors to a cell surface receptor associated with protein tyrosine kinase also results in phosphorylation of STAT. STAT3 in particular has been found to be responsive to interleukin-6 (IL-6) as well as epidermal growth factor (EGF) (Darnell, Jr., J. E., et al., Science, 1994, 264, 1415-1421). Evidence exists suggesting that STAT3 may be regulated by the MAPK pathway. ERK2 induces serine phosphorylation and also associates with STAT3 (Jain, N., et al., Oncogene, 1998, 17, 3157-3167). STAT3 is expressed in most cell types and is also involved in the induction of expression of genes involved in response to tissue injury and inflammation. Aberrant expression of or constitutive expression of STAT3 is associated with a number of disease processes. STAT3 has been found to be constitutively active in myeloma tumor cells, both in culture and in bone marrow mononuclear cells from patients with multiple myeloma. These cells are resistant to Fas-mediated apoptosis and express high levels of Bcl-xL. The STAT3 SH2 domain is required for promoting dimerization. One of the limitations of targeting protein dimerization is the practicality of targeting the dimer interface, which is challenging owing to the planarity of the large surface area.

Signal transducer and activator of transcription 3 (STAT3) is known in the art by various names. Such names include: DNA-binding protein APRF, and acute-phase response factor. The mRNA encoding human STAT3 can be found at Genbank Accession Number NM_003150.3, and is represented by the sequence (SEQ ID NO: 43).

Natural variants of the human STAT3 gene product are known. For example, natural variants of human STAT3 protein can contain one or more amino acid substitutions selected from: R382L, R382Q, OR R382W, and any combinations thereof. Additional variants of human STAT3 protein resulting from alternative splicing are also known in the art, such as: R382W, F384L, F384S, T389I, N395Y, R423Q, N425Y, H437Y, Del-463, S611N, F621V, T622L, V637L, V637M, Del-644, Y657C, P330S, K392R, N646K, K658N, Del-701, or T716M. Therefore, the ASOs of the present disclosure can be designed to reduce or inhibit expression of the natural variants of the STAT3 protein.

SEQ ID NO: 41 is identical to a STAT3 pre-mRNA sequence except that nucleotide “t” in SEQ ID NO: 41 is shown as “u” in pre-mRNA. In certain aspects, the “target nucleic acid” comprises an intron of a STAT3 protein-encoding nucleic acids or naturally occurring variants thereof, and RNA nucleic acids derived therefrom, e.g., pre-mRNA. In other aspects, the target nucleic acid comprises an exon region of a STAT3 protein-encoding nucleic acids or naturally occurring variants thereof, and RNA nucleic acids derived therefrom, e.g., pre-mRNA. In yet other aspects, the target nucleic acid comprises an exon-intron junction of a STAT3 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human STAT3 protein sequence encoded by the STAT3 pre-mRNA is shown as SEQ ID NO: 42. In other aspects, the target nucleic acid comprises an untranslated region of a STAT3 protein-encoding nucleic acids or naturally occurring variants thereof, e.g., 5′ UTR, 3′ UTR, or both.

In yet other aspects, the target nucleic acid comprises an exon-intron junction of a STAT3 protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human STAT3 protein sequence encoded by the STAT3 pre-mRNA is shown as SEQ ID NO: 43. In other aspects, the target nucleic acid comprises an untranslated region of a STAT3 protein-encoding nucleic acids or naturally occurring variants thereof, e.g., 5′ UTR, 3′ UTR, or both.

In some aspects, an ASO of the disclosure hybridizes to a region within the introns of a STAT3 transcript, e.g., SEQ ID NO: 41 or SEQ ID NO: 43. In certain aspects, an ASO of the disclosure hybridizes to a region within the exons of a STAT3 transcript, e.g., SEQ ID NO: 41 or SEQ ID NO: 43. In other aspects, an ASO of the disclosure hybridizes to a region within the exon-intron junction of a STAT3 transcript, e.g., SEQ ID NO: 41 or SEQ ID NO: 43. In some aspects, an ASO of the disclosure hybridizes to a region within a STAT3 transcript (e.g., an intron, exon, or exon-intron junction), e.g., SEQ ID NO: 41 or SEQ ID NO: 43, wherein the ASO has a design according to formula: 5′ A-B-C 3′ as described elsewhere herein.

In some aspects, the ASO targets a mRNA encoding a particular isoform of STAT3 protein (e.g., Isoform 1). In some aspects, the ASO targets all isoforms of STAT3 protein. In other aspects, the ASO targets two isoforms (e.g., Isoform 1 (UniProt ID: P40763-1) and Isoform 2 (UniProt ID: P40763-2), Isoform 2 and Isoform 3 (UniProt ID: P40763-3) of STAT3 protein.

In some aspects, an ASO of the disclosure hybridizes to a region within the introns of a STAT3 transcript, e.g., SEQ ID NO: 41 or SEQ ID NO: 43. In certain aspects, an ASO of the disclosure hybridizes to a region within the exons of a STAT3 transcript, e.g., SEQ ID NO: 41 or SEQ ID NO: 43. In other aspects, an ASO of the disclosure hybridizes to a region within the exon-intron junction of a STAT3 transcript, e.g., SEQ ID NO: 41 or SEQ ID NO: 43. In some aspects, an ASO of the disclosure hybridizes to a region within a STAT3 transcript (e.g., an intron, exon, or exon-intron junction), e.g., SEQ ID NO: 41 or SEQ ID NO: 43, wherein the ASO has a design according to formula: 5′ A-B-C 3′ as described elsewhere herein.

In some aspects, the ASO of the present disclosure hybridizes to multiple target regions within the STAT3 transcript (e.g., genomic sequence, SEQ ID NO: 41). In some aspects, the ASO hybridizes to two different target regions within the STAT3 transcript. In some aspects, the ASO hybridizes to three different target regions within the STAT3 transcript. The sequences of exemplary ASOs that hybridizes to multiple target regions, and the start/end sites of the different target regions are provided in FIG. 1D. In some aspects, the ASOs that hybridizes to multiple regions within the STAT3 transcript (e.g., genomic sequence, SEQ ID NO: 41) are more potent (e.g., having lower EC50) at reducing STAT3 expression compared to ASOs that hybridizes to a single region within the STAT3 transcript (e.g., genomic sequence, SEQ ID NO: 41).

The ASOs of the disclosure comprise a contiguous nucleotide sequence which corresponds to the complement of a region of STAT3 transcript, e.g., a nucleotide sequence corresponding to SEQ ID NO: 41.

In some aspects, the nucleotide sequence of the ASOs of the disclosure or the contiguous nucleotide sequence has at least about 80% sequence identity to a sequence selected from SEQ ID NOs: 889-988 (i.e., the sequences in FIG. 1D), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1D).

In some aspects the ASO (or contiguous nucleotide portion thereof) is selected from, or comprises, one of the sequences selected from the group consisting of SEQ ID NOs: 889-988 to STAT3 or a region of at least 10 contiguous nucleotides thereof, wherein the ASO (or contiguous nucleotide portion thereof) can optionally comprise one, two, three, or four mismatches when compared to the corresponding STAT3 transcript. Non-limiting exemplary ASOs targeting STAT3 gene are shown in FIG. 1D.

In some aspects, the ASO comprises the sequence as set forth in SEQ ID NO: 889 (e.g., ASO-STAT3-2559). In some aspects, the ASO comprises the sequence as set forth in SEQ ID NO: 890 (e.g., ASO-STAT3-2556). In some aspects, the ASO comprises the sequence as set forth in SEQ ID NO: 891 (e.g., ASO-STAT3-2557). In some aspects, the ASO comprises the sequence as set forth in SEQ ID NO: 892 (e.g., ASO-STAT3-1046). In some aspects, the ASO comprises the sequence as set forth in SEQ ID NO: 893 (e.g., ASO-STAT3-351). In some aspects, the ASO comprises the sequence as set forth in SEQ ID NO: 894 (e.g., ASO-STAT3-450). In some aspects, the ASO comprises the sequence as set forth in SEQ ID NO: 895 (e.g., ASO-STAT3-2558). In some aspects, the ASO comprises the sequence as set forth in SEQ ID NO: 896 (e.g., ASO-STAT3-2558). In some aspects, the ASO comprises the sequence as set forth in SEQ ID NO: 897 (e.g., ASO-STAT3-865). In some aspects, the ASO comprises the sequence as set forth in SEQ ID NO: 898 (e.g., ASO-STAT3-894). In some aspects, the ASO comprises the sequence as set forth in SEQ ID NO: 899 (e.g., ASO-STAT3-1778). In some aspects, the ASO comprises the sequence as set forth in SEQ ID NO: 900 (e.g., ASO-STAT3-2558). In some aspects, the ASO comprises the sequence as set forth in SEQ ID NO: 901 (e.g., ASO-STAT3-1482). In some aspects, the ASO comprises the sequence as set forth in SEQ ID NO: 902 (e.g., ASO-STAT3-892). In some aspects, the ASO comprises the sequence as set forth in SEQ ID NO: 903 (e.g., ASO-STAT3-2262). In some aspects, the ASO comprises the sequence as set forth in SEQ ID NO: 904 (e.g., ASO-STAT3-2267). In some aspects, the ASO comprises the sequence as set forth in SEQ ID NO: 905 (e.g., ASO-STAT3-411). In some aspects, the ASO comprises the sequence as set forth in SEQ ID NO: 906 (e.g., ASO-STAT3-2267). In some aspects, the ASO comprises the sequence as set forth in SEQ ID NO: 907 (e.g., ASO-STAT3-896). In some aspects, the ASO comprises the sequence as set forth in SEQ ID NO: 908 (e.g., ASO-STAT3-2555). In some aspects, the ASO comprises the sequence as set forth in SEQ ID NO: 909 (e.g., ASO-STAT3-525). In some aspects, the ASO comprises the sequence as set forth in SEQ ID NO: 910 (e.g., ASO-STAT3-1766). In some aspects, the ASO comprises the sequence as set forth in SEQ ID NO: 911 (e.g., ASO-STAT3-1114). In some aspects, the ASO comprises the sequence as set forth in SEQ ID NO: 912 (e.g., ASO-STAT3-2557). In some aspects, the ASO comprises the sequence as set forth in SEQ ID NO: 913 (e.g., ASO-STAT3-995). In some aspects, the ASO comprises the sequence as set forth in SEQ ID NO: 914 (e.g., ASO-STAT3-2263). In some aspects, the ASO comprises the sequence as set forth in SEQ ID NO: 915 (e.g., ASO-STAT3-511). In some aspects, the ASO comprises the sequence as set forth in SEQ ID NO: 916 (e.g., ASO-STAT3-511). In some aspects, the ASO comprises the sequence as set forth in SEQ ID NO: 917 (e.g., ASO-STAT3-1043). In some aspects, the ASO comprises the sequence as set forth in SEQ ID NO: 918 (e.g., ASO-STAT3-1780). In some aspects, the ASO comprises the sequence as set forth in SEQ ID NO: 919 (e.g., ASO-STAT3-458). In some aspects, the ASO comprises the sequence as set forth in SEQ ID NO: 920 (e.g., ASO-STAT3-894). In some aspects, the ASO comprises the sequence as set forth in SEQ ID NO: 921 (e.g., ASO-STAT3-1779). In some aspects, the ASO comprises the sequence as set forth in SEQ ID NO: 922 (e.g., ASO-STAT3-2274). In some aspects, the ASO comprises the sequence as set forth in SEQ ID NO: 923 (e.g., ASO-STAT3-1039). In some aspects, the ASO comprises the sequence as set forth in SEQ ID NO: 924 (e.g., ASO-STAT3-1238). In some aspects, the ASO comprises the sequence as set forth in SEQ ID NO: 925 (e.g., ASO-STAT3-1239). In some aspects, the ASO comprises the sequence as set forth in SEQ ID NO: 926 (e.g., ASO-STAT3-516). In some aspects, the ASO comprises the sequence as set forth in SEQ ID NO: 927 (e.g., ASO-STAT3-1238). In some aspects, the ASO comprises the sequence as set forth in SEQ ID NO: 928 (e.g., ASO-STAT3-1034). In some aspects, the ASO comprises the sequence as set forth in SEQ ID NO: 929 (e.g., ASO-STAT3-1239). In some aspects, the ASO comprises the sequence as set forth in SEQ ID NO: 930 (e.g., ASO-STAT3-1113). In some aspects, the ASO comprises the sequence as set forth in SEQ ID NO: 931 (e.g., ASO-STAT3-1484). In some aspects, the ASO comprises the sequence as set forth in SEQ ID NO: 932 (e.g., ASO-STAT3-2556). In some aspects, the ASO comprises the sequence as set forth in SEQ ID NO: 933 (e.g., ASO-STAT3-461). In some aspects, the ASO comprises the sequence as set forth in SEQ ID NO: 934 (e.g., ASO-STAT3-2273). In some aspects, the ASO comprises the sequence as set forth in SEQ ID NO: 935 (e.g., ASO-STAT3-1783). In some aspects, the ASO comprises the sequence as set forth in SEQ ID NO: 936 (e.g., ASO-STAT3-891). In some aspects, the ASO comprises the sequence as set forth in SEQ ID NO: 937 (e.g., ASO-STAT3-510). In some aspects, the ASO comprises the sequence as set forth in SEQ ID NO: 938 (e.g., ASO-STAT3-2115). In some aspects, the ASO comprises the sequence as set forth in SEQ ID NO: 939 (e.g., ASO-STAT3-1482). In some aspects, the ASO comprises the sequence as set forth in SEQ ID NO: 940 (e.g., ASO-STAT3-986). In some aspects, the ASO comprises the sequence as set forth in SEQ ID NO: 941 (e.g., ASO-STAT3-893). In some aspects, the ASO comprises the sequence as set forth in SEQ ID NO: 942 (e.g., ASO-STAT3-1237). In some aspects, the ASO comprises the sequence as set forth in SEQ ID NO: 943 (e.g., ASO-STAT3-1111). In some aspects, the ASO comprises the sequence as set forth in SEQ ID NO: 944 (e.g., ASO-STAT3-1236). In some aspects, the ASO comprises the sequence as set forth in SEQ ID NO: 945 (e.g., ASO-STAT3-2557). In some aspects, the ASO comprises the sequence as set forth in SEQ ID NO: 946 (e.g., ASO-STAT3-2264). In some aspects, the ASO comprises the sequence as set forth in SEQ ID NO: 947 (e.g., ASO-STAT3-1234). In some aspects, the ASO comprises the sequence as set forth in SEQ ID NO: 948 (e.g., ASO-STAT3-1241). In some aspects, the ASO comprises the sequence as set forth in SEQ ID NO: 949 (e.g., ASO-STAT3-524). In some aspects, the ASO comprises the sequence as set forth in SEQ ID NO: 950 (e.g., ASO-STAT3-890). In some aspects, the ASO comprises the sequence as set forth in SEQ ID NO: 951 (e.g., ASO-STAT3-1114). In some aspects, the ASO comprises the sequence as set forth in SEQ ID NO: 952 (e.g., ASO-STAT3-1108). In some aspects, the ASO comprises the sequence as set forth in SEQ ID NO: 953 (e.g., ASO-STAT3-409). In some aspects, the ASO comprises the sequence as set forth in SEQ ID NO: 954 (e.g., ASO-STAT3-1356). In some aspects, the ASO comprises the sequence as set forth in SEQ ID NO: 955 (e.g., ASO-STAT3-1231). In some aspects, the ASO comprises the sequence as set forth in SEQ ID NO: 956 (e.g., ASO-STAT3-2267). In some aspects, the ASO comprises the sequence as set forth in SEQ ID NO: 957 (e.g., ASO-STAT3-1238). In some aspects, the ASO comprises the sequence as set forth in SEQ ID NO: 958 (e.g., ASO-STAT3-1237). In some aspects, the ASO comprises the sequence as set forth in SEQ ID NO: 959 (e.g., ASO-STAT3-522). In some aspects, the ASO comprises the sequence as set forth in SEQ ID NO: 960 (e.g., ASO-STAT3-2266). In some aspects, the ASO comprises the sequence as set forth in SEQ ID NO: 961 (e.g., ASO-STAT3-1998). In some aspects, the ASO comprises the sequence as set forth in SEQ ID NO: 962 (e.g., ASO-STAT3-881). In some aspects, the ASO comprises the sequence as set forth in SEQ ID NO: 963 (e.g., ASO-STAT3-513). In some aspects, the ASO comprises the sequence as set forth in SEQ ID NO: 964 (e.g., ASO-STAT3-1107). In some aspects, the ASO comprises the sequence as set forth in SEQ ID NO: 965 (e.g., ASO-STAT3-1235). In some aspects, the ASO comprises the sequence as set forth in SEQ ID NO: 966 (e.g., ASO-STAT3-882). In some aspects, the ASO comprises the sequence as set forth in SEQ ID NO: 967 (e.g., ASO-STAT3-1112). In some aspects, the ASO comprises the sequence as set forth in SEQ ID NO: 968 (e.g., ASO-STAT3-521). In some aspects, the ASO comprises the sequence as set forth in SEQ ID NO: 969 (e.g., ASO-STAT3-1110). In some aspects, the ASO comprises the sequence as set forth in SEQ ID NO: 970 (e.g., ASO-STAT3-1475). In some aspects, the ASO comprises the sequence as set forth in SEQ ID NO: 971 (e.g., ASO-STAT3-894). In some aspects, the ASO comprises the sequence as set forth in SEQ ID NO: 972 (e.g., ASO-STAT3-519). In some aspects, the ASO comprises the sequence as set forth in SEQ ID NO: 973 (e.g., ASO-STAT3-2553). In some aspects, the ASO comprises the sequence as set forth in SEQ ID NO: 974 (e.g., ASO-STAT3-2552). In some aspects, the ASO comprises the sequence as set forth in SEQ ID NO: 975 (e.g., ASO-STAT3-883). In some aspects, the ASO comprises the sequence as set forth in SEQ ID NO: 976 (e.g., ASO-STAT3-842). In some aspects, the ASO comprises the sequence as set forth in SEQ ID NO: 977 (e.g., ASO-STAT3-851). In some aspects, the ASO comprises the sequence as set forth in SEQ ID NO: 978 (e.g., ASO-STAT3-2265). In some aspects, the ASO comprises the sequence as set forth in SEQ ID NO: 979 (e.g., ASO-STAT3-520). In some aspects, the ASO comprises the sequence as set forth in SEQ ID NO: 980 (e.g., ASO-STAT3-985). In some aspects, the ASO comprises the sequence as set forth in SEQ ID NO: 981 (e.g., ASO-STAT3-524). In some aspects, the ASO comprises the sequence as set forth in SEQ ID NO: 982 (e.g., ASO-STAT3-1106). In some aspects, the ASO comprises the sequence as set forth in SEQ ID NO: 983 (e.g., ASO-STAT3-517). In some aspects, the ASO comprises the sequence as set forth in SEQ ID NO: 984 (e.g., ASO-STAT3-1721). In some aspects, the ASO comprises the sequence as set forth in SEQ ID NO: 985 (e.g., ASO-STAT3-1113). In some aspects, the ASO comprises the sequence as set forth in SEQ ID NO: 986 (e.g., ASO-STAT3-992). In some aspects, the ASO comprises the sequence as set forth in SEQ ID NO: 987 (e.g., ASO-STAT3-993). In some aspects, the ASO comprises the sequence as set forth in SEQ ID NO: 988 (e.g., ASO-STAT3-1104).

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 889 to 988. In some aspects the ASO (or contiguous nucleotide portion thereof) is selected from, or comprises, one of the sequences selected from the group consisting of SEQ ID NOs: 889 to 988 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 889 to 988 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding STAT3 transcript. In some aspects the ASO (or contiguous nucleotide portion thereof) is selected from, or comprises, one of the sequences selected from the group consisting of SEQ ID NOs: 889 to 988 except for 1, 2, 3, 4, 5, 6, 7, 8, 9 or 10 substitutions, wherein the substituted ASO can bind to the CEBPb transcript. In some aspects the ASO (or contiguous nucleotide portion thereof) is selected from, or comprises, one of the sequences selected from the group consisting of SEQ ID NOs: 889 to 988 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding STAT3 transcript.

In some aspects, binding of an ASO targeting a STAT3 transcript disclosed herein to a mRNA transcript encoding STAT3 can reduce expression levels and/or activity levels of STAT3.

II.C.5. ASO Targeting NRAS

NRas is an oncogene encoding a membrane protein that shuttles between the Golgi apparatus and the plasma membrane. NRas-encoding genomic DNA can be found at Chromosomal position 1p13.2 (i.e., nucleotides 5001 to 17438 of GenBank Accession No. NG_007572). Specifically, a combination of time-lapse microscopy and photobleaching techniques have revealed that in the absence of palmitoylation, GFP-tagged N-Ras undergoes rapid exchange between the cytosol and ER/Golgi membranes, and that wild-type GFP-N-Ras is recycled to the Golgi complex by a nonvesicular mechanism. N-ras mutations have been described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Oncogenic N-Ras can induce acute myeloid leukemia (AML)- or chronic myelomonocytic leukemia (CMML)-like disease in mice.

Neuroblastoma RAS viral oncogene (NRas) is known in the art by various names. Such names include: GTPase NRas, N-ras protein part 4, neuroblastoma RAS viral (v-ras) oncogene homolog neuroblastoma RAS viral oncogene homolog, transforming protein N-Ras, and v-ras neuroblastoma RAS viral oncogene homolog.

The NRAS gene provides instructions for making a protein called N-Ras that is involved primarily in regulating cell division. The mRNA sequence encoding human NRAS can be found at NCBI Reference sequence NM_002524.5 and is represented by the coding sequence (SEQ ID NO: 53).

Natural variants of the human NRas gene product are known. For example, natural variants of human NRas protein can contain one or more amino acid substitutions selected from: G12D, G13D, T50I, G60E, and any combinations thereof. Additional variants of human NRas protein resulting from alternative splicing are also known in the art, such as: G13R, Q61K, Q61R, and P34L. Therefore, the ASOs of the present disclosure can be designed to reduce or inhibit expression of the natural variants of the STAT3 protein.

SEQ ID NO: 51 is identical to a NRas pre-mRNA sequence except that nucleotide “t” in SEQ ID NO: 51 is shown as “u” in pre-mRNA. In certain aspects, the “target nucleic acid” comprises an intron of a NRas protein-encoding nucleic acids or naturally occurring variants thereof, and RNA nucleic acids derived therefrom, e.g., pre-mRNA. In other aspects, the target nucleic acid comprises an exon region of a NRas protein-encoding nucleic acids or naturally occurring variants thereof, and RNA nucleic acids derived therefrom, e.g., pre-mRNA. In yet other aspects, the target nucleic acid comprises an exon-intron junction of a NRas protein-encoding nucleic acids or naturally occurring variants thereof, and RNA nucleic acids derived therefrom, e.g., pre-mRNA. In some aspects, for example when used in research or diagnostics the “target nucleic acid” can be a cDNA or a synthetic oligonucleotide derived from the above DNA or RNA nucleic acid targets. The human NRas protein sequence encoded by the NRas pre-mRNA is shown as SEQ ID NO: 52. In other aspects, the target nucleic acid comprises an untranslated region of a NRas protein-encoding nucleic acids or naturally occurring variants thereof, e.g., 5′ UTR, 3′ UTR, or both.

In certain aspects, the ASOs of the disclosure also are capable of down-regulating (e.g., reducing or removing) expression of the NRas mRNA or protein. In this regard, the ASO of the disclosure can affect indirect inhibition of NRas protein through the reduction in NRas mRNA levels, typically in a mammalian cell, such as a human cell, such as a tumor cell. In particular, the present disclosure is directed to ASOs that target one or more regions of the NRas pre-mRNA (e.g., intron regions, exon regions, and/or exon-intron junction regions). Unless indicated otherwise, the term “NRas,” as used herein, can refer to NRas from one or more species (e.g., humans, non-human primates, dogs, cats, guinea pigs, rabbits, rats, mice, horses, cattle, and bears).

In some aspects, an ASO of the disclosure hybridizes to a region within the introns of a NRAS transcript, e.g., SEQ ID NO: 51 or SEQ ID NO: 53. In certain aspects, an ASO of the disclosure hybridizes to a region within the exons of a NRAS transcript, e.g., SEQ ID NO: 51 or SEQ ID NO: 53. In other aspects, an ASO of the disclosure hybridizes to a region within the exon-intron junction of a NRAS transcript, e.g., SEQ ID NO: 51 or SEQ ID NO: 53. In some aspects, an ASO of the disclosure hybridizes to a region within a NRAS transcript (e.g., an intron, exon, or exon-intron junction), e.g., SEQ ID NO: 51 or SEQ ID NO: 53, wherein the ASO has a design according to formula: 5′ A-B-C 3′ as described elsewhere herein.

In some aspects, the ASO of the present disclosure hybridizes to multiple target regions within the NRas transcript (e.g., genomic sequence, SEQ ID NO: 51). In some aspects, the ASO hybridizes to two different target regions within the NRas transcript. In some aspects, the ASO hybridizes to three different target regions within the NRas transcript. The sequences of exemplary ASOs that hybridizes to multiple target regions, and the start/end sites of the different target regions are provided in FIG. 1E. In some aspects, the ASOs that hybridizes to multiple regions within the NRas transcript (e.g., genomic sequence, SEQ ID NO: 51) are more potent (e.g., having lower EC50) at reducing NRas expression compared to ASOs that hybridizes to a single region within the NRas transcript (e.g., genomic sequence, SEQ ID NO: 51).

In some aspects, the ASO targets a mRNA encoding a particular isoform of NRAS protein (e.g., Isoform 1, NCBI ID: NP_001229821.1). In some aspects, the ASO targets all isoforms of NRas protein. In other aspects, the ASO targets two isoforms (e.g., Isoform 1 and Isoform 2 (NCBI ID:NP_009089.4), Isoform 2 and Isoform 3 (NCBI ID: NP_001123995), and Isoform 3 and Isoform 4 (NCBI ID: NP_001229820.1)) of NRas protein.

The ASOs of the disclosure comprise a contiguous nucleotide sequence which corresponds to the complement of a region of NRas transcript, e.g., a nucleotide sequence corresponding to SEQ ID NO: 51.

In some aspects, the nucleotide sequence of the ASOs of the disclosure or the contiguous nucleotide sequence has at least about 80% sequence identity to a sequence selected from SEQ ID NOs: 989-1088 (i.e., the sequences in FIG. 1E), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (e.g., FIG. 1E).

In some aspects the ASO (or contiguous nucleotide portion thereof) is selected from, or comprises, one of the sequences selected from the group consisting of SEQ ID NOs: 989-1088 to NRas or a region of at least 10 contiguous nucleotides thereof, wherein the ASO (or contiguous nucleotide portion thereof) can optionally comprise one, two, three, or four mismatches when compared to the corresponding NRas transcript. Non limiting exemplary ASOs targeting NRAS gene can be found at FIG. 1E.

In some aspects, the ASO comprises the sequence as set forth in SEQ ID NO: 989 (e.g., ASO-NRas-180). In some aspects, the ASO comprises the sequence as set forth in SEQ ID NO: 990 (e.g., ASO-NRas-181). In some aspects, the ASO comprises the sequence as set forth in SEQ ID NO: 991 (e.g., ASO-NRas-434). In some aspects, the ASO comprises the sequence as set forth in SEQ ID NO: 992 (e.g., ASO-NRas-617). In some aspects, the ASO comprises the sequence as set forth in SEQ ID NO: 993 (e.g., ASO-NRas-618). In some aspects, the ASO comprises the sequence as set forth in SEQ ID NO: 994 (e.g., ASO-NRas-619). In some aspects, the ASO comprises the sequence as set forth in SEQ ID NO: 995 (e.g., ASO-NRas-620). In some aspects, the ASO comprises the sequence as set forth in SEQ ID NO: 996 (e.g., ASO-NRas-3002). In some aspects, the ASO comprises the sequence as set forth in SEQ ID NO: 997 (e.g., ASO-NRas-617). In some aspects, the ASO comprises the sequence as set forth in SEQ ID NO: 998 (e.g., ASO-NRas-618). In some aspects, the ASO comprises the sequence as set forth in SEQ ID NO: 999 (e.g., ASO-NRas-619). In some aspects, the ASO comprises the sequence as set forth in SEQ ID NO: 1000 (e.g., ASO-NRas-615). In some aspects, the ASO comprises the sequence as set forth in SEQ ID NO: 1001 (e.g., ASO-NRas-616). In some aspects, the ASO comprises the sequence as set forth in SEQ ID NO: 1002 (e.g., ASO-NRas-617). In some aspects, the ASO comprises the sequence as set forth in SEQ ID NO: 1003 (e.g., ASO-NRas-618). In some aspects, the ASO comprises the sequence as set forth in SEQ ID NO: 1004 (e.g., ASO-NRas-619). In some aspects, the ASO comprises the sequence as set forth in SEQ ID NO: 1005 (e.g., ASO-NRas-620). In some aspects, the ASO comprises the sequence as set forth in SEQ ID NO: 1006 (e.g., ASO-NRas-134). In some aspects, the ASO comprises the sequence as set forth in SEQ ID NO: 1007 (e.g., ASO-NRas-176). In some aspects, the ASO comprises the sequence as set forth in SEQ ID NO: 1008 (e.g., ASO-NRas-179). In some aspects, the ASO comprises the sequence as set forth in SEQ ID NO: 1009 (e.g., ASO-NRas-180). In some aspects, the ASO comprises the sequence as set forth in SEQ ID NO: 1010 (e.g., ASO-NRas-181). In some aspects, the ASO comprises the sequence as set forth in SEQ ID NO: 1011 (e.g., ASO-NRas-183). In some aspects, the ASO comprises the sequence as set forth in SEQ ID NO: 1012 (e.g., ASO-NRas-325). In some aspects, the ASO comprises the sequence as set forth in SEQ ID NO: 1013 (e.g., ASO-NRas-337). In some aspects, the ASO comprises the sequence as set forth in SEQ ID NO: 1014 (e.g., ASO-NRas-338). In some aspects, the ASO comprises the sequence as set forth in SEQ ID NO: 1015 (e.g., ASO-NRas-341). In some aspects, the ASO comprises the sequence as set forth in SEQ ID NO: 1016 (e.g., ASO-NRas-378). In some aspects, the ASO comprises the sequence as set forth in SEQ ID NO: 1017 (e.g., ASO-NRas-379). In some aspects, the ASO comprises the sequence as set forth in SEQ ID NO: 1018 (e.g., ASO-NRas-388). In some aspects, the ASO comprises the sequence as set forth in SEQ ID NO: 1019 (e.g., ASO-NRas-389). In some aspects, the ASO comprises the sequence as set forth in SEQ ID NO: 1020 (e.g., ASO-NRas-399). In some aspects, the ASO comprises the sequence as set forth in SEQ ID NO: 1021 (e.g., ASO-NRas-400). In some aspects, the ASO comprises the sequence as set forth in SEQ ID NO: 1022 (e.g., ASO-NRas-401). In some aspects, the ASO comprises the sequence as set forth in SEQ ID NO: 1023 (e.g., ASO-NRas-402). In some aspects, the ASO comprises the sequence as set forth in SEQ ID NO: 1024 (e.g., ASO-NRas-408). In some aspects, the ASO comprises the sequence as set forth in SEQ ID NO: 1025 (e.g., ASO-NRas-421). In some aspects, the ASO comprises the sequence as set forth in SEQ ID NO: 1026 (e.g., ASO-NRas-422). In some aspects, the ASO comprises the sequence as set forth in SEQ ID NO: 1027 (e.g., ASO-NRas-429). In some aspects, the ASO comprises the sequence as set forth in SEQ ID NO: 1028 (e.g., ASO-NRas-490). In some aspects, the ASO comprises the sequence as set forth in SEQ ID NO: 1029 (e.g., ASO-NRas-513). In some aspects, the ASO comprises the sequence as set forth in SEQ ID NO: 1030 (e.g., ASO-NRas-514). In some aspects, the ASO comprises the sequence as set forth in SEQ ID NO: 1031 (e.g., ASO-NRas-520). In some aspects, the ASO comprises the sequence as set forth in SEQ ID NO: 1032 (e.g., ASO-NRas-521). In some aspects, the ASO comprises the sequence as set forth in SEQ ID NO: 1033 (e.g., ASO-NRas-522). In some aspects, the ASO comprises the sequence as set forth in SEQ ID NO: 1034 (e.g., ASO-NRas-524). In some aspects, the ASO comprises the sequence as set forth in SEQ ID NO: 1035 (e.g., ASO-NRas-532). In some aspects, the ASO comprises the sequence as set forth in SEQ ID NO: 1036 (e.g., ASO-NRas-534). In some aspects, the ASO comprises the sequence as set forth in SEQ ID NO: 1037 (e.g., ASO-NRas-535). In some aspects, the ASO comprises the sequence as set forth in SEQ ID NO: 1038 (e.g., ASO-NRas-536). In some aspects, the ASO comprises the sequence as set forth in SEQ ID NO: 1039 (e.g., ASO-NRas-537). In some aspects, the ASO comprises the sequence as set forth in SEQ ID NO: 1040 (e.g., ASO-NRas-539). In some aspects, the ASO comprises the sequence as set forth in SEQ ID NO: 1041 (e.g., ASO-NRas-604). In some aspects, the ASO comprises the sequence as set forth in SEQ ID NO: 1042 (e.g., ASO-NRas-611). In some aspects, the ASO comprises the sequence as set forth in SEQ ID NO: 1043 (e.g., ASO-NRas-612). In some aspects, the ASO comprises the sequence as set forth in SEQ ID NO: 1044 (e.g., ASO-NRas-613). In some aspects, the ASO comprises the sequence as set forth in SEQ ID NO: 1045 (e.g., ASO-NRas-614). In some aspects, the ASO comprises the sequence as set forth in SEQ ID NO: 1046 (e.g., ASO-NRas-615). In some aspects, the ASO comprises the sequence as set forth in SEQ ID NO: 1047 (e.g., ASO-NRas-616). In some aspects, the ASO comprises the sequence as set forth in SEQ ID NO: 1048 (e.g., ASO-NRas-617). In some aspects, the ASO comprises the sequence as set forth in SEQ ID NO: 1049 (e.g., ASO-NRas-618). In some aspects, the ASO comprises the sequence as set forth in SEQ ID NO: 1050 (e.g., ASO-NRas-619). In some aspects, the ASO comprises the sequence as set forth in SEQ ID NO: 1051 (e.g., ASO-NRas-620). In some aspects, the ASO comprises the sequence as set forth in SEQ ID NO: 1052 (e.g., ASO-NRas-622). In some aspects, the ASO comprises the sequence as set forth in SEQ ID NO: 1053 (e.g., ASO-NRas-623). In some aspects, the ASO comprises the sequence as set forth in SEQ ID NO: 1054 (e.g., ASO-NRas-624). In some aspects, the ASO comprises the sequence as set forth in SEQ ID NO: 1055 (e.g., ASO-NRas-690). In some aspects, the ASO comprises the sequence as set forth in SEQ ID NO: 1056 (e.g., ASO-NRas-691). In some aspects, the ASO comprises the sequence as set forth in SEQ ID NO: 1057 (e.g., ASO-NRas-731). In some aspects, the ASO comprises the sequence as set forth in SEQ ID NO: 1058 (e.g., ASO-NRas-835). In some aspects, the ASO comprises the sequence as set forth in SEQ ID NO: 1059 (e.g., ASO-NRas-836). In some aspects, the ASO comprises the sequence as set forth in SEQ ID NO: 1060 (e.g., ASO-NRas-918). In some aspects, the ASO comprises the sequence as set forth in SEQ ID NO: 1061 (e.g., ASO-NRas-922). In some aspects, the ASO comprises the sequence as set forth in SEQ ID NO: 1062 (e.g., ASO-NRas-1072). In some aspects, the ASO comprises the sequence as set forth in SEQ ID NO: 1063 (e.g., ASO-NRas-1074). In some aspects, the ASO comprises the sequence as set forth in SEQ ID NO: 1064 (e.g., ASO-NRas-1313). In some aspects, the ASO comprises the sequence as set forth in SEQ ID NO: 1065 (e.g., ASO-NRas-1475). In some aspects, the ASO comprises the sequence as set forth in SEQ ID NO: 1066 (e.g., ASO-NRas-1617). In some aspects, the ASO comprises the sequence as set forth in SEQ ID NO: 1067 (e.g., ASO-NRas-1618). In some aspects, the ASO comprises the sequence as set forth in SEQ ID NO: 1068 (e.g., ASO-NRas-1621). In some aspects, the ASO comprises the sequence as set forth in SEQ ID NO: 1069 (e.g., ASO-NRas-1622). In some aspects, the ASO comprises the sequence as set forth in SEQ ID NO: 1070 (e.g., ASO-NRas-1623). In some aspects, the ASO comprises the sequence as set forth in SEQ ID NO: 1071 (e.g., ASO-NRas-1956). In some aspects, the ASO comprises the sequence as set forth in SEQ ID NO: 1072 (e.g., ASO-NRas-1957). In some aspects, the ASO comprises the sequence as set forth in SEQ ID NO: 1073 (e.g., ASO-NRas-1958). In some aspects, the ASO comprises the sequence as set forth in SEQ ID NO: 1074 (e.g., ASO-NRas-1959). In some aspects, the ASO comprises the sequence as set forth in SEQ ID NO: 1075 (e.g., ASO-NRas-1962). In some aspects, the ASO comprises the sequence as set forth in SEQ ID NO: 1076 (e.g., ASO-NRas-1965). In some aspects, the ASO comprises the sequence as set forth in SEQ ID NO: 1077 (e.g., ASO-NRas-2113). In some aspects, the ASO comprises the sequence as set forth in SEQ ID NO: 1078 (e.g., ASO-NRas-2114). In some aspects, the ASO comprises the sequence as set forth in SEQ ID NO: 1079 (e.g., ASO-NRas-2122). In some aspects, the ASO comprises the sequence as set forth in SEQ ID NO: 1080 (e.g., ASO-NRas-2417). In some aspects, the ASO comprises the sequence as set forth in SEQ ID NO: 1081 (e.g., ASO-NRas-2419). In some aspects, the ASO comprises the sequence as set forth in SEQ ID NO: 1082 (e.g., ASO-NRas-2759). In some aspects, the ASO comprises the sequence as set forth in SEQ ID NO: 1083 (e.g., ASO-NRas-2760). In some aspects, the ASO comprises the sequence as set forth in SEQ ID NO: 1084 (e.g., ASO-NRas-2761). In some aspects, the ASO comprises the sequence as set forth in SEQ ID NO: 1085 (e.g., ASO-NRas-2886). In some aspects, the ASO comprises the sequence as set forth in SEQ ID NO: 1086 (e.g., ASO-NRas-3557). In some aspects, the ASO comprises the sequence as set forth in SEQ ID NO: 1087 (e.g., ASO-NRas-4027). In some aspects, the ASO comprises the sequence as set forth in SEQ ID NO: 1088 (e.g., ASO-NRas-4082).

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 989 to 1088. In some aspects the ASO (or contiguous nucleotide portion thereof) is selected from, or comprises, one of the sequences selected from the group consisting of SEQ ID NOs: 989 to 1088 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 989 to 1088 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding NRas transcript. In some aspects the ASO (or contiguous nucleotide portion thereof) is selected from, or comprises, one of the sequences selected from the group consisting of SEQ ID NOs: 989 to 1088 except for 1, 2, 3, 4, 5, 6, 7, 8, 9 or 10 substitutions, wherein the substituted ASO can bind to the NRas transcript. In some aspects the ASO (or contiguous nucleotide portion thereof) is selected from, or comprises, one of the sequences selected from the group consisting of SEQ ID NOs: 989 to 1088 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding NRas transcript.

In some aspects, binding of an ASO targeting a NRas transcript disclosed herein to a mRNA transcript encoding NRas can reduce expression levels and/or activity levels of NRas.

II.C.6. ASO Targeting KRAS

KRAS is known in the art by various names. Such names include: KRAS Proto-Oncogene, GTPase; V-Ki-Ras2 Kirsten Rat Sarcoma 2 Viral Oncogene Homolog; GTPase KRas; C-Ki-Ras; K-Ras 2; KRAS2; RASK2; V-Ki-Ras2 Kirsten Rat Sarcoma Viral Oncogene Homolog; Kirsten Rat Sarcoma Viral Proto-Oncogene; Cellular Transforming Proto-Oncogene; Cellular C-Ki-Ras2 Proto-Oncogene; Transforming Protein P21; PR310 C-K-Ras Oncogene; C-Kirsten-Ras Protein; K-Ras P21 Protein; and Oncogene KRAS2.

The sequence for the human KRAS gene can be found at chromosomal location 12p12.1 and under publicly available GenBank Accession Number NC_000012 (25,204,789-25,250,936). The genomic sequence for human wild-type KRAS transcript corresponds to the reverse complement of residues 25,204,789-25,250,936 of NC_000012 (SEQ ID NO: 35). The KRAS G12D genomic sequence provided in SEQ ID NO: 31 differs from SEQ ID NO: 35 in that it has a guanine to adenine substitution at nucleotide position 5,587. An exemplary KRAS G12D mRNA sequence is provided in SEQ ID NO: 33, except that the nucleotide “t” in SEQ ID NO: 33 is shown as “u” in the mRNA. The KRAS G12D mRNA provided in SEQ ID NO: 33 differs from the wild-type mRNA sequence (e.g., GenBank Accession No. NM_004985.5; SEQ ID NO: 37) in that it has a guanine to adenine substitution at nucleotide position 225. The sequence for human KRAS protein can be found under publicly available Accession Numbers: P01116 (canonical sequence), A8K8Z5, B0LPF9, P01118, and Q96D10, each of which is incorporated by reference herein in its entirety.

There are two isoforms of the human KRAS protein (P01116), resulting from alternative splicing. Isoform 2A (Accession Number: P01116-1; SEQ ID NO: 38) is the canonical sequence. It is also known as K-Ras4A. Isoform 2B (Accession Number: P01116-2; also known as K-Ras4B; SEQ ID NO: 36) differs from the canonical sequence as follows: (i) 151-153: RVE→GVD; and (ii) 165-189: QYRLKKISKEEKTPGCVKIKKCIIM (SEQ ID NO:599)→KHKEKMSKDGKKKKKKSKTKCVIM (SEQ ID NO:600). In some aspects, ASOs disclosed herein can reduce or inhibit expression of KRAS protein Isoform 2A, Isoform 2B, or both.

Natural variants of the human KRAS gene product are known. For example, natural variants of human KRAS protein can contain one or more amino acid substitutions selected from: K5E, K5N, G10GG, G10V, G12A, G12C, G12F, G12I, G12L, G12R, G12S, G12V, G13C, G13D, G13E, G13R, G13V, V14L, L19F, T20M, Q22E, Q22H, Q22K, Q22R, Q25H, N26Y, F28L, E31K, D33E, P34L, P34Q, P34R, 136M, R41K, D57N, T58I, A59T, G60D, G60R, G60S, G60V, Q61A, Q61H, Q61K, Q61L, Q61P, Q61R, E63K, S65N, R68S, Y71H, T74A, L79L, R97I, Q99E, M111L, K117N, K117R, D119G, S122F, T144P, A146P, A146T, A146V, K147E, K147T, R149K, L159S, I163S, R164Q, I183N, 184M, or combinations thereof. Natural variants that are specific to KRAS protein Isoform 2B contain one or more amino acid substitutions selected from: V152G, D153V, F156L, F156L, or combinations thereof. The ASOs of the present disclosure can be designed to reduce or inhibit expression of one or more of the variants of the KRAS protein (e.g., any variants known in the art). In some aspects, a KRAS mutant has an amino acid substitution of G12D. In some aspects, the ASOs of the present disclosure target one or more KRAS mutants. In other aspects, a KRAS mutant that the ASOs target is KRAS G12D (SEQ ID NO: 32). Exemplary sequences for KRAS G12D mRNA and KRAS G12D protein are provided in SEQ ID NO: 33 and SEQ ID NO: 32.

In some aspects, a target nucleic acid sequence of an ASO disclosed herein comprises one or more regions of a KRAS pre-mRNA. For example, SEQ ID NO: 31 (described above) is identical to a KRAS pre-mRNA sequence except that nucleotide “t” in SEQ ID NO: 31 is shown as “u” in the pre-mRNA. As used herein, the term “target nucleic acid sequence” refers to a nucleic acid sequence that is complementary to an ASO disclosed herein. In certain aspects, the target nucleic acid sequence comprises an exon region of a KRAS protein-encoding nucleic acids or naturally occurring variants thereof, and RNA nucleic acids derived therefrom, e.g., pre-mRNA. In some aspects, the target nucleic acid sequence comprises an intron of a KRAS protein-encoding nucleic acids or naturally occurring variants thereof, and RNA nucleic acids derived therefrom, e.g., pre-mRNA. In further aspects, the target nucleic acid sequence comprises an exon-intron junction of a KRAS protein-encoding nucleic acids or naturally occurring variants thereof, and RNA nucleic acids derived therefrom, e.g., pre-mRNA. In some aspects, for example, when used in research or diagnostics, the target nucleic acid can be a cDNA or a synthetic oligonucleotide derived from DNA or RNA nucleic acid targets described herein. In some aspects, the target nucleic acid comprises an untranslated region of a KRAS protein-encoding nucleic acids or naturally occurring variants thereof, e.g., 5′ UTR, 3′ UTR, or both.

Accordingly, in some aspects, an ASO disclosed herein hybridizes to an exon region of a KRAS transcript, e.g., SEQ ID NO: 31 or SEQ ID NO: 33. In some aspects, an ASO of the present disclosure hybridizes to an intron region of a KRAS transcript, e.g., SEQ ID NO: 31. In some aspects, an ASO hybridizes to an exon-intron junction of a KRAS transcript, e.g., SEQ ID NO: 31. In some aspects, an ASO of the present disclosure hybridizes to a region within a KRAS transcript (e.g., an intron, exon, or exon-intron junction), e.g., SEQ ID NO: 31, wherein the ASO has a design described elsewhere herein.

In some aspects, a target nucleic sequence of the ASOs disclosed herein is a KRAS mRNA, e.g., SEQ ID NO: 33. Accordingly, in certain aspects, an ASO disclosed herein can hybridize to one or more regions of a KRAS mRNA. In some aspects, ASOs of the present disclosure target mRNA encoding a particular isoform of KRAS protein. In certain aspects, ASOs disclosed herein can target all isoforms of KRAS protein, including any variants thereof (e.g., those described herein). In some aspects, a KRAS protein that can be targeted by ASOs of the present disclosure comprises a G12D amino acid substitution. Non-limiting exemplary ASOs targeting a KRAS transcript is shown at FIG. 1F.

In some aspects, the ASO comprises a sequence selected from the group consisting of SEQ ID NOs: 807 to 820. In some aspects, the ASO comprises the sequence as set forth in SEQ ID NO: 807 (e.g., ASO-KRAS-0004). In some aspects, the ASO comprises the sequence as set forth in SEQ ID NO: 808 (e.g., ASO-KRAS-0005). In some aspects, the ASO comprises the sequence as set forth in SEQ ID NO: 809 (e.g., ASO-KRAS-0006). In some aspects, the ASO comprises the sequence as set forth in SEQ ID NO: 810 (e.g., ASO-KRAS-0007). In some aspects, the ASO comprises the sequence as set forth in SEQ ID NO: 811 (e.g., ASO-KRAS-0008). In some aspects, the ASO comprises the sequence as set forth in SEQ ID NO: 812 (e.g., ASO-KRAS-0009). In some aspects, the ASO comprises the sequence as set forth in SEQ ID NO: 813 (e.g., ASO-KRAS-0010). In some aspects, the ASO comprises the sequence as set forth in SEQ ID NO: 814 (e.g., ASO-KRAS-0011). In some aspects, the ASO comprises the sequence as set forth in SEQ ID NO: 815 (e.g., ASO-KRAS-0012). In some aspects, the ASO comprises the sequence as set forth in SEQ ID NO: 816 (e.g., ASO-KRAS-0013). In some aspects, the ASO comprises the sequence as set forth in SEQ ID NO: 817 (e.g., ASO-KRAS-0014). In some aspects, the ASO comprises the sequence as set forth in SEQ ID NO: 818 (e.g., ASO-KRAS-0015). In some aspects, the ASO comprises the sequence as set forth in SEQ ID NO: 819 (e.g., ASO-KRAS-0016). In some aspects, the ASO comprises the sequence as set forth in SEQ ID NO: 820 (e.g., ASO-KRAS-0017). In some aspects, the ASO comprises a sequence selected from the group consisting of SEQ ID NOs: 821-835. In some aspects, the ASO comprises the sequence as set forth in SEQ ID NO: 821 (e.g., ASO-KRAS-0018). In some aspects, the ASO comprises the sequence as set forth in SEQ ID NO: 822 (e.g., ASO-KRAS-0019). In some aspects, the ASO comprises the sequence as set forth in SEQ ID NO: 823 (e.g., ASO-KRAS-0020). In some aspects, the ASO comprises the sequence as set forth in SEQ ID NO: 824 (e.g., ASO-KRAS-0021). In some aspects, the ASO comprises the sequence as set forth in SEQ ID NO: 825 (e.g., ASO-KRAS-0022). In some aspects, the ASO comprises the sequence as set forth in SEQ ID NO: 826 (e.g., ASO-KRAS-0023). In some aspects, the ASO comprises the sequence as set forth in SEQ ID NO: 827 (e.g., ASO-KRAS-0024). In some aspects, the ASO comprises the sequence as set forth in SEQ ID NO: 828 (e.g., ASO-KRAS-0025). In some aspects, the ASO comprises the sequence as set forth in SEQ ID NO: 829 (e.g., ASO-KRAS-0026). In some aspects, the ASO comprises the sequence as set forth in SEQ ID NO: 830 (e.g., ASO-KRAS-0027). In some aspects, the ASO comprises the sequence as set forth in SEQ ID NO: 831 (e.g., ASO-KRAS-0028). In some aspects, the ASO comprises the sequence as set forth in SEQ ID NO: 832 (e.g., ASO-KRAS-0029). In some aspects, the ASO comprises the sequence as set forth in SEQ ID NO: 833 (e.g., ASO-KRAS-0030). In some aspects, the ASO comprises the sequence as set forth in SEQ ID NO: 834 (e.g., ASO-KRAS-0031). In some aspects, the ASO comprises the sequence as set forth in SEQ ID NO: 835 (e.g., ASO-KRAS-0032). In some aspects, the ASO comprises a sequence selected from the group consisting of SEQ ID NOs: 836-851. In some aspects, the ASO comprises the sequence as set forth in SEQ ID NO: 836 (e.g., ASO-KRAS-0033). In some aspects, the ASO comprises the sequence as set forth in SEQ ID NO: 837 (e.g., ASO-KRAS-0034). In some aspects, the ASO comprises the sequence as set forth in SEQ ID NO: 838 (e.g., ASO-KRAS-0035). In some aspects, the ASO comprises the sequence as set forth in SEQ ID NO: 839 (e.g., ASO-KRAS-0036). In some aspects, the ASO comprises the sequence as set forth in SEQ ID NO: 840 (e.g., ASO-KRAS-0037). In some aspects, the ASO comprises the sequence as set forth in SEQ ID NO: 841 (e.g., ASO-KRAS-0038). In some aspects, the ASO comprises the sequence as set forth in SEQ ID NO: 842 (e.g., ASO-KRAS-0039). In some aspects, the ASO comprises the sequence as set forth in SEQ ID NO: 843 (e.g., ASO-KRAS-0040). In some aspects, the ASO comprises the sequence as set forth in SEQ ID NO: 844 (e.g., ASO-KRAS-0041). In some aspects, the ASO comprises the sequence as set forth in SEQ ID NO: 845 (e.g., ASO-KRAS-0042). In some aspects, the ASO comprises the sequence as set forth in SEQ ID NO: 846 (e.g., ASO-KRAS-0043). In some aspects, the ASO comprises the sequence as set forth in SEQ ID NO: 847 (e.g., ASO-KRAS-0044). In some aspects, the ASO comprises the sequence as set forth in SEQ ID NO: 848 (e.g., ASO-KRAS-0045). In some aspects, the ASO comprises the sequence as set forth in SEQ ID NO: 849 (e.g., ASO-KRAS-0046). In some aspects, the ASO comprises the sequence as set forth in SEQ ID NO: 850 (e.g., ASO-KRAS-0047). In some aspects, the ASO comprises the sequence as set forth in SEQ ID NO: 851 (e.g., ASO-KRAS-0048). In some aspects, the ASO comprises a sequence selected from the group consisting of SEQ ID NOs: 852-868. In some aspects, the ASO comprises the sequence as set forth in SEQ ID NO: 852 (e.g., ASO-KRAS-0049). In some aspects, the ASO comprises the sequence as set forth in SEQ ID NO: 853 (e.g., ASO-KRAS-0050). In some aspects, the ASO comprises the sequence as set forth in SEQ ID NO: 854 (e.g., ASO-KRAS-0051). In some aspects, the ASO comprises the sequence as set forth in SEQ ID NO: 855 (e.g., ASO-KRAS-0052). In some aspects, the ASO comprises the sequence as set forth in SEQ ID NO: 856 (e.g., ASO-KRAS-0053). In some aspects, the ASO comprises the sequence as set forth in SEQ ID NO: 857 (e.g., ASO-KRAS-0054). In some aspects, the ASO comprises the sequence as set forth in SEQ ID NO: 858 (e.g., ASO-KRAS-0055). In some aspects, the ASO comprises the sequence as set forth in SEQ ID NO: 859 (e.g., ASO-KRAS-0056). In some aspects, the ASO comprises the sequence as set forth in SEQ ID NO: 860 (e.g., ASO-KRAS-0057). In some aspects, the ASO comprises the sequence as set forth in SEQ ID NO: 861 (e.g., ASO-KRAS-0058). In some aspects, the ASO comprises the sequence as set forth in SEQ ID NO: 862 (e.g., ASO-KRAS-0059). In some aspects, the ASO comprises the sequence as set forth in SEQ ID NO: 863 (e.g., ASO-KRAS-0060). In some aspects, the ASO comprises the sequence as set forth in SEQ ID NO: 864 (e.g., ASO-KRAS-0061). In some aspects, the ASO comprises the sequence as set forth in SEQ ID NO: 865 (e.g., ASO-KRAS-0062). In some aspects, the ASO comprises the sequence as set forth in SEQ ID NO: 866 (e.g., ASO-KRAS-0063). In some aspects, the ASO comprises the sequence as set forth in SEQ ID NO: 867 (e.g., ASO-KRAS-0064). In some aspects, the ASO comprises the sequence as set forth in SEQ ID NO: 868 (e.g., ASO-KRAS-0065). In some aspects, the ASO comprises a sequence selected from the group consisting of SEQ ID NOs: 869-888. In some aspects, the ASO comprises the sequence as set forth in SEQ ID NO: 869 (e.g., ASO-KRAS-0066). In some aspects, the ASO comprises the sequence as set forth in SEQ ID NO: 870 (e.g., ASO-KRAS-0067). In some aspects, the ASO comprises the sequence as set forth in SEQ ID NO: 871 (e.g., ASO-KRAS-0068). In some aspects, the ASO comprises the sequence as set forth in SEQ ID NO: 872 (e.g., ASO-KRAS-0069). In some aspects, the ASO comprises the sequence as set forth in SEQ ID NO: 873 (e.g., ASO-KRAS-0070). In some aspects, the ASO comprises the sequence as set forth in SEQ ID NO: 874 (e.g., ASO-KRAS-0071). In some aspects, the ASO comprises the sequence as set forth in SEQ ID NO: 875 (e.g., ASO-KRAS-0072). In some aspects, the ASO comprises the sequence as set forth in SEQ ID NO: 876 (e.g., ASO-KRAS-0073). In some aspects, the ASO comprises the sequence as set forth in SEQ ID NO: 877 (e.g., ASO-KRAS-0074). In some aspects, the ASO comprises the sequence as set forth in SEQ ID NO: 878 (e.g., ASO-KRAS-0075). In some aspects, the ASO comprises the sequence as set forth in SEQ ID NO: 879 (e.g., ASO-KRAS-0076). In some aspects, the ASO comprises the sequence as set forth in SEQ ID NO: 880 (e.g., ASO-KRAS-0077). In some aspects, the ASO comprises the sequence as set forth in SEQ ID NO: 881 (e.g., ASO-KRAS-0078). In some aspects, the ASO comprises the sequence as set forth in SEQ ID NO: 882 (e.g., ASO-KRAS-0079). In some aspects, the ASO comprises the sequence as set forth in SEQ ID NO: 883 (e.g., ASO-KRAS-0080). In some aspects, the ASO comprises the sequence as set forth in SEQ ID NO: 884 (e.g., ASO-KRAS-0081). In some aspects, the ASO comprises the sequence as set forth in SEQ ID NO: 885 (e.g., ASO-KRAS-0082). In some aspects, the ASO comprises the sequence as set forth in SEQ ID NO: 886 (e.g., ASO-KRAS-0083). In some aspects, the ASO comprises the sequence as set forth in SEQ ID NO: 887 (e.g., ASO-KRAS-0084). In some aspects, the ASO comprises the sequence as set forth in SEQ ID NO: 888 (e.g., ASO-KRAS-0085).

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 807 to 888. In some aspects the ASO (or contiguous nucleotide portion thereof) is selected from, or comprises, one of the sequences selected from the group consisting of SEQ ID NOs: 807 to 888 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 807 to 888 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding KRAS transcript. In some aspects the ASO (or contiguous nucleotide portion thereof) is selected from, or comprises, one of the sequences selected from the group consisting of SEQ ID NOs: 807 to 888 except for 1, 2, 3, 4, 5, 6, 7, 8, 9 or 10 substitutions, wherein the substituted ASO can bind to the KRAS transcript. In some aspects the ASO (or contiguous nucleotide portion thereof) is selected from, or comprises, one of the sequences selected from the group consisting of SEQ ID NOs: 807 to 888 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding KRAS transcript.

In some aspects, binding of an ASO targeting a KRAS transcript disclosed herein to a mRNA transcript encoding KRAS can reduce expression levels and/or activity levels of KRAS.

II.C.7 ASO Targeting Pmp22

Peripheral myelin protein 22 (PMP22) is also known as growth arrest-specific protein 3 (GAS-3), is encoded by the PMP22 gene. PMP22 is a 22 kDa transmembrane glycoprotein made up of 160 amino acids, and is mainly expressed in the Schwann cells of the peripheral nervous system. Schwann cells show high expression of PMP22, where it can constitute 2-5% of total protein content in compact myelin. Compact myelin is the bulk of the peripheral neuron's myelin sheath, a protective fatty layer that provides electrical insulation for the neuronal axon. The level of PMP22 expression is relatively low in the central nervous system of adults.

In humans, the PMP22 gene is located on chromosome 17p11.2 and spans approximately 40 kb. The gene contains six exons conserved in both humans and rodents, two of which are 5′ untranslated exons (1a and 1b) and result in two different RNA transcripts with identical coding sequences. The two transcripts differ in their 5′ untranslated regions and have their own promoter regulating expression. The remaining exons (2 to 5) include the coding region of the PMP22 gene, and are joined together after post-transcriptional modification (i.e. alternative splicing). The PMP22 protein is characterized by four transmembrane domains, two extracellular loops (ECL1 and ECL2), and one intracellular loop. ECL1 has been suggested to mediate a homophilic interaction between two PMP22 proteins, whereas ECL2 has been shown to mediate a heterophilic interaction between PMP22 protein and Myelin protein zero (MPZ or MP0).

PMP22 plays an essential role in the formation and maintenance of compact myelin. When Schwann cells come into contact with a neuronal axon, expression of PMP22 is significantly up-regulated, whereas PMP22 is down-regulated during axonal degeneration or transection. PMP22 has shown association with zonula-occludens 1 and occludin, proteins that are involved in adhesion with other cells and the extracellular matrix, and also support functioning of myelin. Along with cell adhesion function, PMP22 is also up-regulated during Schwann cell proliferation, suggesting a role in cell-cycle regulation. PMP22 is detectable in non-neural tissues, where its expression has been shown to serve as growth-arrest-specific (gas-3) function.

Improper gene dosage of the PMP22 gene can cause aberrant protein synthesis and function of myelin sheath. Since the components of myelin are stoichiometrically set, any irregular expression of a component can cause destabilization of myelin and neuropathic disorders. Alterations of PMP22 gene expression are associated with a variety of neuropathies, such as Charcot-Marie-Tooth type 1A (CMT1A), Dejerine-Sottas disease, and Hereditary Neuropathy with Liability to Pressure Palsy (HNPP). Too much PMP22 (e.g. caused by gene duplication) results in CMT1A. Gene duplication of PMP22 is the most common genetic cause of CMT where the overproduction of PMP22 results in defects in multiple signaling pathways and dysfunction of transcriptional factors like KNOX20, SOX10 and EGR2.

The sequence for the human PMP22 gene can be found under publicly available as NCBI RefSeq Acc. No. NM_000304. Alternative RefSeq mRNA transcripts have accession numbers NM_001281455, NM-001281456, NM-153321, and NM_153322, respectively. The human PMP22 gene is found at chromosome location 17p12 at 15,229,777-15,265,326.

The sequence for the human PMP22 pre-mRNA transcript (SEQ ID NO: 264) corresponds to the reverse complement of residues 15,229,777-15,265,326, of chromosome location 17p12. The PMP22 mRNA sequence (GenBank Accession No. NM_000304.4) is provided in SEQ ID NO: 58. The sequence for human PMP22 protein can be found under publicly available Uniprot Accession Number Q01453 (canonical sequence, SEQ ID NO: 60). Potential PMP22 isoforms have Uniprot Accession Numbers A8MU75, J3KQW0, A0A2R8Y5L5, J3KT36, and J3QS08, respectively. The publicly available contents of the database entries corresponding to accession numbers disclosed herein are incorporated by reference in their entireties.

Natural variants of the human PMP22 gene product are known. For example, natural variants of human PMP22 protein can contain one or more amino acid substitutions selected from L16P, S22F, Δ25-26, D37V, V65F, S72L, S79C, G93R, L105R, G107V, T118N, L147R, H12Q, L16P, L19P, M69K, L71P, S72L, S72P, S72W, S76I, S79P, L80P, L80R, Δ84, G100E, G100R, L105R, C109R, S149R, G150C, G150D, R157W, S22F, V30M, A67T, S23T, W28R, A67P, Δ115-118, and any combination thereof.

The ASOs of the present disclosure can be designed to reduce or inhibit expression of the natural variants of the PMP22 protein.

An example of a target nucleic acid sequence of the ASOs is PMP22 pre-mRNA. SEQ ID NO: 58 represents a human PMP22 genomic sequence (i.e., reverse complement of nucleotides 15,229,777-15,265,326, complement, of chromosome 17p12). SEQ ID NO: 58 is identical to a PMP22 pre-mRNA sequence except that nucleotide “t” in SEQ ID NO: 58 is shown as “u” in pre-mRNA.

In some aspects, the ASO comprises a contiguous nucleotide sequence of 10 to 30 nucleotides in length that is complementary to a nucleic acid sequence within nucleotides 1 to 1828 of a PMP22 transcript corresponding to a nucleotide sequence as set forth in SEQ ID NO: 264 (PMP22 full mRNA transcript) or nucleotides 208 to 690 of a PMP22 transcript corresponding to a nucleotide sequence as set forth in SEQ ID NO: 59 (PMP22 coding sequence).

In some aspects, the contiguous nucleotide sequence is at least about 80%, at least about 85%, at least about 90%, at least about 95%, or about 100% complementary to the nucleic acid sequence within the PMP22 transcript. In some aspects, the ASO is capable of reducing PMP22 protein expression in a human cell (e.g., a Schwan cell), wherein the human cell expresses the PMP22 protein.

In some aspects, the PMP22 protein expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to PMP22 protein expression in a human cell that is not exposed to the ASO.

In some aspects, the ASO is capable of reducing a level of PMP22 mRNA in a human cell (e.g., an immune cell), wherein the human cell expresses the PMP22 mRNA. In some aspects, the level of PMP22 mRNA is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to the level of the PMP22 mRNA in a human cell that is not exposed to the ASO.

In certain aspects, the target nucleic acid comprises an intron of a PMP22 protein-encoding nucleic acids or naturally occurring variants thereof, and RNA nucleic acids derived therefrom, e.g., pre-mRNA. In other aspects, the target nucleic acid comprises an exon region of a PMP22 protein-encoding nucleic acids or naturally occurring variants thereof, and RNA nucleic acids derived therefrom, e.g., pre-mRNA. In yet other aspects, the target nucleic acid comprises an exon-intron junction of a PMP22 protein-encoding nucleic acids or naturally occurring variants thereof, and RNA nucleic acids derived therefrom, e.g., pre-mRNA. In some aspects, for example when used in research or diagnostics the target nucleic acid can be a cDNA or a synthetic oligonucleotide derived from the above DNA or RNA nucleic acid targets. The human PMP22 coding sequence (CDS) is shows as SEQ ID NO:59, and protein sequence encoded by the coding sequence in the PMP22 pre-mRNA is shown as SEQ ID NO: 60. In other aspects, the target nucleic acid comprises an untranslated region of a PMP22 protein-encoding nucleic acids or naturally occurring variants thereof, e.g., 5′ UTR, 3′ UTR, or both.

In some aspects, an ASO of the disclosure hybridizes to a region within the introns of a PMP22 transcript, e.g., SEQ ID NO: 58. In certain aspects, an ASO of the disclosure hybridizes to a region within the exons of a PMP22 transcript, e.g., SEQ ID NO: 58. In other aspects, an ASO of the disclosure hybridizes to a region within the exon-intron junction of a PMP22 transcript, e.g., SEQ ID NO: 58.

In some aspects, the ASO comprises a contiguous nucleotide sequence (e.g., 10 to 30 nucleotides in length, e.g., 20 nucleotides in length) that are complementary to a nucleic acid sequence within a PMP22 transcript, e.g., a region corresponding to SEQ ID NO: 264. In some aspects, the ASO comprises a contiguous nucleotide sequence that hybridizes to a nucleic acid sequence, or a region within the sequence, of a PMP22 transcript (target region), wherein the contiguous nucleotide sequence is complementary to a nucleic acid sequence within (i) a 5′ untranslated region (UTR); (ii) a coding region; or (iii) a 3′ UTR of the PMP22 transcript. In some aspects, the contiguous nucleotide sequence is complementary to a nucleic acid sequence comprising (i) nucleotides 1-173 of SEQ ID NO: 58 (exon 1); (ii) nucleotides 174-285 of SEQ ID NO: 58 (exon 2); (iii) nucleotides 286-385 of SEQ ID NO: 58 (exon 3); (iv) nucleotides 386-526 of SEQ ID NO: 58 (exon 4); (v) 527-1828 of SEQ ID NO: 58 (exon 5); (vi) 200-300 of SEQ ID NO: 58, (vii) nucleotides 200-400 of SEQ ID NO: 58; (viii) nucleotides 500-600 of SEQ ID NO: 58; (ix) nucleotides 600-700 of SEQ ID NO: 58; (x) nucleotides 600-800 of SEQ ID NO: 58; (xi) 1200-1300 of SEQ ID NO: 58; (xii) 400-600 of SEQ ID NO: 58; (xiii) nucleotides 800-1000 of SEQ ID NO: 58; (xiv) nucleotides 1000-1200 of SEQ ID NO: 58; (xv) nucleotides 1200-1400 of SEQ ID NO: 58; (xvi) nucleotides 1400-1600 of SEQ ID NO: 58; or (xvii) 1600-1800 of SEQ ID NO:58.

In some aspects, the contiguous nucleotide sequence is complementary to a nucleic acid sequence comprising nucleotides 152-168 (SEQ ID NO: 62), 225-244 (SEQ ID NO:63), 227-246 (SEQ ID NO:64), 235-254 (SEQ ID NO:65), 265-284 (SEQ ID NO:66), 271-290 (SEQ ID NO:67), 380-399 (SEQ ID NO:68), 383-402 (SEQ ID NO:69), 385-404 (SEQ ID NO:70), 418-437 (SEQ ID NO:71), 479-498 (SEQ ID NO:72), 583-602 (SEQ ID NO:73), 671-690 (SEQ ID NO:74), 672-691 (SEQ ID NO:75), 673-692 (SEQ ID NO:76), 674-693 (SEQ ID NO:77), 675-691 (SEQ ID NO:78), 676-691 (SEQ ID NO:79), 678-693 (SEQ ID NO:80), 939-958 (SEQ ID NO:81), 940-959 (SEQ ID NO:82), 1127-1146 (SEQ ID NO:83), 1130-1149 (SEQ ID NO:84), 1293-1312 (SEQ ID NO:85), 1316-1335 (SEQ ID NO:86), 1317-1336 (SEQ ID NO:87), 1319-1338 (SEQ ID NO:88), 1365-1384 (SEQ ID NO:89), 1404-1423 (SEQ ID NO:90), 1604-1623 (SEQ ID NO:91), 1605-1624 (SEQ ID NO:92), 1611-1630 (SEQ ID NO:93), 1612-1631 (SEQ ID NO:94), or 1679-1688 (SEQ ID NO:263) of SEQ ID NO: 58. In some aspects, the contiguous nucleotide sequence comprises a nucleotide sequence complementary to a nucleic acid sequence comprising nucleotides 152-168 (SEQ ID NO:62), 235-254 (SEQ ID NO:65), 385-404 (SEQ ID NO:70), 479-498 (SEQ ID NO:72), 672-691 (SEQ ID NO:75), 675-691 (SEQ ID NO:78), 939-958 (SEQ ID NO:81), 1130-1149 (SEQ ID NO:84), 1293-1312 (SEQ ID NO:85), 1365-1384 (SEQ ID NO:89), 1404-1423 (SEQ ID NO:90), or 1605-1624 (SEQ ID NO:92) of SEQ ID NO: 58.

In some aspects, the target region corresponds to a 16-mer nucleotide sequence corresponding to positions 208-223, 209-224, 210-225, 211-226, 212-227, 213-228, 214-229, 215-230, 216-231, 217-232, 218-233, 219-234, 220-235, 221-236, 222-237, 223-238, 224-239, 225-240, 226-241, 227-242, 228-243, 229-244, 230-245, 231-246, 232-247, 233-248, 234-249, 235-250, 236-251, 237-252, 238-253, 239-254, 240-255, 241-256, 242-257, 243-258, 244-259, 245-260, 246-261, 247-262, 248-263, 249-264, 250-265, 251-266, 252-267, 253-268, 254-269, 255-270, 256-271, 257-272, 258-273, 259-274, 260-275, 261-276, 262-277, 263-278, 264-279, 265-280, 266-281, 267-282, 268-283, 269-284, 270-285, 271-286, 272-287, 273-288, 274-289, 275-290, 276-291, 277-292, 278-293, 279-294, 280-295, 281-296, 282-297, 283-298, 284-299, 285-300, 286-301, 287-302, 288-303, 289-304, 290-305, 291-306, 292-307, 293-308, 294-309, 295-310, 296-311, 297-312, 298-313, 299-314, 300-315, 301-316, 302-317, 303-318, 304-319, 305-320, 306-321, 307-322, 308-323, 309-324, 310-325, 311-326, 312-327, 313-328, 314-329, 315-330, 316-331, 317-332, 318-333, 319-334, 320-335, 321-336, 322-337, 323-338, 324-339, 325-340, 326-341, 327-342, 328-343, 329-344, 330-345, 331-346, 332-347, 333-348, 334-349, 335-350, 336-351, 337-352, 338-353, 339-354, 340-355, 341-356, 342-357, 343-358, 344-359, 345-360, 346-361, 347-362, 348-363, 349-364, 350-365, 351-366, 352-367, 353-368, 354-369, 355-370, 356-371, 357-372, 358-373, 359-374, 360-375, 361-376, 362-377, 363-378, 364-379, 365-380, 366-381, 367-382, 368-383, 369-384, 370-385, 371-386, 372-387, 373-388, 374-389, 375-390, 376-391, 377-392, 378-393, 379-394, 380-395, 381-396, 382-397, 383-398, 384-399, 385-400, 386-401, 387-402, 388-403, 389-404, 390-405, 391-406, 392-407, 393-408, 394-409, 395-410, 396-411, 397-412, 398-413, 399-414, 400-415, 401-416, 402-417, 403-418, 404-419, 405-420, 406-421, 407-422, 408-423, 409-424, 410-425, 411-426, 412-427, 413-428, 414-429, 415-430, 416-431, 417-432, 418-433, 419-434, 420-435, 421-436, 422-437, 423-438, 424-439, 425-440, 426-441, 427-442, 428-443, 429-444, 430-445, 431-446, 432-447, 433-448, 434-449, 435-450, 436-451, 437-452, 438-453, 439-454, 440-455, 441-456, 442-457, 443-458, 444-459, 445-460, 446-461, 447-462, 448-463, 449-464, 450-465, 451-466, 452-467, 453-468, 454-469, 455-470, 456-471, 457-472, 458-473, 459-474, 460-475, 461-476, 462-477, 463-478, 464-479, 465-480, 466-481, 467-482, 468-483, 469-484, 470-485, 471-486, 472-487, 473-488, 474-489, 475-490, 476-491, 477-492, 478-493, 479-494, 480-495, 481-496, 482-497, 483-498, 484-499, 485-500, 486-501, 487-502, 488-503, 489-504, 490-505, 491-506, 492-507, 493-508, 494-509, 495-510, 496-511, 497-512, 498-513, 499-514, 500-515, 501-516, 502-517, 503-518, 504-519, 505-520, 506-521, 507-522, 508-523, 509-524, 510-525, 511-526, 512-527, 513-528, 514-529, 515-530, 516-531, 517-532, 518-533, 519-534, 520-535, 521-536, 522-537, 523-538, 524-539, 525-540, 526-541, 527-542, 528-543, 529-544, 530-545, 531-546, 532-547, 533-548, 534-549, 535-550, 536-551, 537-552, 538-553, 539-554, 540-555, 541-556, 542-557, 543-558, 544-559, 545-560, 546-561, 547-562, 548-563, 549-564, 550-565, 551-566, 552-567, 553-568, 554-569, 555-570, 556-571, 557-572, 558-573, 559-574, 560-575, 561-576, 562-577, 563-578, 564-579, 565-580, 566-581, 567-582, 568-583, 569-584, 570-585, 571-586, 572-587, 573-588, 574-589, 575-590, 576-591, 577-592, 578-593, 579-594, 580-595, 581-596, 582-597, 583-598, 584-599, 585-600, 586-601, 587-602, 588-603, 589-604, 590-605, 591-606, 592-607, 593-608, 594-609, 595-610, 596-611, 597-612, 598-613, 599-614, 600-615, 601-616, 602-617, 603-618, 604-619, 605-620, 606-621, 607-622, 608-623, 609-624, 610-625, 611-626, 612-627, 613-628, 614-629, 615-630, 616-631, 617-632, 618-633, 619-634, 620-635, 621-636, 622-637, 623-638, 624-639, 625-640, 626-641, 627-642, 628-643, 629-644, 630-645, 631-646, 632-647, 633-648, 634-649, 635-650, 636-651, 637-652, 638-653, 639-654, 640-655, 641-656, 642-657, 643-658, 644-659, 645-660, 646-661, 647-662, 648-663, 649-664, 650-665, 651-666, 652-667, 653-668, 654-669, 655-670, 656-671, 657-672, 658-673, 659-674, 660-675, 661-676, 662-677, 663-678, 664-679, 665-680, 666-681, 667-682, 668-683, 669-684, 670-685, 671-686, 672-687, 673-688, 674-689, 675-690 of SEQ ID NO: 58.

In some aspects, the target region corresponds to a 16-mer nucleotide between positions 208 and 690 of SEQ ID NO: 58. In some aspects, the target region corresponds to a 17-mer nucleotide between positions 208 and 690 of SEQ ID NO: 58. In some aspects, the target region corresponds to a 18-mer nucleotide sequence between positions 208 and 690 of SEQ ID NO: 58. In some aspects, the target region corresponds to a 19-mer nucleotide sequence between positions 208 and 690 of SEQ ID NO: 58. In some aspects, the target region corresponds to a 20-mer nucleotide sequence between positions 208 and 690 of SEQ ID NO: 58. In some aspects, the target region corresponds to a 16-mer, 17-mer, 18-mer, 19-mer or 20-mer target region disclosed above ±10, ±20, ±30, ±40, ±50, ±60, ±70, ±80, or ±90 nucleotides at the 3′ end and/or the 5′ end.

In some aspects, the ASO is not ATCTTCAATCAACAGC (SEQ ID NO: 61).

In some aspects, the ASO is ATCTTCAATCAACAGC (SEQ ID NO: 61).

In some aspects, the ASO of the present disclosure hybridizes to multiple target regions within the PMP22 transcript (e.g., genomic sequence, SEQ ID NO: 58). In some aspects, the ASO hybridizes to two different target regions within the PMP22 transcript. In some aspects, the ASO hybridizes to three different target regions within the PMP22 transcript. In some aspects, the ASOs that hybridizes to multiple regions within the PMP22 transcript (e.g., genomic sequence, SEQ ID NO: 58) are more potent (e.g., having lower EC50) at reducing PMP22 expression compared to ASOs that hybridizes to a single region within the PMP22 transcript (e.g., genomic sequence, SEQ ID NO: 58).

In some aspects, the ASO is capable of down-regulating (e.g., reducing or removing) expression of the PMP22 mRNA or protein both in humans and in rodents (e.g., mice or rats). In some aspects, any ASO described herein is part of a conjugate, comprising the ASO covalently linked to at least one non-nucleotide or non-polynucleotide.

The ASOs of the disclosure comprise a contiguous nucleotide sequence which corresponds to the complement of a region of PMP22 transcript, e.g., a nucleotide sequence corresponding to SEQ ID NO: 58.

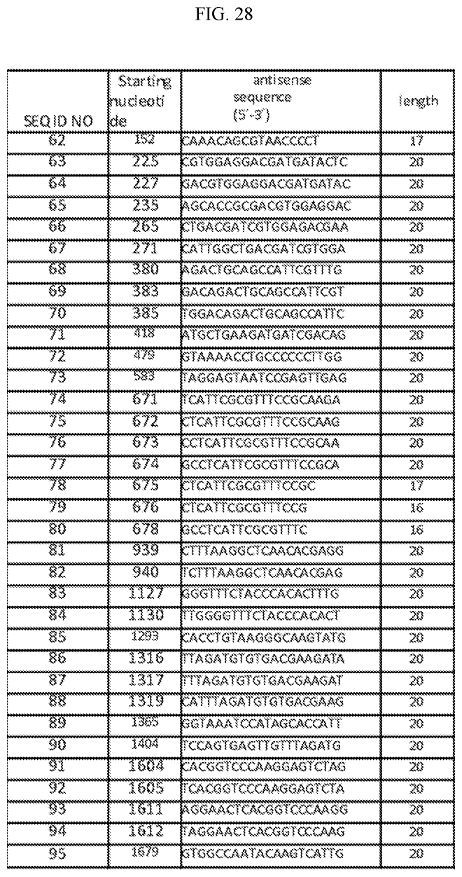

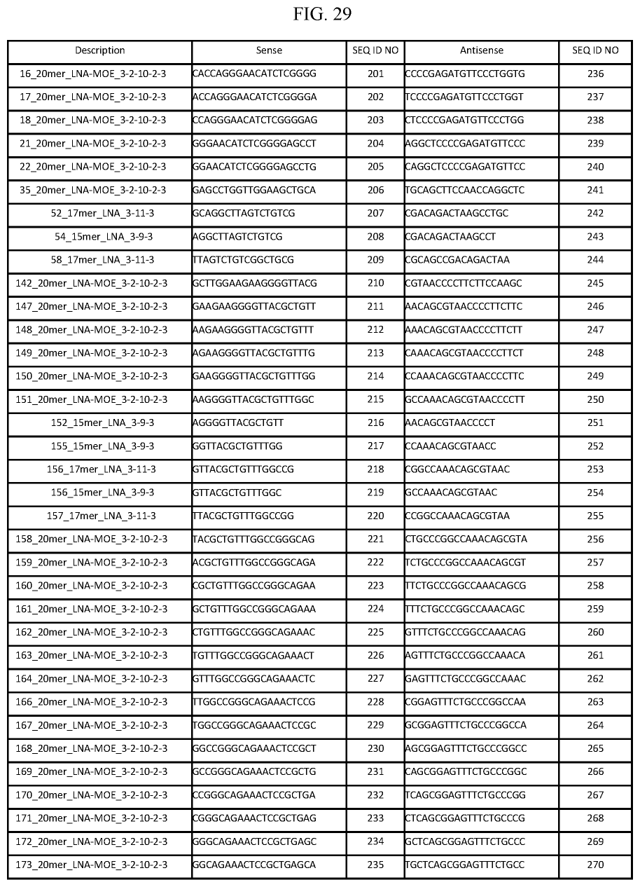

In some aspects, the nucleotide sequence of the ASOs of the disclosure or the contiguous nucleotide sequence has at least about 80% sequence identity to a sequence selected from SEQ ID NOs: 62-95 or 201-270 (i.e., the antisense sequences in FIGS. 28 and 29), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some aspects, the ASO has a design described elsewhere herein or a chemical structure shown elsewhere herein (i.e., the antisense sequences in FIGS. 28 and 29).

In some aspects the ASO (or contiguous nucleotide portion thereof) is selected from, or comprises, one of the sequences selected from the group consisting of SEQ ID NOs: 62-95 or 201-270 to PMP22 or a region of at least 10 contiguous nucleotides thereof, wherein the ASO (or contiguous nucleotide portion thereof) can optionally comprise one, two, three, or four mismatches when compared to the corresponding PMP22 transcript. Non-limiting exemplary ASOs targeting PMP22 gene are shown in FIGS. 28 and 29.

In some aspects, the ASO comprises the sequence as set forth in SEQ ID NO: 65. In some aspects, the ASO comprises the sequence as set forth in SEQ ID NO: 66. In some aspects, the ASO comprises the sequence as set forth in SEQ ID NO: 67. In some aspects, the ASO comprises the sequence as set forth in SEQ ID NO: 68. In some aspects, the ASO comprises the sequence as set forth in SEQ ID NO: 69. In some aspects, the ASO comprises the sequence as set forth in SEQ ID NO: 70. In some aspects, the ASO comprises the sequence as set forth in SEQ ID NO: 71. In some aspects, the ASO comprises the sequence as set forth in SEQ ID NO: 72. In some aspects, the ASO comprises the sequence as set forth in SEQ ID NO: 73. In some aspects, the ASO comprises the sequence as set forth in SEQ ID NO: 74. In some aspects, the ASO comprises the sequence as set forth in SEQ ID NO: 75. In some aspects, the ASO comprises the sequence as set forth in SEQ ID NO: 76. In some aspects, the ASO comprises the sequence as set forth in SEQ ID NO: 77. In some aspects, the ASO comprises the sequence as set forth in SEQ ID NO: 78. In some aspects, the ASO comprises the sequence as set forth in SEQ ID NO: 79. In some aspects, the ASO comprises the sequence as set forth in SEQ ID NO: 80. In some aspects, the ASO comprises the sequence as set forth in SEQ ID NO: 81. In some aspects, the ASO comprises the sequence as set forth in SEQ ID NO: 82. In some aspects, the ASO comprises the sequence as set forth in SEQ ID NO: 83. In some aspects, the ASO comprises the sequence as set forth in SEQ ID NO: 84. In some aspects, the ASO comprises the sequence as set forth in SEQ ID NO: 85. In some aspects, the ASO comprises the sequence as set forth in SEQ ID NO: 86. In some aspects, the ASO comprises the sequence as set forth in SEQ ID NO: 87. In some aspects, the ASO comprises the sequence as set forth in SEQ ID NO: 88. In some aspects, the ASO comprises the sequence as set forth in SEQ ID NO: 89. In some aspects, the ASO comprises the sequence as set forth in SEQ ID NO: 90. In some aspects, the ASO comprises the sequence as set forth in SEQ ID NO: 91. In some aspects, the ASO comprises the sequence as set forth in SEQ ID NO: 92. In some aspects, the ASO comprises the sequence as set forth in SEQ ID NO: 93. In some aspects, the ASO comprises the sequence as set forth in SEQ ID NO: 94. In some aspects, the ASO comprises the sequence as set forth in SEQ ID NO: 95. In some aspects, the ASO comprises the sequence as set forth in SEQ ID NO: 201. In some aspects, the ASO comprises the sequence as set forth in SEQ ID NO: 202. In some aspects, the ASO comprises the sequence as set forth in SEQ ID NO: 203. In some aspects, the ASO comprises the sequence as set forth in SEQ ID NO: 204. In some aspects, the ASO comprises the sequence as set forth in SEQ ID NO: 205. In some aspects, the ASO comprises the sequence as set forth in SEQ ID NO: 206. In some aspects, the ASO comprises the sequence as set forth in SEQ ID NO: 207. In some aspects, the ASO comprises the sequence as set forth in SEQ ID NO: 208. In some aspects, the ASO comprises the sequence as set forth in SEQ ID NO: 209. In some aspects, the ASO comprises the sequence as set forth in SEQ ID NO: 210. In some aspects, the ASO comprises the sequence as set forth in SEQ ID NO: 211. In some aspects, the ASO comprises the sequence as set forth in SEQ ID NO: 212. In some aspects, the ASO comprises the sequence as set forth in SEQ ID NO: 213. In some aspects, the ASO comprises the sequence as set forth in SEQ ID NO: 214. In some aspects, the ASO comprises the sequence as set forth in SEQ ID NO: 215. In some aspects, the ASO comprises the sequence as set forth in SEQ ID NO: 216. In some aspects, the ASO comprises the sequence as set forth in SEQ ID NO: 217. In some aspects, the ASO comprises the sequence as set forth in SEQ ID NO: 218. In some aspects, the ASO comprises the sequence as set forth in SEQ ID NO: 219. In some aspects, the ASO comprises the sequence as set forth in SEQ ID NO: 220. In some aspects, the ASO comprises the sequence as set forth in SEQ ID NO: 221. In some aspects, the ASO comprises the sequence as set forth in SEQ ID NO: 222. In some aspects, the ASO comprises the sequence as set forth in SEQ ID NO: 223. In some aspects, the ASO comprises the sequence as set forth in SEQ ID NO: 224. In some aspects, the ASO comprises the sequence as set forth in SEQ ID NO: 225. In some aspects, the ASO comprises the sequence as set forth in SEQ ID NO: 226. In some aspects, the ASO comprises the sequence as set forth in SEQ ID NO: 227. In some aspects, the ASO comprises the sequence as set forth in SEQ ID NO: 228. In some aspects, the ASO comprises the sequence as set forth in SEQ ID NO: 229. In some aspects, the ASO comprises the sequence as set forth in SEQ ID NO: 230. In some aspects, the ASO comprises the sequence as set forth in SEQ ID NO: 231. In some aspects, the ASO comprises the sequence as set forth in SEQ ID NO: 232. In some aspects, the ASO comprises the sequence as set forth in SEQ ID NO: 233. In some aspects, the ASO comprises the sequence as set forth in SEQ ID NO: 234. In some aspects, the ASO comprises the sequence as set forth in SEQ ID NO: 235. In some aspects, the ASO comprises the sequence as set forth in SEQ ID NO: 236. In some aspects, the ASO comprises the sequence as set forth in SEQ ID NO: 237. In some aspects, the ASO comprises the sequence as set forth in SEQ ID NO: 238. In some aspects, the ASO comprises the sequence as set forth in SEQ ID NO: 239. In some aspects, the ASO comprises the sequence as set forth in SEQ ID NO: 240. In some aspects, the ASO comprises the sequence as set forth in SEQ ID NO: 241. In some aspects, the ASO comprises the sequence as set forth in SEQ ID NO: 242. In some aspects, the ASO comprises the sequence as set forth in SEQ ID NO: 243. In some aspects, the ASO comprises the sequence as set forth in SEQ ID NO: 244. In some aspects, the ASO comprises the sequence as set forth in SEQ ID NO: 245. In some aspects, the ASO comprises the sequence as set forth in SEQ ID NO: 246. In some aspects, the ASO comprises the sequence as set forth in SEQ ID NO: 247. In some aspects, the ASO comprises the sequence as set forth in SEQ ID NO: 248. In some aspects, the ASO comprises the sequence as set forth in SEQ ID NO: 249. In some aspects, the ASO comprises the sequence as set forth in SEQ ID NO: 250. In some aspects, the ASO comprises the sequence as set forth in SEQ ID NO: 251. In some aspects, the ASO comprises the sequence as set forth in SEQ ID NO: 252. In some aspects, the ASO comprises the sequence as set forth in SEQ ID NO: 253. In some aspects, the ASO comprises the sequence as set forth in SEQ ID NO: 254. In some aspects, the ASO comprises the sequence as set forth in SEQ ID NO: 255. In some aspects, the ASO comprises the sequence as set forth in SEQ ID NO: 256. In some aspects, the ASO comprises the sequence as set forth in SEQ ID NO: 257. In some aspects, the ASO comprises the sequence as set forth in SEQ ID NO: 258. In some aspects, the ASO comprises the sequence as set forth in SEQ ID NO: 259. In some aspects, the ASO comprises the sequence as set forth in SEQ ID NO: 260. In some aspects, the ASO comprises the sequence as set forth in SEQ ID NO: 261. In some aspects, the ASO comprises the sequence as set forth in SEQ ID NO: 262. In some aspects, the ASO comprises the sequence as set forth in SEQ ID NO: 263. In some aspects, the ASO comprises the sequence as set forth in SEQ ID NO: 264. In some aspects, the ASO comprises the sequence as set forth in SEQ ID NO: 265. In some aspects, the ASO comprises the sequence as set forth in SEQ ID NO: 266. In some aspects, the ASO comprises the sequence as set forth in SEQ ID NO: 267. In some aspects, the ASO comprises the sequence as set forth in SEQ ID NO: 268. In some aspects, the ASO comprises the sequence as set forth in SEQ ID NO: 269. In some aspects, the ASO comprises the sequence as set forth in SEQ ID NO: 270.

In some aspects the ASO comprises or consists of a sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence set forth in SEQ ID NOs: 62-95 and 201-270. In some aspects the ASO (or contiguous nucleotide portion thereof) is selected from, or comprises, one of the sequences selected from the group consisting of SEQ ID NOs: 62-95 and 201-270 or a region of at least 8, 9, 10, 11, 12, 13, 14, 15, 16, 17, 18, 19, or 20 contiguous nucleotides thereof. In some aspects the ASO (or contiguous nucleotide portion thereof) is selected from, or comprises, one of the sequences selected from the group consisting of SEQ ID NOs: 62-95 and 201-270 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four mismatches when compared to the corresponding PMP22 transcript. In some aspects the ASO (or contiguous nucleotide portion thereof) is selected from, or comprises, one of the sequences selected from the group consisting of SEQ ID NOs: 62-95 and 201-270 except for 1, 2, 3, 4, 5, 6, 7, 8, 9 or 10 substitutions, wherein the substituted ASO can bind to the PMP22 transcript. In some aspects the ASO (or contiguous nucleotide portion thereof) is selected from, or comprises, one of the sequences selected from the group consisting of SEQ ID NOs: 62-95 and 201-270 or a region of at least 8, 9, 10, 11, 12, 13, 14, 15, 16, 17, 18, 19, or 20 contiguous nucleotides thereof, wherein the ASO (or contiguous nucleotide portion thereof) can optionally comprise one, two, three, or four additional 5′ and/or 3′ nucleotides complementary to the corresponding PMP22 transcript.

In some aspects, binding of an ASO targeting a PMP22 transcript disclosed herein to a mRNA transcript encoding PMP22 can reduce expression levels and/or activity levels of PMP22.

II.D. EVs, e.g., Exosomes

The EVs (e.g., exosomes) of the present disclosure can have a diameter between about 20 and about 300 nm. In certain aspects, an EV (e.g., exosome) of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV (e.g., exosome) described herein can be measured according to methods known in the art.

EVs (e.g., exosomes) of the present disclosure comprise a bi-lipid membrane (“exosome membrane” or “EV membrane”), comprising an interior surface (luminal surface) and an exterior surface. The interior surface faces the inner core of the EV (e.g., exosome), i.e., the lumen of the EV.

The EV or exosome membrane comprises lipids and fatty acids. Exemplary lipids comprise phospholipids, glycolipids, fatty acids, sphingolipids, phosphoglycerides, sterols, cholesterols, and phosphatidylserines. The EV or exosome membrane comprises an inner leaflet and an outer leaflet. The composition of the inner and outer leaflet can be determined by transbilayer distribution assays known in the art, see, e.g., Kuypers et al., Biohim Biophys Acta 1985 819:170.

In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine. In some aspects, the EV or exosome membrane comprises one or more polysaccharides, such as glycan. Glycans on the surface of the EV or exosomes can serve as an attachment to a maleimide moiety or a linker that connect the glycan and a maleimide moiety. The glycan can be present on one or more proteins on the surface of an EV (e.g., exosome), for example, a Scaffold X, such as a PTGFRN polypeptide, or on the lipid membrane of the EV (e.g., exosome). Glycans can be modified to have thiofucose that can serve as a functional group for attaching a maleimide moiety to the glycans. In some aspects, the Scaffold X can be modified to express a high number of glycan to allow additional attachments on the EV (e.g., exosome).

II.D.1. Scaffold Moieties

In some aspects, the biologically active molecule is attached to the surface or to the lumen of the EV (e.g., exosome) via a maleimide moiety. In some aspects, the biologically active molecule is attached to a scaffold moiety (e.g., Scaffold X) on the external surface or on the luminal surface of the EV (e.g., exosome) via a maleimide moiety.

In certain aspects, the one or more moieties are introduced into the EV (e.g., exosome) by transfection. In some aspects, the one or more moieties can be introduced into the EV (e.g., exosome) using synthetic macromolecules such as cationic lipids and polymers (Papapetrou et al., Gene Therapy 12: S118-S130 (2005)). In certain aspects, chemicals such as calcium phosphate, cyclodextrin, or polybrene, can be used to introduce the one or more moieties to the EV (e.g, exosome).

In some aspects, one or more scaffold moieties can be CD47, CD55, CD49, CD40, CD133, CD59, glypican-1, CD9, CD63, CD81, integrins, selectins, lectins, cadherins, other similar polypeptides known to those of skill in the art, or any combination thereof.

In other aspects, one or more scaffold moieties are expressed in the membrane of the EVs (e.g., exosomes) by recombinantly expressing the scaffold moieties in the producer cells. The EVs (e.g., exosomes) obtained from the producer cells can be further modified to be conjugated to a maleimide moiety or to a linker. In other aspects, the scaffold moiety, Scaffold X and/or Scaffold Y, is deglycosylated. In some aspects, the scaffold moiety, Scaffold X and/or Scaffold Y, is highly glycosylated, e.g., higher than naturally-occurring Scaffold X and/or Scaffold Y under the same condition.

II.D.1.a. Scaffold X

Various modifications or fragments of the scaffold moiety can be used for the aspects of the present disclosure. For example, scaffold moieties modified to have enhanced affinity to a binding agent can be used for generating surface-engineered EVs (e.g., exosomes) that can be purified using the binding agent. Scaffold moieties modified to be more effectively targeted to EVs (e.g., exosomes) and/or membranes can be used. Scaffold moieties modified to comprise a minimal fragment required for specific and effective targeting to EV (e.g., exosome) membranes can be also used. In some aspects, scaffold moieties can be linked to the maleimide moiety as described herein. In other aspects, scaffold moieties are not linked to the maleimide moiety.

Scaffold moieties can be engineered synthetically or recombinantly, e.g., to be expressed as a fusion protein, e.g., fusion protein of Scaffold X to another moiety. For example, the fusion protein can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to another moiety. In case of the fusion protein, the second moiety can be a natural peptide, a recombinant peptide, a synthetic peptide, or any combination thereof. In other aspects, the scaffold moieties can be CD9, CD63, CD81, PDGFR, GPI anchor proteins, lactadherin, LAMP2, or LAMP2B, or any combination thereof. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); or any combination thereof.

In some aspects, the surface (e.g., Scaffold X)-engineered EVs (e.g., exosomes) described herein demonstrate superior characteristics compared to EVs (e.g., exosomes) known in the art. For example, surface (e.g., Scaffold X)-engineered contain modified proteins more highly enriched on their external surface or luminal surface of the EV (e.g., exosome) than naturally occurring EVs (e.g., exosomes) or the EVs (e.g., exosomes) produced using conventional EV (e.g., exosome) proteins. Moreover, the surface (e.g., Scaffold X)-engineered EVs (e.g., exosomes) of the present disclosure can have greater, more specific, or more controlled biological activity compared to naturally occurring EVs (e.g., exosomes) or the EVs (e.g., exosomes) produced using conventional EV (e.g., exosome) proteins.

In some aspects, the Scaffold X comprises Prostaglandin F2 receptor negative regulator (the PTGFRN polypeptide). The PTGFRN polypeptide can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full-length amino acid sequence of the human PTGFRN polypeptide (Uniprot Accession No. Q9P2B2) is shown below.

PTGFRN polypeptide
(SEQ ID NO: 301)
MGRLASRPLLLALLSLALCRGRVVRVPTATLVRVV
GTELVIPCNVSDYDGPSEQNFDWSFSSLGSSFVEL
ASTWEVGFPAQLYQERLQRGEILLRRTANDAVELH
IKNVQPSDQGHYKCSTPSTDATVQGNYEDTVQVKV
LADSLHVGPSARPPPSLSLREGEPFELRCTAASAS
PLHTHLALLWEVHRGPARRSVLALTHEGRFHPGLG
YEQRYHSGDVRLDTVGSDAYRLSVSRALSADQGSY
RCIVSEWIAEQGNWQEIQEKAVEVATVVIQPSVLR
AAVPKNVSVAEGKELDLTCNITTDRADDVRPEVTW
SFSRMPDSTLPGSRVLARLDRDSLVHSSPHVALSH
VDARSYHLLVRDVSKENSGYYYCHVSLWAPGHNRS
WHKVAEAVSSPAGVGVTWLEPDYQVYLNASKVPGF
ADDPTELACRVVDTKSGEANVRFTVSWYYRMNRRS
DNVVTSELLAVMDGDWTLKYGERSKQRAQDGDFIF
SKEHTDTFNFRIQRTTEEDRGNYYCVVSAWTKQRN
NSWVKSKDVFSKPVNIFWALEDSVLVVKARQPKPF
FAAGNTFEMTCKVSSKNIKSPRYSVLIMAEKPVGD
LSSPNETKYIISLDQDSVVKLENWTDASRVDGVVL
EKVQEDEFRYRMYQTQVSD
AGLYRCMVTAWSPVRGSLWREAATSLSNPIEIDFQ
TSGPIFNASVHSDTPSVIRGDLIKLFCIITVEGAA
LDPDDMAFDVSWFAVHSFGLDKAPVLLSSLDRKGI
VTTSRRDWKSDLSLERVSVLEFLLQVHGSEDQDFG
NYYCSVTPWVKSPTGSWQKEAEIHSKPVFITVKMD
VLNAFKYPLLIGVGLSTVIGLLSCLIGYCSSHWCC
KKEVQETRRERRRLMSMEMD

The PTGFRN polypeptide contains a signal peptide (amino acids 1 to 25 of SEQ ID NO: 301), the extracellular domain (amino acids 26 to 832 of SEQ ID NO: 301), a transmembrane domain (amino acids 833 to 853 of SEQ ID NO: 301), and a cytoplasmic domain (amino acids 854 to 879 of SEQ ID NO: 301). The mature PTGFRN polypeptide consists of SEQ ID NO: 301 without the signal peptide, i.e., amino acids 26 to 879 of SEQ ID NO: 301. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 301. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 302, a fragment of the PTGFRN polypeptide. In other aspects, the Scaffold X comprises the amino acid sequence of SEQ ID NO: 302, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 302 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 302.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 301, amino acids 833 to 853 of SEQ ID NO: 301, SEQ ID NO: 302, or SEQ ID NO: 301. In other aspects, the Scaffold X comprises the amino acid sequence of amino acids 26 to 879 of SEQ ID NO: 301, amino acids 833 to 853 of SEQ ID NO: 301, SEQ ID NO: 302, or SEQ ID NO: 301, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of amino acids 26 to 879 of SEQ ID NO: 301, amino acids 833 to 853 of SEQ ID NO: 301, SEQ ID NO: 302, or SEQ ID NO: 301, and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of amino acids 26 to 879 of SEQ ID NO: 1, amino acids 833 to 853 of SEQ ID NO: 301, SEQ ID NO: 302, or SEQ ID NO: 301.

In some aspects, the Scaffold X comprises, consists, or consists essentially of the amino sequence set forth in SEQ ID NOS: 301 (PTGFRN protein), 302 (amino acids 687-878 of full length PTGFRN), 303 (BSG protein), 304 (IGSF8 protein), 305 (ITGB1 protein), 306 (ITGA4 protein), 307 (SLC3A2 protein), or a functional fragment thereof.

In other aspects, the Scaffold X comprises the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein, wherein the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the corresponding mature BSG protein, IGSF8 protein, IGSF3 protein, ITGB1 protein, SLC3A2 protein, ITGA4 protein, ATP1A1 protein, ATP1A2 protein, ATP1A3 protein, ATP1A4 protein, ATP1A5 protein, ATP2B1 protein, ATP2B2 protein, ATP2B3 protein, ATP2B4 protein, or IGSF2 protein (without the signal peptide). In some aspects, the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein lacks one or more functional or structural domains, such as IgV.

Non-limiting examples of other Scaffold X proteins can be found at U.S. Pat. No. 10,195,290B1, issued Feb. 5, 2019, which is incorporated by reference in its entirety, the ATP transporter proteins: ATP1A1, ATP1A2, ATP1A3, ATP1A4, ATP1B3, ATP2B1, ATP2B2, and ATP2B4), CD9, CD63, CD81, PDGFR, GPI anchor proteins, lactadherin, LAMP2, and LAMP2B.

In some aspects, a Scaffold X comprises Basigin (the BSG protein; SEQ ID NO: 303). The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21. Amino acids 138-323 are the extracellular domain, amino acids 324 to 344 are the transmembrane domain, and amino acids 345 to 385 are the cytoplasmic domain.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of human BSG protein. In some aspects, the fragments of Basigin polypeptide lack one or more functional or structural domains, such as IgV, e.g., amino acids 221 to 315 of human BSG protein.

In some aspects, a Scaffold X comprises Immunoglobulin superfamily member 8 (IgSF8 or the IGSF8 protein; SEQ ID NO: 304), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot. The human IGSF8 protein has a signal peptide (amino acids 1 to 27 of human IGSF8 protein), an extracellular domain (amino acids 28 to 579 of human IGSF8 protein), a transmembrane domain (amino acids 580 to 600 of human IGSF8 protein), and a cytoplasmic domain (amino acids 601 to 613 of human IGSF8 protein).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of human IGSF8 protein. In some aspects, the IGSF8 protein lack one or more functional or structural domains, such as IgV. In other aspects, the Scaffold X comprises the amino acid sequence of human IGSF8 protein, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of human IGSF8 protein and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of human IGSF8 protein.

In some aspects, a Scaffold X for the present disclosure comprises Immunoglobulin superfamily member 3 (IgSF3 or the IGSF3 protein; SEQ ID NO: 309), which is also known as Glu-Trp-Ile EWI motif-containing protein 3 (EWI-3). The human IGSF3 protein has a signal peptide (amino acids 1 to 19 of the IGSF3 protein), an extracellular domain (amino acids 20 to 1124 of the IGSF3 protein), a transmembrane domain (amino acids 1125 to 1145 of the IGSF3 protein), and a cytoplasmic domain (amino acids 1146 to 1194 of the IGSF3 protein).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of the IGSF3 protein. In some aspects, the IGSF3 protein lack one or more functional or structural domains, such as IgV.

In some aspects, a Scaffold X for the present disclosure comprises Integrin beta-1 (the ITGB1 protein; SEQ ID NO: 305), which is also known as Fibronectin receptor subunit beta, Glycoprotein IIa (GPIIA), VLA-4 subunit beta, or CD29. The human ITGB1 protein has a signal peptide (amino acids 1 to 20 of the human ITGB1 protein), an extracellular domain (amino acids 21 to 728 of the human ITGB1 protein), a transmembrane domain (amino acids 729 to 751 of the human ITGB1 protein), and a cytoplasmic domain (amino acids 752 to 798 of the human ITGB1 protein).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of the human ITGB1 protein. In some aspects, the ITGB1 protein lack one or more functional or structural domains, such as IgV.

In other aspects, the Scaffold X comprises the ITGA4 protein (SEQ ID NO: 306), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the human ITGB1 protein without the signal peptide (amino acids 1 to 33 of the human ITGB1 protein). In some aspects, the ITGA4 protein lacks one or more functional or structural domains, such as IgV.

In other aspects, the Scaffold X comprises the SLC3A2 protein (SEQ ID NO: 307), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the SLC3A2 protein without the signal peptide. In some aspects, the SLC3A2 protein lacks one or more functional or structural domains, such as IgV.

In other aspects, the Scaffold X comprises the ATP1A1 protein (SEQ ID NO: 310), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A1 protein without the signal peptide. In some aspects, the ATP1A1 protein lacks one or more functional or structural domains, such as IgV.

In other aspects, the Scaffold X comprises the ATP1A2 protein (SEQ ID NO: 311), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A2 protein without the signal peptide. In some aspects, the ATP1A2 protein lacks one or more functional or structural domains, such as IgV.

In other aspects, the Scaffold X comprises the ATP1A3 protein (SEQ ID NO: 312), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A3 protein without the signal peptide. In some aspects, the ATP1A3 protein lacks one or more functional or structural domains, such as IgV.

In other aspects, the Scaffold X comprises the ATP1A4 protein (SEQ ID NO: 313), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A4 protein without the signal peptide. In some aspects, the ATP1A4 protein lacks one or more functional or structural domains, such as IgV.

In other aspects, the Scaffold X comprises the ATP1B3 protein (SEQ ID NO: 314), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP1A5 protein without the signal peptide. In some aspects, the ATP1A5 protein lacks one or more functional or structural domains, such as IgV.

In other aspects, the Scaffold X comprises the ATP2B1 protein (SEQ ID NO: 315), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B1 protein without the signal peptide. In some aspects, the ATP2B1 protein lacks one or more functional or structural domains, such as IgV.

In other aspects, the Scaffold X comprises the ATP2B2 protein (SEQ ID NO: 316), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B2 protein without the signal peptide. In some aspects, the ATP2B2 protein lacks one or more functional or structural domains, such as IgV.

In other aspects, the Scaffold X comprises the ATP2B3 protein (SEQ ID NO: 317), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B3 protein without the signal peptide. In some aspects, the ATP2B3 protein lacks one or more functional or structural domains, such as IgV.

In other aspects, the Scaffold X comprises the ATP2B4 protein (SEQ ID NO: 318), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the ATP2B4 protein without the signal peptide. In some aspects, the ATP2B4 protein lacks one or more functional or structural domains, such as IgV.

In other aspects, the Scaffold X comprises the IGSF2 protein (SEQ ID NO: 308), which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the IGSF2 protein without the signal peptide. In some aspects, the IGSF2 protein lacks one or more functional or structural domains, such as IgV.

Non-limiting examples of other Scaffold X proteins can be found at U.S. Pat. No. 10,195,290B1, issued Feb. 5, 2019, which is incorporated by reference in its entirety.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety to the luminal surface and the external surface of the EV (e.g., exosome) at the same time. For example, the PTGFRN polypeptide can be used to link one or more biologically active molecules indirectly through a maleimide moiety or directly to a maleimide moiety or a linker to the luminal surface in addition to the external surface of the EV (e.g., exosome). Therefore, in certain aspects, Scaffold X can be used for dual purposes.

In other aspects, the EVs (e.g., exosomes) of the present disclosure comprises a higher number of Scaffold X proteins compared to the naturally-occurring EVs (e.g., exosomes). In some aspects, the EVs (e.g., exosomes) of the disclosure comprise at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold higher number of Scaffold X (e.g., a PTGFRN polypeptide) compared to the naturally-occurring EV (e.g., exosome). The number of Scaffold X, e.g., PTGFRN polypeptide, on the EV (e.g., exosome) of the present disclosure is at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 4000, at least about 5000, at least about 6000, at least about 7000, at least about 8000, at least about 9000, or at least about 10000. In some aspects, the number of Scaffold X, e.g., PTGFRN polypeptide, on the EV (e.g., exosome) of the present disclosure is from about 100 to about 100,000, from about 200 to about 9000, from about 300 to about 9000, from about 400 to about 9000, from about 500 to about 9000, from about 600 to about 8000, from about 800 to about 8000, from about 900 to about 8000, from about 1000 to about 8000, from about 1100 to about 8000, from about 1200 to about 8000, from about 1300 to about 8000, from about 1400 to about 8000, from about 1500 to about 8000, from about 1600 to about 8000, from about 1700 to about 8000, from about 1800 to about 8000, from about 1900 to about 8000, from about 2000 to about 8000, from about 2100 to about 8000, from about 2200 to about 8000, from about 2300 to about 8000, from about 2400 to about 8000, from about 2500 to about 8000, from about 2600, from about 2700 to about 8000, from about 2800 to about 8000, from about 2900 to about 8000, from about 3000 to about 8000, from about 4000 to about 8000, from about 5000 to about 8000, from about 6000 to about 8000, from about 7000 to about 8000, from about 8000, from 7000 to about 9000, or from about 6000 to about 10000. In some aspects, the number of Scaffold X, e.g., PTGFRN polypeptide, on the EV (e.g., exosome) of the present disclosure is from about 5000 to about 8000, e.g., about 5000, about 6000, about 7000, or about 8000. In some aspects, the number of Scaffold X, e.g., PTGFRN polypeptide, on the EV (e.g., exosome) of the present disclosure is from about 6000 to about 8000, e.g., about 6000, about 7000, or about 8000. In some aspects, the number of Scaffold X, e.g., PTGFRN polypeptide, on the EV (e.g., exosome) of the present disclosure is from about 4000 to about 9000, e.g., about 4000, about 5000, about 6000, about 7000, about 8000, about 9000.

In some aspects, the Scaffold X is or comprises a PTGFRN protein fragment of SEQ ID NO: 319, 320, 321, 322, 323, or 324; a BSG protein fragment of SEQ ID NO: 326, 327, or 328; or a IGSF8 protein fragment of SEQ ID NO: 330, 331, 332, or 333. In some aspects, the Scaffold X is or comprises a PTGFRN protein without its signal peptide, i.e., a PTGFRN protein or a fragment thereof without its 21 N-terminal amino acids (MGRLASRPLLLALLSLALCRG; SEQ ID NO: 325). In some aspects, the Scaffold X is or comprises a BSG protein without its signal peptide, i.e., a BSG protein or a fragment thereof without its 18 N-terminal amino acids (MAAALFVLLGFALLGTHG; SEQ ID NO: 329). In some aspects, the Scaffold X is or comprises an IGSF8 protein without its signal peptide, i.e., a IGSF8 protein or a fragment thereof without its 27 N-terminal amino acids (MGALRPTLLPPSLPLLLLLMLGMGCWA; SEQ ID NO: 334).

Scaffold X proteins and fragments thereof are disclosed in the sequence listing.

II.D.1.b. Scaffold Y

In some aspects, EVs (e.g., exosomes) of the present disclosure comprise an internal space (i.e., lumen) that is different from that of the naturally occurring EVs (e.g., exosomes). For example, the EV (e.g., exosome) can be changed such that the composition on the luminal surface of the EV (e.g., exosome) has the protein, lipid, or glycan content different from that of the naturally-occurring EVs (e.g., exosomes).

In some aspects, engineered EVs (e.g., exosomes) can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the exosome. Various modifications or fragments of the EV (e.g., exosome) protein that can be expressed on the luminal surface of the EV (e.g, exosome) can be used for the aspects of the present disclosure.

In some aspects, the EV (e.g, exosome) proteins that can change the luminal surface of the EV (e.g, exosome) include, but are not limited to the MARCKS protein, MARCKSL1 protein, BASP1 protein, or any combination thereof. In some aspects, the Scaffold Y comprises Brain Acid Soluble Protein 1 (the BASP1 protein). The BASP1 protein is also known as 22 kDa neuronal tissue-enriched acidic protein or neuronal axonal membrane protein NAP-22. The full-length human BASP1 protein sequence (isomer 1) is shown below. An isomer produced by an alternative splicing is missing amino acids 88 to 141 from the BASP1 protein of SEQ ID NO: 403.

The BASP1 protein
(SEQ ID NO: 403)
MGGKLSKKKKGYNVNDEKAKEKDKKAEGAATEEEG
TPKESEPQAAAEPAEAKEGKEKPDQDAEGKAEEKE
GEKDAAAAKEEAPKAEPEKTEGAAEAKAEPPKAPE
QEQAAPGPAAGGEAPKAAEAAAAPAESAAPAAGEE
PSKEEGEPKKTEAPAAPAAQETKSDGAPASDSKPG
SSEAAPSSKETPAATEAPSSTPKAQGPAASAEEPK
PVEAPAANSDQTVTVKE

The mature BASP1 protein sequence is missing the first Met from SEQ ID NO: 403 and thus contains amino acids 2 to 227 of SEQ ID NO: 403.

In some aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 403. In some aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 403. In other aspects, a Scaffold Y useful for the present disclosure comprises the amino acid sequence of SEQ ID NO: 403, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of SEQ ID NO: 403 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 403.

In certain aspects, the protein sequence of any of SEQ ID NOs: 1-109 disclosed in PCT/US2018/061679 is sufficient to be a Scaffold Y for the present disclosure (e.g., scaffold moiety linked to a linker).

In certain aspects, a Scaffold Y useful for the present disclosure comprises a peptide with the MGXKLSKKK or GXKLSKKK, where X is alanine or any other amino acid (SEQ ID NO: 404). In some aspects, an EV (e.g., exosome) comprises a peptide with sequence of (M)(Gπ)(ξ)(Φ/π)(S/A/G/N)(+)(+) or (G)(π)(ξ)(Φ/π)(S/A/G/N)(+)(+), wherein each parenthetical position represents an amino acid, and wherein a is any amino acid selected from the group consisting of (Pro, Gly, Ala, Ser), 4 is any amino acid selected from the group consisting of (Asn, Gln, Ser, Thr, Asp, Glu, Lys, His, Arg), 4 is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In further aspects, an EV (e.g., exosome) described herein (e.g., engineered exosome) comprises a peptide with sequence of (M)(G)(π)(X)(Φ/π)(π)(+)(+) or (G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). See Aasland et al., FEBS Letters 513 (2002) 141-144 for amino acid nomenclature.

In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of the sequences disclosed in U.S. Pat. No. 10,195,290B1, issued Feb. 5, 2019.

Scaffold Y-engineered exosomes described herein can be produced from a cell transformed with any sequence set forth in PCT/US2018/061679 (SEQ ID NO: 4-109).

In other aspects, the EVs (e.g., exosomes) of the present disclosure comprises a higher number of Scaffold Y proteins compared to the naturally-occurring EVs (e.g., exosomes). In some aspects, the EVs (e.g., exosomes) of the disclosure comprise at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold higher number of Scaffold Y (e.g., a BASP-1 polypeptide) compared to the naturally-occurring EV (e.g., exosome). The number of Scaffold Y, e.g., BASP-1 polypeptide, on the EV (e.g., exosome) of the present disclosure is at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 4000, at least about 5000, at least about 6000, at least about 7000, at least about 8000, at least about 9000, or at least about 10000. In some aspects, the number of Scaffold Y, e.g., a BASP-1 polypeptide, on the EV (e.g., exosome) of the present disclosure is from about 100 to about 100,000, from about 200 to about 9000, from about 300 to about 9000, from about 400 to about 9000, from about 500 to about 9000, from about 600 to about 8000, from about 800 to about 8000, from about 900 to about 8000, from about 1000 to about 8000, from about 1100 to about 8000, from about 1200 to about 8000, from about 1300 to about 8000, from about 1400 to about 8000, from about 1500 to about 8000, from about 1600 to about 8000, from about 1700 to about 8000, from about 1800 to about 8000, from about 1900 to about 8000, from about 2000 to about 8000, from about 2100 to about 8000, from about 2200 to about 8000, from about 2300 to about 8000, from about 2400 to about 8000, from about 2500 to about 8000, from about 2600, from about 2700 to about 8000, from about 2800 to about 8000, from about 2900 to about 8000, from about 3000 to about 8000, from about 4000 to about 8000, from about 5000 to about 8000, from about 6000 to about 8000, from about 7000 to about 8000, from about 8000, from 7000 to about 9000, or from about 6000 to about 10000. In some aspects, the number of Scaffold Y, e.g., a BASP-1 polypeptide, on the EV (e.g., exosome) of the present disclosure is from about 5000 to about 8000, e.g., about 5000, about 6000, about 7000, or about 8000. In some aspects, the number of Scaffold Y, e.g., a BASP-1 polypeptide, on the EV (e.g., exosome) of the present disclosure is from about 6000 to about 8000, e.g., about 6000, about 7000, or about 8000. In some aspects, the number of Scaffold Y, e.g., a BASP-1 polypeptide, on the EV (e.g., exosome) of the present disclosure is from about 4000 to about 9000, e.g., about 4000, about 5000, about 6000, about 7000, about 8000, about 9000.

In some aspects, the Scaffold Y useful for the present disclosure comprises an “N-terminus domain” (ND) and an “effector domain” (ED), wherein the ND and/or the ED are associated with the luminal surface of the EV, e.g., an exosome. In some aspects, the Scaffold Y useful for the present disclosure comprises an intracellular domain, a transmembrane domain, and an extracellular domain; wherein the intracellular domain comprises an “N-terminus domain” (ND) and an “effector domain” (ED); wherein the ND and/or the ED are associated with the luminal surface of the EV, e.g., an exosome. As used herein the term “associated with” refers to the interaction between a scaffold protein of the present disclosure with the luminal surface of the EV, e.g., and exosome, that does not involve covalent linking to a membrane component. For example, the scaffolds useful for the present disclosure can be associated with the luminal surface of the EV, e.g., via a lipid anchor (e.g., myristic acid), and/or a polybasic domain that interacts electrostatically with the negatively charged head of membrane phospholipids. In other aspects, the Scaffold Y comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the Scaffold Y comprises an N-terminus domain (ND) and an effector domain (ED), wherein the ND is associated with the luminal surface of the EV, and the ED is associated with the luminal surface of the EV by an ionic interaction, wherein the ED comprises at least two, at least three, at least four, at least five, at least six, or at least seven contiguous lysines (Lys) in sequence.

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)n, wherein n is an integer between 1 and 10.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids.

In some aspects, the ED comprises K, KK, KKK, KKKK (SEQ ID NO: 405), KKKKK (SEQ ID NO: 406), R, RR, RRR, RRRR (SEQ ID NO: 407); RRRRR (SEQ ID NO: 408), KR, RK, KKR, KRK, RKK, KRR, RRK, (K/R)(K/R)(K/R)(K/R) (SEQ ID NO: 409), (K/R)(K/R)(K/R)(K/R)(K/R) (SEQ ID NO: 410), or any combination thereof. In some aspects, the ED comprises KK, KKK, KKKK (SEQ ID NO: 405), KKKKK (SEQ ID NO: 406), or any combination thereof. In some aspects, the ND comprises the amino acid sequence as set forth in G:X2:X3:X4:X5:X6, wherein G represents Gly; wherein “:” represents a peptide bond; wherein each of the X2 to the X6 independently represents an amino acid; and wherein the X6 represents a basic amino acid. In some aspects, the X6 amino acid is selected is selected from the group consisting of Lys, Arg, and His. In some aspects, the X5 amino acid is selected from the group consisting of Pro, Gly, Ala, and Ser. In some aspects, the X2 amino acid is selected from the group consisting of Pro, Gly, Ala, and Ser. In some aspects, the X4 is selected from the group consisting of Pro, Gly, Ala, Ser, Val, Ile, Leu, Phe, Trp, Tyr, Gln, and Met.

In some aspects, the Scaffold Y comprises an N-terminus domain (ND) and an effector domain (ED), wherein the ND comprises the amino acid sequence as set forth in G:X2:X3:X4:X5:X6, wherein G represents Gly; wherein “:” represents a peptide bond; wherein each of the X2 to the X6 is independently an amino acid; wherein the X6 comprises a basic amino acid, and wherein the ED is linked to X6 by a peptide bond and comprises at least one lysine at the N terminus of the ED.

In some aspects, the ND of the Scaffold Y comprises the amino acid sequence of G:X2:X3:X4:X5:X6, wherein G represents Gly; “:” represents a peptide bond; the X2 represents an amino acid selected from the group consisting of Pro, Gly, Ala, and Ser; the X3 represents any amino acid; the X4 represents an amino acid selected from the group consisting of Pro, Gly, Ala, Ser, Val, Ile, Leu, Phe, Trp, Tyr, Gln, and Met; the X5 represents an amino acid selected from the group consisting of Pro, Gly, Ala, and Ser; and the X6 represents an amino acid selected from the group consisting of Lys, Arg, and His.

In some aspects, the X3 amino acid is selected from the group consisting of Asn, Gln, Ser, Thr, Asp, Glu, Lys, His, and Arg.

In some aspects, the ND and ED are joined by a linker. In some aspects, the linker comprises one or more amino acids. In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence. When the ND and ED are joined by a linker, the ED comprise at least two lysines, at least three lysines, at least four lysines, at least five lysines, at least six lysines, or at least seven lysines.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the linker is a glycine/serine linker. In some aspects, the peptide linker is glycine/serine linker according to the formula [(Gly)n-Ser]m where n is any integer from 1 to 100 and m is any integer from 1 to 100. In other aspects, the glycine/serine linker is according to the formula [(Gly)x-Sery]z wherein x in an integer from 1 to 4, y is 0 or 1, and z is an integers from 1 to 50. In some aspects, the peptide linker comprises the sequence Gn, where n can be an integer from 1 to 100. In some aspects, the peptide linker can comprise the sequence (GlyAla)n, wherein n is an integer between 1 and 100. In other aspects, the peptide linker can comprise the sequence (GlyGlySer)n, wherein n is an integer between 1 and 100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

In some aspects, the Scaffold Y comprises ND-ED, wherein: ND comprises G:X2:X3:X4:X5:X6; wherein: G represents Gly; “:” represents a peptide bond; X2 represents an amino acid selected from the group consisting of Pro, Gly, Ala, and Ser; X3 represents any amino acid; X4 represents an amino acid selected from the group consisting of Pro, Gly, Ala, Ser, Val, Ile, Leu, Phe, Trp, Tyr, Glu, and Met; X5 represents an amino acid selected from the group consisting of Pro, Gly, Ala, and Ser; X6 represents an amino acid selected from the group consisting of Lys, Arg, and His; “-” represents an optional linker; and ED is an effector domain comprising (i) at least two contiguous lysines (Lys), which is linked to the X6 by a peptide bond or one or more amino acids or (ii) at least one lysine, which is directly linked to the X6 by a peptide bond.

In some aspects, the X2 amino acid is selected from the group consisting of Gly and Ala. In some aspects, the X3 amino acid is Lys. In some aspects, the X4 amino acid is Leu or Glu. In some aspects, the X5 amino acid is selected from the group consisting of Ser and Ala. In some aspects, the X6 amino acid is Lys. In some aspects, the X2 amino acid is Gly, Ala, or Ser; the X3 amino acid is Lys or Glu; the X4 amino acid is Leu, Phe, Ser, or Glu; the X5 amino acid is Ser or Ala; and X6 amino acid is Lys. In some aspects, the “-” linker comprises a peptide bond or one or more amino acids.

In some aspects, the ED in the scaffold protein comprises Lys (K), KK, KKK, KKKK (SEQ ID NO: 405), KKKKK (SEQ ID NO: 406), Arg (R), RR, RRR, RRRR (SEQ ID NO: 407); RRRRR (SEQ ID NO: 408), KR, RK, KKR, KRK, RKK, KRR, RRK, (K/R)(K/R)(K/R)(K/R) (SEQ ID NO: 409), (K/R)(K/R)(K/R)(K/R)(K/R) (SEQ ID NO: 410), or any combination thereof.

In some aspects, the Scaffold Y comprises an amino acid sequence selected from the group consisting of (i) GGKLSKK (SEQ ID NO: 411), (ii) GAKLSKK (SEQ ID NO: 412), (iii) GGKQSKK (SEQ ID NO: 413), (iv) GGKLAKK (SEQ ID NO: 414), or (v) any combination thereof.

In some aspects, the ND in the Scaffold Y comprises an amino acid sequence selected from the group consisting of (i) GGKLSK (SEQ ID NO: 415), (ii) GAKLSK (SEQ ID NO: 416), (iii) GGKQSK (SEQ ID NO: 417), (iv) GGKLAK (SEQ ID NO: 418), or (v) any combination thereof and the ED in the scaffold protein comprises (i) K, KK, KKK, KKKG (SEQ ID NO: 419), KKKGY (SEQ ID NO: 420), KKKGYN (SEQ ID NO: 421), KKKGYNV (SEQ ID NO: 422), KKKGYNVN (SEQ ID NO: 423), KKKGYS (SEQ ID NO: 424), KKKGYG (SEQ ID NO: 425), KKKGYGG (SEQ ID NO: 426), KKKGS (SEQ ID NO: 427), KKKGSG (SEQ ID NO: 428), KKKGSGS (SEQ ID NO: 429), KKKS (SEQ ID NO: 430), KKKSG (SEQ ID NO: 431), KKKSGG (SEQ ID NO: 432), KKKSGGS (SEQ ID NO: 433), KKKSGGSG (SEQ ID NO: 434), KKSGGSGG (SEQ ID NO: 435), KKKSGGSGGS (SEQ ID NO: 436), KRFSFKKS (SEQ ID NO: 437).

In some aspects, the polypeptide sequence of a Scaffold Y useful for the present disclosure consists of an amino acid sequence selected from the group consisting of (i) GGKLSKK (SEQ ID NO: 411), (ii) GAKLSKK (SEQ ID NO: 412), (iii) GGKQSKK (SEQ ID NO: 413), (iv) GGKLAKK (SEQ ID NO: 414), or (v) any combination thereof.

In some aspects, the Scaffold Y comprises an amino acid sequence selected from the group consisting of (i) GGKLSKKK (SEQ ID NO: 438), (ii) GGKLSKKS (SEQ ID NO: 439), (iii) GAKLSKKK (SEQ ID NO: 440), (iv) GAKLSKKS (SEQ ID NO: 441), (v) GGKQSKKK (SEQ ID NO: 442), (vi) GGKQSKKS (SEQ ID NO: 443), (vii) GGKLAKKK (SEQ ID NO: 444), (viii) GGKLAKKS (SEQ ID NO: 445), and (ix) any combination thereof.

In some aspects, the polypeptide sequence of a Scaffold Y useful for the present disclosure consists of an amino acid sequence selected from the group consisting of (i) GGKLSKKK (SEQ ID NO: 438), (ii) GGKLSKKS (SEQ ID NO: 439), (iii) GAKLSKKK (SEQ ID NO: 440), (iv) GAKLSKKS (SEQ ID NO: 441), (v) GGKQSKKK (SEQ ID NO: 442), (vi) GGKQSKKS (SEQ ID NO: 443), (vii) GGKLAKKK (SEQ ID NO: 444), (viii) GGKLAKKS (SEQ ID NO: 445), and (ix) any combination thereof.

In some aspects, the Scaffold Y is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least 31, at least about 32, at least about 33, at least about 34, at least about 35, at least about 36, at least about 37, at least about 38, at least about 39, at least about 39, at least about 40, at least about 41, at least about 42, at least about 43, at least about 44, at least about 50, at least about 46, at least about 47, at least about 48, at least about 49, at least about 50, at least about 55, at least about 60, at least about 65, at least about 70, at least about 75, at least about 80, at least 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 335, at least about 340, at least about 345, or at least about 350 amino acids in length.

In some aspects, the Scaffold Y is between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250, between about 250 and about 260, between about 260 and about 270, between about 270 and about 280, between about 280 and about 290, between about 290 and about 300, between about 300 and about 310, between about 310 and about 320, between about 320 and about 330, between about 330 and about 340, or between about 340 and about 250 amino acids in length. In some aspects, the Scaffold Y comprises (i) GGKLSKKKKGYNVN (SEQ ID NO: 446), (ii) GAKLSKKKKGYNVN (SEQ ID NO: 447), (iii) GGKQSKKKKGYNVN (SEQ ID NO: 448), (iv) GGKLAKKKKGYNVN (SEQ ID NO: 449), (v) GGKLSKKKKGYSGG (SEQ ID NO: 450), (vi) GGKLSKKKKGSGGS (SEQ ID NO: 451), (vii) GGKLSKKKKSGGSG (SEQ ID NO: 452), (viii) GGKLSKKKSGGSGG (SEQ ID NO: 453), (ix) GGKLSKKSGGSGGS (SEQ ID NO: 454), (x) GGKLSKSGGSGGSV (SEQ ID NO: 455), or (xi) GAKKSKKRFSFKKS (SEQ ID NO: 456).

In some aspects, the polypeptide sequence of a Scaffold Y useful for the present disclosure consists of (i) GGKLSKKKKGYNVN (SEQ ID NO: 446), (ii) GAKLSKKKKGYNVN (SEQ ID NO: 447), (iii) GGKQSKKKKGYNVN (SEQ ID NO: 448), (iv) GGKLAKKKKGYNVN (SEQ ID NO: 449), (v) GGKLSKKKKGYSGG (SEQ ID NO: 450), (vi) GGKLSKKKKGSGGS (SEQ ID NO: 451), (vii) GGKLSKKKKSGGSG (SEQ ID NO: 452), (viii) GGKLSKKKSGGSGG (SEQ ID NO: 453), (ix) GGKLSKKSGGSGGS (SEQ ID NO: 454), (x) GGKLSKSGGSGGSV (SEQ ID NO: 455), or (xi) GAKKSKKRFSFKKS (SEQ ID NO: 465).

Non-limiting examples of the Scaffold Y useful for the present disclosure are listed below. In some aspects, the Scaffold Y comprises an amino acid sequence set forth in SEQ ID NO: 457 to 567. In some aspects, the Scaffold Y consists of an amino acid sequence set forth in SEQ ID NO: 457 to 567.

In some aspects, the Scaffold Y useful for the present disclosure does not contain an N-terminal Met. In some aspects, the Scaffold Y comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table.

| Modification | Modifying Group |
|---|---|
| S-Palmitoylation | 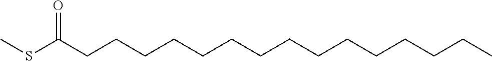 [SMILES: CCCCCCCCCCCCCCCC(=O)SC] |
| N-Palmitoylation | 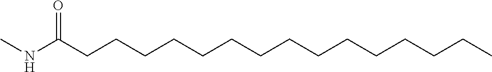 [SMILES: CCCCCCCCCCCCCCCC(=O)NC] |
| N-Myristoylation | 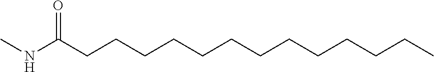 [SMILES: CCCCCCCCCCCCCC(=O)NC] |
| O-Acylation | 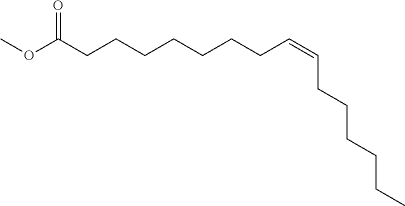 [SMILES: CCCCCC/C=C\CCCCCCCC(=O)OC] |
| Farnesylation | 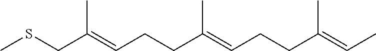 [SMILES: C/C=C(\C)CC/C=C(\C)CC/C=C(\C)CSC] |
| Geranylgeranylation | 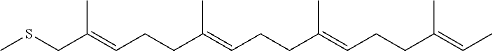 [SMILES: C/C=C(\C)CC/C=C(\C)CC/C=C(\C)CC/C=C(\C)CSC] |
| Cholesterol | 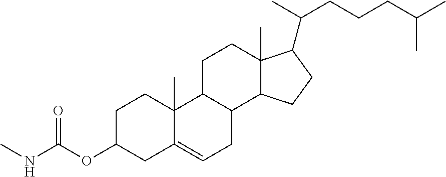 [SMILES: CNC(=O)OC1CCC2(C)C(=CCC3C2CCC2(C)C(C(C)CCCC(C)C)CCC32)C1] |

### III. Methods of Making

EVs (e.g., exosomes) of the present disclosure can be produced by chemical synthesis, recombinant DNA technology, biochemical or enzymatic fragmentation of larger molecules, combinations of the foregoing or by any other method. In one aspect, the present disclosure provides a method of conjugating a biologically active molecule to an EV (e.g., exosome). The method comprises linking a biologically active molecule to an EV (e.g., exosome) via a maleimide moiety as described above.

Besides amine-reactive compounds, those having chemical groups that form bonds with sulfhydryls (—SH) are the most common crosslinkers and modification reagents for protein and other bioconjugate techniques. Sulfhydryls, also called thiols, exist in proteins in the side-chain of cysteine (Cys, C) amino acids. Pairs of cysteine sulfhydryl groups are often linked by disulfide bonds (—S—S—) within or between polypeptide chains as the basis of native tertiary or quaternary protein structure. Typically, only free or reduced sulfhydryl groups (—SH) [rather than sulfur atoms in disulfide bonds] are available for reaction with thiol-reactive compounds.

Sulfhydryl groups are useful targets for protein conjugation and labeling. First, sulfhydryls are present in most proteins but are not as numerous as primary amines; thus, crosslinking via sulfhydryl groups is more selective and precise. Second, sulfhydryl groups in proteins are often involved in disulfide bonds, so crosslinking at these sites typically does not significantly modify the underlying protein structure or block binding sites. Third, the number of available (i.e., free) sulfhydryl groups can be easily controlled or modified; they can be generated by reduction of native disulfide bonds, or they can be introduced into molecules through reaction with primary amines using sulfhydryl-addition reagents, such as 2-iminothiolane (Traut's Reagent), SATA, SATP, or SAT(PEG). Finally, combining sulfhydryl-reactive groups with amine-reactive groups to make heterobifunctional crosslinkers provides greater flexibility and control over crosslinking procedures. For example, using 3-Maleimido-propionic NHS ester, which contains a maleimide group and an NHS ester, the NHS ester can be used to label the primary amines (—NH2) of proteins, amine-modified oligonucleotides, and other amine-containing molecules. The maleimide group will react with a thiol group to form a covalent bond, enabling the connection of biomolecule with a thiol.

The maleimide group reacts specifically with sulfhydryl groups when the pH of the reaction mixture is between 6.5 and 7.5; the result is formation of a stable thioether linkage that is not reversible (i.e., the bond cannot be cleaved with reducing agents). In more alkaline conditions (pH>8.5), the reaction favors primary amines and also increases the rate of hydrolysis of the maleimide group to a non-reactive maleamic acid. Maleimides do not react with tyrosines, histidines or methionines.

Thiol-containing compounds, such as dithiothreitol (DTT) and beta-mercaptoethanol (BME), must be excluded from reaction buffers used with maleimides because they will compete for coupling sites. For example, if DTT were used to reduce disulfides in a protein to make sulfhydryl groups available for conjugation, the DTT would have to be thoroughly removed using a desalting column before initiating the maleimide reaction. Interestingly, the disulfide-reducing agent TCEP does not contain thiols and does not have to be removed before reactions involving maleimide reagents.

Excess maleimides can be quenched at the end of a reaction by adding free thiols. EDTA can be included in the coupling buffer to chelate stray divalent metals that otherwise promote oxidation of sulfhydryls (non-reactive).

In one aspect, the linking comprises treating the EV (e.g., exosome) with a reducing agent. Suitable reducing agents include, for example, TCEP (Tris(2-carboxyethyl)phosphine), DTT (dithiothreitol), BME (2-mercaptoethanol), a thiolating agent, and any combination thereof. The thiolating agent can comprise, e.g., Traut's reagent (2-iminothiolane).

After the treatment with the reducing agent, the linking reaction further comprises bringing the reduced EV (e.g., exosome) in contact with the maleimide moiety. In one aspect, the maleimide moiety is linked to a biologically active molecule prior to the linking to the EV (e.g., exosome). In some aspects, the maleimide moiety is further attached to a linker to connect the maleimide moiety to the biologically active molecule. Accordingly, in some aspects, one or more linkers or spacers are interposed between the maleimide moiety and the biologically active molecule.

In some aspects, EVs disclosed herein (e.g., exosomes) can be produced from a cell grown in vitro or a body fluid of a subject. When exosomes are produced from in vitro cell culture, various producer cells, e.g., HEK293 cells, CHO cells, and MSCs, can be used. In certain aspects, the producer cell is HEK293 cells. In some aspects, a producer cell is not a dendritic cell, macrophage, B cell, mast cell, neutrophil, Kupffer-Browicz cell, cell derived from any of these cells, or any combination thereof.

Human embryonic kidney 293 cells, also often referred to as HEK 293, HEK-293, 293 cells, or less precisely as HEK cells, are a specific cell line originally derived from human embryonic kidney cells grown in tissue culture.

HEK 293 cells were generated in 1973 by transfection of cultures of normal human embryonic kidney cells with sheared adenovirus 5 DNA in Alex van der Eb's laboratory in Leiden, the Netherlands. The cells were cultured and transfected by adenovirus. Subsequent analysis has shown that the transformation was brought about by inserting ˜4.5 kilobases from the left arm of the viral genome, which became incorporated into human chromosome 19.

A comprehensive study of the genomes and transcriptomes of HEK 293 and five derivative cell lines compared the HEK 293 transcriptome with that of human kidney, adrenal, pituitary and central nervous tissue. The HEK 293 pattern most closely resembled that of adrenal cells, which have many neuronal properties.

HEK 293 cells have a complex karyotype, exhibiting two or more copies of each chromosome and with a modal chromosome number of 64. They are described as hypotriploid, containing less than three times the number of chromosomes of a haploid human gamete. Chromosomal abnormalities include a total of three copies of the X chromosome and four copies of chromosome 17 and chromosome 22.

Variants of HEK293 cells useful to produce EVs include, but are not limited to, HEK 293F, HEK 293FT, and HEK 293T.

### IV. Therapeutic Uses

The present disclosure provides methods of treating a disease or condition is a subject in need thereof comprising administering a composition comprising EVs (e.g., exosomes) of the present disclosure to the subject. The present disclosure also provides methods of preventing or ameliorating the symptoms of a disease or condition is a subject in need thereof comprising administering a composition comprising EVs (e.g., exosomes) of the present disclosure to the subject. Also provided are methods to diagnose a disease or condition in a subject in need thereof comprising administering a composition comprising EVs (e.g., exosomes) of the present disclosure to the subject.

In one aspect, the disease or disorder is a cancer, an inflammatory disease, a neurodegenerative disorder, a central nervous disease or a metabolic disease.

Present disclosure also provides methods of preventing and/or treating a disease or disorder in a subject in need thereof, comprising administering an EV (e.g., exosome) disclosed herein to the subject. In some aspects, a disease or disorder that can be treated with the present methods comprises a cancer, graft-versus-host disease (GvHD), autoimmune disease, infectious diseases, or fibrotic diseases. In some aspects, the treatment is prophylactic. In other aspects, the EVs (e.g., exosomes) for the present disclosure are used to induce an immune response. In other aspects, the EVs (e.g., exosomes) for the present disclosure are used to vaccinate a subject.

In some aspects, the disease or disorder is a cancer. When administered to a subject with a cancer, in certain aspects, EVs (e.g., exosomes) of the present disclosure can up-regulate an immune response and enhance the tumor targeting of the subject's immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, an EV (e.g., exosome) is administered in an amount and for a time sufficient to convert a “cold tumor” into a “hot tumor,” i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. In other The term “distal tumor” or “distant tumor” refers to a tumor that has spread from the original (or primary) tumor to distant organs or distant tissues, e.g., lymph nodes. In some aspects, the EVs (e.g., exosomes) of the disclosure treats a tumor after the metastatic spread.

In some aspects, the disease or disorder is a graft-versus-host disease (GvHD). In some aspects, the disease or disorder that can be treated with the present disclosure is an autoimmune disease. Non-limiting examples of autoimmune diseases include: multiple sclerosis, peripheral neuritis, Sjogren's syndrome, rheumatoid arthritis, alopecia, autoimmune pancreatitis, Behcet's disease, Bullous pemphigoid, Celiac disease, Devic's disease (neuromyelitis optica), Glomerulonephritis, IgA nephropathy, assorted vasculitides, scleroderma, diabetes, arteritis, vitiligo, ulcerative colitis, irritable bowel syndrome, psoriasis, uveitis, systemic lupus erythematosus, and combinations thereof.

In some aspects, the disease or disorder is an infectious disease. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, Staphylococcus aureus, Mycobacterium tuberculosis, Chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikunga virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, the EVs (e.g., exosomes) are administered intravenously to the circulatory system of the subject. In some aspects, the EVs (e.g., exosomes) are infused in suitable liquid and administered into a vein of the subject.

In some aspects, the EVs (e.g., exosomes) are administered intra-arterialy to the circulatory system of the subject. In some aspects, the EVs (e.g., exosomes) are infused in suitable liquid and administered into an artery of the subject.

In some aspects, the EVs (e.g., exosomes) are administered to the subject by intrathecal administration. In some aspects, the EVs (e.g., exosomes) are administered via an injection into the spinal canal, or into the subarachnoid space so that it reaches the cerebrospinal fluid (CSF).

In some aspects, the EVs (e.g., exosomes) are administered intratumorally into one or more tumors of the subject.

In some aspects, the EVs (e.g., exosomes) are administered to the subject by intranasal administration. In some aspects, the EVs (e.g., exosomes) can be insufflated through the nose in a form of either topical administration or systemic administration. In certain aspects, the EVs (e.g., exosomes) are administered as nasal spray.

In some aspects, the EVs (e.g., exosomes) are administered to the subject by intraperitoneal administration. In some aspects, the EVs (e.g., exosomes) are infused in suitable liquid and injected into the peritoneum of the subject. In some aspects, the intraperitoneal administration results in distribution of the EVs (e.g., exosomes) to the lymphatics. In some aspects, the intraperitoneal administration results in distribution of the EVs (e.g., exosomes) to the thymus, spleen, and/or bone marrow. In some aspects, the intraperitoneal administration results in distribution of the EVs (e.g., exosomes) to one or more lymph nodes. In some aspects, the intraperitoneal administration results in distribution of the EVs (e.g., exosomes) to one or more of the cervical lymph node, the inguinal lymph node, the mediastinal lymph node, or the sternal lymph node. In some aspects, the intraperitoneal administration results in distribution of the EVs (e.g., exosomes) to the pancreas.

In some aspects, the EVs (e.g., exosomes) are administered to the subject by periocular administration. In some aspects, the EVs (e.g., exosomes) are injected into the periocular tissues. Periocular drug administration includes the routes of subconjunctival, anterior sub-Tenon's, posterior sub-Tenon's, and retrobulbar administration.

### V. Pharmaceutical Compositions and Methods of Administration

The present disclosure also provides pharmaceutical compositions comprising EVs (e.g., exosomes) described herein that are suitable for administration to a subject. The pharmaceutical compositions generally comprise a plurality of EVs (e.g., exosomes) comprising a biologically active molecule covalently linked to the plurality of EVs (e.g., exosomes) via a maleimide moiety and a pharmaceutically-acceptable excipient or carrier in a form suitable for administration to a subject. Pharmaceutically acceptable excipients or carriers are determined in part by the particular composition being administered, as well as by the particular method used to administer the composition. Accordingly, there is a wide variety of suitable formulations of pharmaceutical compositions comprising a plurality of EVs (e.g., exosomes). (See, e.g., Remington's Pharmaceutical Sciences, Mack Publishing Co., Easton, Pa. 18th ed. (1990)). The pharmaceutical compositions are generally formulated sterile and in full compliance with all Good Manufacturing Practice (GMP) regulations of the U.S. Food and Drug Administration. In some aspects, the pharmaceutical composition comprises one or more chemical compounds, such as for example, small molecules covalently linked to an EV (e.g., exosome) described herein.

In some aspects, a pharmaceutical composition comprises one or more therapeutic agents and an EV (e.g., exosome) described herein. In certain aspects, the EVs (e.g., exosomes) are co-administered with of one or more additional therapeutic agents, in a pharmaceutically acceptable carrier. In some aspects, the pharmaceutical composition comprising the EV (e.g., exosome) is administered prior to administration of the additional therapeutic agents. In other aspects, the pharmaceutical composition comprising the EV (e.g., exosome) is administered after the administration of the additional therapeutic agents. In further aspects, the pharmaceutical composition comprising the EV (e.g., exosome) is administered concurrently with the additional therapeutic agents.

Provided herein are pharmaceutical compositions comprising an EV (e.g., exosome) of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an EV (e.g., exosome) described herein. In certain aspects, the EVs (e.g., exosomes) are co-administered with of one or more additional therapeutic agents, in a pharmaceutically acceptable carrier. In some aspects, the pharmaceutical composition comprising the EV (e.g., exosome) is administered prior to administration of the additional therapeutic agents. In other aspects, the pharmaceutical composition comprising the EV (e.g., exosome) is administered after the administration of the additional therapeutic agents. In further aspects, the pharmaceutical composition comprising the EV (e.g., exosome) is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs (e.g., exosomes) of the present disclosure can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising EVs (e.g., exosomes) is administered intravenously, e.g. by injection. The EVs (e.g., exosomes) can optionally be administered in combination with other therapeutic agents that are at least partly effective in treating the disease, disorder or condition for which the EVs (e.g., exosomes) are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs (e.g., exosomes) of the present disclosure in an effective amount and in an appropriate solvent with one or a combination of ingredients enumerated herein, as desired. Generally, dispersions are prepared by incorporating the EVs (e.g., exosomes) into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs (e.g., exosomes) can be administered in the form of a depot injection or implant preparation which can be formulated in such a manner to permit a sustained or pulsatile release of the EVs (e.g., exosomes).

Systemic administration of compositions comprising EVs (e.g., exosomes) of the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs (e.g., exosomes) of the present disclosure is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising EVs (e.g., exosomes) of the present disclosure is administered as a liquid suspension. In certain aspects, the pharmaceutical composition is administered as a formulation that is capable of forming a depot following administration. In certain preferred aspects, the depot slowly releases the EVs (e.g., exosomes) into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs (e.g., exosomes) described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs (e.g., exosomes) described herein. In some aspects, the dosage form is formulated as a liquid suspension for intravenous injection. In some aspects, the dosage form is formulated as a liquid suspension for intratumoral injection.

In certain aspects, the preparation of EVs (e.g., exosomes) of the present disclosure is subjected to radiation, e.g., X rays, gamma rays, beta particles, alpha particles, neutrons, protons, elemental nuclei, UV rays in order to damage residual replication-competent nucleic acids.

In certain aspects, the preparation of EVs (e.g., exosomes) of the present disclosure is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of EVs (e.g., exosomes) of the present disclosure is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or

The EVs (e.g., exosomes) of the present disclosure may be used concurrently with other drugs. To be specific, the EVs (e.g., exosomes) of the present disclosure may be used together with medicaments such as hormonal therapeutic agents, chemotherapeutic agents, immunotherapeutic agents, medicaments inhibiting the action of cell growth factors or cell growth factor receptors and the like.

### VI. Kits

The present disclosure also provides kits, or products of manufacture comprising one or more EVs (e.g., exosomes) of the present disclosure and optionally instructions for use. In some aspects, the kit, or product of manufacture contains a pharmaceutical composition described herein which comprises at least one EV (e.g., exosome) of the present disclosure, and instructions for use. In some aspects, the kit, or product of manufacture comprises at least one EV (e.g., exosome) of the present disclosure or a pharmaceutical composition comprising the EVs (e.g., exosomes) in one or more containers. One skilled in the art will readily recognize that the EVs (e.g., exosomes) of the present disclosure, pharmaceutical composition comprising the EVs (e.g., exosomes) of the present disclosure, or combinations thereof can be readily incorporated into one of the established kit formats which are well known in the art.

In some aspects, the kit, or product of manufacture comprises EVs (e.g., exosomes) one or more biologically active molecules, reagents to covalently attach the one or more biologically active molecules to the EVs (e.g., exosomes) via a maleimide moiety, or any combination thereof, and instructions to conduct the reaction to covalently attach the one or more biologically active molecules to the EVs (e.g., exosomes) via a maleimide moiety.

In some aspects, the kit comprises reagents to conjugate a biologically active molecule to an EV (e.g., exosome) via a maleimide moiety, and instructions to conduct the conjugation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooke, Antisense drug Technology: Principles, Strategies and Applications, 2nd Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

The following examples are provided for illustrative purposes only, and are not to be construed as limiting the scope or content of the invention in any way. The practice of the current invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); Green & Sambrook et al., Molecular Cloning: A Laboratory Manual, 4th Edition (Cold Spring Harbor Laboratory Press, 2012); Colowick & Kaplan, Methods In Enzymology (Academic Press); Remington: The Science and Practice of Pharmacy, 22nd Edition (Pharmaceutical Press, 2012); Sundberg & Carey, Advanced Organic Chemistry: Parts A and B, 5th Edition (Springer, 2007).

### Example 1

To generate exosomes described herein, human embryonic kidney (HEK) cell line (HEK293SF) will be used. The cells will be stably transfected with Scaffold X and/or Scaffold Y linked to an agent of interest (e.g., antigen, adjuvant, or immune modulator). For example, CD40L-expressing exosomes can be generated by transfecting HEK293SF cells with CD40L-GFP PTGFRN fusion molecules, which express as a monomer or as a forced trimer.

Upon transfection, HEK293SF cells will be grown to high density in chemically defined medium for 7 days. Conditioned cell culture media will be then collected and centrifuged at 300-800×g for 5 minutes at room temperature to remove cells and large debris. Media supernatant will be supplemented with 1000 U/L BENZONASE® and incubated at 37° C. for 1 hour in a water bath. Supernatant will be collected and centrifuged at 16,000×g for 30 minutes at 4° C. to remove residual cell debris and other large contaminants. Supernatant will then be ultracentrifuged at 133,900×g for 3 hours at 4° C. to pellet the exosomes. Supernatant will be discarded and any residual media will be aspirated from the bottom of the tube. The pellet will be resuspended in 200-1000 μL PBS (—Ca —Mg).

To further enrich exosome populations, the pellet will be processed via density gradient purification (sucrose or OPTIPREP™).

The gradient will be spun at 200,000×g for 16 hours at 4° C. in a 12 mL Ultra-Clear (344059) tube placed in a SW 41 Ti rotor to separate the exosome fraction.

The exosome layer will then be gently removed from the top layer and diluted in ˜32.5 mL PBS in a 38.5 mL Ultra-Clear (344058) tube and ultracentrifuged again at 133,900×g for 3 hours at 4° C. to pellet the purified exosomes. The resulting pellet will be resuspended in a minimal volume of PBS (˜200 μL) and stored at 4° C.

For OPTIPREP™ gradient, a 3-tier sterile gradient will be prepared with equal volumes of 10%, 30%, and 45% OPTIPREP™ in a 12 mL Ultra-Clear (344059) tube for a SW 41 Ti rotor. The pellet will be added to the OPTIPREP™ gradient and ultracentrifuged at 200,000×g for 16 hours at 4° C. to separate the exosome fraction. The exosome layer will then be gently collected from the top ˜3 mL of the tube.

The exosome fraction will be diluted in −32 mL PBS in a 38.5 mL Ultra-Clear (344058) tube and ultracentrifuged at 133,900×g for 3 hours at 4° C. to pellet the purified exosomes. The pelleted exosomes will then be resuspended in a minimal volume of PBS (˜200 μL) and stored at 4° C. until ready to be used.

### Example 2

### In Vitro Analysis of KRAS mRNA and/or KRAS Protein Reduction

Exemplary ASOs disclosed herein were designed to specifically target KRAS transcript encoding the KRAS protein with a G12D mutation. See FIG. 1F. ASOs targeting any other target gene disclosed herein (e.g., NLRP3, STAT6, CEBP/β, STAT3, or NRAS) can also be used with similar methods. The disclosed ASOs will be tested for their ability to knockdown KRAS mRNA and/or KRAS protein expression in reporter cell lines containing the wild-type (WT) or G12D allele of human KRAS mRNA upstream of Renilla luciferase. To control for general cellular toxicity, the cell lines will also contain firefly luciferase. KRAS specific siRNA will be used as positive control.

Briefly, reporter cell lines expressing the WT or G12D mutant KRAS protein will be grown in cell culture media and seeded onto a 96 well plate. Then, the cells will be treated with different concentrations of EVs (e.g., exosomes) comprising one or more ASOs disclosed herein (“EV-ASO”). Approximately 3 days after EV-ASO treatment, the cells will be harvested and RNA and/or protein will be purified from the cells. Then, the KRAS mRNA and/or KRAS protein expression levels in the cells will be quantified using assays such as, qPCR and Western blot.

### Example 3

### In Vivo Analysis of KRAS mRNA/KRAS Protein Reduction

To evaluate the potency of EVs (e.g., exosomes) comprising one or more of the ASOs disclosed herein in reducing KRAS mRNA and/or KRAS protein level in vivo, a tumor mice model will be used. ASOs targeting any other target gene disclosed herein (e.g., NLRP3, STAT6, CEBP/β, STAT3, or NRAS) can also be used with similar methods. The ASOs disclosed herein will be administered to the tumor mice at various dosing regimens. The mice will be monitored for tumor growth periodically. The mice will eventually be sacrificed and the KRAS mRNA and/or KRAS protein levels will be assessed in various cells.

### Example 4

### In Vitro Analysis of NLRP3 mRNA and/or NLRP3 Protein Reduction

Exemplary ASOs disclosed herein were designed to specifically target NLRP3 transcript. See FIG. 1A. The disclosed ASOs will be tested for their ability to knockdown NLRP3 mRNA and/or NLRP3 protein expression in reporter cell lines containing a human NLRP3 coding sequence upstream of reporter. NLRP3-specific siRNA will be used as positive control.

Briefly, the reporter cell lines expressing NLRP3 will be grown in cell culture media and seeded onto a 96 well plate. Then, the cells will be treated with different concentrations of EVs (e.g., exosomes) comprising one or more ASOs disclosed herein (“EV-ASO”). Methods for producing such EVs are provided elsewhere in the present disclosure. Approximately 3 days after EV-ASO treatment, the cells will be harvested and RNA and/or protein will be purified from the cells. Then, the NLRP3 mRNA and/or NLRP3 protein expression levels in the cells will be quantified using assays such as, qPCR and Western blot.

### Example 5

### NLRP3 ASO Design

Mouse and human ASOs were designed to target NLRP3 (Gene ID No. 114548) expression. Target sequences were selected using the reference sequences NM_004895 for human NLRP3 and NM_145827.4 for mouse NLRP3. A list of possible ASOs were generated for each gene by tilling of ASOs across the entire length of the nascent transcript. ASOs having 15, 16, 17, 18, 19, or 20 nucleobases in length were generated.

ASOs were prioritized based on the following properties: must hit all splice forms; low self-dimerization energy (on-target activity); no GGGG motif (SEQ ID NO: 598) (can cause synthesis issues); less than 3 CpG dinucleotides in the oligo (potential immunostimulation); less than 8 bases of palindromic sequence (potential dimerization & immunostimulation); more than 2 mismatch and no more than 17 contiguous bases in an off-target hit to any gene, including known miRNA and lncRNA, and both nascent and mature transcripts; no overlap with repetitive sequences; and no overlap with SNPs of greater than or equal to 0.01 MAF in the general population. Additional criteria included Predicted species cross reactivity (e.g., human, cyno, rhesus, rat, mouse transcripts); and an off target (OT) filter less than or equal to 3 mismatch (mm) in mature transcripts, less than or equal to 3 mm in lnc transcripts, less than or equal to 3 mm in miRNAs, and less than or equal to 3 mm in nascent transcripts.

### Example 6

### In Vivo Analysis of NLRP3 mRNA/NLRP3 Protein Reduction

To evaluate the potency of EVs (e.g., exosomes) comprising one or more of the ASOs disclosed herein in reducing NLRP3 mRNA and/or NLRP3 protein level in vivo, a fibrosis mouse-model will be used. The ASOs disclosed herein will be administered to the mice at various dosing regimens. The mice will be monitored for symptoms of fibrosis. The mice will eventually be sacrificed and the NLRP3 mRNA and/or NLRP3 protein levels will be assessed in various cells.

### Example 7

### Functional Assay in Human Primary Monocytes and Macrophages

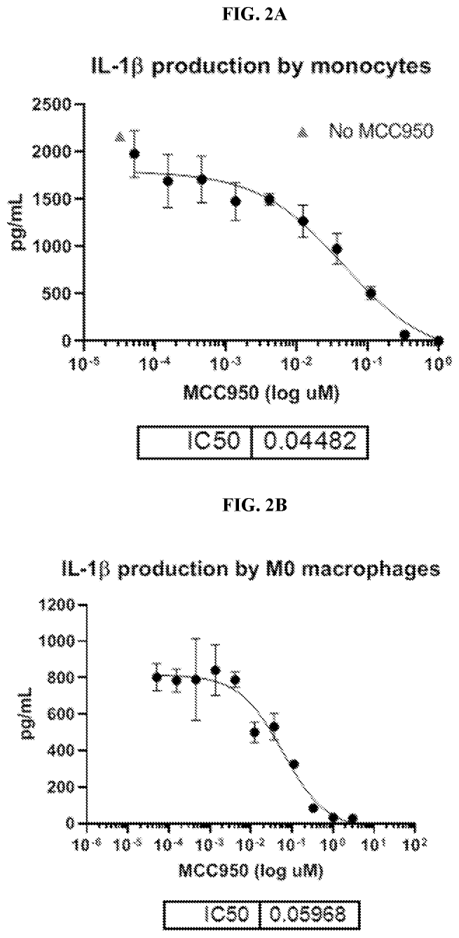

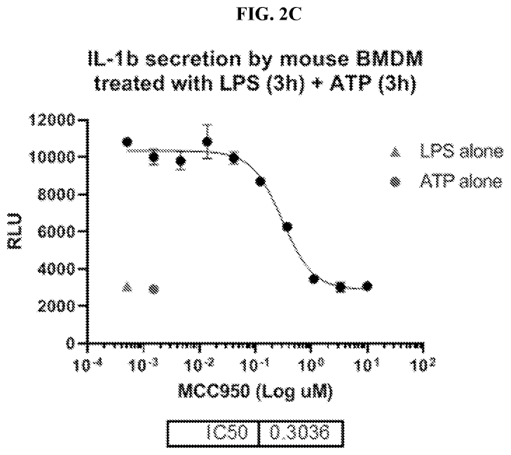

Activation of the NLRP3 pathway induces IL-1β production by human monocytes and macrophages. Activation of the NLRP3 pathway can be achieved by 3 hours priming with 200 ng/mL LPS followed by overnight incubation with 5 mM ATP, as demonstrated using monocytes isolated from human whole blood, as well as M0 macrophages that were matured in M-CSF for 6 days using the monocytes. The induction of IL-1β production can be inhibited by MCC950 and IC50 values of treatment with the free drug (FIGS. 2A-2B). IL-1β concentrations are determined using AlphaLISA assay.

Similar to IL-1β production by human cells following activation of the NLRP3 pathway, mouse bone marrow-derived macrophages also produce IL-1β which can be achieved by 3 hours priming with 200 ng/mL LPS followed by 3 hours incubation with 5 mM ATP (FIG. 2C).

### Example 8

### In Vivo Peritonitis Model

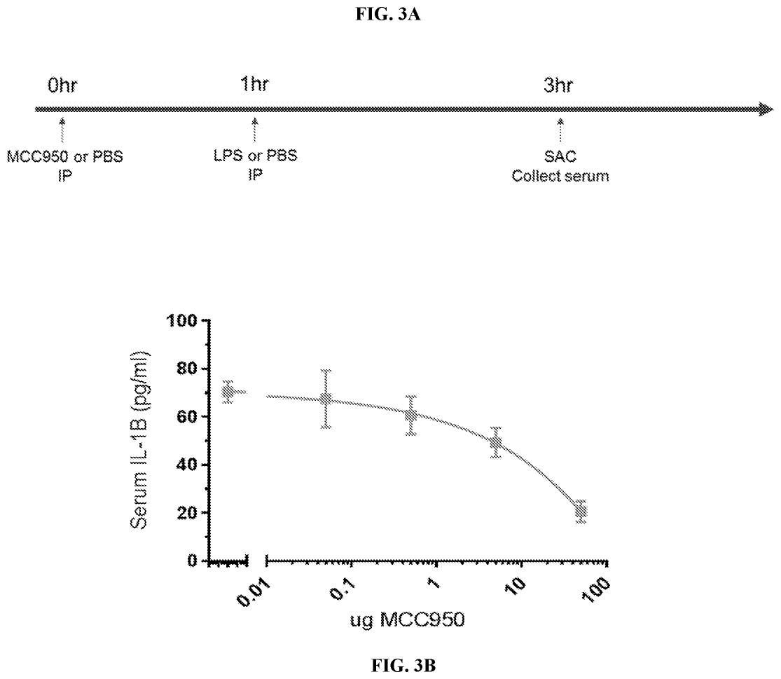

Intraperitoneal LPS challenge induces the production of IL-1β in mice, which can be detected in the systemic circulation 3 hours post-challenge. The induction of IL-1β in the serum of LPS-challenged mice can be inhibited by pre-treatment with MCC950 administered intraperitoneally, 1 hour prior to challenged (FIGS. 3A-3B).

### Example 9

### In Vitro Analysis of mRNA and/or Protein Reduction

Exemplary ASOs disclosed herein were designed to specifically target the STAT6 transcript (FIG. 1B) or the CEBP/β transcript (FIG. 1C). The disclosed ASOs will be tested for their ability to knockdown STAT6 or CEBP/β mRNA and/or STAT6 or CEBP/β protein expression in reporter cell lines containing a human STAT6 or CEBP/β coding sequence upstream of reporter. STAT6- or CEBP/β-specific siRNA will be used as positive control.

Briefly, the reporter cell lines expressing STAT6 or CEBP/β will be grown in cell culture media and seeded onto a 96 well plate. Then, the cells will be treated with different concentrations of EVs (e.g., exosomes) comprising one or more ASOs disclosed herein (“EV-ASO”). Methods for producing such EVs are provided elsewhere in the present disclosure. Approximately 3 days after EV-ASO treatment, the cells will be harvested and RNA and/or protein will be purified from the cells. Then, the STAT6 or CEBP/β mRNA and/or STAT6 or CEBP/p protein expression levels in the cells will be quantified using assays such as, qPCR and Western blot.

A lead ASO will be selected first by using in silico selection based on alternative transcript cross reactivity, species cross reactivity, specificity for gene of interest, presence of SNPs within ASO, length of ASO, location diversity, toxic motifs, and predicted binding affinity. Next, the ASOs will be screened for the ability to knock down (by at least 50% at 2 nM, and less than 20% knock down of GAPDH at 20 nM) target gene expression in cell lines transfected with the target sequence (STAT6 or CEBP/β mRNA). ASOs will then be assayed for target gene knock down potency in primary macrophages from at least two donors. Housekeeping gene expression stability, diversity of sequence location, and expression of predicted off-targets after treatment will also be observed. Optimal ASOs having the highest reprogramming activity (gene expression changes, cytokine production, T cell suppression) in primary macrophages will be selected as the lead ASOs.

### Example 10

### ASO Design

Mouse and human ASOs were designed to target STAT6 (Gene ID No. 6778) or CEBP/β (Gene ID No. 1051) expression. Target STAT6 sequences were selected using the reference sequences NM_001178078.2 for human STAT6 and NM_009284.2 for mouse STAT6. Target CEBP/β sequences were selected using the reference sequences NM_001285878.1 for human CEBP/β and NM_009883.4 for mouse CEBP/β. A list of possible ASOs were generated for each gene by tilling of ASOs across the entire length of the nascent transcript. ASOs having 15, 16, 17, 18, 19, or 20 nucleobases in length were generated.

ASOs were prioritized based on the following properties: must hit all splice forms; low self-dimerization energy (on-target activity); no GGGG motif (can cause synthesis issues); less than 3 CpG dinucleotides in the oligo (potential immunostimulation); less than 8 bases of palindromic sequence (potential dimerization & immunostimulation); more than 2 mismatch and no more than 17 contiguous bases in an off-target hit to any gene, including known miRNA and lncRNA, and both nascent and mature transcripts; no overlap with repetitive sequences; and no overlap with SNPs of greater than or equal to 0.01 MAF in the general population. Additional criteria included Predicted species cross reactivity (e.g., human, cyno, rhesus, rat, mouse transcripts); and an off target (OT) filter less than or equal to 3 mismatch (mm) in mature transcripts, less than or equal to 3 mm in lnc transcripts, less than or equal to 3 mm in miRNAs, and less than or equal to 3 mm in nascent transcripts.

### Example 11

### ASO Loading on Exosomes

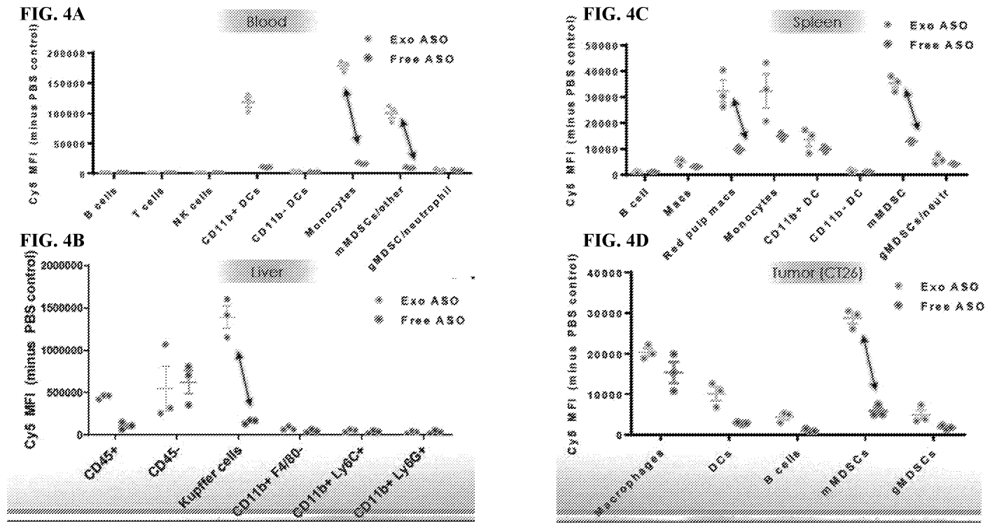

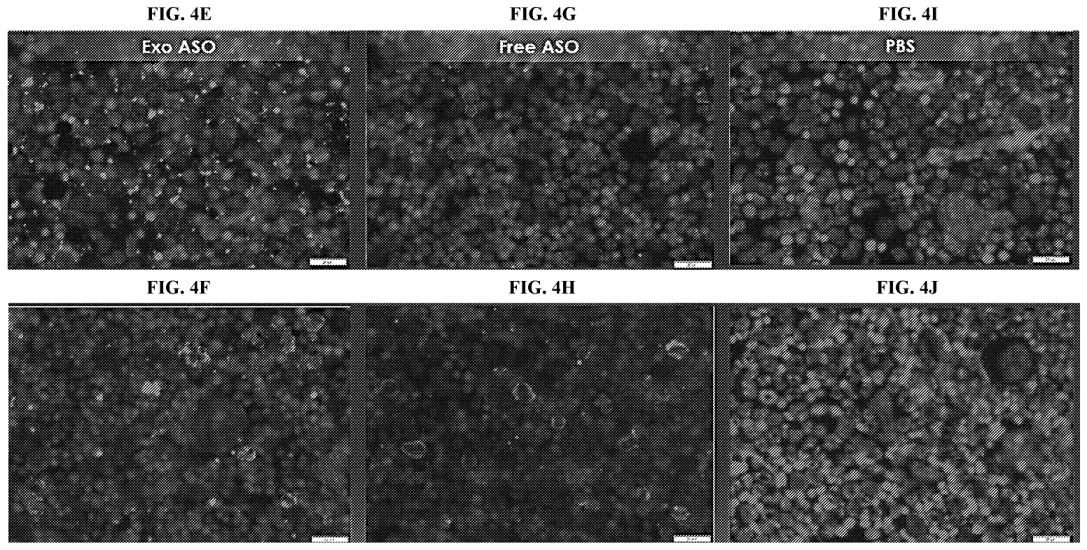

Mice were treated intravenously with a single dose of 2E11 exosomes loaded with a reporter ASO (“exo ASO”) or with a single dose of free reporter ASO (“free ASO”). One hour following administration, increased exo ASO uptake was ubserved in CD11b⁺ dendritic cells, monocytes, and mMDSCs in the blood (FIG. 4A); Kupffer cells in the liver (FIG. 4B); red pulp macs, monocytes, and mMDSCs in the spleen (FIG. 4C); and dendritic cells and mMDSCs in tumor tissue (FIG. 4D), as evidenced by MFI, relative to the localization of free ASO. Uptake of exo ASO was also higher in bone marrow (FIGS. 4E-4F) as compared to uptake of free ASO and negative controls (FIGS. 4G-4J).

### Example 12

### Exo-STAT6-ASO and Exo-CEBP/β-ASO Are Capable of Repolarizing M2 Macrophages

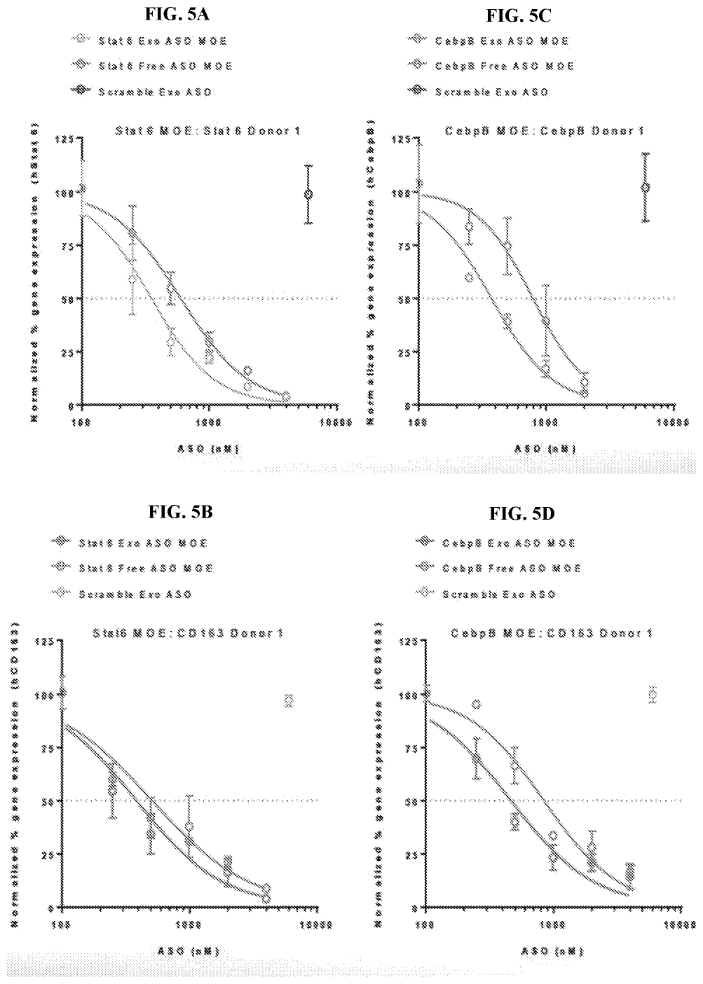

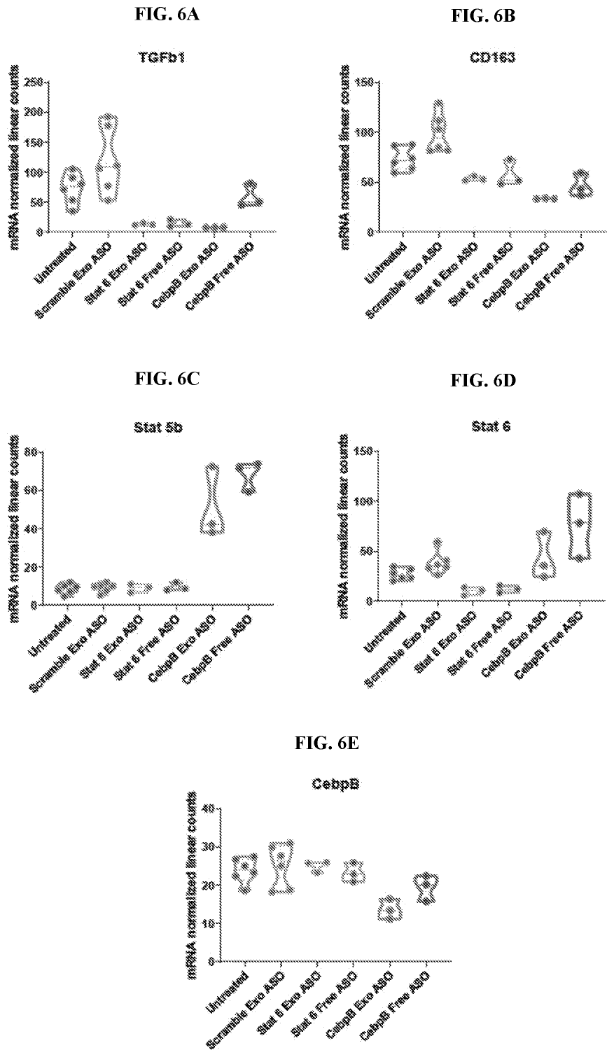

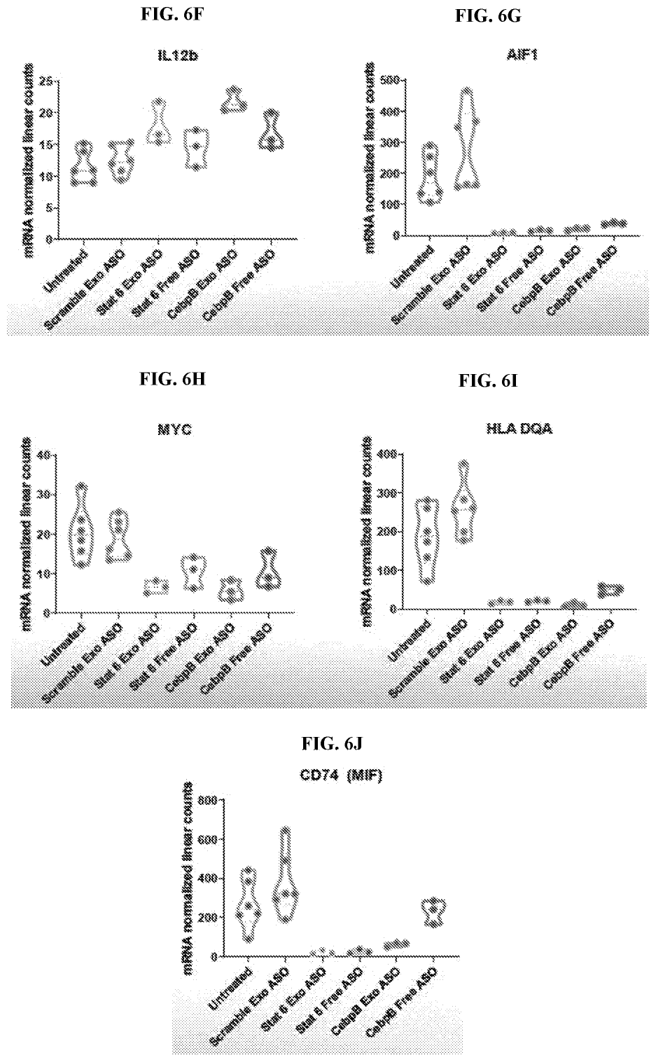

Primary human macrophages were polarized with IL4/IL10/TGFβ treatment and treated with increasing concentrations of Exo-STAT6-ASO or Exo-CEBP/β-ASO. In vitro treatment of primary human macrophages with Exo-STAT6-ASO or Exo-CEBP/β-ASO induces dose-dependent knockdown of STAT6 (FIG. 5A) or CEBP/β (FIG. 5C), respectively, as well as the downregulation of an M2 macrophage gene, CD163 (FIGS. 5B and 5D). Potency was found to be slightly higher using the Exo-ASOs as compared to the free ASOs. In addition, various M2 genes were downregulated and various M1 genes were upregulated following treatment with Exo-STAT6-ASO or Exo-CEBP/β-ASO (FIGS. 6A-6J).

### Example 13

### Exo-STAT6-ASO and Exo-CEBP/β-ASO Target Gene Knockdown in CD11b Cells

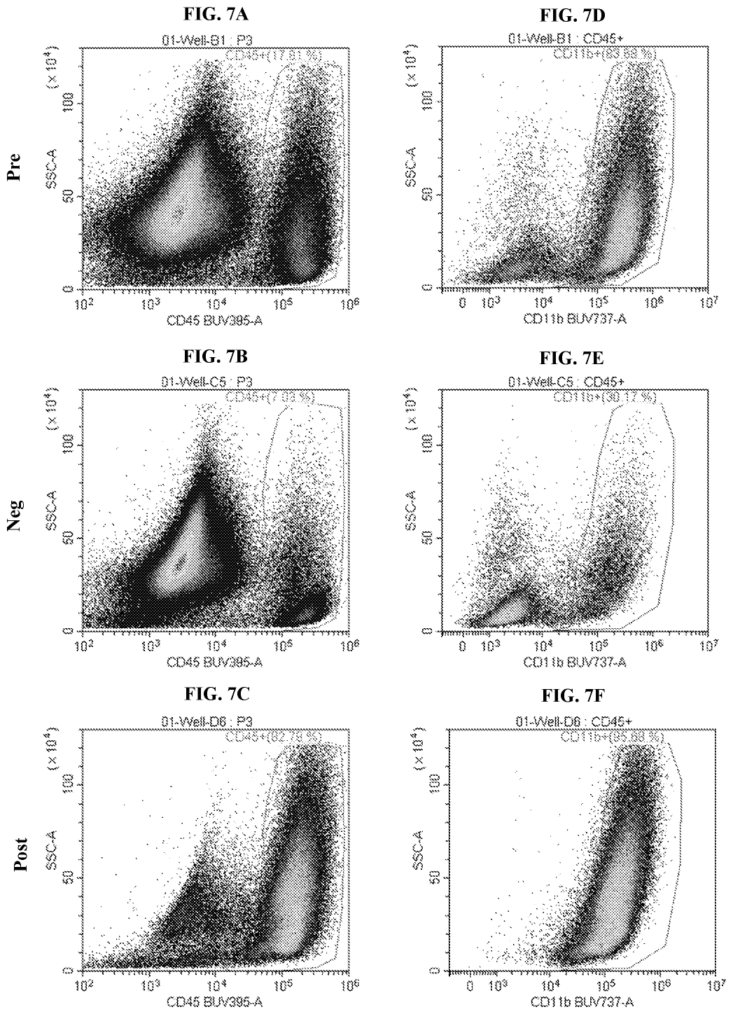

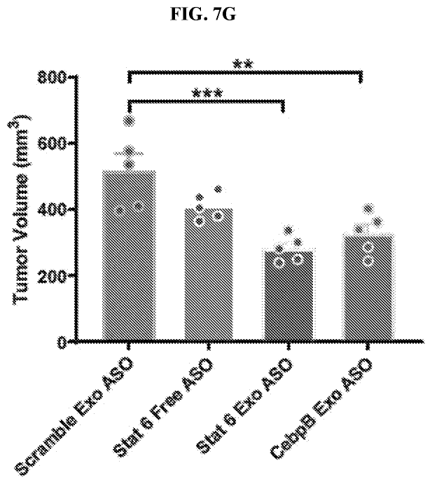

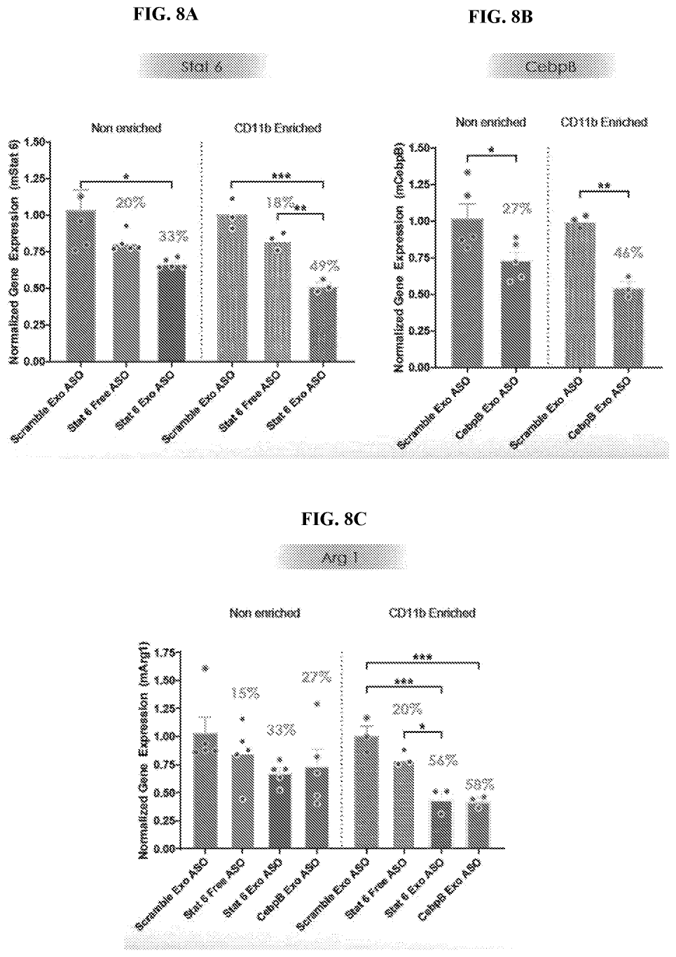

In vivo, the primary recipient cell for Exo-STAT6-ASO and Exo-CEBP/β-ASO is CD11b cells. To further measure the uptake and known-down efficiency of the Exo-ASOs, mice were treated with Exo-STAT6-ASO or Exo-CEBP/β-ASO and sacrificed. CD11b⁺ cells were then isolated and enriched (FIGS. 7A-7F). Though not the endpoint, tumor volume was significantly lower in Exo-STAT6-ASO treated mice and Exo-CEBP/β-ASO treated mice relative to mice treated with a scramble Exo-ASO control, and mice treated with Exo-STAT6-ASO or Exo-CEBP/β-ASO tended to have smaller tumors than mice treated with STAT6 free ASO (FIG. 7G). Exo-ASO target gene knockdown was more pronounced in the CD11b-enriched cells than non-enriched cells following treatment with Exo-STAT6-ASO (FIG. 8A) or Exo-CEBP/β-ASO (FIG. 8B). In addition, both Exo-ASOs were effective at reducing Arg1 expression to a greater extent in CD11b-enriched cells than non-enriched cells (FIG. 8C).

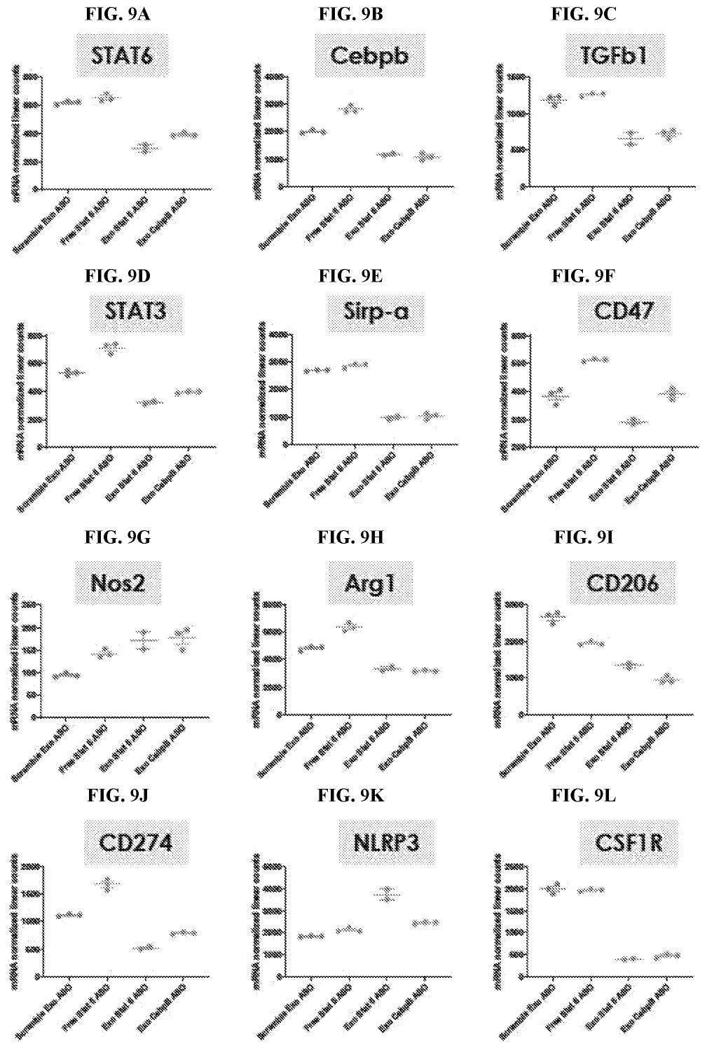

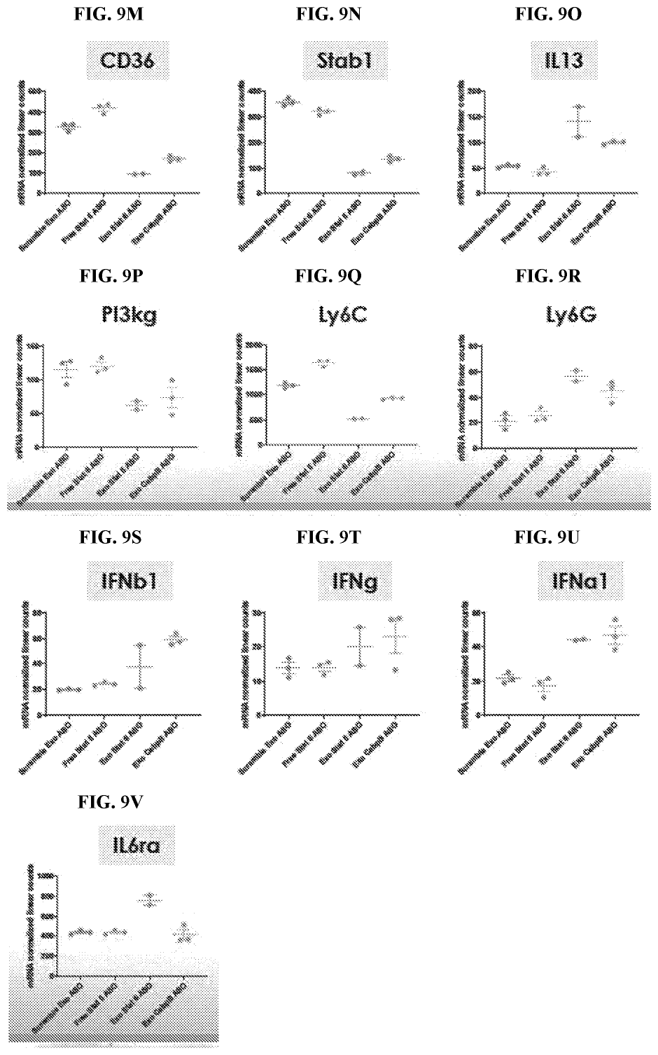

CD11b-enriched cells treated with either Exo-STAT6-ASO or Exo-CEBP/β-ASO also showed macrophage reprogramming as evidenced by upregulation of various M1 genes and downregulation of various M2 genes (FIGS. 9A-9V).

### Example 14

### Treatment of Fibrosis Using Exo-STAT6-ASO and/or Exo-CEBP/β-ASO

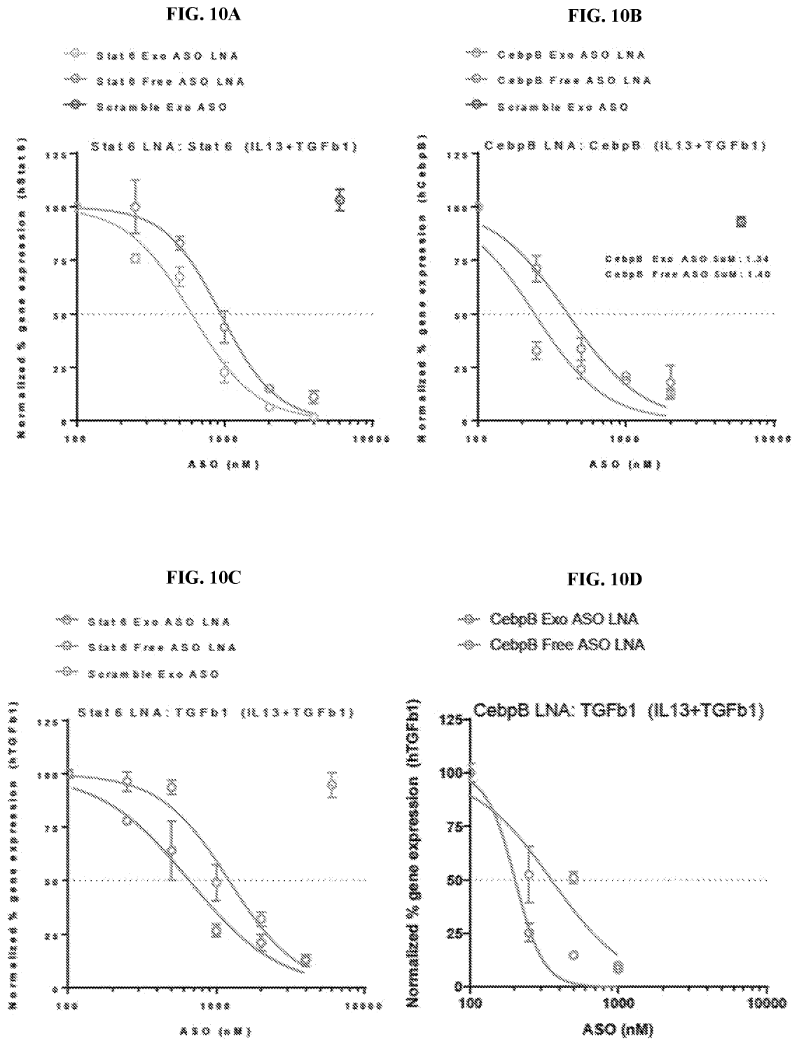

Excessive M2 macrophage activation leads to the continuous production of TGFβ and growth factors that promote proliferation of myofibroblasts, activation of EMT/EndoMT, and extracellular matrix deposition. M2 macrophages represent a break point between wound healing and exacerbation of pro-fibrotic process. To test whether Exo-STAT6-ASO or Exo-CEBP/β-ASO could be used to treat fibrosis in a subject, primary human M2 macrophages were polarized with IL-13/TGFβ treatment, which are drivers of fibrosis. Cells were then exposed to increasing concentrations of free STAT6 ASO (FIGS. 10A and 10C), free CEBP/β ASO (FIGS. 10B and 10D), Exo-STAT6-ASO (FIGS. 10A and 10C), or Exo-CEBP/β-ASO (FIGS. 10B and 10D); and assayed for expression of the target gene (STAT6, FIG. 10A; CEBP/β, FIG. 10B) or expression of TGFβ1 (FIGS. 10C-10D).

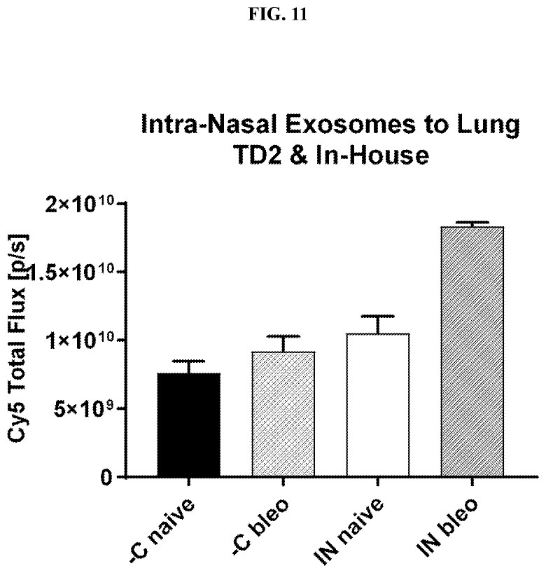

To test the feasibility of Exo-ASO delivery in vivo using intra-nasal administration, 6-weak old mice were treated with bleomycin to induce pulmonary fibrosis. Two weeks later, mice were administered Exo-ASO-Cy5 intranasally, and the mice were sacrificed 4 hours post-administration. Bleomycin induced mice administered Exo-ASO-Cy5 showed increased total flux of Cy5 relative to naïve mice administered Exo-ASO-Cy5 (“IN naïve”) and relative to naïve and treated mice administered a PBS negative control (“−C”) (FIG. 11).

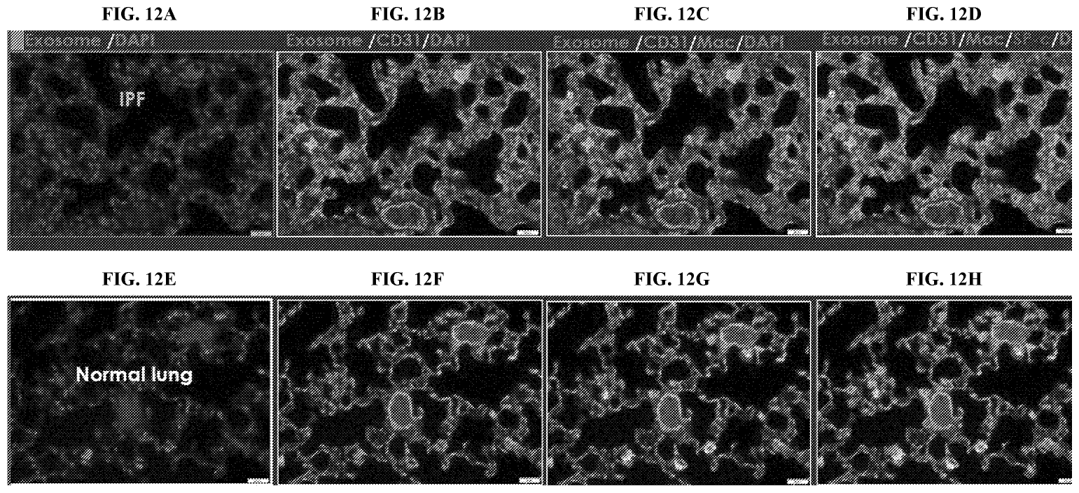

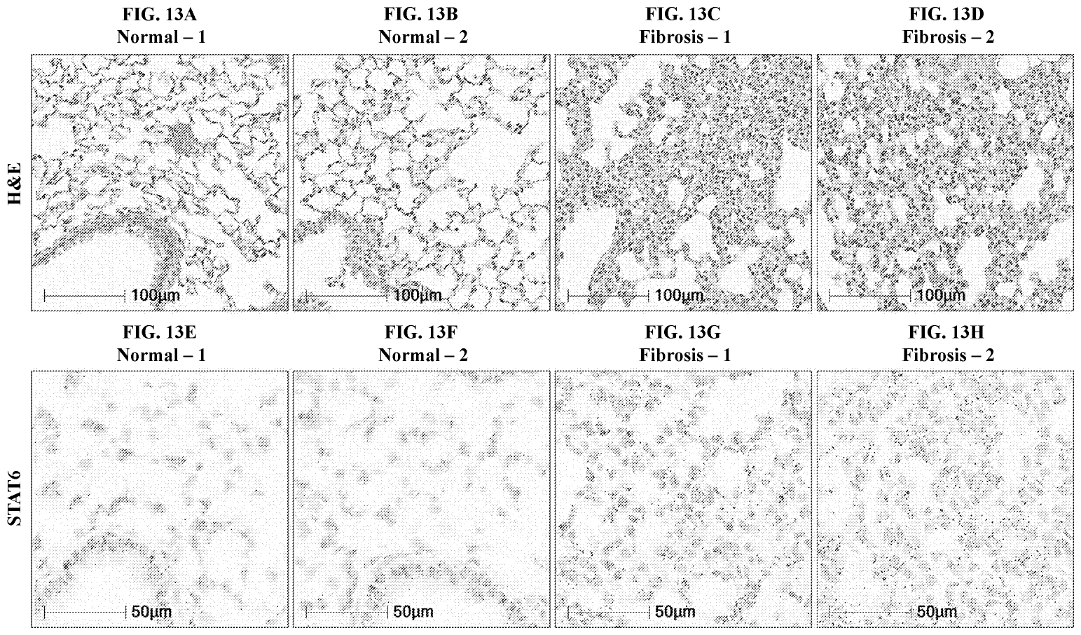

Exosome uptake was observed by lung macrophages and lung capillary endothelial cells in both normal lung and induced-pulmonary fibrosis lungs tissue (FIGS. 12A-12H and 13A-13H).

### Example 15

### Treatment of a Hepatocarcinoma Mouse-Model Using Exo-STAT6-ASO and/or Exo-CEBP/β-ASO

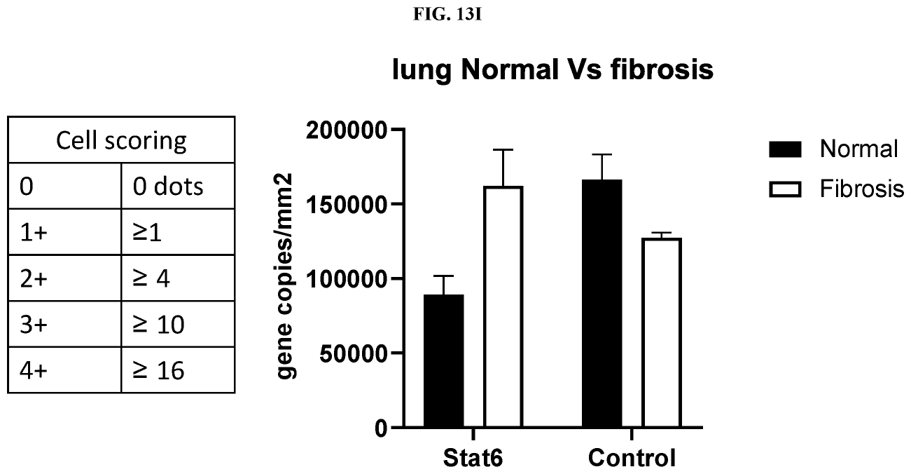

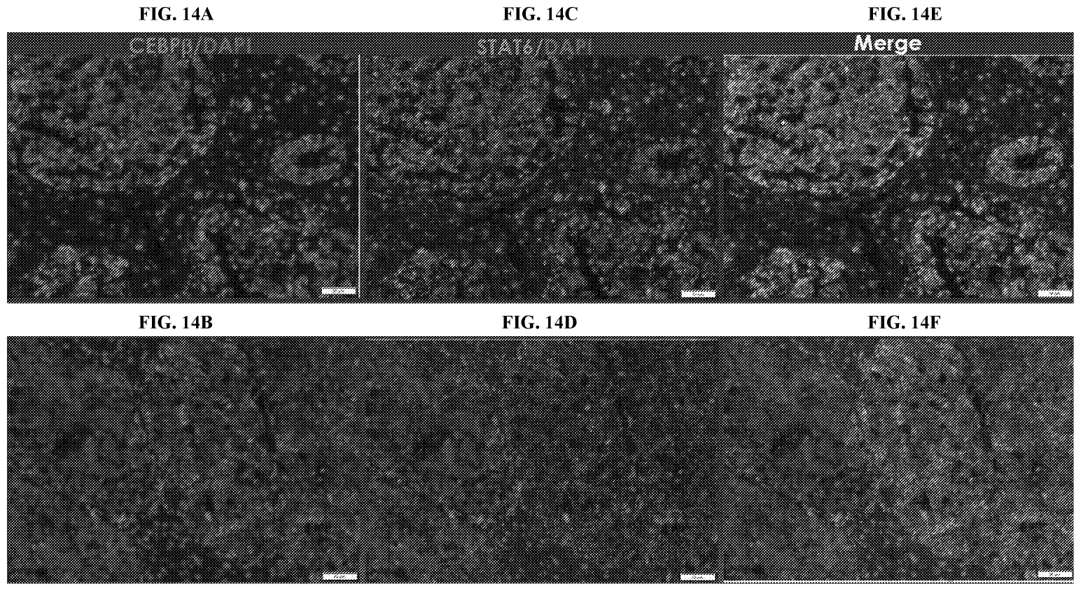

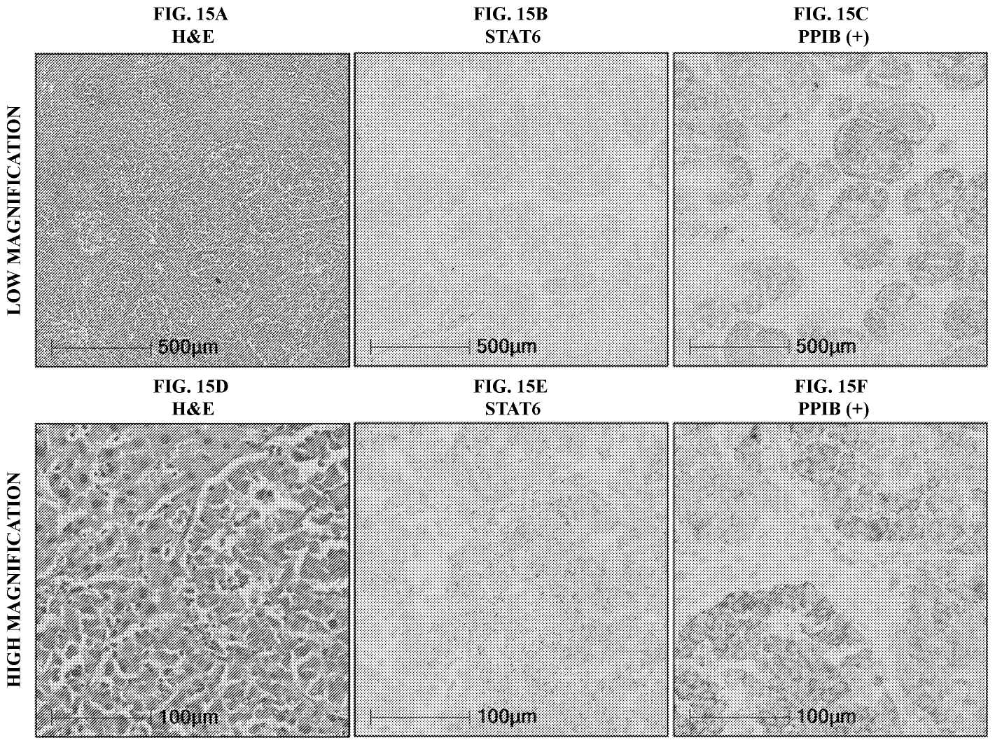

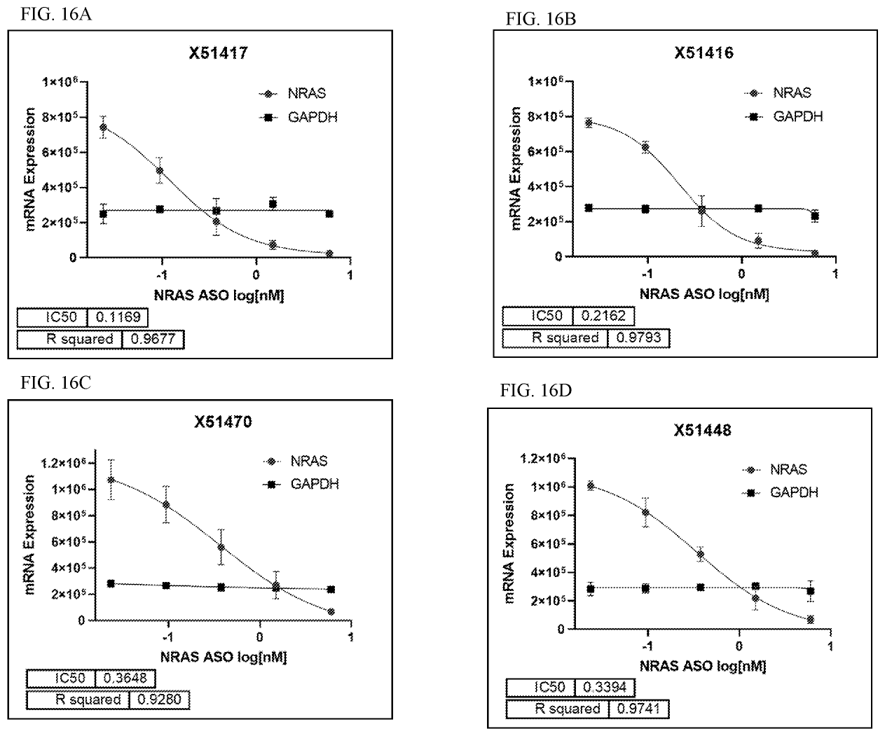

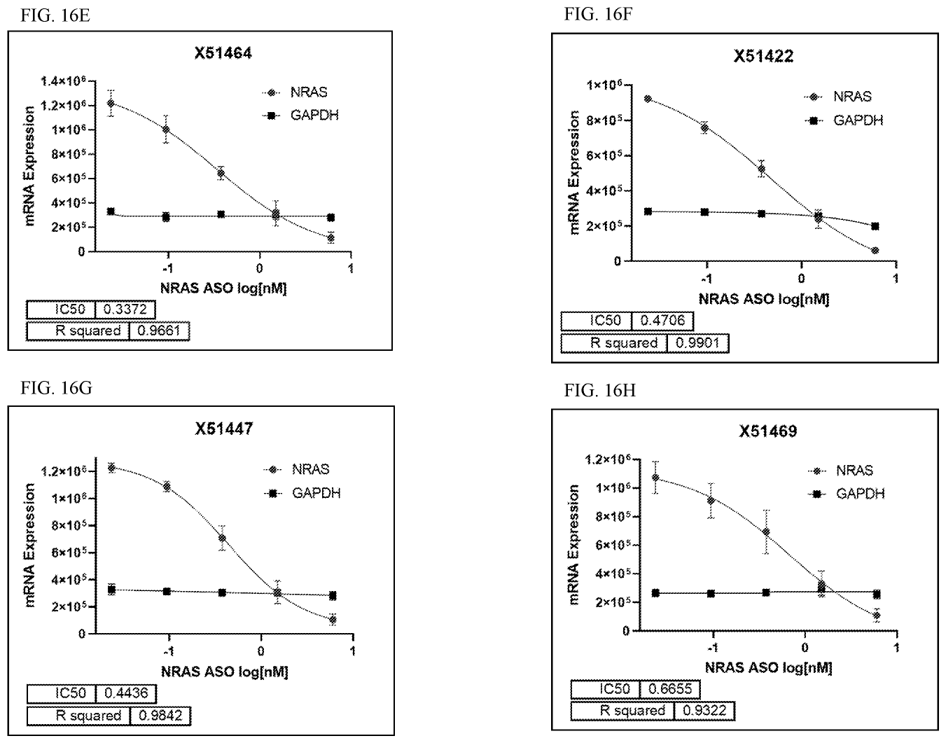

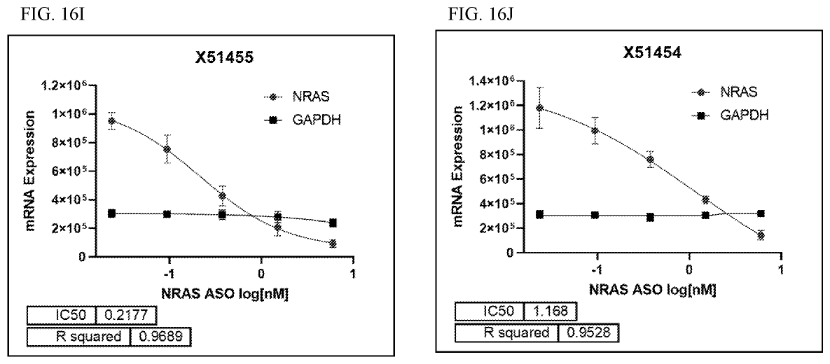

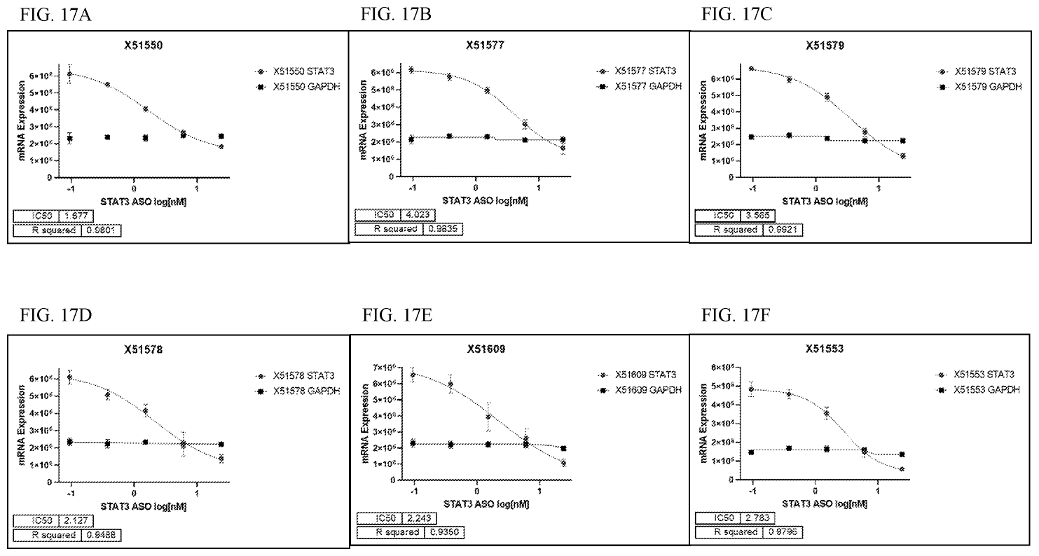

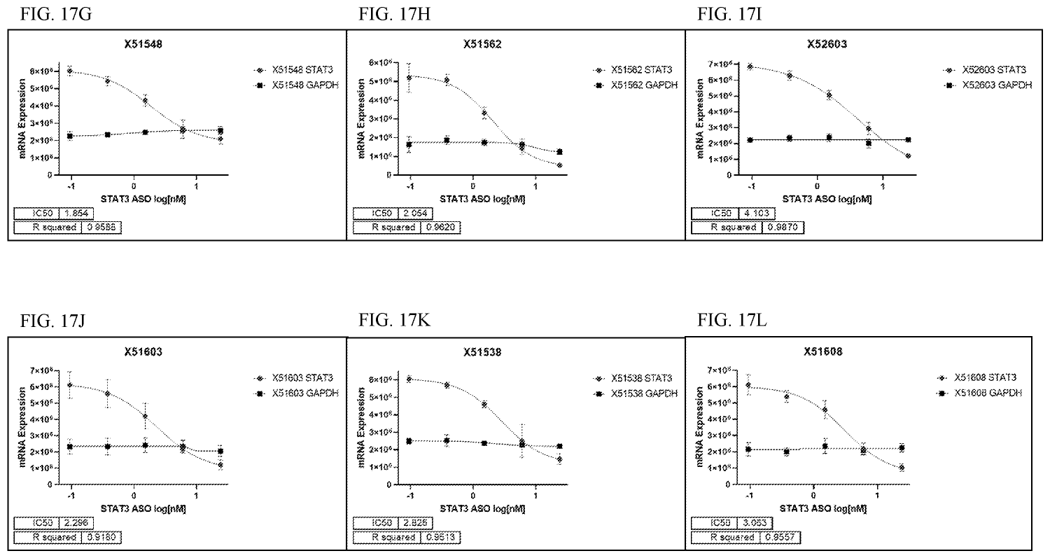

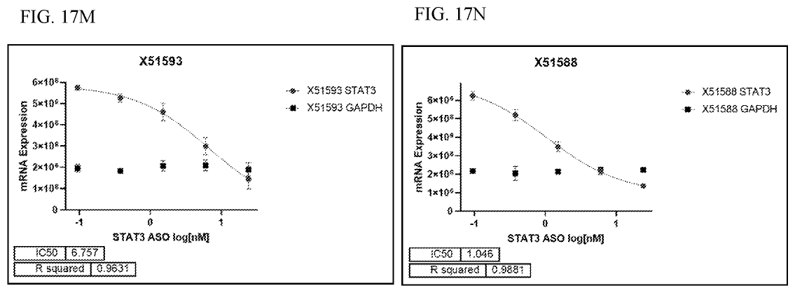

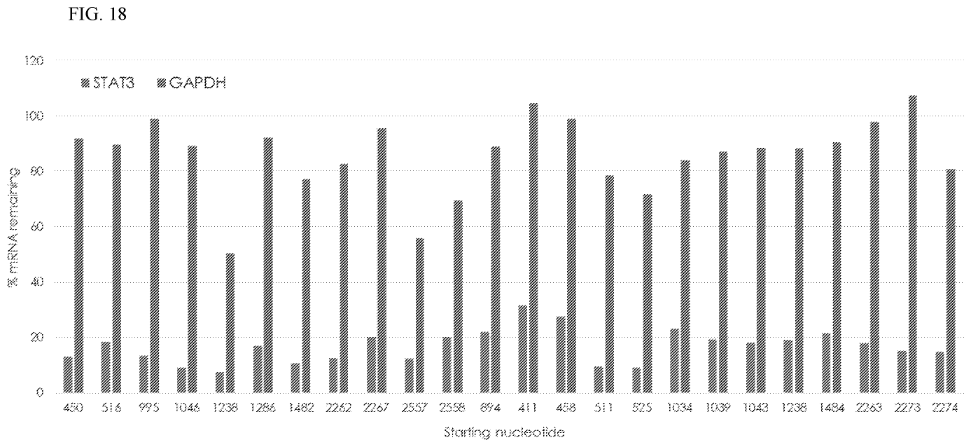

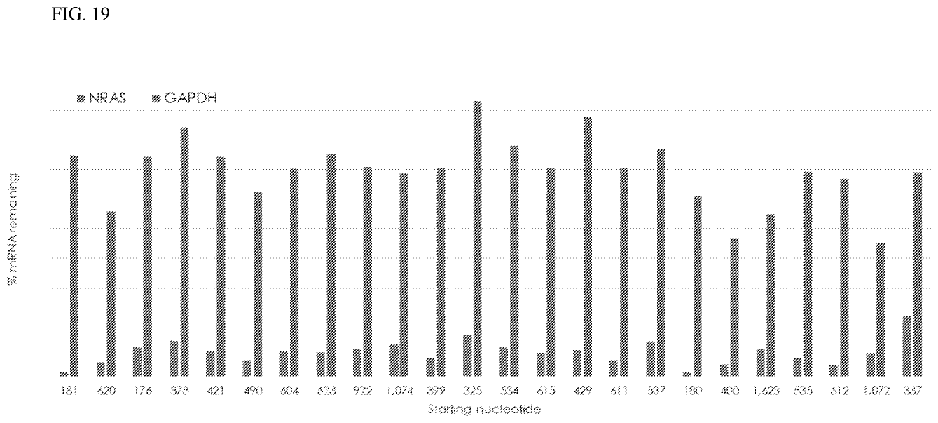

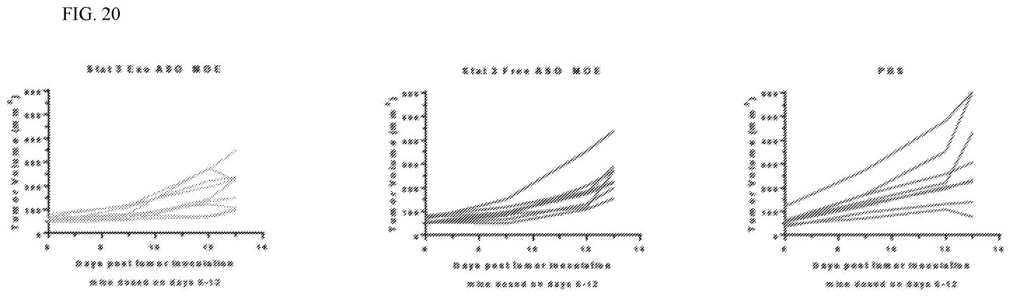

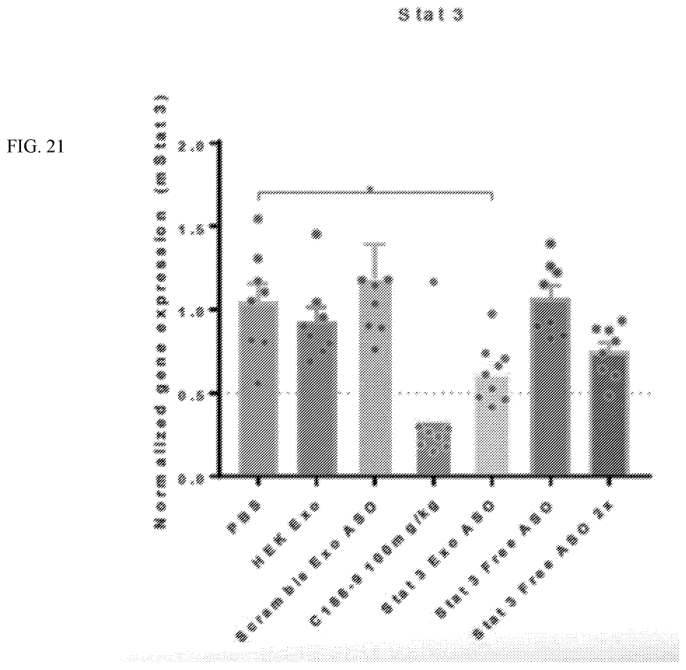

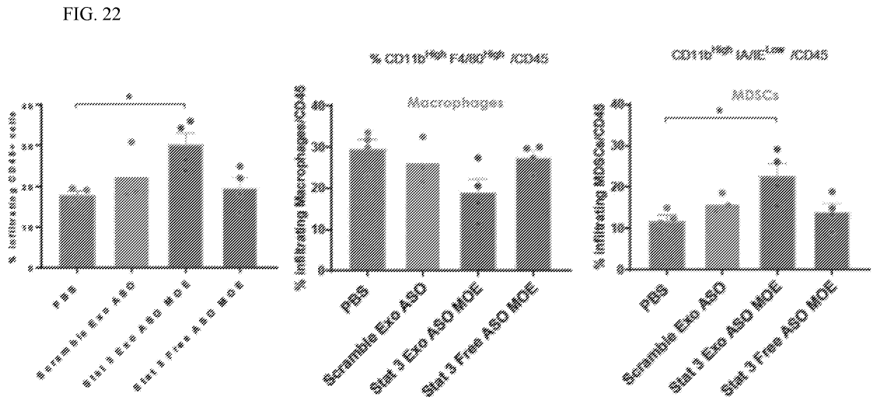

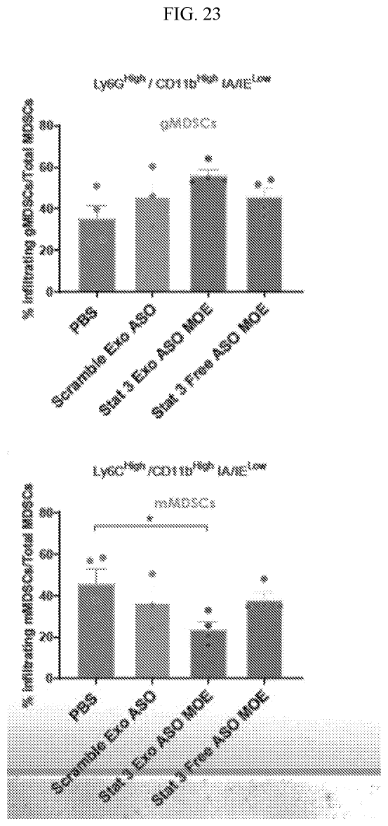

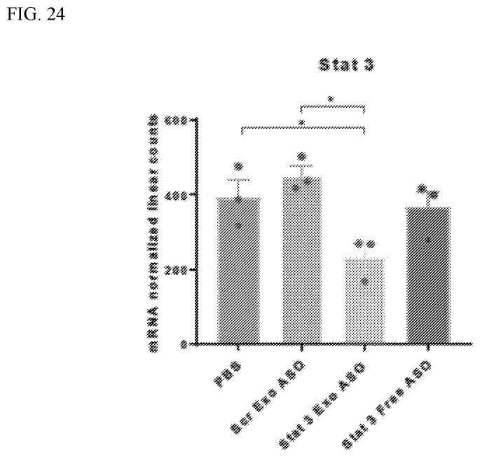

Hepa1-6 mice will be used to test the in vivo efficacy of Exo-STAT6-ASO and Exo-CEBP/β-ASO for treating a tumor. The Hepa1-6 line is an orthotopic mouse model of hepatocarcinoma. Samples were obtained from CRO and analyzed by in situ hybridization for expression of STAT6 and CEBP/β (FIGS. 14A-14F and 15A-15F).

### Example 16

### Analysis of STAT3 mRNA Expression

The ASOs of the present disclosure were tested for their ability to reduce STAT3 mRNA expression in IL-6 stimulated PANC-1 cells. The PANC-1 cells were grown in cell culture media and seeded onto a 96 well plate at a density of 20,000 cells/well. ASOs were tested in two separate cohorts: 2 doses or 5 doses were given to the PANC-1 cells (See TABLE 3) at final concentrations of 20 nM and 2 nM, respectively. The assay used Lipofectamine2000 transfection and a 48 hr treatment cycle, with incubation at 37° C. and 5% C02. Analysis of the mRNA expression was then carried out using a branched DNA assay.

TABLE 3

| | | | 5 point DR data | | | | | |
|---|---|---|---|---|---|---|---|---|
| | | | STAT3 | STAT3 | 2 point DR data | | | |
| | | | PANC1 | Max | STAT3 | STAT3 | GAPDH | |
| SEQ ID | starting | | IC50 | inihibition | Inhibition | Inhibition | Inhibition | |
| NO: | base | length | Prism | 24 nM | 20 nM | 2 nM | 20 nM | Notes |
| 103 | 1046 | 15 | 0.465 | 89.93 | 95.8 | 82.5 | 10.00 | 1 CpG, 7 × 1 mm, |
| | | | | | | | | 261 × 2 mm hits |
| 115 | 2267 | 15 | 0.796 | 78.99 | 90.8 | 65.2 | 3.00 | rat, 2 CpG, 3 × 1 mm, |
| | | | | | | | | 143 × 2 mm hits |
| 138 | 1238 | 16 | 1.306 | 85.33 | 86.8 | 53.0 | 9.00 | 2 CpG, 51 × 2 mm hits |
| 112 | 1482 | 16 | 1.041 | 86.32 | 91.0 | 70.0 | −25.00 | 1 CpG, 1 GGGG, |
| | | | | | | | | 90 × 2 mm hits |
| 105 | 450 | 20 | 1.471 | 85.61 | 91.6 | 79.4 | 13.00 | 1 CpG |
| 137 | 516 | 20 | 1.803 | 79.46 | 87.4 | 53.0 | −2.00 | 1 CpG |
| 124 | 995 | 20 | 1.597 | 86.46 | 83.3 | 62.6 | 13.00 | 1 CpG |
| 114 | 2262 | 20 | 0.814 | 84.94 | 87.0 | 66.4 | 10.00 | rat, 2 CpG |
| 102 | 2557 | 20 | 0.473 | 78.02 | 89.5 | 82.7 | 16.00 | 2 CpG, 1 GGGG |
| 107 | 2558 | 20 | 0.037 | 70.92 | 87.7 | 79.0 | 8.00 | 2 CpG, 1 GGGG |
| 116 | 411 | 20 | 1.854 | 69.78 | 85.5 | 64.5 | 16.00 | 1 CpG |
| 120 | 525 | 20 | 2.054 | 87.43 | 93.4 | 62.8 | 12.00 | 2 CpG |
| 130 | 458 | 20 | 1.677 | 72.16 | 80.7 | 59.3 | 13.00 | 1 CpG |
| 134 | 1039 | 20 | 2.127 | 77.94 | 82.8 | 54.3 | 10.00 | 1 CpG |
| 135 | 1238 | 20 | 1.046 | 78.59 | 84.2 | 53.9 | 4.00 | 2 CpG |
| 133 | 2274 | 20 | 2.243 | 81.73 | 90.7 | 55.9 | 14.00 | 1 CpG |
| 125 | 2263 | 20 | 2.296 | 81.67 | 85.1 | 61.7 | 14.00 | 2 CpG |
| 126 | 511 | 20 | 2.783 | 87.71 | 90.7 | 61.4 | 13.00 | 1 CpG |
| 131 | 894 | 17 | 2.825 | 75.20 | 90.7 | 58.9 | 12.00 | 2 CpG, 9 × 2 mm hits |
| 145 | 2273 | 20 | 3.063 | 85.73 | 89.2 | 50.2 | 17.00 | 1 CpG |
| 128 | 1043 | 20 | 3.565 | 79.56 | 81.1 | 61.1 | 19.00 | 1 CpG |
| 139 | 1034 | 20 | 4.023 | 72.50 | 83.4 | 52.8 | 10.00 | 1 CpG |
| Control | 1286 | 20 | 4.103 | 81.62 | 84.5 | 49.6 | 15.00 | no cyno, 9 × 2 mm hits |
| 142 | 1484 | 20 | 6.757 | 76.10 | 80.0 | 51.7 | 9.00 | 1 CpG, 1 GGGG |

### Example 17

### Analysis of NRas mRNA Expression

The ASOs of the present disclosure were tested for their ability to reduce NRas mRNA expression in HEK-293 cells. The HEK-293 cells were grown in cell culture media and seeded onto a 96 well plate at a density of 20,000 cells/well. ASOs were tested in two separate cohorts: 2 doses or 5 doses were given to the HEK-293 cells (See TABLES 4, 5 and 6) at final concentrations of 20 nM and 2 nM respectively. The assay used Lipofectamine2000 transfection and a 48 hr treatment cycle, with incubation at 37° C. and 5% C02. Analysis of the mRNA expression was then carried out using a branched DNA assay. For example, SEQ ID NO: 201 has shown a 98.45% NRas expression inhibition (TABLE 6).

TABLE 4
HEK293 cells: dual-dose

| SEQ ID | MV | SD | MV | |
|---|---|---|---|---|
| NO: | 20 nM | 20 nM | 2 nM | SD 2 nM |
| 201 | 3.5 | 0.4 | 5.8 | 2.3 |
| 274 | 9.6 | 2.5 | 18.6 | 10.2 |
| 239 | 7.8 | 0.7 | 17.9 | 2.2 |
| 200 | 2.8 | 1.0 | 7.8 | 3.1 |
| 272 | 8.7 | 0.6 | 16.5 | 5.4 |
| 264 | 10.4 | 1.1 | 24.7 | 4.3 |
| 232 | 4.4 | 1.3 | 9.8 | 2.2 |
| 281 | 16.2 | 3.4 | 26.4 | 4.1 |
| 248 | 6.0 | 1.4 | 21.8 | 6.1 |
| 252 | 8.3 | 1.4 | 15.2 | 3.9 |
| 254 | 5.8 | 1.1 | 15.6 | 4.6 |
| 236 | 9.8 | 2.1 | 19.4 | 2.6 |
| 273 | 8.1 | 1.0 | 24.3 | 5.2 |
| 257 | 8.6 | 3.7 | 25.9 | 2.8 |
| 227 | 11.2 | 1.3 | 25.1 | 4.3 |
| 218 | 10.7 | 0.4 | 19.1 | 3.6 |
| 253 | 6.5 | 1.0 | 11.6 | 2.0 |
| 206 | 8.7 | 2.6 | 20.2 | 5.7 |
| 231 | 6.4 | 1.3 | 20.2 | 3.9 |
| 247 | 9.8 | 2.0 | 21.3 | 12.5 |
| 223 | 18.7 | 1.1 | 23.9 | 3.3 |
| 238 | 7.6 | 1.9 | 28.8 | 1.9 |
| 250 | 8.6 | 2.1 | 27.3 | 2.7 |
| 224 | 14.4 | 3.8 | 39.6 | 4.7 |

TABLE 5
HEK293 cells: dose-response

| SEQ ID | IC20 | IC50 | IC80 | max. inhib. |
|---|---|---|---|---|
| NO: | [nM]: | [nM]: | [nM]: | [%]: |
| 200 | 0.041 | 0.160 | 0.666 | 97.6 |
| 201 | 0.024 | 0.100 | 0.455 | 97.2 |
| 254 | 0.067 | 0.336 | 2.109 | 93.4 |
| 232 | 0.048 | 0.245 | 1.589 | 93.2 |
| 248 | 0.051 | 0.288 | 2.401 | 91.0 |
| 206 | 0.117 | 0.471 | 2.584 | 90.9 |
| 231 | 0.180 | 0.538 | 2.096 | 90.4 |
| 253 | 0.108 | 0.461 | 2.788 | 90.4 |
| 239 | 0.027 | 0.159 | 1.463 | 90.4 |
| 238 | 0.139 | 0.646 | 4.794 | 88.9 |
| 272 | 0.034 | 0.166 | 1.294 | 88.9 |
| 247 | 0.122 | 0.561 | 4.144 | 88.8 |
| 264 | 0.039 | 0.229 | 2.434 | 88.5 |
| 252 | 0.072 | 0.296 | 1.951 | 88.4 |
| 218 | 0.139 | 0.427 | 1.944 | 88.0 |
| 236 | 0.053 | 0.349 | 4.780 | 87.3 |
| 257 | 0.069 | 0.364 | 3.671 | 87.3 |
| 274 | 0.019 | 0.131 | 2.077 | 86.8 |
| 250 | 0.173 | 0.715 | 5.683 | 86.3 |
| 227 | 0.087 | 0.413 | 4.046 | 86.3 |
| 281 | 0.061 | 0.263 | 2.224 | 86.2 |
| 185 | 0.074 | 0.349 | 3.802 | 85.4 |
| 223 | 0.121 | 0.581 | 35.387 | 80.8 |
| 224 | 0.639 | 1.931 | #N/A | 72.0 |

TABLE 6

| | | | | | 2D |
|---|---|---|---|---|---|
| | NRAS | GAPDH | | | GAPDH |
| SEQ ID | max | max | starting | | 20 nM |
| NO: | inhibition | inhibition | nucleotide | length | inhibition |
| 201 | 98.45 | 45.30 | 181 | 15 | 16.86 |
| 206 | 94.95 | 44.21 | 620 | 15 | 40.87 |
| 218 | 90.00 | 25.71 | 176 | 20 | 28.11 |
| 227 | 87.95 | 18.30 | 378 | 20 | 24.56 |
| 236* | 91.51 | 40.06 | 421 | 20 | 34.05 |
| 239 | 94.35 | 37.62 | 490 | 20 | 39.01 |
| 252 | 91.49 | 29.98 | 604 | 20 | 13.40 |
| 264 | 91.73 | 26.13 | 623 | 20 | 10.43 |
| 272 | 90.39 | 40.47 | 922 | 20 | 23.00 |
| 274 | 89.15 | 35.71 | 1074 | 20 | 24.93 |
| 231 | 93.63 | 29.21 | 399 | 20 | 36.92 |
| 223 | 85.75 | 18.44 | 325 | 20 | 20.53 |
| 247 | 90.09 | 25.35 | 534 | 20 | 25.39 |
| 257 | 91.79 | 30.55 | 615 | 20 | 21.09 |
| 238 | 90.92 | 26.33 | 429 | 20 | 22.48 |
| 253 | 94.40 | 29.34 | 611 | 20 | 40.44 |
| 250 | 88.02 | 33.44 | 537 | 20 | 27.30 |
| 200 | 98.68 | 38.68 | 180 | 15 | 33.52 |
| 232* | 95.91 | 53.16 | 400 | 20 | 36.10 |
| 281 | 90.44 | 50.48 | 1623 | 20 | 23.62 |
| 248 | 93.72 | 37.96 | 535 | 20 | 30.77 |
| 254 | 96.11 | 32.96 | 612 | 20 | 20.10 |
| 273 | 92.14 | 54.95 | 1072 | 20 | 26.53 |

### Example 18

### Construction of an Exosome

To generate exosomes described herein, human embryonic kidney (HEK) cell line (e.g., HEK293SF) will be used. The cells will be stably transfected with Scaffold X, Scaffold Y, and/or anchoring moiety linked to an agent of interest (e.g., antigen, adjuvant, or immune modulator). For example, CD40L-expressing exosomes can be generated by transfecting HEK293SF cells with CD40L-GFP PTGFRN fusion molecules, which express as a monomer or as a forced trimer.

Upon transfection, HEK cells will be grown to high density in chemically defined medium for 7 days. Conditioned cell culture media will be then collected and centrifuged at 300-800×g for 5 minutes at room temperature to remove cells and large debris. Media supernatant will be supplemented with 1000 U/L BENZONASE® and incubated at 37° C. for 1 hour in a water bath. Supernatant will be collected and centrifuged at 16,000×g for 30 minutes at 4° C. to remove residual cell debris and other large contaminants. Supernatant will then be ultracentrifuged at 133,900×g for 3 hours at 4° C. to pellet the exosomes. Supernatant will be discarded and any residual media will be aspirated from the bottom of the tube. The pellet will be resuspended in 200-1000 μL PBS (—Ca —Mg).

To further enrich exosome populations, the pellet will be processed via density gradient purification (sucrose or OPTIPREP™).

The gradient will be spun at 200,000×g for 16 hours at 4° C. in a 12 mL Ultra-Clear (344059) tube placed in a SW 41 Ti rotor to separate the exosome fraction.

The exosome layer will then be gently removed from the top layer and diluted in ˜32.5 mL PBS in a 38.5 mL Ultra-Clear (344058) tube and ultracentrifuged again at 133,900×g for 3 hours at 4° C. to pellet the purified exosomes. The resulting pellet will be resuspended in a minimal volume of PBS (˜200 μL) and stored at 4° C.

For OPTIPREP™ gradient, a 3-tier sterile gradient will be prepared with equal volumes of 10%, 30%, and 45% OPTIPREP™ in a 12 mL Ultra-Clear (344059) tube for a SW 41 Ti rotor. The pellet will be added to the OPTIPREP™ gradient and ultracentrifuged at 200,000×g for 16 hours at 4° C. to separate the exosome fraction. The exosome layer will then be gently collected from the top ˜3 mL of the tube.

The exosome fraction will be diluted in −32 mL PBS in a 38.5 mL Ultra-Clear (344058) tube and ultracentrifuged at 133,900×g for 3 hours at 4° C. to pellet the purified exosomes. The pelleted exosomes will then be resuspended in a minimal volume of PBS (˜200 μL) and stored at 4° C. until ready to be used.

### Example 19

### Effect of ASO Linker Structure on Potency and Amount Loaded in Engineered or Native Exosomes

The effect of ASO linker structure on amount of ASO loaded in engineered or native exosomes, as well as the effect of ASO linker structure on ASO potency were evaluated.

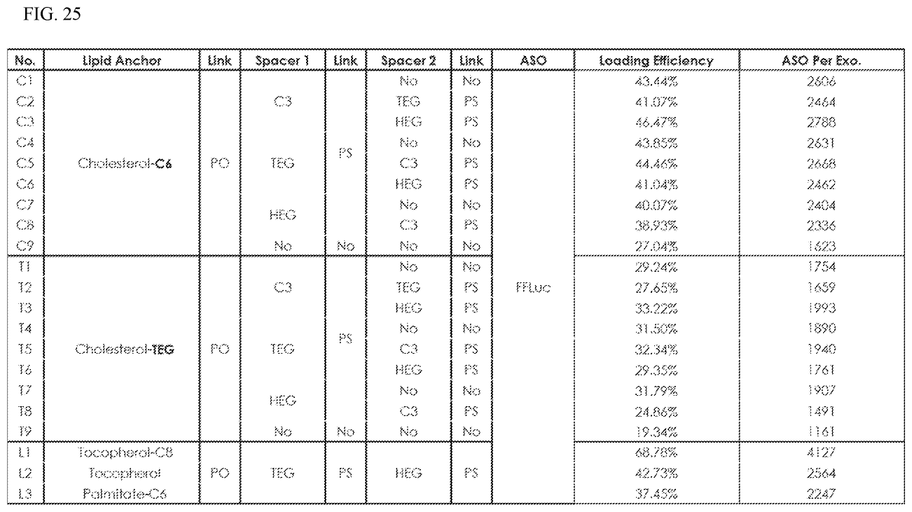

Loading effectiveness of a fixed ASO sequence conjugated to different linkers was evaluated in engineered exosomes. Exosomes over-expressing PTGFRN were produced in HEK293 cells and purified. ASO payloads were loaded on to the surface of exosomes by mixing. Accordingly, exosomes at a concentration of 1×10¹³ P/mL were mixed 1:1 with 100 uM ASO with sequence for firefly luciferase (FFLuc) that had been conjugated to different linker structures. The loading efficiency was calculated as the percentage of starting ASOs that became coupled to the exosomes. FIG. 25 shows the structures of the constructs used. Constructs C1 to C9 used a cholesterol-C6 lipid anchor. Constructs T1 to T9 used a cholesterol-TEG lipid anchor. Construct L1 used s tocopherol-C8 lipid anchors whereas constructs L2 and L3 used a tocopherol palitate-C6 lipid anchor. Other linker components indicated in FIG. 25 are: phosphodiester (PO), phosphothioate (PS), hexamethylene (C6), trimethylene (C3), triethylene glycol (TEG), and hexaethylene glycol (HEG). The results indicated that the mount of ASO molecules loaded per engineered exosome was affected by linker structure (FIG. 25). In general, loading efficiency was higher for constructs with the cholesterol-C6 anchor than for constructs with the cholesterol-TEG anchort. The highest loading efficacy, 68.78%, was observed for constructs with a tocopherol-C8 lipid anchor, corresponding to a load of 4,167 ASO units per exosome.

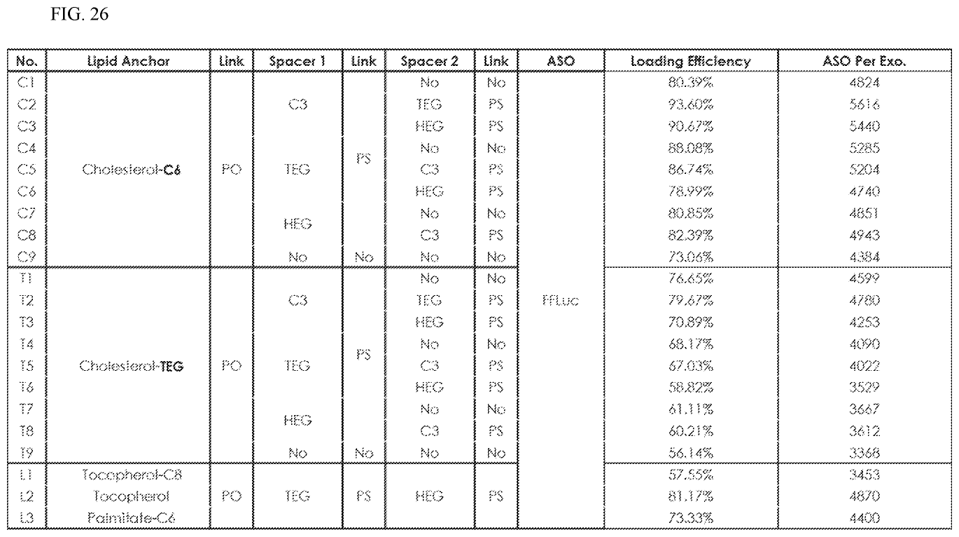

The loading effectiveness of the same fixed ASO sequence conjugated to different linkers evaluated in FIG. 25 using engineered exosomes was also evaluated using native exosomes. Native exosomes were produced in HEK293 cells and purified. Exosomes at a concentration of 1×10¹³ P/mL were mixed 1:1 with 100 uM ASO with a sequence for firefly luciferase that had been conjugated to different linker structures. The loading efficiency was calculate as the percentage of starting ASOs that became coupled to the exosomes. As in the case of engineered exosomes, the amount of ASO molecules loaded per native exosome was affected by linker structure (FIG. 26). Again, constructs with a cholesterol-C6 lipid anchor showed higher loading efficacy than constructs with a cholesterol-TEG lipid anchor. Loading efficacies over 90% were observed for constructs C2 and C3 (93.6% and 90.67%), corresponding to 5,616 and 5,440 ASO units per exosome, respectively. Thus, the maximum load efficacies and number of ASO per exosome were significantly higher in native exosomes than in engineered exosomes.

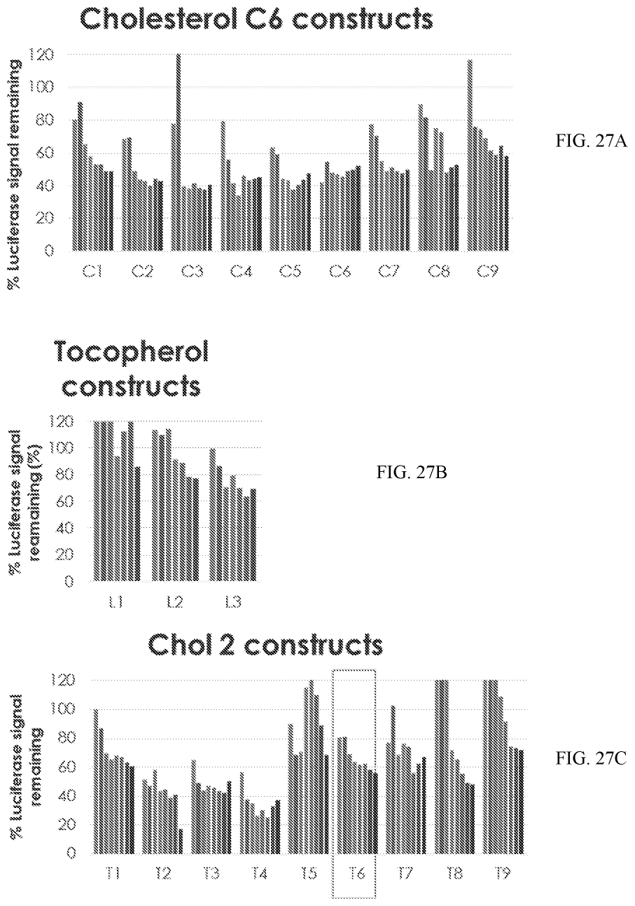

The activity of the constructs loaded onto native exosomes was also evaluated. Native exosomes were produced in HEK293 cells and purified. Exosomes at concentration of 1°10¹³ P/mL were mixed 1:1 with 100 uM ASO with a sequence for firefly luciferase conjugated to different linkers (see FIGS. 25 and 26). The in vitro potency of the exoASO constructs was evaluated in H1299 cells expressing firefly and Renilla luciferase. Potency was measured by the amount of luminescence remaining after incubation with the cells. Using T6 (boxed) as the reference, the data was normalized to the amount of ASO per sample as measured with Ribogreen. The potenty of C₁-C₉ constructs (FIG. 27A) and T1-T9 constructs (FIG. 27C) was higher than for L1-L3 constructs (FIG. 27B).

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that may be cited throughout this application are hereby expressly incorporated by reference in their entirety for any purpose, as are the references cited therein.

What is claimed is:

1. An extracellular vesicle (EV) comprising a biologically active molecule (BAM) covalently linked to the EV via an anchoring moiety (AM), wherein the anchoring moiety comprises:
[AM]-[Linker]n-[BAM] Formula (I)
wherein n is any integer.

2. The extracellular vesicle of claim 1, wherein n is any number between 0 and 10.

3. The extracellular vesicle of claim 1 or 2, wherein the anchoring moiety comprises a sterol, GM1, a lipid, a vitamin, a small molecule, a peptide, or a combination thereof.

4. The extracellular vesicle of any one of claims 1 to 3, wherein the anchoring moiety comprises at least 6 carbon atoms, at least 7 carbon atoms, at least 8 carbon atoms, at least 9 carbon atoms, at least 10 carbon atoms, at least 11 carbon atoms, at least 12 carbon atoms, at least 13 carbon atoms, at least 14 carbon atoms, at least 15 carbon atoms, at least 16 carbon atoms, at least 17 carbon atoms, at least 18 carbon atoms, at least 19 carbon atoms, at least 20 carbon atoms, at least 25 carbon atoms, at least 30 carbon atoms, at least 35 carbon atoms, at least 40 carbon atoms, at least 45 carbon atoms, at least 50 carbon atoms, at least 55 carbon atoms, at least 60 carbon atoms, at least 65 carbon atoms, at least 70 carbon atoms, at least 75 carbon atoms, at least 80 carbon atoms, at least 85 carbon atoms, or at least 90 carbon atoms.

5. The extracellular vesicle of any one of claims 1 to 4, wherein the anchoring moiety comprises a sterol, a steroid, a hopanoid, a hydroxysteroid, a secosteroid, an analog thereof, or any combination thereof.

6. The extracellular vesicle of any one of claims 1 to 4, wherein the anchoring moiety comprises ergosterol, 7-dehydrocholesterol, cholesterol, 24S-hydroxycholesterol, lanosterol, cycloartenol, fucosterol, saringosterol, campesterol, β-sitosterol, sitostanol, coprostanol, avenasterol, stigmasterol, or any combination thereof.

7. The extracellular vesicle of claim 6, wherein the anchoring moiety is a cholesterol or derivative having a structure selected from the group consisting of

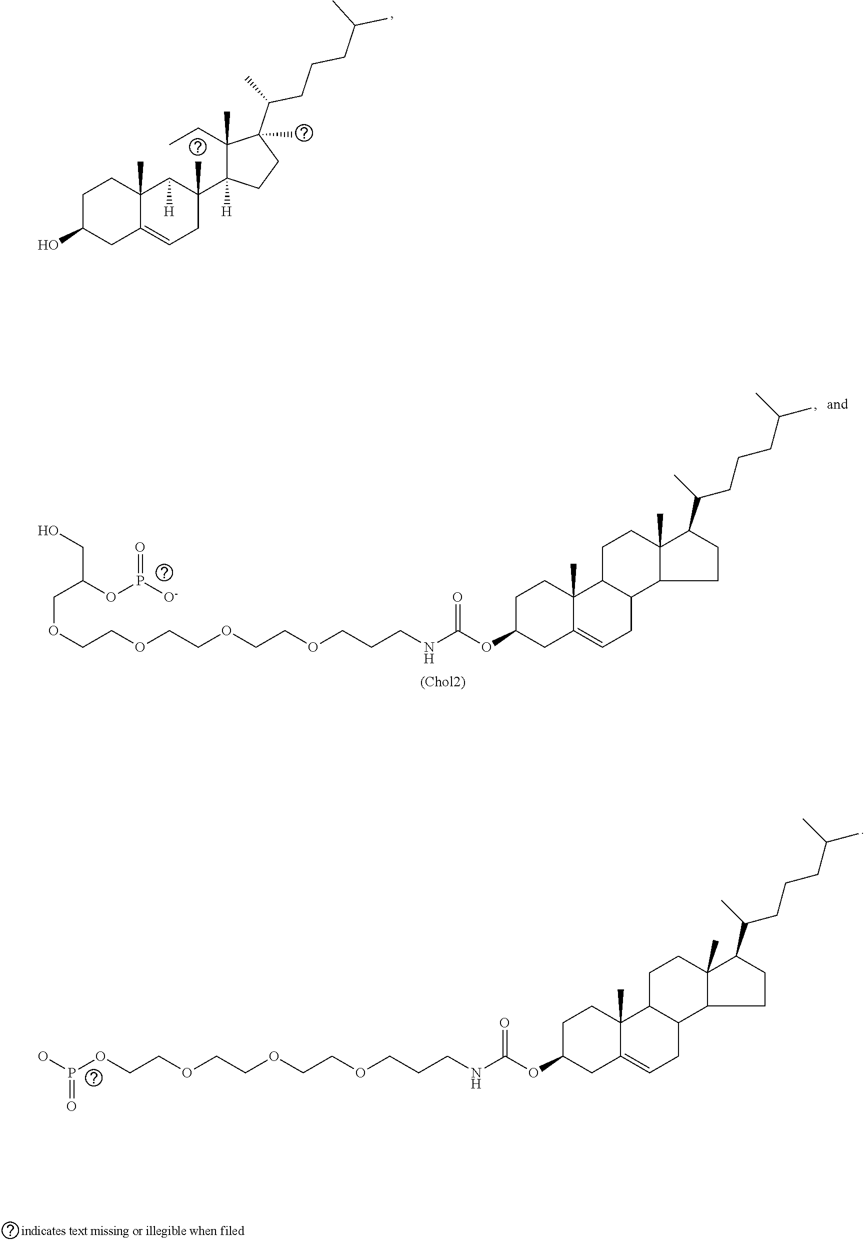

8. The extracellular vesicle of any one of claims 1 to 5, wherein the anchoring moiety comprises a steroid, which is dihydrotestosterone, uvaol, hecigenin, diosgenin, progesterone, cortisol, or any combination thereof.

9. The extracellular vesicle of any one of claims 1 to 4, wherein the anchoring moiety comprises a lipid.

10. The extracellular vesicle of claim 9, wherein the anchoring moiety comprises a C₂-C₆₀ chain.

11. The extracellular vesicle of claim 9, wherein the anchoring moiety comprises C₄-C₄₀, C₂-C₃₈, C₂-C₃₆, C₂-C₃₄, C₂-C₃₂, C₂-C₃₀, C₄-C₃₀, C₂-C₂₈, C₄-C₂₈, C₂-C₂₆, C₄-C₂₆, C₂-C₂₄, C₄-C₂₄, C₆-C₂₄, C₈-C₂₄, C₁₀-C₂₄, C₂-C₂₂, C₄-C₂₂, C₆-C₂₂, C₈-C₂₂, C₁₀-C₂₂, C₂-C₂₀, C₄-C₂₀, C₆-C₂₀, C₈-C₂₀, C₁₀-C₂₀, C₂-C₁₈, C₄-C₁₈, C₆-C₁₈, C₈-C₁₈, C₁₀-C₁₈, C₁₂-C₁₈, C₁₄-C₁₈, C₁₆-C₁₈, C₂-C₁₆, C₄-C₁₆, C₆-C₁₆, C₈-C₁₆, C₁₀-C₁₆, C₁₂-C₁₆, C₁₄-C₁₆, C₂-C₁₅, C₄-C₁₅, C₆-C₁₅, C₈-C₁₅, C₉-C₁₅, C₁₀-C₁₅, C₁₁-C₁₅, C₁₂-C₁₅, C₁₃-C₁₅, C₂-C₁₄, C₄-C₁₄, C₆-C₁₄, C₈-C₁₄, C₉-C₁₄, C₁₀-C₁₄, C₁₁-C₁₄, C₁₂-C₁₄, C₂-C₁₃, C₄-C₁₃, C₆-C₁₃, C₇-C₁₃, C₈-C₁₃, C₉-C₁₃, C₁₀-C₁₃, C₁₀-C₁₃, C₁₁-C₁₃, C₂-C₁₃, C₄-C₁₂, C₆-C₁₂, C₇-C₁₂, C₈-C₁₂, C₉-C₁₂, C₁₀-C₁₂, C₂-C₁₁, C₄-C₁₁, C₆-C₁₁, C₇-C₁₁, C₈-C₁₁, C₉-C₁₁, C₂-C₁₀, C₄-C₁₀, C₂-C₉, C₄-C₉, C₂-C₈, C₂-C₇, C₄-C₇, C₂-C₆, or C₄-C₆ chain.

12. The extracellular vesicle of any one of claims 9 to 11, wherein the anchoring moiety comprises a straight chain fatty acid, a branched fatty acid, an unsaturated fatty acid, a monounsaturated fatty acid, a polyunsaturated fatty acid, a hydroxyl fatty acid, a polycarboxylic acid, or any combination thereof.

13. The extracellular vesicle of claim 12, wherein the anchoring moiety comprises a straight chain fatty acid, which is butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid, heptacosanoic acid, or any combination thereof.

14. The extracellular vesicle of claim 12, wherein the anchoring moiety comprises a branched fatty acid, which is isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, α-heptylundecanoic acid, 2-decyltetradecanoic acid, 2-undecyltetradecanoic acid, 2-decylpentadecanoic acid, 2-undecylpentadecanoic acid, Fine oxocol 1800 acid (product of Nissan Chemical Industries, Ltd.), anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid, and 26-methyloctacosanoic acid, or any combination thereof.

15. The extracellular vesicle of claim 12, wherein the anchoring moiety comprises an unsaturated fatty acid, which is 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid, 7,10,13,16,19-docosapentaenoic acid, 4,7,10,13,16,19-docosahexaenoic acid, or any combination thereof.

16. The extracellular vesicle of claim 12, wherein the anchoring moiety comprises a hydroxy fatty acid, which is α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid, or any combination thereof.

17. The extracellular vesicle of claim 12, wherein the anchoring moiety comprises a polycarboxylic acid, which is oxalic acid, malonic acid, succinic acid, glutaric acid, adipic acid, pimelic acid, suberic acid, azelaic acid, sebacic acid, D,L-malic acid, or any combination thereof.

18. The extracellular vesicle of any one of claims 1 to 4, wherein the anchoring moiety comprises a phospholipid.

19. The extracellular vesicle of claim 18, wherein the phospholipid is phosphatidyl choline, phosphatidyl ethanolamine, phosphatidyl glycerol, phosphatidyl serine, phosphatidic acid, 2 lysophosphatidyl choline, sphingomyelin, or any combination thereof.

20. The extracellular vesicle of claim 18, wherein the phospholipid is phosphatidylethanolamines, which is dilauroylphosphatidyl ethanolamine, dimyristoylphosphatidyl ethanolamine, dipalmitoylphosphatidyl ethanolamine, distearoylphosphatidyl ethanolamine, dioleoylphosphatidyl ethanolamine, 1-palmitoyl-2-oleylphosphatidyl ethanolamine, 1-oleyl-2-palmitoylphosphatidyl ethanolamine, dierucoylphosphatidyl ethanolamine, or any combination thereof.

21. The extracellular vesicle of claim 18, wherein the phospholipid is phosphatidyl glycerol, which is dilauroylphosphatidyl glycerol, dimyristoylphosphatidyl glycerol, dipalmitoylphosphatidyl glycerol, distearoylphosphatidyl glycerol, dioleoylphosphatidyl glycerol, 1-palmitoyl-2-oleyl-phosphatidyl glycerol, 1-oleyl-2-palmitoyl-phosphatidyl glycerol, dierucoylphosphatidyl glycerol, or any combination thereof.

22. The extracellular vesicle of claim 18, wherein the phospholipid is phosphatidyl serine, which is dilauroylphosphatidyl serine, dimyristoylphosphatidyl serine, dipalmitoylphosphatidyl serine, distearoylphosphatidyl serine, dioleoylphosphatidyl serine, 1-palmitoyl-2-oleyl-phosphatidyl serine, 1-oleyl-2-palmitoyl-phosphatidyl serine, dierucoylphosphatidyl serine, or any combination thereof.

23. The extracellular vesicle of claim 18, wherein the phospholipid is phosphatidic acid, which is dilauroylphosphatidic acid, dimyristoylphosphatidic acid, dipalmitoylphosphatidic acid, distearoylphosphatidic acid, dioleoylphosphatidic acid, 1-palmitoyl-2-oleylphosphatidic acid, 1-oleyl-2-palmitoyl-phosphatidic acid, dierucoylphosphatidic acid, or any combination thereof.

24. The extracellular vesicle of claim 18, wherein the phospholipid is phosphatidyl inositol, which is dilauroylphosphatidyl inositol, dimyristoylphosphatidyl inositol, dipalmitoylphosphatidyl inositol, distearoylphosphatidyl inositol, dioleoylphosphatidyl inositol, 1-palmitoyl-2-oleyl-phosphatidyl inositol, 1-oleyl-2-palmitoyl-phosphatidyl inositol, dierucoylphosphatidyl inositol, or any combination thereof.

25. The extracellular vesicle of claim 18, wherein the phospholipid is a symmetric phospholipid, which is 1,2 dipropionyl sn-glycero 3 phosphocholine (03:0 PC); 1,2 dibutyryl sn glycero 3 phosphocholine (04:0 PC); 1,2 dipentanoyl sn glycero 3 phosphocholine (05:0 PC); 1,2 dihexanoyl sn glycero 3 phosphocholine (06:0 PC), 1,2 diheptanoyl sn glycero 3 phosphocholine (07:0 PC); 1,2 dioctanoyl sn glycero 3 phosphocholine (08:0 PC); 1,2 dinonanoyl sn glycero 3 phosphocholine (09:0 PC); 1,2 didecanoyl sn glycero 3 phosphocholine (10:0 PC); 1,2 diundecanoyl sn glycero 3 phosphocholine (11:0 PC, DUPC); 1,2 dilauroyl sn glycero 3 phosphocholine (12:0 PC); 1,2 ditridecanoyl sn glycero 3 phosphocholine (13:0 PC); 1,2 dimyristoyl sn glycero 3 phosphocholine (14:0 PC, DMPC); 1,2 dipentadecanoyl sn glycero 3 phosphocholine (15:0 PC; 1,2 dipalmitoyl sn glycero 3 phosphocholine (16:0 PC, DPPCQ 1,2 diphytanoyl sn glycero 3 phosphocholine (4ME 16:0 PC); 1,2 diheptadecanoyl sn glycero 3 phosphocholine (17:0 PC); 1,2 distearoyl sn glycero 3 phosphocholine (18:0 PC, DSPC); 1,2 dinonadecanoyl sn glycero 3 phosphocholine (19:0 PC); 1,2 diarachidoyl sn glycero 3 phosphocholine (20:0 PC); 1,2 dihenarachidoyl sn glycero 3 phosphocholine (21:0 PC); 1,2 dibehenoyl sn glycero 3 phosphocholine (22:0 PC); 1,2 ditricosanoyl sn glycero 3 phosphocholine (23:0 PC); 1,2 dilignoceroyl sn glycero 3 phosphocholine (24:0 PC); 1,2 dimyristoleoyl sn glycero 3 phosphocholine (14:1 (Δ9-Cis) PC); 1,2 dimyristelaidoyl sn glycero 3 phosphocholine (14:1 (Δ9-Trans) PC); 1,2 dipalmitoleoyl sn glycero 3 phosphocholine (16:1 (Δ9-Cis) PC); 1,2 dipalmitelaidoyl sn glycero 3 phosphocholine (16:1 (Δ9-Trans) PC); 1,2 dipetroselenoyl sn glycero 3 phosphocholine (18:1 (Δ6-Cis) PC); 1,2 dioleoyl sn glycero 3 phosphocholine (18:1 (Δ9-Cis) PC, DOPC); 1,2 dielaidoyl sn glycero 3 phosphocholine (18:1 (Δ9-Trans) PC); 1,2 dilinoleoyl sn glycero 3 phosphocholine (18:2 (Cis) PC, DLPC); 1,2 dilinolenoyl sn glycero 3 phosphocholine (18:3 (Cis) PC, DLnPC); 1,2 dieicosenoyl sn glycero 3 phosphocholine (20:1 (Cis) PC); 1,2 diarachidonoyl sn glycero 3 phosphocholine (20:4 (Cis) PC, DAPC); 1,2 dierucoyl sn glycero 3 phosphocholine (22:1 (Cis) PC); 1,2 didocosahexaenoyl sn glycero 3 phosphocholine (22:6 (Cis) PC, DHAPC); 1,2 dinervonoyl sn glycero 3 phosphocholine (24:1 (Cis) PC); 1,2 dihexanoyl sn glycero 3 phosphoethanolamine (06:0 PE); 1,2 dioctanoyl sn glycero 3 phosphoethanolamine (08:0 PE); 1,2 didecanoyl sn glycero 3 phosphoethanolamine (10:0 PE); 1,2 dilauroyl sn glycero 3 phosphoethanolamine (12:0 PE), 1,2 dimyristoyl sn glycero 3 phosphoethanolamine (14:0 PE); 1,2 dipentadecanoyl sn glycero 3 phosphoethanolamine (15:0 PE); 1,2 dipalmitoyl sn glycero 3 phosphoethanolamine (16:0 PE); 1,2 diphytanoyl sn glycero 3 phosphoethanolamine (4ME 16:0 PE); 1,2 diheptadecanoyl sn glycero 3 phosphoethanolamine (17:0 PE); 1,2 distearoyl sn glycero 3 phosphoethanolamine (18:0 PE, DSPE); 1,2 dipalmitoleoyl sn glycero 3 phosphoethanolamine (16:1 PE); 1,2 dioleoyl sn glycero 3 phosphoethanolamine (18:1 (Δ9-Cis) PE, DOPE); 1,2 dielaidoyl sn glycero 3 phosphoethanolamine (18:1 (Δ9-Trans) PE); 1,2 dilinoleoyl sn glycero 3 phosphoethanolamine (18:2 PE, DLPE); 1,2 dilinolenoyl sn glycero 3 phosphoethanolamine (18:3 PE, DLnPE); 1,2 diarachidonoyl sn glycero 3 phosphoethanolamine (20:4 PE, DAPE); 1,2 didocosahexaenoyl sn glycero 3 phosphoethanolamine (22:6 PE, DHAPE); 1,2 di O octadecenyl sn glycero 3 phosphocholine (18:0 Diether PC); 1,2 dioleoyl sn glycero 3 phospho rac (1 glycerol) sodium salt (DOPG), or any combination thereof.

26. The extracellular vesicle of claim 18, wherein the phospholipid is a asymmetric phospholipid, which is 1 myristoyl 2 palmitoyl sn glycero 3 phosphocholine (14:0-16:0 PC, MPPC); 1 myristoyl 2 stearoyl sn glycero 3 phosphocholine (14:0-18:0 PC, MSPC); 1 palmitoyl 2 acetyl sn glycero 3 phosphocholine (16:0-02:0 PC); 1 palmitoyl 2 myristoyl sn glycero 3 phosphocholine (16:0-14:0 PC, PMPC); 1 palmitoyl 2 stearoyl sn glycero 3 phosphocholine (16:0-18:0 PC, PSPC); 1 palmitoyl 2 oleoyl sn glycero 3 phosphocholine (16:0-18:1 PC, POPC); 1 palmitoyl 2 linoleoyl sn glycero 3 phosphocholine (16:0-18:2 PC, PLPC); 1 palmitoyl 2 arachidonoyl sn glycero 3 phosphocholine (16:0-20:4 PC); 1 palmitoyl 2 docosahexaenoyl sn glycero 3 phosphocholine (14:0-22:6 PC); 1 stearoyl 2 myristoyl sn glycero 3 phosphocholine (18:0-14:0 PC, SMPC); 1 stearoyl 2 palmitoyl sn glycero 3 phosphocholine (18:0-16:0 PC, SPPC); 1 stearoyl 2 oleoyl sn glycero 3 phosphocholine (18:0-18:1 PC, SOPC); 1 stearoyl 2 linoleoyl sn glycero 3 phosphocholine (18:0-18:2 PC); 1 stearoyl 2 arachidonoyl sn glycero 3 phosphocholine (18:0-20:4 PC); 1 stearoyl 2 docosahexaenoyl sn glycero 3 phosphocholine (18:0-22:6 PC); 1 oleoyl 2 myristoyl sn glycero 3 phosphocholine (18:1-14:0 PC, OMPC); 1 oleoyl 2 palmitoyl sn glycero 3 phosphocholine (18:1-16:0 PC, OPPC); 1 oleoyl 2 stearoyl sn glycero 3 phosphocholine (18:1-18:0 PC, OSPC); 1 palmitoyl 2 oleoyl sn glycero 3 phosphoethanolamine (16:0-18:1 PE, POPE); 1 palmitoyl 2 linoleoyl sn glycero 3 phosphoethanolamine (16:0-18:2 PE); 1 palmitoyl 2 arachidonoyl sn glycero 3 phosphoethanolamine (16:0-20:4 PE); 1 palmitoyl 2 docosahexaenoyl sn glycero 3 phosphoethanolamine (16:0-22:6 PE); 1 stearoyl 2 oleoyl sn glycero 3 phosphoethanolamine (18:0-18:1 PE); 1 stearoyl 2 linoleoyl sn glycero 3 phosphoethanolamine (18:0-18:2 PE); 1 stearoyl 2 arachidonoyl sn glycero 3 phosphoethanolamine (18:0-20:4 PE); 1 stearoyl 2 docosahexaenoyl sn glycero 3 phosphoethanolamine (18:0-22:6 PE); 1 oleoyl 2 cholesterylhemisuccinoyl sn glycero 3 phosphocholine (OChemsPC), or any combination thereof.

27. The extracellular vesicle of claim 8, wherein the phospholipid is a lysolipid.

28. The extracellular vesicle of claim 27, wherein the phospholipid is a lysoglycerophospholipid, a lysoglycosphingoliopid, a lysophosphatidylcholine, a lysophosphatidylethanolamine, a lysophosphatidylinositol, a lysophosphatidylserine, or any combination thereof.

29. The extracellular vesicle of claim 27, wherein the phospholipid is 1 hexanoyl 2 hydroxy sn glycero 3 phosphocholine (06:0 Lyso PC); 1 heptanoyl 2 hydroxy sn glycero 3 phosphocholine (07:0 Lyso PC); 1 octanoyl 2 hydroxy sn glycero 3 phosphocholine (08:0 Lyso PC); 1 nonanoyl 2 hydroxy sn glycero 3 phosphocholine (09:0 Lyso PC); 1 decanoyl 2 hydroxy sn glycero 3 phosphocholine (10:0 Lyso PC); 1 undecanoyl 2 hydroxy sn glycero 3 phosphocholine (11:0 Lyso PC); 1 lauroyl 2 hydroxy sn glycero 3 phosphocholine (12:0 Lyso PC); 1 tridecanoyl 2 hydroxy sn glycero 3 phosphocholine (13:0 Lyso PC); 1 myristoyl 2 hydroxy sn glycero 3 phosphocholine (14:0 Lyso PC); 1 pentadecanoyl 2 hydroxy sn glycero 3 phosphocholine (15:0 Lyso PC); 1 palmitoyl 2 hydroxy sn glycero 3 phosphocholine (16:0 Lyso PC); 1 heptadecanoyl 2 hydroxy sn glycero 3 phosphocholine (17:0 Lyso PC); 1 stearoyl 2 hydroxy sn glycero 3 phosphocholine (18:0 Lyso PC); 1 oleoyl 2 hydroxy sn glycero 3 phosphocholine (18:1 Lyso PC); 1 nonadecanoyl 2 hydroxy sn glycero 3 phosphocholine (19:0 Lyso PC); 1 arachidoyl 2 hydroxy sn glycero 3 phosphocholine (20:0 Lyso PC); 1 behenoyl 2 hydroxy sn glycero 3 phosphocholine (22:0 Lyso PC); 1 lignoceroyl 2 hydroxy sn glycero 3 phosphocholine (24:0 Lyso PC); 1 hexacosanoyl 2 hydroxy sn glycero 3 phosphocholine (26:0 Lyso PC); 1 myristoyl 2 hydroxy sn glycero 3 phosphoethanolamine (14:0 Lyso PE); 1 palmitoyl 2 hydroxy sn glycero 3 phosphoethanolamine (16:0 Lyso PE); 1 stearoyl 2 hydroxy sn glycero 3 phosphoethanolamine (18:0 Lyso PE); 1 oleoyl 2 hydroxy sn glycero 3 phosphoethanolamine (18:1 Lyso PE); 1 hexadecyl sn glycero 3 phosphocholine (C16 Lyso PC); or any combination thereof.

30. The extracellular vesicle of any one of claims 1 to 5, wherein the anchoring moiety comprises a vitamin.

31. The extracellular vesicle of any one of claims 1 to 5, wherein the anchoring moiety comprises vitamin D, vitamin K, vitamin E, or any combination thereof.

32. The extracellular vesicle of any one of claims 1 to 31, wherein the anchoring moiety further comprises a linker between the biologically active molecule and the anchoring moiety.

33. The extracellular vesicle of claim 32, wherein the linker comprises a non-cleavable linker.

34. The extracellular vesicle of claim 33, wherein the non-cleavable linker comprises polyethylene glycol (PEG), glycerol, alkyl, succinimide, maleimide, or any combination thereof.

35. The extracellular vesicle of claim 33, wherein the non-cleavable linker comprises polyethylene glycol (PEG) characterized by a formula R3-(O—CH₂—CH₂)ₙ— or R3-(0-CH₂—CH₂)ₙ—O—, wherein R³ being hydrogen, methyl or ethyl and n is an integer between 2 and 200.

36. The extracellular vesicle of claim 33, wherein the non-cleavable linker comprises diethylene glycol, triethylene glycol, tetraethylene glycol (TEG), hexaethylene glycol (HEG), pentaethylene glycol, or any combination thereof.

37. The extracellular vesicle of any one of claims 32 to 34, wherein the linker comprises a polyglycerol (PG) having the formula ((R3-O—(CH₂—CHOH—CH₂O)ₙ—), wherein R³ is hydrogen, methyl or ethyl, and n is an integer between 3 and 200.

38. The extracellular vesicle of any one of claims 32 to 34, wherein the linker comprises a diglycerol, triglycerol, tetraglycerol (TG), pentaglycerol, a hexaglycerol (HG), or any combination thereof.

39. The extracellular vesicle of any one of claims 32 to 34, wherein the linker comprises alkyl.

40. The extracellular vesicle of any one of claims 32 to 34, wherein the linker comprises alkenyl, alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, Aryl, heteroaryl, heterocyclyl, cycloalkyl, cycloalkenyl, alkylarylalkyl, alkylarylalkenyl, alkylarylalkynyl, alkenylarylalkyl, alkenyl Reyl alkenyl, alkenyl aryl alkynyl, alkynyl aryl alkyl, alkynyl aryl alkenyl, alkynyl aryl alkynyl, alkyl heteroaryl alkyl, alkyl heteroaryl alkyl, alkyl heteroaryl alkenyl, alkyl heteroaryl alkynyl, alkenyl heteroaryl alkyl, alkenyl heteroaryl alkenyl, alkenyl heteroaryl alkynyl, alkynyl Heteroarylalkyl, alkynylheteroarylalkenyl, alkynylheteroarylalkynyl, alkylheterocyclylalkyl, alkylheterocyclylalkenyl, alkylheterocyclylalkynyl, alkenylheterocyclylalkyl, alkenylheterocyclylalkenyl, alkenylheterocyclylalkynyl, or any combination thereof.

41. The extracellular vesicle of claim 32, wherein the linker comprises a cleavable linker.

42. The extracellular vesicle of claim 41, wherein the cleavable linker is a redox cleavable linker, a reactive oxygen species cleavable linker, a pH dependent cleavable linker, an enzymatic cleavable linker, a protease cleavable linker, an esterase cleavable linker, a phosphatase cleavable linker, a photoactivated cleavable linker, a self-immolative linker, or any combination thereof.

43. The extracellular vesicle of claim 41, wherein the cleavable linker is a self-immolative linker.

44. The extracellular vesicle of claim 41, wherein the cleavable linker is a cinnamyl group, a naphthyl group, a biphenyl group, a heterocyclic ring, a homoaromatic group, coumarin, furan, thiophene, thiazole, oxazole, isoxazole, pyrrole, pyrazole, pyridine, imidazone, triazole, or any combination thereof.

45. The extracellular vesicle of any one of claims 41 to 44, wherein the linker has the formula:
-Aₐ-Y_{y}-
wherein each -A- is independently an amino acid unit, a is independently an integer from 1 to 12; -Y- is a spacer unit, and y is 0, 1, or 2.

46. The extracellular vesicle of claim 45, wherein -Aₐ- is a dipeptide, a tripeptide, a tetrapeptide, a pentapeptide, or a hexapeptide.

47. The extracellular vesicle of claim 46, wherein a is 2 and -Aₐ- is selected from the group consisting of valine-alanine, valine-citrulline, phenylalanine-lysine, N-methylvaline-citrulline, cyclohexylalanine-lysine, and beta-alanine-lysine.

48. The extracellular vesicle of claim 46, wherein said -Aₐ- is valine-alanine or valine-citrulline.

49. The extracellular vesicle of any one of claims 45 to 48, wherein y is 1.

50. The extracellular vesicle of any one of claims 45 to 49, wherein -Y- is a self-immolative spacer.

51. The extracellular vesicle of claim 50, wherein -Y_{y}- has the formula (V): 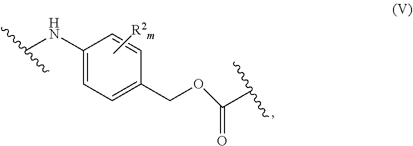
wherein each R² is independently C₁₋₈ alkyl, —O—(C₁₋₈ alkyl), halogen, nitro, or cyano; and m is an integer from 0 to 4.

52. The extracellular vesicle of claim 51, wherein m is 0, 1, or 2.

53. The extracellular vesicle of claim 51, wherein m is 0.

54. The extracellular vesicle of any one of claims 45 to 53, wherein the cleavable linker is valine-alanine-p-aminobenzylcarbamate or valine-citrulline-p-aminobenzylcarbamate.

55. The extracellular vesicle of any one of claims 45 to 49, wherein -Y- is a non self-immolative spacer.

56. The extracellular vesicle of claim 55, wherein the non self-immolative spacer is -Gly- or -Gly-Gly-.

57. The extracellular vesicle of any one of claims 1 to 4, wherein the anchoring moiety comprises:

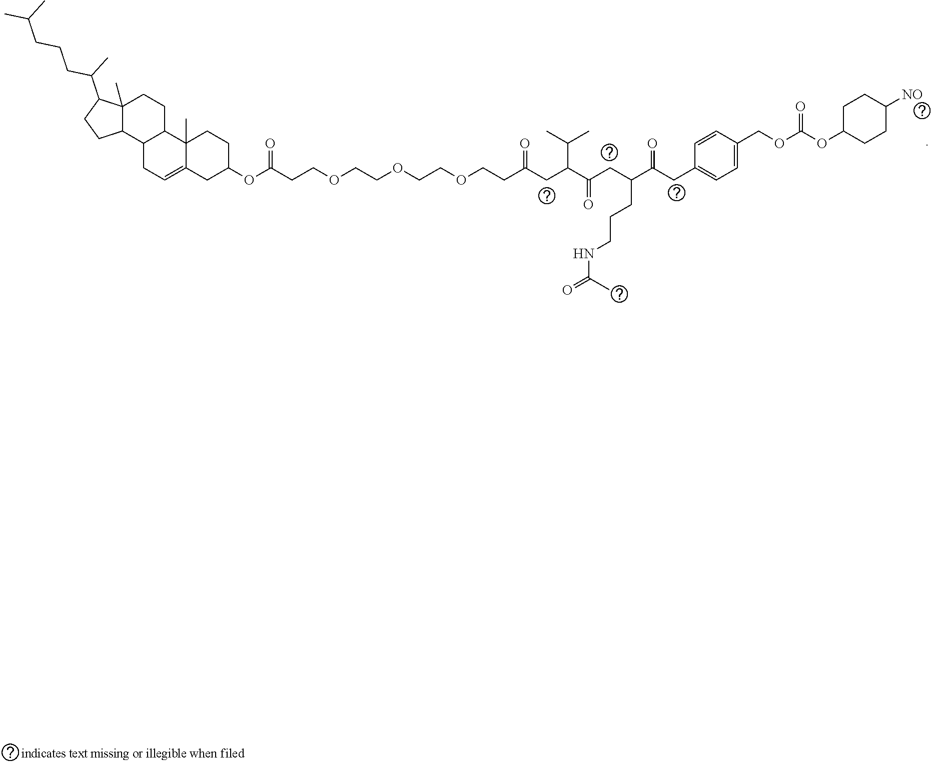

58. The extracellular vesicle of any one of claims 1 to 5, which comprises (i) an anchoring moiety selected from the group consisting of SEQ ID NOS: 301-334 and 401-567, and (ii) a linker selected from the linker combinations of TABLE 1 or TABLE 2.

59. The extracellular vesicle of any one of claims 1 to 5, wherein the anchoring moiety comprises a scaffold protein.

60. The extracellular vesicle of any one of claims 1 to 59, further comprising a scaffold moiety.

61. The extracellular vesicle of claim 59 or 60, wherein the anchoring moiety and/or the scaffold moiety is scaffold X.

62. The extracellular vesicle of claim 61, wherein the Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins); a functional fragment thereof, and any combination thereof.

63. The extracellular vesicle of claim 61, wherein the Scaffold X is PTGFRN protein or a functional fragment thereof.

64. The extracellular vesicle of claim 61, wherein the Scaffold X comprises an amino acid sequence as set forth in SEQ ID NO:302.

65. The extracellular vesicle of claim 61, wherein the Scaffold X comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO:302.

66. The extracellular vesicle of claim 59 or 60, wherein the anchoring moiety and/or the scaffold moiety is scaffold Y.

67. The extracellular vesicle of claim 66, wherein the Scaffold Y is a scaffold protein that is capable of anchoring the biologically active molecule on the luminal surface of the extracellular vesicle and/or on the exterior surface of the extracellular vesicle.

68. The extracellular vesicle of claim 66 or 67, wherein the Scaffold Y is selected from the group consisting of myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a functional fragment thereof, and any combination thereof.

69. The extracellular vesicle of any one of claims 66 to 68, wherein the Scaffold Y is a BASP1 protein or a functional fragment thereof.

70. The extracellular vesicle of any one of claims 66 to 69, wherein the Scaffold Y comprises an N terminus domain (ND) and an effector domain (ED), wherein the ND and/or the ED are associated with the luminal surface of the EV.

71. The extracellular vesicle of claim 70, wherein the ND is associated with the luminal surface of the exosome via myristoylation.

72. The extracellular vesicle of claim 70 or 71, wherein the ED is associated with the luminal surface of the exosome by an ionic interaction.

73. The extracellular vesicle of any one of claims 70 to 72, wherein the ED comprises (i) a basic amino acid or (ii) two or more basic amino acids in sequence, wherein the basic amino acid is selected from the group consisting of Lys, Arg, His, and any combination thereof.

74. The extracellular vesicle of claim 73, wherein the basic amino acid is (Lys)n, wherein n is an integer between 1 and 10.

75. The extracellular vesicle of any one of claims 70 to 74, wherein the ED comprises Lys (K), KK, KKK, KKKK (SEQ ID NO: 405), KKKKK (SEQ ID NO: 406), Arg (R), RR, RRR, RRRR (SEQ ID NO: 407); RRRRR (SEQ ID NO: 408), KR, RK, KKR, KRK, RKK, KRR, RRK, (K/R)(K/R)(K/R)(K/R) (SEQ ID NO: XX), (K/R)(K/R)(K/R)(K/R)(K/R) (SEQ ID NO: XX), or any combination thereof.

76. The extracellular vesicle of any one of claims 70 to 75, wherein the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

77. The extracellular vesicle of claim 76, wherein:
(i) the X2 is selected from the group consisting of Pro, Gly, Ala, and Ser;
(ii) the X4 is selected from the group consisting of Pro, Gly, Ala, Ser, Val, Ile, Leu, Phe, Trp, Tyr, Gln and Met;
(iii) the X5 is selected from the group consisting of Pro, Gly, Ala, and Ser;
(iv) the X6 is selected from the group consisting of Lys, Arg, and His; or
(v) any combination of (i)-(iv).

78. The extracellular vesicle of any one of claims 70 to 77, wherein the ND comprises the amino acid sequence of G:X2:X3:X4:X5:X6, wherein
(i) G represents Gly;
(ii) “:” represents a peptide bond;
(iii) the X2 is an amino acid selected from the group consisting of Pro, Gly, Ala, and Ser;
(iv) the X3 is an amino acid;
(v) the X4 is an amino acid selected from the group consisting of Pro, Gly, Ala, Ser, Val, Ile, Leu, Phe, Trp, Tyr, Gln and Met;
(vi) the X5 is an amino acid selected from the group consisting of Pro, Gly, Ala, and Ser; and
(vii) the X6 is an amino acid selected from the group consisting of Lys, Arg, and His.

79. The extracellular vesicle of any one of claims 76 to 78, wherein the X3 is selected from the group consisting of Asn, Gln, Ser, Thr, Asp, Glu, Lys, His, and Arg.

80. The extracellular vesicle of any one of claims 70 to 79, wherein the ND and the ED are joined by a linker.

81. The extracellular vesicle of claim 80, wherein the linker comprises one or more amino acids.

82. The method of any one of claims 70 to 81, wherein the ND comprises an amino acid sequence selected from the group consisting of (i) GGKLSKK (SEQ ID NO: 411), (ii) GAKLSKK (SEQ ID NO: 412), (iii) GGKQSKK (SEQ ID NO: 413), (iv) GGKLAKK (SEQ ID NO: 414), (v) GGKLSK (SEQ ID NO: 415), or (vi) any combination thereof.

83. The extracellular vesicle of claim 82, wherein the ND comprises an amino acid sequence selected from the group consisting of (i) GGKLSKKK (SEQ ID NO: 438), (ii) GGKLSKKS (SEQ ID NO: 439), (iii) GAKLSKKK (SEQ ID NO: 440), (iv) GAKLSKKS (SEQ ID NO: 441), (v) GGKQSKKK (SEQ ID NO: 442), (vi) GGKQSKKS (SEQ ID NO: 443), (vii) GGKLAKKK (SEQ ID NO: 444), (viii) GGKLAKKS (SEQ ID NO: 445), and (ix) any combination thereof.

84. The extracellular vesicle of any one of claims 70 to 83, wherein the ND comprises the amino acid sequence GGKLSKK (SEQ ID NO: 411).

85. The extracellular vesicle of any one of claims 70 to 84, wherein the Scaffold Y is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

86. The extracellular vesicle of any one of claims 70 to 85, wherein the Scaffold Y comprises (i) GGKLSKKKKGYNVN (SEQ ID NO: 446), (ii) GAKLSKKKKGYNVN (SEQ ID NO: 447), (iii) GGKQSKKKKGYNVN (SEQ ID NO: 448), (iv) GGKLAKKKKGYNVN (SEQ ID NO: 449), (v) GGKLSKKKKGYSGG (SEQ ID NO: 450), (vi) GGKLSKKKKGSGGS (SEQ ID NO: 451), (vii) GGKLSKKKKSGGSG (SEQ ID NO: 452), (viii) GGKLSKKKSGGSGG (SEQ ID NO: 453), (ix) GGKLSKKSGGSGGS (SEQ ID NO: 454), (x) GGKLSKSGGSGGSV (SEQ ID NO: 455), or (xi) GAKKSKKRFSFKKS (SEQ ID NO: 456).

87. The extracellular vesicle of any one of claims 70 to 85, wherein the Scaffold Y consists of (i) GGKLSKKKKGYNVN (SEQ ID NO: 446), (ii) GAKLSKKKKGYNVN (SEQ ID NO: 447), (iii) GGKQSKKKKGYNVN (SEQ ID NO: 448), (iv) GGKLAKKKKGYNVN (SEQ ID NO: 449), (v) GGKLSKKKKGYSGG (SEQ ID NO: 450), (vi) GGKLSKKKKGSGGS (SEQ ID NO: 451), (vii) GGKLSKKKKSGGSG (SEQ ID NO: 452), (viii) GGKLSKKKSGGSGG (SEQ ID NO: 453), (ix) GGKLSKKSGGSGGS (SEQ ID NO: 454), (x) GGKLSKSGGSGGSV (SEQ ID NO: 455), or (xi) GAKKSKKRFSFKKS (SEQ ID NO: 456).

88. The extracellular vesicle of any one of claim 70 to 87, wherein the Scaffold Y does not comprise Met at the N terminus.

89. The extracellular vesicle of any one of claims 70 to 88, wherein the Scaffold Y comprises a myristoylated amino acid residue at the N terminus of the scaffold protein.

90. The extracellular vesicle of claim 89, wherein the amino acid residue at the N terminus of the Scaffold Y is Gly.

91. The extracellular vesicle of any one of claims 1 to 90, wherein the biologically active molecule is linked to the anchoring moiety and/or the scaffold moiety on the exterior surface of the EV.

92. The extracellular vesicle of any one of claims 1 to 90, wherein the biologically active molecule is linked to the anchoring moiety and/or the scaffold moiety on the luminal surface of the EV.

93. The extracellular vesicle of any one of claims 1 to 92, wherein the biologically active molecule is a polypeptide, a peptide, a polynucleotide (DNA and/or RNA), a chemical compound, or any combination thereof.

94. The extracellular vesicle of claim 93, wherein the biologically active molecule is a chemical compound.

95. The extracellular vesicle of claim 94, wherein the chemical compound is a small molecule.

96. The extracellular vesicle of any one of claims 1 to 95, wherein the biologically active molecule comprises an antisense oligonucleotide (ASO), a siRNA, a miRNA, a shRNA, a nucleic acid, or any combination thereof.

97. The extracellular vesicle of any one of claims 1 to 96, wherein the biologically active molecule comprises a peptide, a protein, an antibody or an antigen binding fragment thereof, or any combination thereof.

98. The extracellular vesicle of claim 97, wherein the antigen binding fragment thereof comprises scFv, (scFv)2, Fab, Fab′, F(ab′)2, F(ab1)2, Fv, dAb, and Fd fragment, diabodys, antibody-related polypeptide, or any fragment thereof.

99. The extracellular vesicle of claim 96, wherein the biologically active molecule comprises an ASO.

100. The extracellular vesicle of claim 99, wherein the ASO targets a transcript.

101. The extracellular vesicle of claim 100, wherein the transcript is a STAT6 transcript, a CEBP/β transcript, a STAT3 transcript, a KRAS transcript, a NRAS transcript, an NLPR3 transcript, a PMP22 transcript, or any combination thereof.

102. The extracellular vesicle of claim 101, wherein the STAT6 transcript comprises SEQ ID NO: 13.

103. The extracellular vesicle of claim 102, wherein the ASO comprises a sequence selected from the group consisting of SEQ ID NO: 601 to SEQ ID NO: 703.

104. The extracellular vesicle of claim 101, wherein the CEBP/β transcript comprises SEQ ID NO: 23.

105. The extracellular vesicle of claim 104, wherein the ASO comprises a sequence selected from the group consisting of SEQ ID NO: 704 to SEQ ID NO: 806.

106. The extracellular vesicle of claim 101, wherein the STAT3 transcript comprises SEQ ID NO: 43.

107. The extracellular vesicle of claim 106, wherein the ASO comprises a sequence selected from the group consisting of SEQ ID NO: 889 to SEQ ID NO: 988.

108. The extracellular vesicle of claim 101, wherein the NRAS transcript comprises SEQ ID NO: 53.

109. The extracellular vesicle of claim 108, wherein the ASO comprises a sequence selected from the group consisting of SEQ ID NO: 989 to SEQ ID NO: 1088.

110. The extracellular vesicle of claim 101, wherein the NLPR3 transcript comprises SEQ ID NO: 3.

111. The extracellular vesicle of claim 110, wherein the ASO comprises a sequence selected from the group consisting of SEQ ID NO: 101 to SEQ ID NO: 200.

112. The extracellular vesicle of claim 101, wherein the KRAS transcript is a KRAS mutant transcript.

113. The extracellular vesicle of claim 101, wherein the KRAS mutant is KRAS G12D.

114. The extracellular vesicle of claim 112, wherein the KRAS transcript comprises SEQ ID NO: 32.

115. The extracellular vesicle of claim 114, wherein the ASO comprises a sequence selected from the group consisting of SEQ ID NO: 807 to SEQ ID NO: 888.

116. The extracellular vesicle of claim 101, wherein the PMP22 transcript comprises SEQ ID NO: 58.

117. The extracellular vesicle of claim 116, wherein the ASO comprises a sequence selected from the group consisting of SEQ ID NOS: 62-95 and 201-270.

118. The extracellular vesicle of any one of claims 1 to 117, wherein the EV is an exosome.

119. A pharmaceutical composition comprising the extracellular vesicle of any one of claims 1 to 118 and a pharmaceutically acceptable carrier.

120. A method of conjugating a biologically active molecule to an EV, comprising linking an anchoring moiety to the EV.

121. A kit comprising the EV of any one of claim 1 to 118 and instructions for use.

122. A kit comprising reagents to conjugate a biologically active molecule to an EV, and instructions to conduct the conjugation, thereby making the EV of any one of claims 1 to 118.

123. A method of treating or preventing a disease or disorder in a subject in need thereof comprising administering the EV of any one of claims 1 to 118 to the subject.

124. The method of claim 121, wherein the disease or disorder is a cancer, an inflammatory disorder, a neurodegenerative disorder, a central nervous diseases, or a metabolic disease.

125. The method of claim 121 or 122, wherein the EV is administered intravenously, intraperitoneally, nasally, orally, intramuscularly, subcutaneously, parenterally, or intratumorally.
